(19) United States
(12) Patent Application Publication
(10) Pub. No.: US 2015/0239889 A1
(43) Pub. Date: Aug. 27, 2015

(54) PYRAZOLOPYRIMIDINE COMPOUND

(71) Applicant: MITSUBISHI TANABE PHARMA CORPORATION, Osaka-shi, Osaka (JP)

(72) Inventors: Tatsuo Nakajima, Osaka-shi (JP); Takashi Goi, Osaka-shi (JP); Atsushi Kawata, Osaka-shi (JP); Masakatsu Sugahara, Osaka-shi (JP); Shuhei Yamakoshi, Osaka-shi (JP)

(73) Assignee: MITSUBISHI TANABE PHARMA CORPORATION, Osaka-shi, Osaka (JP)

(21) Appl. No.: 14/422,909

(22) PCT Filed: Aug. 22, 2013
(86) PCT No.: PCT/JP2013/072461

§ 371 Date: Feb. 20, 2015

(30) Foreign Application Priority Data
Aug. 23, 2012 (JP) 2012-184320

(51) Int. Cl. C07D 487/04

(52) U.S. Cl. CPC C07D 487/04

(57) ABSTRACT

Provided is a pyrazolopyrimidine compound represented by formula (I) having an HIF-PHD inhibitory effect, or a pharmaceutically acceptable salt thereof.

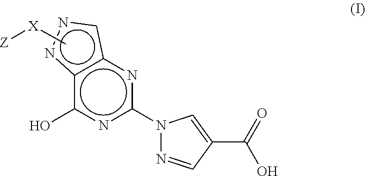

[In the formula,

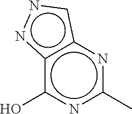

represents an optionally substituted 7-hydroxypyrazolo[4,3-d]pyrimidine-5-yl,
X represents a simple bond or an optionally substituted straight-chain alkylene,
Z represents hydrogen atom, or formula (i), formula (ii) or formula (iii)

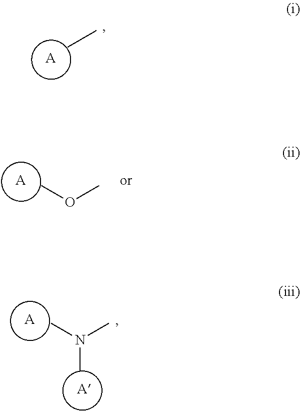

and
rings A and A′ are independently an optionally substituted aryl, an optionally substituted heteroaryl, an optionally substituted alicyclic hydrocarbon, or an optionally substituted non-aromatic heterocycle.]

### TECHNICAL FIELD

The present invention relates to a novel pyrazolopyrimidine compound useful as a medicament having an excellent HIF-PHD inhibitory effect.

### BACKGROUND ART

Anemia refers to a state where red blood cells and hemoglobin in blood is low and often presents with symptoms, such as fatigue, shortness of breath, palpitations, dizziness, facial pallor. Causes of anemia may be classified as decreased production of red blood cells (ineffective hematopoiesis wherein hematopoietic cells do not make enough normal red blood cells, reduction of hematopoietic cells, reduction of hematopoietic factors (such as erythropoietin)); increased destruction (hemolysis); and increased blood loss (bleeding).

Erythropoietin (EPO) is a hematopoietic factor that is secreted from the kidneys and promotes red blood cell production by acting on erythroid stem cells in the bone marrow. In a patient having lowered renal function, such as chronic renal failure, decreased EPO production in the kidney was known to cause anemia (renal anemia) due to reduced production of red blood cells.

As a treatment for renal anemia, supplemental therapy of recombinant human EPO has provided great contribution in the improvement of QOL (Quality of life) with improvement of symptoms associated with anemia and avoidance of routine blood transfusion. However, there are problems as pointed out that recombinant human EPO is a biological agent and involves expensive medical care, that it is not convenient because of its dosage form as an injectable formulation, and that it has antigenicity.

As typical factors that promote transcription of the EPO, hypoxia inducible factor (HIF) was known. HIF is a major factor involved in the gene expression induced by low oxygen concentration and is a heterodimer consisting of α and β subunits. Under normal oxygen concentration, HIF is ubiquitinated wherein proline in a subunit is hydroxylated by hypoxia-inducible factor-prolyl hydroxylase (HIF-PHD) and combined to von Hippel-Lindau (VHL) protein. On the other hand, under low oxygen concentration, HIF does not undergo hydroxylation by HIF-PHD, and thus, not ubiquitinated, but binds to hypoxia response element (HRE) in the nucleus to promote transcription of the EPO gene located at the downstream.

There are three isoforms of HIF-PHD, i.e., HIF-PHD1, HIF-PHD2, HIF-PHD3. Under normal oxygen concentration, HIF-PHD2 plays in the proline hydroxylation of HIF. HIF-PHD1 and HIF-PHD3 are also involved in the proline hydroxylation of HIF in certain types of cell and tissue. Thus, it is possible to increase the production of EPO by the inhibition of proline hydroxylation activity of HIF-PHD to stabilize HIF with preventing its ubiquitination. Therefore, inhibitors of HIF-PHD are promising as a medicament for the treatment of anemia.

Other diseases that are expected for improvement thereof, by stabilizing HIF and inhibiting HIF-PHD, include ischemic heart diseases (angina, myocardial infarction, etc.), ischemic cerebrovascular disorders (cerebral infarction, cerebral embolism, transient ischemic attack, etc.), chronic renal failure (ischemic nephropathy, tubulointerstitial disorders, etc.), diabetes complications (diabetic wounds, etc.), cognitive impairment (dementia, Alzheimer's disease, Parkinson's disease, Huntington's disease, etc.) and the like.

WO2010/093727 (Patent Document 1) discloses that a compound of the following structure has a prolyl hydroxylase inhibiting activity, and therefore, may be used for the treatment of diseases by activation of prolyl hydroxylase activity.

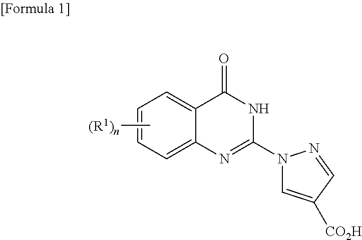

### PRIOR ART DOCUMENTS

### Patent Literature

Patent literature 1: WO2010/093727

### Non-Patent Literature

Non-patent literature 1:
Experimental Cell Research, Volume 318, pp. 1068-1073 (2012)
Non-patent literature 2:
The EMBO Journal, volume 22, pp. 4082-4090 (2003)
Non-patent literature 3:
Molecular Cell, Volume 30, pp. 393-402 (2008)

### SUMMARY OF THE INVENTION

### Problems to be Solved by the Invention

The present invention provides a novel pyrazolopyrimidine compound having hypoxia-inducible factor-prolyl hydroxylase (hereinafter also referred to as HIF-PHD) inviting effect, a method for the production thereof, a use thereof, as well as a pharmaceutical composition comprising said compound.

### Means for Solving the Problems

The present invention relates to a compound represented by the formula (I):

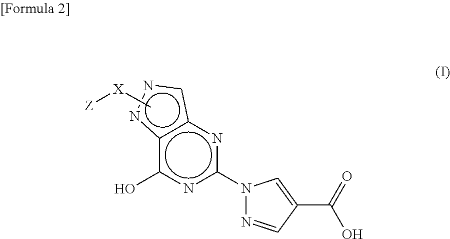

wherein,

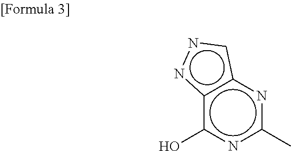

represents an optionally substituted 7-nydroxypyrazolo[4,3-d]pyrimidin-5-yl;
X represents a single bond or an optionally substituted straight chain alkylene;
Z represents hydrogen atom or the formula (i), (ii) or (iii):

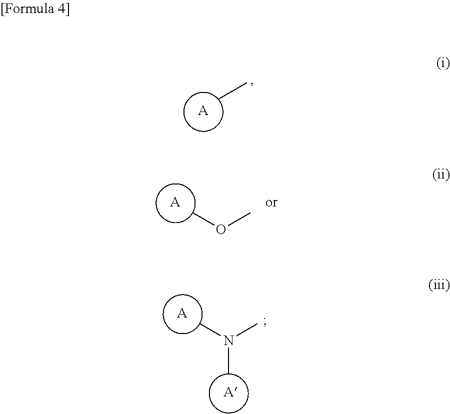

ring A and ring A′ are each independently an optionally substituted aryl, an optionally substituted heteroaryl, an optionally substituted alicyclic hydrocarbon or an optionally substituted non-aromatic heterocycle,
   or a pharmaceutically acceptable salt thereof.

The present invention also relates to a method for the treatment or prevention of diseases associated with HIF-PHD (e.g., renal anemia) which comprises administering to a patient a therapeutically effective amount of a compound of the formula (I) (hereinafter also referred to as compound (I)) or a pharmaceutically acceptable salt thereof.

The present invention also relates to a pharmaceutical composition comprising the compound (I) or a pharmaceutically acceptable salt thereof as an active ingredient and to use for the production thereof.

Furthermore, the present invention relates to a process for the production of the compound (I) or a pharmaceutically acceptable salt thereof.

### Effect of the Invention

The compound of the formula (I) or a pharmaceutically acceptable salt thereof, as well as a pharmaceutical composition containing the same as an active ingredient, exhibits excellent HIF-PHD inhibition, and therefore, is useful for the treatment and the prevention of diseases associated with HIF-PHD, such as renal anemia.

### MODE FOR CARRYING OUT THE INVENTION

Definitions of the terms as used herein are as follows.

The term “alkyl” means a straight or branched saturated hydrocarbon chain having 1 to 6 carbon atoms (C₁-C₆) and includes methyl, ethyl, propyl, isopropyl, butyl, t-butyl, isobutyl, and branched-chain isomers thereof.

The term “alkenyl” means a straight or branched unsaturated hydrocarbon chain having 2 to 6 carbon atoms (C₂-C₆) having one carbon-carbon double bond and includes vinyl, propenyl, isopropenyl, butenyl, and branched-chain isomers thereof.

The term “alkylene” means a straight or branched divalent saturated hydrocarbon chain having 1 to 6 carbon atoms (C₁-C₆) and includes methylene, ethylene, propylene, trimethylene, butylene, tetramethylene, pentamethylene, 1,1,2,2-tetramethyl ethylene, and branched-chain isomers thereof.

The term “straight chain alkylene” means a straight saturated divalent hydrocarbon chain having 1 to 6 carbon atoms (C₁-C₆) and includes methylene, ethylene, trimethylene, tetramethylene, and pentamethylene.

The term “cycloalkyl” means a monocyclic alicyclic hydrocarbon group having 3 to 8 carbons (C₃-C₈) in the ring and includes cyclopropyl, cyclobutyl, cyclopentyl, cyclohexyl, cycloheptyl, and cyclooctyl.

The term “cycloalkenyl” means a monocyclic alicyclic unsaturated hydrocarbon group having 3 to 8 carbon atoms (C₃-C₈) as well as one carbon-carbon double bond in the ring and includes cyclopropenyl, cyclobutenyl, cyclopentenyl, cyclohexenyl, cycloheptenyl, and cyclooctenyl.

The term “alicyclic hydrocarbon” means a monocyclic, bicyclic or tricyclic alicyclic hydrocarbon having 3 to 14 carbon atoms (C₃-C₁₄) in the ring and includes cycloalkyl of 3 to 8 carbon atoms (C₃-C₁₄) such as cyclopropyl, cyclobutyl, cyclopentyl, cyclohexyl, cycloheptyl, cyclooctyl; bicycloalkyl of 8 to 12 carbon atoms (C₈-C₁₂) such as bicyclooctyl, bicyclononyl, bicyclodecyl; bicyclic alicyclic hydrocarbon group of 8 to 12 carbon atoms such as spiroalkyl of 8 to 12 carbon atoms (C₈-C₁₂) (spirooctyl, supirononyl, supirodecyl, supiroundecyl, etc.); tricyclic alicyclic hydrocarbon of 10 to 14 carbon atoms (C₁₀-C₁₄) such as adamantyl.

The terms “halogen” and “halogeno”, respectively, mean fluorine atom, chlorine atom, bromine atom or iodine atom.

The term “alkoxy” means a group in which an oxygen atom is connected to a straight or branched alkyl of 1 to 6 carbon atoms (C₁-C₆) and includes methoxy, ethoxy, propoxy, isopropoxy, butoxy, t-butoxy, isobutoxy, and branched-chain isomers thereof.

The term “halogenoalkyl” and “halogenoalkoxy”, respectively, mean alkyl and alkoxy substituted with 1 to 7 halogen atoms.

The term “fluoroalkyl” and “fluoroalkoxy”, respectively, mean alkyl and alkoxy substituted with 1 to 7 fluorine atoms.

The term “aryl” refers to a monocyclic or a bicyclic aromatic hydrocarbon group having 6 to 11 carbon atoms (C₆-C₁₁) in the ring and includes monocyclic aryls such as phenyl; bicyclic aryls optionally partially saturated having 9 to 11 carbon atoms (C₉-C₁₁) in the ring and includes naphthyl, tetrahydronaphthyl, indenyl, indanyl.

The term “heteroaryl” means a 5 to 11-membered monocyclic or bicyclic aromatic heterocyclic group containing 1 to 4 hetero atoms selected from oxygen, sulfur or nitrogen atom, in addition to carbon atoms and includes 5 to 6-membered monocyclic heteroaryl containing 1 to 4 hetero atoms selected from oxygen, sulfur or nitrogen atom, in addition to carbon atoms, such as pyrrolyl, furyl, thienyl, pyrazolyl, imidazolyl, oxazolyl, isoxazolyl, thiazolyl, isothiazolyl, thiadiazolyl, pyridyl, pyrimidinyl, pyridazinyl; and 8 to 11-membered bicyclic heteroaryl containing 1 to 4 hetero atoms selected from oxygen, sulfur or nitrogen atom, in addition to carbon atoms, such as indolyl, indolinyl, isoindolinyl, indazolyl, benzofuranyl, dihydrobenzofuranyl, dihydroisobenzofuranyl, benzothiophenyl, dihydrobenzothiophenyl, dihydroisobenzothiophenyl, benzoxazolyl, dihydrobenzoxazolyl, benzothiazolyl, dihydrobenzothiazolyl, quinolyl, tetrahydroquinolyl, isoquinolyl, tetrahydroisoquinolyl, naphthyridinyl, tetrahydronaphthyridinyl, quinoxalinyl, tetrahydroquinoxalinyl, quinazolinyl.

The term “non-aromatic heterocycle” means a 4 to 7-membered monocyclic non-aromatic heterocyclic group containing 1 to 4 hetero atoms selected from oxygen, sulfur or nitrogen atoms, in addition to carbon atoms and includes pyrrolidinyl, piperidinyl, tetrahydrofuryl, tetrahydropyranyl, tetrahydrothienyl, morpholinyl and the like.

The term “nitrogen-containing non-aromatic heterocycle” means a non-aromatic heterocycle as defined above containing at least one nitrogen atom and includes pyrrolidinyl, piperidinyl, morpholinyl and the like.

The term “aryloxy” means a group wherein oxygen atom is connected to the aryl as deified above and includes phenoxy, naphthyloxy, and tetrahydronaphthyloxy and the like.

The terms “halogenophenyl”, “halogenoaryloxy” and halogenophenoxy” mean, respectively, phenyl, aryloxy and phenoxy as defined above substituted with 1, 2 or 3 halogen atoms.

Detail definitions for each symbols in formula (I) are as follows.

In the formula, “optionally substituted 7-hydroxypyrazolo[4,3-d]pyrimidin-5-yl” represented by

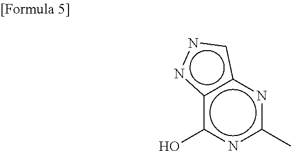

may be substituted with one substituent group. For such substituent group, alkyl, cycloalkyl alkyl, fluoroalkyl, cycloalkyl, halogen or cyano is preferable, and alkyl, cycloalkyl or halogen is especially preferable.

For straight chain alkylene in “optionally substituted straight chain alkylene” represented by X, C₁-C₆ straight chain alkylene is preferable, and methylene, ethylene or trimethylene is more preferable, and methylene is especially preferable.

For the single bond or straight chain alkylene in “single bond or an optionally substituted straight chain alkylene” represented by X, a single bond or C₁-C₆ straight chain alkylene is preferable, and a single bond, methylene, ethylene or trimethylene is more preferable, and a single bond or methylene is especially preferable.

The number of the substituent group for “optionally substituted straight chain alkylene” represented by X may be one or more (e.g., 1, 2 or 3). For such substituent group, alkyl, halogenoalkyl, cycloalkyl, halogen or an optionally substituted phenyl, such as phenyl optionally substituted with 1, 2 or 3 halogens, is preferable, and alkyl is especially preferable.

Z is preferably represented by the formula (i), (ii) or (iii), and the formula (i) is especially preferable.

Specific examples for aryl in “optionally substituted aryl” represented by ring A or ring A′ include phenyl, naphthyl, tetrahydronaphthyl, and indanyl; and phenyl or naphthyl is more preferable, and phenyl is especially preferable.

Specific examples for heteroaryl in “optionally substituted heteroaryl” represented by ring A or ring A′ include thienyl, pyridyl, indolyl, and quinolyl; and thienyl or pyridyl is more preferable.

Specific examples for alicyclic hydrocarbon in “optionally substituted alicyclic hydrocarbon” represented by ring A or ring A′ include cyclopropyl, cyclopentyl, cyclohexyl, cycloheptyl, cyclooctyl, spiro[5.2]octyl, spiro[5.3]nonyl, and adamantyl; and monocyclic or bicyclic alicyclic hydrocarbon, such as cyclohexyl, cycloheptyl, cyclooctyl or spiro[5.2]octyl, is more preferable.

Specific examples for non-aromatic heterocycle in “optionally substituted non-aromatic heterocycle” represented by ring A or ring A′ include pyrrolidinyl, piperidinyl, and tetrahydropyranyl; and pyrrolidinyl or piperidinyl is more preferable.

Preferred examples for aryl, heteroaryl, alicyclic hydrocarbon and non-aromatic heterocycle in “optionally substituted aryl, an optionally substituted heteroaryl, an optionally substituted alicyclic hydrocarbon or an optionally substituted non-aromatic heterocycle” represented by ring A or ring A′ include monocyclic or bicyclic aryl, monocyclic or bicyclic heteroaryl, monocyclic, bicyclic or tricyclic alicyclic hydrocarbon, and monocyclic non-aromatic heterocycle. Specific examples include phenyl, naphthyl, tetrahydronaphthyl, indanyl, thienyl, pyridyl, indolyl, quinolyl, cyclopropyl, cyclopentyl, cyclohexyl, cycloheptyl, cyclooctyl, spiro[5.2]octyl, spiro[5.3]nonyl, adamantyl, pyrrolidinyl, piperidinyl and tetrahydropyranyl, more preferably, monocyclic aryl or monocyclic or bicyclic alicyclic hydrocarbon, particularly, phenyl, cyclohexyl, cycloheptyl, cyclooctyl or spiro[5.2]octyl.

The number of the substituent group for “optionally substituted aryl”, “optionally substituted heteroaryl”, “optionally substituted alicyclic hydrocarbon” and “optionally substituted non-aromatic heterocycle” represented by ring A or ring A′ may be one or more, for example, 1 to 5, preferably, 1, 2 or 3. Preferred Examples for such substituents include an optionally substituted alkyl, such as alkyl optionally substituted with 1 to 7 (preferably, 1, 2 or 3) substituent groups selected from halogen, halogenoaryl (preferably, halogenophenyl), aryloxy (preferably, tetrahydronaphthyloxy) or halogenoaryloxy (preferably, halogenophenylphenoxy); an optionally substituted alkenyl, such as alkenyl optionally substituted with 1 to 7 (preferably, 1, 2 or 3) halogen; an optionally substituted cycloalkyl, such as cycloalkyl optionally substituted with 1 to 7 (preferably, 1, 2 or 3) substituent groups selected from alkyl, halogenoalkyl or halogen; an optionally substituted alkoxy, such as alkoxy optionally substituted with 1 to 7 (preferably, 1, 2 or 3) substituent groups selected from aryl (preferably, phenyl) or halogen; halogen; cyano; an optionally substituted phenyl, such as phenyl optionally substituted with 1, 2 or 3 substituent groups selected from alkyl, halogenoalkyl, cycloalkyl, alkoxy, halogenoalkoxy, cyano or halogen; an optionally substituted phenoxy, such as phenoxy optionally substituted with 1, 2 or 3 substituent groups selected from alkyl, halogenoalkyl or halogen; an optionally substituted heteroaryl, such as heteroaryl (preferably, pyridyl) optionally substituted with 1, 2 or 3 substituent groups selected from alkyl, halogenoalkyl or halogen; and an optionally substituted non-aromatic heterocycle, such as non-aromatic heterocycle (preferably, pyrrolidinyl or piperidinyl) optionally substituted with 1 to 5 substituent groups selected from alkyl, halogenoalkyl or oxo.

In a preferred embodiment of the invention, X is connected to “optionally substituted 7-hydroxypyrazolo[4,3-d]pyrimidin-5-yl” at the 1-position or 2-position.

In another preferred embodiment of the invention, the compound of the present invention is represented by the following formula (I-A)

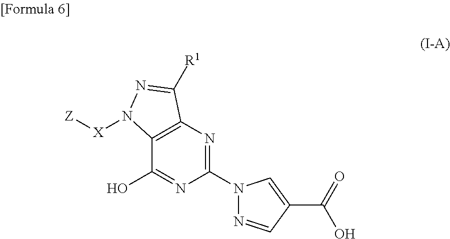

wherein R¹ is hydrogen atom, alkyl, fluoroalkyl, cycloalkyl, halogen or cyano, and the other symbols are as defined above.

In another preferred embodiment of the invention, the compound of the present invention is represented by the following formula (I-B)

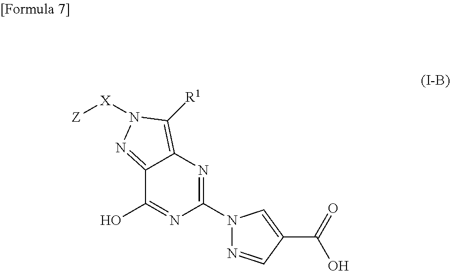

wherein the symbols are as defined above.

In another embodiment of the present invention, R¹ is preferably, hydrogen atom, alkyl (preferably, methyl, ethyl, isopropyl, and methyl is especially preferable), fluoroalkyl (preferably, trifluoromethyl), cycloalkyl (preferably, cyclopropyl) or halogen (preferably, fluorine atom, chlorine atom). Preferred is hydrogen atom, alkyl, cycloalkyl or halogen, and hydrogen atom is especially preferable.

Preferred Examples of the straight chain alkylene in “optionally substituted straight chain alkylene” represented by X include C₁-C₆ straight chain alkylene, and methylene, ethylene or trimethylene is more preferable, and methylene is especially preferable.

For the single bond or straight chain alkylene in “single bond or an optionally substituted straight chain alkylene” represented by X, a single bond or C₁-C₆ straight chain alkylene is preferable, and a single bond, methylene, ethylene or trimethylene is more preferable, and a single bond or methylene is especially preferable.

The number of the substituent group for “optionally substituted straight chain alkylene” represented by X may be one or more (e.g., 1, 2 or 3). For such substituent group, alkyl, halogenoalkyl, cycloalkyl, halogen or an optionally substituted phenyl, such as phenyl optionally substituted with 1, 2 or 3 halogens, is preferable, and alkyl is especially preferable.

Z is preferably represents the formula (i), (ii) or (iii), and the formula (i) is especially preferable.

Specific examples for aryl in “optionally substituted aryl” represented by ring A or ring A′ include phenyl, naphthyl, tetrahydronaphthyl, and indanyl; and phenyl or naphthyl is more preferable, and phenyl is especially preferable.

Specific examples for heteroaryl in “optionally substituted heteroaryl” represented by ring A or ring A′ include thienyl, pyridyl, indolyl, and quinolyl; and thienyl or pyridyl is more preferable.

Specific examples for alicyclic hydrocarbon in “optionally substituted alicyclic hydrocarbon” represented by ring A or ring A′ include cyclopropyl, cyclopentyl, cyclohexyl, cycloheptyl, cyclooctyl, spiro[5.2]octyl, spiro[5.3]nonyl, and adamantyl; and more preferably, monocyclic or bicyclic alicyclic hydrocarbon such as cyclohexyl, cycloheptyl, cyclooctyl or spiro[5.2]octyl.

Specific examples for non-aromatic heterocycle in “optionally substituted non-aromatic heterocycle” represented by ring A or ring A′ include pyrrolidinyl, piperidinyl, and tetrahydropyranyl; and pyrrolidinyl or piperidinyl is more preferable.

Preferred examples for aryl, heteroaryl, alicyclic hydrocarbon or non-aromatic heterocycle in “optionally substituted aryl, an optionally substituted heteroaryl, an optionally substituted alicyclic hydrocarbon or an optionally substituted non-aromatic heterocycle” represented by ring A or ring A′ include monocyclic or bicyclic aryl, monocyclic or bicyclic heteroaryl, monocyclic, bicyclic or tricyclic alicyclic hydrocarbon, and monocyclic non-aromatic heterocycle. Specific examples include phenyl, naphthyl, tetrahydronaphthyl, indanyl, thienyl, pyridyl, indolyl, quinolyl, cyclopropyl, cyclopentyl, cyclohexyl, cycloheptyl, cyclooctyl, spiro[5.2]octyl, spiro[5.3]nonyl, adamantyl, pyrrolidinyl, piperidinyl and tetrahydropyranyl, and monocyclic aryl or monocyclic or bicyclic alicyclic hydrocarbon is more preferable, and phenyl, cyclohexyl, cycloheptyl, cyclooctyl or spiro[5.2]octyl is specifically preferable.

The number of the substituent group for “optionally substituted aryl”, “optionally substituted heteroaryl”, “optionally substituted alicyclic hydrocarbon” and “optionally substituted non-aromatic heterocycle” represented by ring A or ring A′, each independently, may be one or more, for example, 1 to 5, and 1, 2 or 3 is preferable. Preferred Examples for such substituents include an optionally substituted alkyl, such as alkyl optionally substituted with 1 to 7 (preferably, 1, 2 or 3) substituent groups selected from halogen, halogenoaryl (preferably, halogenophenyl), aryloxy (preferably, tetrahydronaphthyloxy) or halogenoaryloxy (preferably, halogenophenylphenoxy); an optionally substituted alkenyl, such as alkenyl optionally substituted with 1 to 7 (preferably, 1, 2 or 3) halogen; an optionally substituted cycloalkyl, such as cycloalkyl optionally substituted with 1 to 7 (preferably, 1, 2 or 3) substituent groups selected from alkyl, halogenoalkyl or halogen; an optionally substituted alkoxy, such as alkoxy optionally substituted with 1 to 7 (preferably, 1, 2 or 3) substituent groups selected from aryl (preferably, phenyl) or halogen; halogen; cyano; an optionally substituted phenyl, such as phenyl optionally substituted with 1, 2 or 3 substituent groups selected from alkyl, halogenoalkyl, cycloalkyl, alkoxy, halogenoalkoxy, cyano or halogen; an optionally substituted phenoxy, such as phenoxy optionally substituted with 1, 2 or 3 substituent groups selected from alkyl, halogenoalkyl or halogen; an optionally substituted heteroaryl, such as heteroaryl (preferably, pyridyl) optionally substituted with 1, 2 or 3 substituent groups selected from alkyl, halogenoalkyl or halogen; and an optionally substituted non-aromatic heterocycle, such as non-aromatic heterocycle (preferably, pyrrolidinyl or piperidinyl) optionally substituted with 1 to 5 substituent groups selected from alkyl, halogenoalkyl or oxo.

In yet another embodiment of the invention, the compound of the present invention is preferably represented by the following formula (I-C):

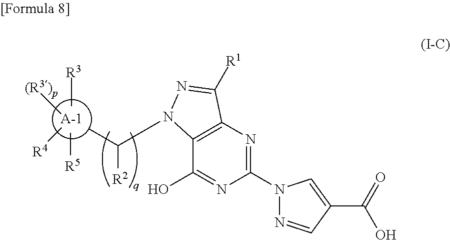

wherein
ring A-1 is aryl, heteroaryl, alicyclic hydrocarbon or non-aromatic heterocycle;
R² is hydrogen atom, alkyl, halogenoalkyl, cycloalkyl, phenyl or halogenophenyl;
R³, R^{3′}, and R⁴ are each independently hydrogen atom, an optionally substituted alkyl, an optionally substituted alkenyl, an optionally substituted cycloalkyl, an optionally substituted alkoxy, halogen or cyano;
R⁵ is hydrogen atom, an optionally substituted alkyl, an optionally substituted alkenyl, an optionally substituted cycloalkyl, an optionally substituted alkoxy, halogen, cyano, an optionally substituted phenyl, an optionally substituted phenoxy, an optionally substituted monocyclic heteroaryl or non-aromatic heterocycle;
p represents 0 or 1;
q represents 0 or 1; and
R¹ is as defined above.

In this embodiment, preferably,
ring A-1 is a monocyclic or bicyclic C₆-C₁₁ aryl (more preferably, phenyl, naphthyl, tetrahydronaphthyl or indanyl, especially preferably, phenyl or naphthyl); a monocyclic 5 to 6-membered heteroaryl containing 1 to 4 hetero atoms selected from oxygen, sulfur or nitrogen atom, in addition to carbon atoms (more preferably, thienyl or pyridyl); a monocyclic, bicyclic or tricyclic C₃-C₁₄ alicyclic hydrocarbon (more preferably, cyclopropyl, cyclopentyl, cyclohexyl, cycloheptyl, cyclooctyl, spiro[5.2]octyl, spiro[5.3]nonyl or adamantyl); or a monocyclic 4 to 7 membered non-aromatic heterocycle containing 1 to 4 hetero atoms selected from oxygen, sulfur or nitrogen atom, in addition to carbon atoms (more preferably, pyrrolidinyl or piperidinyl);
R¹ is hydrogen atom, C₁-C₆ alkyl (more preferably, methyl, ethyl or isopropyl, and methyl is especially preferable), C₁-C₆ fluoroalkyl (more preferably, trifluoromethyl), C₃-C₈ cycloalkyl (more preferably, cyclopropyl) or halogen (more preferably, fluorine atom or chlorine atom);
R² is hydrogen atom, C₁-C₆ alkyl (more preferably, methyl, ethyl or isopropyl, and methyl is especially preferable), C₃-C₈ cycloalkyl (more preferably, cyclopropyl) or halogenophenyl (more preferably, chlorophenyl);
R³, R^{3′} and R⁴ are each independently hydrogen atom; C₁-C₆ alkyl optionally substituted with 1 to 7 (preferably, 1, 2 or 3) substituent groups selected from halogen, halogenophenyl, tetrahydronaphthyloxy or halogenophenoxy; C₃-C₈ cycloalkyl; C₁-C₆ alkoxy optionally substituted with 1 to 7 (preferably, 1, 2 or 3) halogen; or halogen; and
R⁵ is hydrogen atom; C₁-C₆ alkyl optionally substituted with 1 to 7 (preferably, 1, 2 or 3) substituent groups selected from halogen, halogenophenyl, tetrahydronaphthyloxy or halogenophenoxy; C₃-C₈ cycloalkyl; C₁-C₆ alkoxy optionally substituted with 1 to 7 (preferably, 1, 2 or 3) halogen; halogen; phenyl optionally substituted with 1, 2 or 3 substituent groups selected from C₁-C₆ alkyl, C₁-C₆ halogencalkyl, C₃-C₈ cycloalkyl, C₁-C₆ alkoxy, C₁-C₆ halogenoalkoxy, cyano or halogen; phenoxy optionally substituted with 1, 2 or 3 substituent groups selected from C₁-C₆ alkyl, C₁-C₆ halogenoalkyl or halogen; 5 to 6-membered monocyclic heteroaryl containing 1 to 4 hetero atoms selected from oxygen, sulfur or nitrogen atom, in addition to carbon atoms, optionally substituted with 1, 2 or 3 substituent groups selected from C₁-C₆ alkyl, C₁-C₆ halogenoalkyl or halogen; or 4 to 7-membered monocyclic non-aromatic heterocycle containing 1 to 4 hetero atoms selected from oxygen, sulfur or nitrogen atoms, in addition to carbon atoms, (preferably, pyrrolidinyl or piperidinyl) optionally substituted with 1 to 5 (preferably, 1, 2 or 3) substituent groups selected from C₁-C₆ alkyl, C₁-C₆ halogenoalkyl or oxo.

In this embodiment, more preferably,
R³ and R⁴ are each independently hydrogen atom, C₁-C₆ alkyl, C₁-C₆ halogenoalkyl, C₃-C₈ cycloalkyl, C₁-C₆ alkoxy, C₁-C₆ halogenoalkoxy, or halogen;
R⁵ is hydrogen atom; C₁-C₆ alkyl; C₁-C₆ halogenoalkyl; C₃-C₈ cycloalkyl; C₁-C₆ alkoxy; C₁-C₆ halogenoalkoxy; halogen; phenyl optionally substituted with 1, 2 or 3 substituent groups selected from C₁-C₆ alkyl, C₁-C₆ halogenoalkyl, C₃-C₆ cycloalkyl, C₁-C₆ alkoxy, C₁-C₆ halogenoalkoxy, cyano or halogen; or pyridyl optionally substituted with 1, 2 or 3 substituent groups selected from C₁-C₆ alkyl, C₁-C₆ halogenoalkyl or halogen;
p is 0; and
q is 1.

In this embodiment, especially preferably, R¹ is hydrogen atom, and R² is hydrogen atom or alkyl.

In this embodiment, especially preferably, ring A-1 is phenyl, naphthyl or cycloalkyl (preferably, cyclohexyl, cycloheptyl, cyclooctyl), and R⁵ is hydrogen atom.

In another preferred embodiment of the present invention, ring A-1 is phenyl, naphthyl, tetrahydronaphthyl, indanyl, thienyl, pyridyl, cyclopropyl, cyclopentyl, cyclohexyl, cycloheptyl, cyclooctyl, spiro[5.2]octyl, spiro[5.3]nonyl, adamantyl, pyrrolidinyl or piperidinyl;
R¹ is hydrogen atom, C₁-C₆ alkyl (more preferably, methyl, ethyl or isopropyl, and methyl is especially preferable), C₁-C₆ fluoroalkyl (more preferably, trifluoromethyl), C₃-C₈ cycloalkyl (more preferably, cyclopropyl) or halogen (more preferably, fluorine or a chlorine atom);
R² is hydrogen atom, C₁-C₆ alkyl (more preferably, methyl, ethyl or isopropyl, especially preferably, methyl), C₃-C₈ cycloalkyl (more preferably, cyclopropyl) or halogenophenyl (more preferably, chlorophenyl);
R³, R^{3′} and R⁴ are each independently hydrogen atom; C₁-C₆ alkyl optionally substituted with 1 to 7 (preferably, 1, 2 or 3) substituent groups selected from halogen, halogenophenyl, tetrahydronaphthyloxy or halogenophenoxy; C₃-C₈ cycloalkyl; C₁-C₆ alkoxy optionally substituted with 1 to 7 (preferably, 1, 2 or 3) halogens; or halogen;
R⁵ is hydrogen atom; C₁-C₆ alkyl optionally substituted with 1 to 7 (preferably, 1, 2 or 3) substituent groups selected from halogen, halogenophenyl, tetrahydronaphthyloxy or halogenophenoxy; C₃-C₈ cycloalkyl; C₁-C₆ alkoxy optionally substituted with 1 to 7 (preferably, 1, 2 or 3) halogens; halogen; phenyl optionally substituted with 1, 2 or 3 substituent groups selected from C₁-C₆ alkyl, C₁-C₆ halogenoalkyl, C₃-C₈ cycloalkyl, C₁-C₆ alkoxy, C₁-C₆ halogenoalkoxy, cyano or halogen; phenoxy optionally substituted with 1, 2 or 3 substituent groups selected from C₁-C₆ alkyl, C₁-C₆ halogenoalkyl or halogen; pyridyl optionally substituted with 1, 2 or 3 substituent groups selected from C₁-C₆ alkyl, C₁-C₆ halogenoalkyl or halogen; or non-aromatic heterocycle selected from pyrrolidinyl or piperidinyl optionally substituted with 1 to 5 (preferably, 1, 2 or 3) substituent groups selected from C₁-C₆ alkyl, C₁-C₆ halogenoalkyl or oxo;
p is 0 or 1; and
q is 0 or 1.

In this embodiment, more preferably,
ring A-1 is phenyl, naphthyl, cyclohexyl, cycloheptyl, cyclooctyl or spiro[5.2]octyl;
R₁ is hydrogen atom;

R² is hydrogen atom or C₁-C₆ alkyl (more preferably, methyl or ethyl, and methyl is especially preferable);
R³, R^{3′} and R⁴ are each independently hydrogen atom; C₁-C₆ alkyl optionally substituted with 1 to 7 (preferably, 1, 2 or 3) halogens; C₁-C₆ alkoxy optionally substituted with 1 to 7 (preferably, 1, 2 or 3) halogens; or halogen;
R⁵ is hydrogen atom; C₁-C₆ alkyl optionally substituted with 1 to 7 (preferably, 1, 2 or 3) halogens; C₁-C₆ alkoxy optionally substituted with 1 to 7 (preferably, 1, 2 or 3) halogens; halogen; or phenyl optionally substituted with 1, 2 or 3 halogens; and
p is 1.

In this embodiment, especially preferably,
ring A-1 is phenyl, cyclohexyl, cycloheptyl or cyclooctyl;
R³, R^{3′} and R⁴ are each independently hydrogen atom; C₁-C₆ alkyl optionally substituted with 1 to 7 (preferably, 1, 2 or 3) halogens; or halogen; and
R⁵ is hydrogen atom; C₁-C₆ alkyl optionally substituted with 1 to 7 (preferably, 1, 2 or 3) halogens; halogen; or phenyl optionally substituted with 1, 2 or 3 halogens.

In another preferred embodiment of the present invention,
ring A-1 is a monocyclic, bicyclic or tricyclic C₃-C₁₄ alicyclic hydrocarbon (more preferably, cyclopropyl, cyclopentyl, cyclohexyl, cycloheptyl, cyclooctyl, spiro[5.2]octyl, spiro[5.3]nonyl or adamantyl);
R¹ is hydrogen atom;
R² is hydrogen atom or a C₁-C₆ alkyl (more preferably, methyl);
R³, R^{3′} and R⁴ are each independently hydrogen atom, C₁-C₆ alkyl, C₁-C₆ halogenoalkyl or halogen;
R⁵ is hydrogen atom; C₁-C₆ alkyl; C₁-C₆ halogenoalkyl; halogen, phenyl optionally substituted with 1, 2 or 3 halogens; or phenoxy optionally substituted with 1, 2 or 3 substituent group selected from C₁-C₆ alkyl, C₁-C₆ halogenoalkyl or halogen;
p is 1; and
q is 0 or 1.

In this embodiment, more preferably, ring A-1 is an monocyclic or bicyclic C₃-C₁₂ alicyclic hydrocarbon (more preferably, cyclohexyl, cycloheptyl, cyclooctyl or spiro[5.2]octyl);
R¹ is hydrogen atom;
R² is hydrogen atom;
R³, R^{3′}, R⁴ and R⁵ are each independently hydrogen atom or C₁-C₆ alkyl;
p is 1; and
q is 0 or 1.

In another preferred embodiment of the invention, the compound of the present invention is represented by the following formula (I-D):

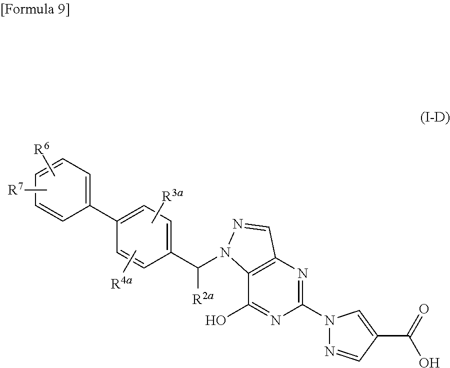

wherein
R^{2a} represents hydrogen atom or an alkyl, and
R^{3a}, R⁴, R⁶ and R⁷ are each independently hydrogen atom, alkyl, alkoxy or halogen.

In this embodiment, preferably, R^{2a} is hydrogen atom or methyl, R^{3a} and R⁶ are each independently hydrogen atom, methyl, methoxy or fluorine atom, and R^{4a} and R⁷ are each hydrogen atom.

In another preferred embodiment of the invention, the compound of the present invention is represented by the following formula (I-E):

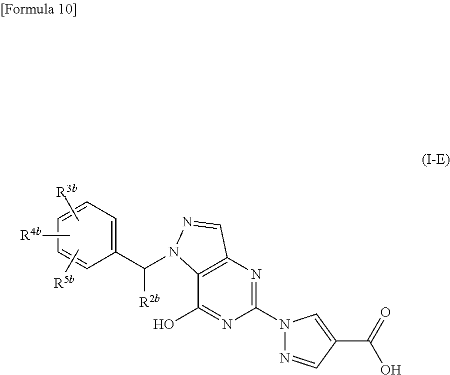

wherein
R^{2b} represents hydrogen atom, alkyl or cycloalkyl, and
R^{3b}, R^{4b}, and R^{5b} are each independently hydrogen atom, alkyl, halogenoalkyl, cycloalkyl, alkoxy, halogenoalkoxy or halogen.

In this embodiment, preferably,
R^{2b} is hydrogen atom, C₁-C₆ alkyl (preferably, methyl, ethyl or isopropyl, and methyl is especially preferable), or C₃-C₈ cycloalkyl (preferably, cyclopropyl); and
R^{3b}, R^{4b} and R^{5b} are each independently hydrogen atom, C₁-C₆ alkyl, C₁-C₆ halogenoalkyl (preferably, C₁-C₆ fluoroalkyl), C₃-C₈ cycloalkyl, C₁-C₆ alkoxy, C₁-C₆ halogenoalkoxy (preferably, C₁-C₆ fluoroalkoxy) or halogen.

Examples for compounds (I) or a pharmaceutically acceptable salt thereof of the present invention include the compounds as described in the Examples or pharmaceutically acceptable salts thereof, and preferred examples for such compound are as follows:
1-(7-hydroxy-1-{1-[4-(trifluoromethyl)phenyl]ethyl}-1H-pyrazolo[4,3-d]pyrimidin-5-yl)-1H-pyrazole-4-carboxylic acid;
1-{1-[1-(4-chlorophenyl)ethyl]-7-hydroxy-1H-pyrazolo[4,3-d]pyrimidin-5-yl}-1H-pyrazole-4-carboxylic acid;
1-{1-[1-(3,4-dichlorophenyl)ethyl]-7-hydroxy-1H-pyrazolo[4,3-d]pyrimidin-5-yl}-1H-pyrazole-4-carboxylic acid;
1-[7-hydroxy-1-(2-naphthylmethyl)-1H-pyrazolo[4,3-d]pyrimidin-5-yl]-1H-pyrazole-4-carboxylic acid;
1-{7-hydroxy-1-[1-(2-naphthyl)ethyl]-1H-pyrazolo[4,3-d]pyrimidin-5-yl}-1H-pyrazole-4-carboxylic acid;
1-[1-(biphenyl-4-ylmethyl)-7-hydroxy-1H-pyrazolo[4,3-d]pyrimidin-5-yl]-1H-pyrazole-4-carboxylic acid;
1-{1-[(2′-fluorobiphenyl-4-yl)methyl]-7-hydroxy-1H-pyrazolo[4,3-d]pyrimidin-5-yl}-1H-pyrazole-4-carboxylic acid;
1-{1-[(3-fluorobiphenyl-4-yl)methyl]-7-hydroxy-1H-pyrazolo[4,3-d]pyrimidin-5-yl}-1H-pyrazole-4-carboxylic acid;
1-{1-[(2-fluorobiphenyl-4-yl)methyl]-7-hydroxy-1H-pyrazolo[4,3-d]pyrimidin-5-yl}-1H-pyrazole-4-carboxylic acid;
1-{1-[(2,2′-difluorobiphenyl-4-yl)methyl]-7-hydroxy-1H-pyrazolo[4,3-d]pyrimidin-5-yl}-1H-pyrazole-4-carboxylic acid;
1-{1-[(2′-fluoro-2-methylbiphenyl-4-yl)methyl]-7-hydroxy-1H-pyrazolo[4,3-d]pyrimidin-5-yl}-1H-pyrazole-4-carboxylic acid;
1-{1-[(2′-fluoro-2-methoxybiphenyl-4-yl)methyl]-7-hydroxy-1H-pyrazolo[4,3-d]pyrimidin-5-yl}-1H-pyrazole-4-carboxylic acid;
1-{1-[1-biphenyl-4-ylethyl]-7-hydroxy-1H-pyrazolo[4,3-d]pyrimidin-5-yl}-1H-pyrazole-4-carboxylic acid;
1-[1-(cyclohexylmethyl)-7-hydroxy-1H-pyrazolo[4,3-d]pyrimidin-5-yl]-1H-pyrazole-4-carboxylic acid;
1-{7-hydroxy-1-[(trans-4-methylcyclohexyl)methyl]-1H-pyrazolo[4,3-d]pyrimidin-5-yl}-1H-pyrazole-4-carboxylic acid;
1-{7-hydroxy-1-[(trans-4-phenylcyclohexyl)methyl]-1H-pyrazolo[4,3-d]pyrimidin-5-yl}-1H-pyrazole-4-carboxylic acid;
1-(1-{[trans-4-(4-chlorophenyl)cyclohexyl]methyl}-7-hydroxy-1H-pyrazolo[4,3-d]pyrimidin-5-yl)-1H-pyrazole-4-carboxylic acid;
1-[1-(cycloheptylmethyl)-7-hydroxy-1H-pyrazolo[4,3-d]pyrimidin-5-yl]-1H-pyrazole-4-carboxylic acid;
1-[1-(cyclooctylmethyl)-7-hydroxy-1H-pyrazolo[4,3-d]pyrimidin-5-yl]-1H-pyrazole-4-carboxylic acid;
1-{1-[1-(2,5-dichlorophenyl)ethyl]-7-hydroxy-1H-pyrazolo[4,3-d]pyrimidin-5-yl}-1H-pyrazole-4-carboxylic acid;
1-(1-{1-[3-fluoro-4-(trifluoromethyl)phenyl]ethyl}-7-hydroxy-1H-pyrazolo[4,3-d]pyrimidin-5-yl)-1H-pyrazole-4-carboxylic acid;
1-{1-[1-(2,5-dimethylphenyl)ethyl]-7-hydroxy-1H-pyrazolo[4,3-d]pyrimidin-5-yl}-1H-pyrazole-4-carboxylic acid;
1-{1-[1-(5-fluoro-2-methylphenyl)ethyl]-7-hydroxy-1H-pyrazolo[4,3-d]pyrimidin-5-yl}-1H-pyrazole-4-carboxylic acid;
1-{1-[1-(3-fluoro-5-methylphenyl)ethyl]-7-hydroxy-1H-pyrazolo[4,3-d]pyrimidin-5-yl}-1H-pyrazole-4-carboxylic acid;
1-{1-[1-(4-fluoro-3-methylphenyl)ethyl]-7-hydroxy-1H-pyrazolo[4,3-d]pyrimidin-5-yl}-1H-pyrazole-4-carboxylic acid;
1-{1-[1-(3,5-dimethylphenyl)ethyl]-7-hydroxy-1H-pyrazolo[4,3-d]pyrimidin-5-yl}-1H-pyrazole-4-carboxylic acid;
1-(1-{1-[4-chloro-3-(trifluoromethyl)phenyl]ethyl}-7-hydroxy-1H-pyrazolo[4,3-d]pyrimidin-5-yl)-1H-pyrazole-4-carboxylic acid;
1-(1-{1-[3-fluoro-4-(trifluoromethoxy)phenyl]ethyl}-7-hydroxy-1H-pyrazolo[4,3-d]pyrimidin-5-yl)-1H-pyrazole-4-carboxylic acid;
1-(1-{1-[3-chloro-4-(trifluoromethoxy)phenyl]ethyl}-7-hydroxy-1H-pyrazolo[4,3-d]pyrimidin-5-yl)-1H-pyrazole-4-carboxylic acid;
1-[7-hydroxy-1-(3,3,5,5-tetramethylcyclohexyl)-1H-pyrazolo[4,3-d]pyrimidin-5-yl]-1H-pyrazole-4-carboxylic acid;
1-(1-cycloheptyl-7-hydroxy-1H-pyrazolo[4,3-d]pyrimidin-5-yl)-1H-pyrazole-4-carboxylic acid;
1-(1-cyclooctyl-7-hydroxy-1H-pyrazolo[4,3-d]pyrimidin-5-yl)-1H-pyrazole-4-carboxylic acid;
1-{7-hydroxy-1-[trans-3-methylcyclohexyl]-1H-pyrazolo[4,3-d]pyrimidin-5-yl}-1H-pyrazole-4-carboxylic acid;
1-{7-hydroxy-1-[cis-3-methylcyclohexyl]-1H-pyrazolo[4,3-d]pyrimidin-5-yl}-1H-pyrazole-4-carboxylic acid;
1-{r-1-[t-3,t-5-dimethylcyclohexyl]-7-hydroxy-1H-pyrazolo[4,3-d]pyrimidin-5-yl}-1H-pyrazole-4-carboxylic acid; and
1-{7-hydroxy-1-[cis-3,3,5-trimethylcyclohexyl]-1H-pyrazolo[4,3-d]pyrimidin-5-yl}-1H-pyrazole-4-carboxylic acid;
   or pharmaceutically acceptable salts thereof.

More preferably, examples for the compound are as follows:
1-{1-[1-(3,4-dichlorophenyl)ethyl]-7-hydroxy-1H-pyrazolo[4,3-d]pyrimidin-5-yl}-1H-pyrazole-4-carboxylic acid;
1-[1-(biphenyl-4-ylmethyl)-7-hydroxy-1H-pyrazolo[4,3-d]pyrimidin-5-yl]-1H-pyrazole-4-carboxylic acid;
1-{1-[(2,2′-difluorobiphenyl-4-yl)methyl]-7-hydroxy-1H-pyrazolo[4,3-d]pyrimidin-5-yl}-1H-pyrazole-4-carboxylic acid;
1-[1-(cycloheptylmethyl)-7-hydroxy-1H-pyrazolo[4,3-d]pyrimidin-5-yl]-1H-pyrazole-4-carboxylic acid;
1-{1-[1-(2,5-dichlorophenyl)ethyl]-7-hydroxy-1H-pyrazolo[4,3-d]pyrimidin-5-yl}-1H-pyrazole-4-carboxylic acid;
1-(1-{1-[3-fluoro-4-(trifluoromethyl)phenyl]ethyl}-7-hydroxy-1H-pyrazolo[4,3-d]pyrimidin-5-yl)-1H-pyrazole-4-carboxylic acid;
1-{1-[1-(2,5-dimethylphenyl)ethyl]-7-hydroxy-1H-pyrazolo[4,3-d]pyrimidin-5-yl}-1H-pyrazole-4-carboxylic acid;
1-{1-[1-(5-fluoro-2-methylphenyl)ethyl]-7-hydroxy-1H-pyrazolo[4,3-d]pyrimidin-5-yl}-1H-pyrazole-4-carboxylic acid;
1-{1-[1-(3-fluoro-5-methylphenyl)ethyl]-7-hydroxy-1H-pyrazolo[4,3-d]pyrimidin-5-yl}-1H-pyrazole-4-carboxylic acid;
1-{1-[1-(4-fluoro-3-methylphenyl)ethyl]-7-hydroxy-1H-pyrazolo[4,3-d]pyrimidin-5-yl}-1H-pyrazole-4-carboxylic acid;
1-{1-[1-(3,5-dimethylphenyl)ethyl]-7-hydroxy-1H-pyrazolo[4,3-d]pyrimidin-5-yl}-1H-pyrazole-4-carboxylic acid;
1-(1-{1-[4-chloro-3-(trifluoromethyl)phenyl]ethyl}-7-hydroxy-1H-pyrazolo[4,3-d]pyrimidin-5-yl)-1H-pyrazole-4-carboxylic acid;
1-(1-{1-[3-fluoro-4-(trifluoromethoxy)phenyl]ethyl}-7-hydroxy-1H-pyrazolo[4,3-d]pyrimidin-5-yl)-1H-pyrazole-4-carboxylic acid;
1-(1-{1-[3-chloro-4-(trifluoromethoxy)phenyl]ethyl}-7-hydroxy-1H-pyrazolo[4,3-d]pyrimidin-5-yl)-1H-pyrazole-4-carboxylic acid;
1-[7-hydroxy-1-(3,3,5,5-tetramethylcyclohexyl)-1H-pyrazolo[4,3-d]pyrimidin-5-yl]-1H-pyrazole-4-carboxylic acid;
1-(1-cycloheptyl-7-hydroxy-1H-pyrazolo[4,3-d]pyrimidin-5-yl)-1H-pyrazole-4-carboxylic acid;
1-(1-cyclooctyl-7-hydroxy-1H-pyrazolo[4,3-d]pyrimidin-5-yl)-1H-pyrazole-4-carboxylic acid;
1-(7-hydroxy-1-[trans-3-methylcyclohexyl]-1H-pyrazolo[4,3-d]pyrimidin-5-yl}-1H-pyrazole-4-carboxylic acid;
1-{7-hydroxy-1-[cis-3-methylcyclohexyl]-1H-pyrazolo[4,3-d]pyrimidin-5-yl}-1H-pyrazole-4-carboxylic acid;
1-{r-1-[t-3,t-5-dimethylcyclohexyl]-7-hydroxy-1H-pyrazolo[4,3-d]pyrimidin-5-yl}-1H-pyrazole-4-carboxylic acid; and
1-{7-hydroxy-1-[cis-3,3,5-trimethylcyclohexyl]-1H-pyrazolo[4,3-d]pyrimidin-5-yl}-1H-pyrazole-4-carboxylic acid;
   or pharmaceutically acceptable salts thereof.

The compound (I) may exist in a form of tautomer or a mixture of tautomers. The compound (I) of the present invention may exist in a form of stereoisomer, such as diastereomer, enantiomer, or a mixture of stereoisomers. The compound (I) of the present invention may be a mixture of tautomers or stereoisomers, or respective pure or substantially pure isomer or stereoisomer.

If the compound (I) is obtainable in a form of diastereomer or enantiomer, it may be isolated by conventional methods known in the art, such as chromatography, fractional crystallization.

The pharmaceutically acceptable salts of the compounds (I) include salts with alkali metal such as lithium, sodium, potassium; salts with Group 2 metal such as calcium, ammonium; salts with aluminum or zinc; salts with amine such as ammonia, choline, diethanolamine, lysine, ethylenediamine, t-butylamine, t-octylamine, tris(hydroxymethyl)aminomethane, N-methyl-glucosamine, triethanolamine, dehydroabietylamine; salts with an inorganic acid such as hydrogen chloride, hydrogen bromide, hydrogen iodide, sulfuric acid, nitric acid, phosphoric acid; salts with an organic acid such as formic acid, acetic acid, propionic acid, oxalic acid, malonic acid, succinic acid, fumaric acid, maleic acid, lactic acid, malic salt, tartaric acid, citric acid, methanesulfonic acid, ethanesulfonic acid, benzenesulfonic acid; and salts with an acidic amino acid such as aspartic acid, glutamic acid.

Furthermore, the pharmaceutically acceptable salts of the compounds (I) includes inner salts, hydrates and solvates thereof.

The compound (I) or a pharmaceutically acceptable salt thereof of the present invention may be administered orally or parenterally in a form of pharmaceutical formulation as conventionally used, such as tablets, granules, capsules, powders, injectable solutions, inhalants.

The dose of the compound (I) or a pharmaceutically acceptable salt thereof of the present invention is generally 0.001 to 500 mg/kg, preferably 0.1 to 100 mg/kg, although it should vary depending on the mode of administration, the age, weight and condition of the patient.

The compound of the present invention has a HIF-PHD inhibitory effect, and therefore, is suitable for treating or preventing diseases associated with HIF-PHD.

Therefore, the compound of the present invention is useful for the prevention or treatment of anemia [renal anemia associated with renal failure, due to hematopoietic abnormalities of bone marrow, due to deficiency of iron, vitamin B₁₂ or folic acid, due to bleeding caused by accidents or surgery, associated with chronic inflammation such as autoimmune diseases, malignant tumors, chronic infections, transformation abnormality and the like, associated with endocrine diseases such as hypothyroidism, autoimmune polyglandular syndrome, IA diabetes, abnormal uterine bleeding and the like, associated with chronic heart failure, associated with ulcer, associated with liver disease, associated with senile anemia, associated with drug-induced anemia, associated with chemotherapy]; ischemic heart disease (angina pectoris, myocardial infarction, etc.); ischemic cerebrovascular disease (cerebral infarction, cerebral embolism, transient ischemic attack, etc.); chronic renal failure (ischemic nephropathy, tubulointerstitial damage, etc.); diabetes complications (diabetic wounds, etc.); cognitive impairment (dementia, Alzheimer's disease, Parkinson's disease, Huntington's disease, etc.); and other diseases expected to be improved by stabilizing HIF.

The method for the treatment or prevention comprising administering an effective amount of the compound (I) or a pharmaceutically acceptable salt thereof to a patient (subject of such treatment or prevention) is also applied for the object of the invention as described above and encompassed within the scope of the present invention.

The use of the compound (I) of the present invention or a pharmaceutically acceptable salt thereof in the manufacture of a medicament having HIF-PHD inhibitory effect is also applied for the object of the invention as described above and encompassed within the scope of the present invention.

According to the present invention, the compound (I) or a pharmaceutically acceptable salt thereof may be prepared by the following procedures, but not limited thereto.

In each process for the compound (I) as described below, in case where the functional group in the compound is required to be protected, such protection may be carried out appropriately using conventional methods. General descriptions of protecting groups and their use are described in T. W. Greene et al, “Protecting Groups in Organic Synthesis”, John Wiley & Sons, New York, 2006. Such protecting group may be removed in a subsequent step appropriately using a conventional method.

The compound of formula (I):

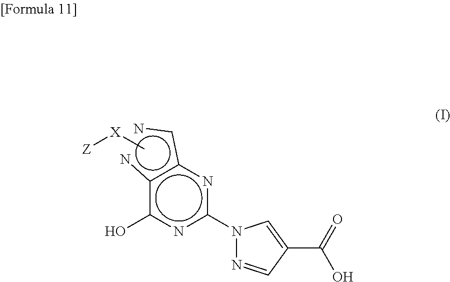

wherein the symbols are as defined above, of the present invention can be prepared by hydrolyzing a compound represented by the formula (1):

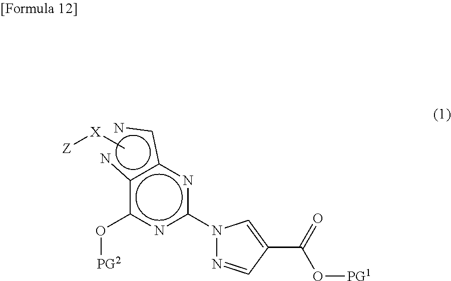

wherein

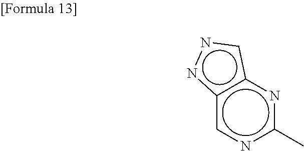

is an optionally substituted pyrazolo[4,3-d]pyrimidin-5-yl;
PG¹ is a protecting group for carboxylic acid;
PG² is a protecting group for hydroxy; and
the other symbols are as defined above.

Examples for the protective group represented by PG¹ include alkyl. Examples for the protective group represented by PG² include alkyl.

Hydrolysis of the compound of formula (1) wherein PG¹ is alkyl and PG² is alkyl can be carried out in a suitable solvent, in the presence of a base.

Examples of the base include alkali metal hydroxide such as lithium hydroxide, sodium hydroxide, potassium hydroxide; and alkali metal alkoxide such as sodium methoxide, sodium ethoxide. The solvent may be any one which does not affect the reaction and includes ethers such as tetrahydrofuran, 1,2-dimethoxyethane, 1,4-dioxane; alcohols such as methanol, ethanol, 2-propanol, t-butanol; water; and a mixed solvent thereof. The reaction can be carried out at a temperature of 20° C. to 100° C., especially preferably, 40° C. to 80° C.

Production of Intermediate Compounds

Intermediate compounds of the present invention (1) can be prepared, for example, according to the following Schemes A, B, C, D, E, F, G, H, J, K and L.

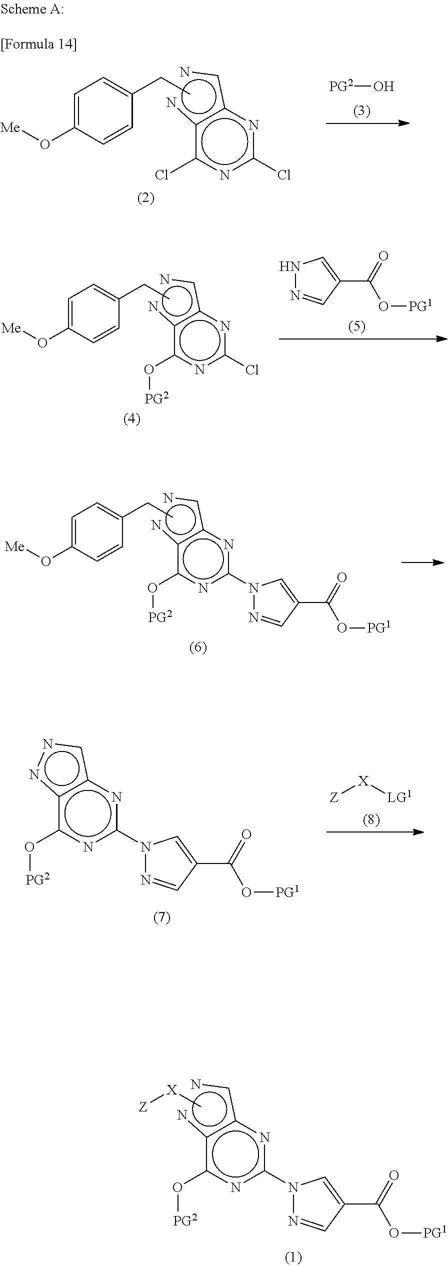

wherein LG¹ is a leaving group, and the other symbols are as defined above. Examples for such leaving group represented by LG¹ include halogen such as chlorine atom, bromine atom, iodine atom.

Compound (2) is reacted with Compound (3) to obtain Compound (4), which is then reacted with Compound (5) to obtain Compound (6).

The p-methoxybenzyl of Compound (6) is removed to obtain Compound (7), which is then reacted with Compound (8) to obtain the intermediate compound (1) of interest.

The reaction of Compound (2) with Compound (3) can be carried out in a suitable solvent, in the presence of a base.

Examples for the base include alkali metal hydroxide such as lithium hydroxide, sodium hydroxide, potassium hydroxide; alkali metal hydride such as sodium hydride; alkali metal salts of Compound (3) such as lithium salt of Compound (3), sodium salt of Compound (3); and potassium salt of Compound (3). The solvent may be any one which does not affect the reaction and includes ethers such as tetrahydrofuran, 1,2-dimethoxyethane, 1,4-dioxane; a solvent amount of Compound (3); or a mixed solvent thereof. The reaction proceeds suitably at a temperature of −20° C. to 100° C., particularly 0° C. to 50° C.

The reaction of Compound (4) with Compound (5) can be carried out in the presence of a palladium catalyst and a base, with or without a ligand, in a suitable solvent.

Examples for such palladium catalyst includes tris(dibenzylideneacetone)dipalladium(0), tetrakis(triphenylphosphine)palladium(0), palladium acetate(II), palladium(II) chloride, bis(triphenylphosphine)palladium(II) dichloride, [1,1′-bis(diphenylphosphino)ferrocene]palladium(II) dichloride, bis(di-t-butyl(4-dimethylaminophenyl)phosphine)palladium(II) dichloride.

Examples for such base include alkali metal phosphates such as trisodium phosphate, disodium hydrogen phosphate, tripotassium phosphate; and alkali metal fluorides such as potassium fluoride, cesium fluoride. Examples for such ligand include phosphine ligands such as 2-di-t-butylphosphino-2′,4′,6′-triisopropylbiphenyl, 2-dicyclohexylphosphino-2′,6′-dimethoxybiphenyl, 2-di-t-butylphosphino-3,4,5,6-tetramethyl-2′,4′,6′-triisopropyl-1,1′-biphenyl. The solvent may be any one which does not affect the reaction and includes ethers such as tetrahydrofuran, 1,2-dimethoxyethane, 1,4-dioxane; alcohols such as t-butanol; aromatic hydrocarbon such as toluene, xylene; and a mixed solvent thereof. The reaction suitably proceeds at a temperature of 20° C. to 180° C., particularly 60° C. to 150° C. In addition, the reaction can be suitably carried out at elevated temperature (e.g. 100° C. to 180° C.) using microwave irradiation.

The reaction to remove the p-methoxybenzyl group of compound (6) can be carried out by any conventional reaction to remove p-methoxybenzyl.

Specifically, for example, the reaction can be carried out in the presence of a solvent amount of an acid, such as trifluoroacetic acid. This reaction suitably proceeds at a temperature of 20° C. to 80° C., particularly 40° C. to 70° C.

The reaction of Compound (7) with Compound (8) can be carried out in a suitable solvent in the presence of a base.

Examples for such base include alkali metal carbonates such as sodium carbonate, sodium hydrogen carbonate, potassium carbonate, cesium carbonate; alkali metal phosphate such as trisodium phosphate, disodium hydrogen phosphate, tripotassium phosphate; alkali metal hydride such as sodium hydride. The solvent may be any which does not affect the reaction and include alkyl nitriles such as acetonitrile, propionitrile; amides such as N,N-dimethylformamide, N,N-dimethylacetamide; and a mixture thereof. The reaction proceeds suitably at a temperature of 0° C. to 100° C., particularly 20° C. to 80° C.

Scheme B:

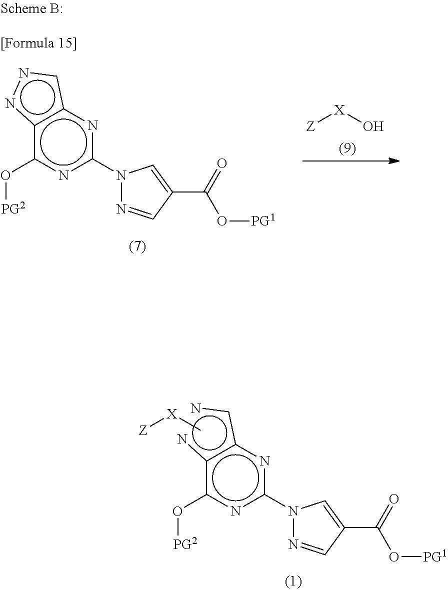

wherein the symbols are as defined above.

Compound (7) can be reacted with Compound (9) to obtain intermediate Compound (1) of interest.

The reaction of Compound (7) with Compound (9) can be carried out in a suitable solvent, in the presence of an azodicarboxylic acid derivative and a phosphine derivative.

Examples for such azodicarboxylic acid derivative include dialkyl esters of azodicarboxlic acid such as diethyl azodicarboxylate, diisopropyl azodicarboxylate; azodicarboxamide such as N,N,N′,N′-tetramethyl azodicarboxamide. Examples for such phosphine derivative include triarylphosphines such as triphenylphosphine; trialkylphosphines such as tributyl phosphine. The solvent may be any which does not affect the reaction and include ethers such as terrahydrofuran, 1,2-dimethoxyethane, 1,4-dioxane; or a mixed solvent thereof. The reaction proceeds suitably at a temperature of −20° C. to 100° C., particularly 0° C. to 80° C.

For one example of the intermediate compounds of the formula (1), a compound represented by the formula (1-a):

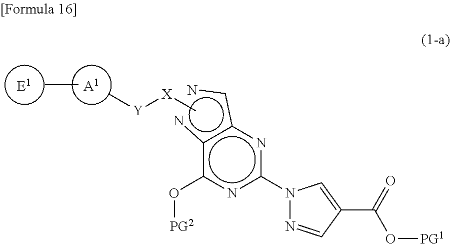

wherein,

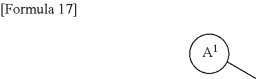

represents an optionally substituted aryl or an optionally substituted heteroaryl;

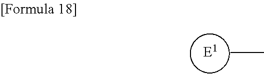

represents an optionally substituted aryl or an optionally substituted heteroaryl;
Y is a single bond, oxygen atom or the following formula

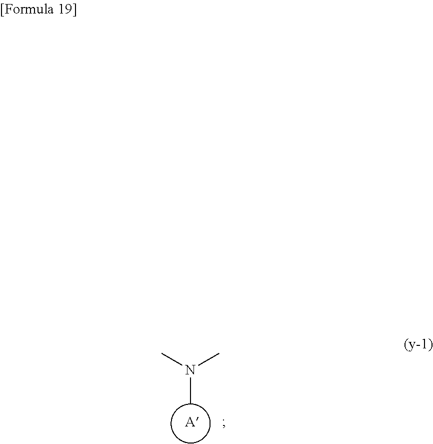

and
the other symbols are as defined above, can be prepared, for example, according to the following scheme C.

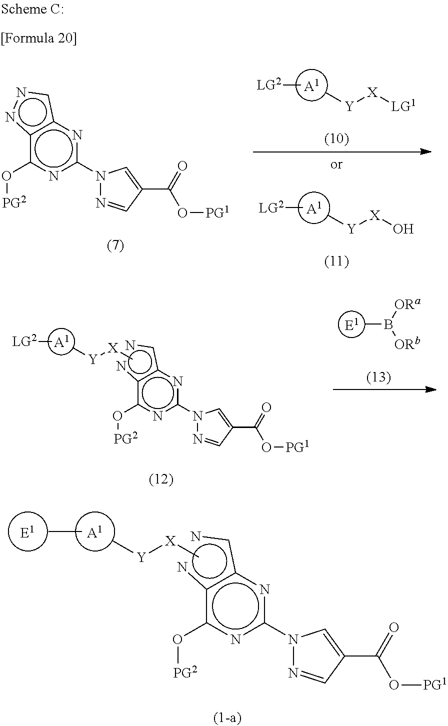

wherein LG² is a leaving group, R^{a} and R^{b} are each independently hydrogen atom or alkyl or R^{a} and R^{b} are joined together to form an alkylene group, and the other symbols are as defined above.

Examples for such leaving group represented by LG² include halogen such as chlorine atom, bromine atom, or iodine atom.

Compound (7) is reacted with Compound (10) or Compound (11) to obtain Compound (12), which is then reacted with Compound (13) to obtain intermediate Compound (1-a) of interest.

The reaction of Compound (7) with Compound (10) can be carried out in the same manner as the reaction of Compound (7) and Compound (8) in the above Scheme A.

The reaction of Compound (7) with Compound (11) can be carried out in the same manner as the reaction of Compound (7) with Compound (9) in the above Scheme B.

The reaction of Compound (12) with Compound (13) can be carried out in the same manner as the reaction of Compound (4) and Compound (5) in the above of Scheme A.

For one example of the intermediate compounds of the formula (1), a compound represented by the formula (1-b):

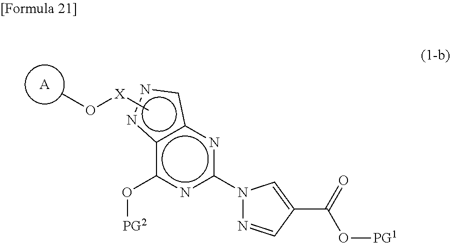

wherein the symbols are as defined above,
can be prepared, for example, according to the following scheme D.

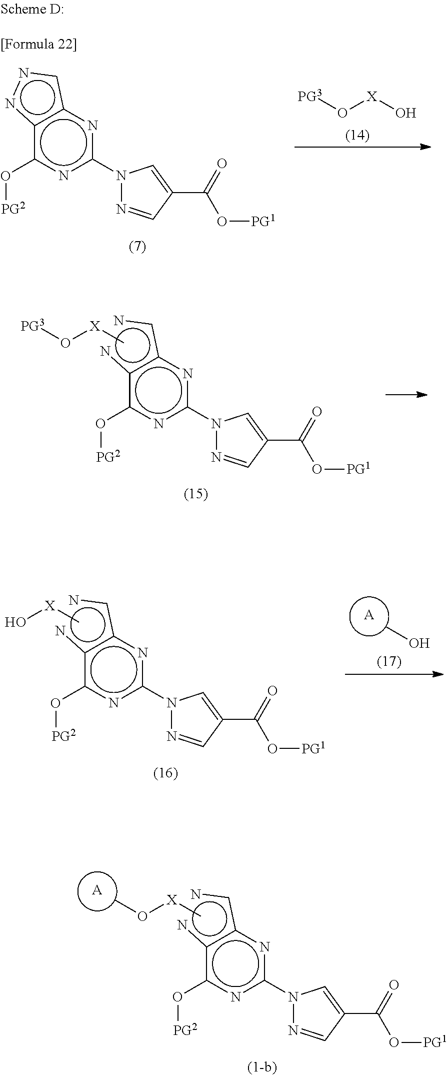

wherein, PG³ is a protecting group for hydroxy, and the other symbols are as defined above.

Examples for such protecting group for hydroxy represented by PG³ include trialkylsilyl such as t-butyldimethylsilyl.

Compound (7) is reacted with Compound (14) to obtain Compound (15).

PG³ of Compound (15) is removed to obtain Compound (16), which is then reacted with Compound (17) to obtain intermediate Compounds (1-b) of interest.

The reaction of Compound (7) with Compound (14) can be carried out in the same manner as the reaction of Compound (7) with Compound (9) in the above Scheme B.

The removal of the protecting group PG³ of Compound (15) can be carried out by any conventional procedure, such as acid treatment, fluoride treatment, depending on the type of the protecting group.

The reaction of Compound (16) with Compound (17) can be carried out in the same manner as the reaction of Compound (7) with Compound (9) in the above Scheme B.

For one example of the intermediate compounds of the formula (1), a compound represented by the formula (1-c):

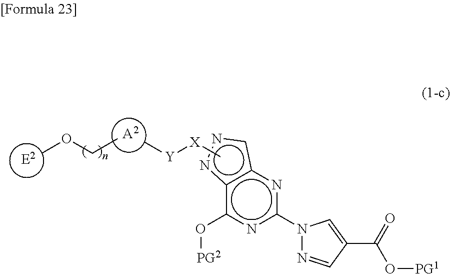

wherein,

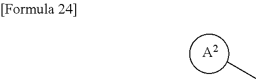

represents an optionally substituted alicyclic hydrocarbon;

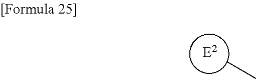

represents an optionally substituted aryl;
n is 0 or 1; and
the other symbols are as defined above, can be prepared, for example, according to the following scheme E.

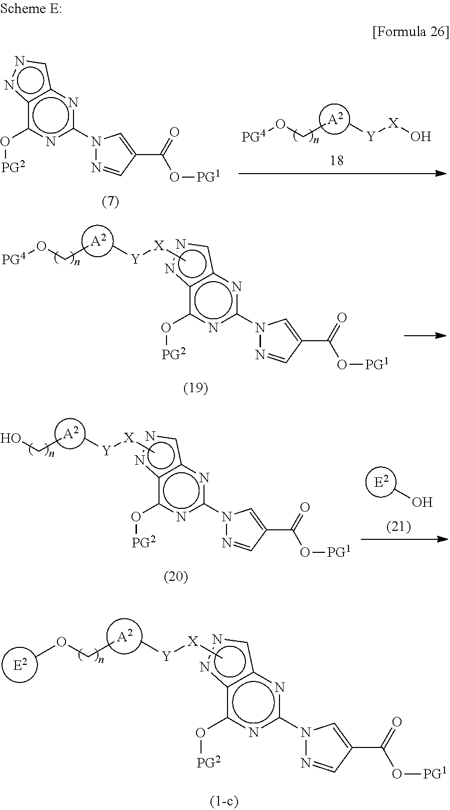

wherein, PG⁴ is a protecting group for hydroxy, and the other symbols are as defined above.

Examples of such protecting group for hydroxy represented by PG⁴ include triakylsily such as t-butyldimethylsilyl.

Compound (7) is reacted with Compound (18) to obtain Compound (19).

PG⁴ of Compound (19) is removed to obtained Compound (20), which is then reacted with Compound (2) to obtain intermediate Compounds (1-c) of interest.

The reaction of Compound (7) with Compound (18) can be carried out in the same manner as the reaction of Compound (7) with Compound (9) in the above Scheme B.

The removal of protecting group PG⁴ of Compound (19) can be carried out by any conventional procedure, such as acid treatment, fluoride treatment, depending on the type of the protecting group.

The reaction of Compound (20) with Compound (21) can be carried out in the same manner as the reaction of Compound (7) with Compound (9) in the above Scheme B.

For one example of the intermediate compounds of the formula (1), a compound represented by the formula (1-d):

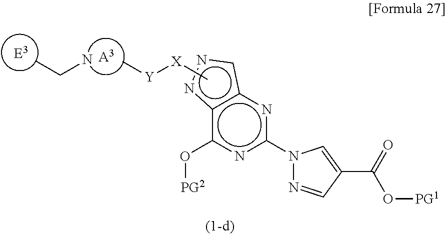

wherein,

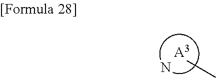

represents an optionally substituted nitrogen-containing non-aromatic heterocycle;

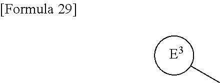

represents an optionally substituted aryl; and
the other symbols are as defined above,
can be prepared, for example, according to the following scheme F.

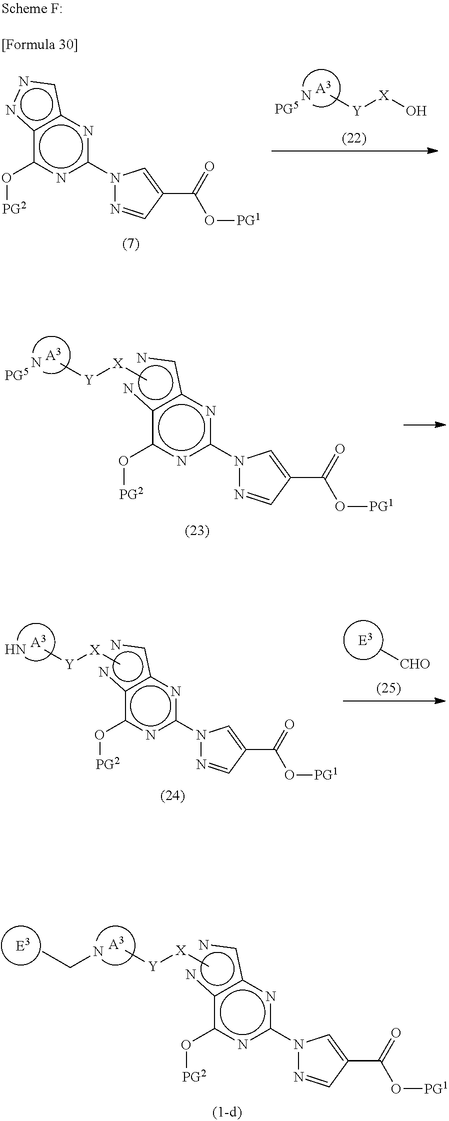

wherein, PG⁵ is a protecting group for amino, and the other symbols are as defined above.

Examples for such protecting group for amino represented by PG⁵ include alkoxycarbonyl such as t-butoxycarbonyl.

Compound (7) is reacted with Compound (22) to obtain Compound (23).

PG⁵ of Compound (23) is removed to obtain Compound (24), which is then reacted with Compound (25) to obtain intermediate Compounds (1-d) of interest.

The reaction of Compound (7) with Compound (22) can be carried out in the same manner as the reaction of Compound (7) with Compound (9) in the above Scheme B.

The Removal of protecting group PG⁵ of Compound (23) can be carried out by any conventional procedure, such as acid treatment, base treatment, depending on the type of the protecting group.

The reaction of Compound (24) with Compound (25) can be carried out in a suitable solvent, in the present of a reducing agent.

Examples for such reducing agent include alkali metal borohydride such as sodium triacetoxyborohydride, sodium cyanoborohydride. The solvent may be any which does not affect the reaction and include halogenohydrocarbon such as dichloromethane, chloroform, 1,2-dichloro ethane; ethers such as tetrahydrofuran, 1,2-dimethoxyethane, 1,4-dioxane; or a mixed solvent thereof. The reaction proceeds suitably at a temperature of −20° C. to 80° C., particularly 0° C. to 60° C.

For one example of the intermediate compounds of the formula (1), a compound represented by the formula (1-e):

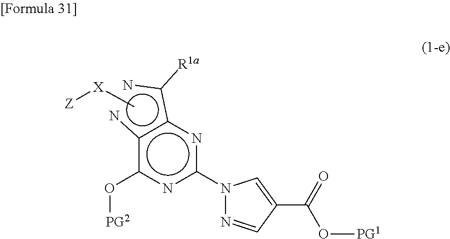

wherein
R^{1a} represents halogen, and the other symbols are as defined above,
can be prepared, for example, according to the following scheme G.

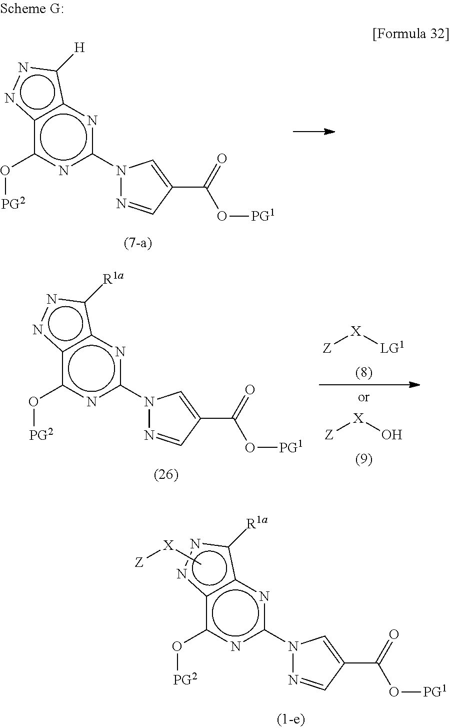

wherein the symbols are as defined above.

Compound (7-a) can be reacted with a halogenating agent to obtain Compound (26), which is then reacted with Compound (8) or Compound (9) to obtain intermediate Compound (1-e) of interest.

The reaction of Compound (7-a) can be carried out with a halogenating agent corresponding to the type of R^{1a} to be introduced, in a suitable solvent.

A compound having fluorine atom as R^{1a} can be prepared, for example, by treating the compound with a fluorinating agent such as 1-chloromethyl-4-fluoro-1,4-diazoniabicyclo[2.2.2]octane bis(tetrafluoroborate). The solvent may be any which does not affect the reaction and include alkyl nitriles such as acetonitrile, propionitrile; alkyl carboxylic acid such as acetic acid; or a mixed solvent thereof. The reaction proceeds suitably at a temperature of 20° C. to 120° C., particularly 50° C. to 100° C.

A compound having chlorine atom as R^{1a} can be prepared, for example, by treating the compound with a chlorinating agent such as N-chlorosuccinimide. The solvent may be any which does not affect the reaction and include alkyl nitriles such as acetonitrile, propionitrile. The reaction proceeds suitably at a temperature of 20° C. to 120° C., particularly 50° C. to 100° C.

A compound having bromine atom as R^{1a} can be prepared, for example, by treating the compound with a brominating agent such as N-bromosuccinimide. The solvent may be any which does not affect the reaction and include alkyl nitriles such as acetonitrile, propionitrile. The reaction proceeds suitably at a temperature of 20° C. to 120° C., particularly 50° C. to 100° C.

A compound having iodine atom as R^{1a} can be prepared, for example, by treating the compound with a iodinating agent such as N-iodosuccinimide. The solvent may be any which does not affect the reaction and include alkyl nitriles such as acetonitrile, propionitrile. The reaction proceeds suitably at a temperature of 20° C. to 120° C., particularly 50° C. to 100° C.

The reaction of Compound (26) with Compound (8) can be carried out in the same manner as the reaction of Compound (7) with Compound (8) in the above Scheme A.

The reaction of Compound (26) with Compound (9) can be carried out in the same manner as the reaction of Compound (7) with Compound (9) in the above Scheme B.

For one example of the intermediate compounds of the formula (1), a compound represented by the formula (1-f):

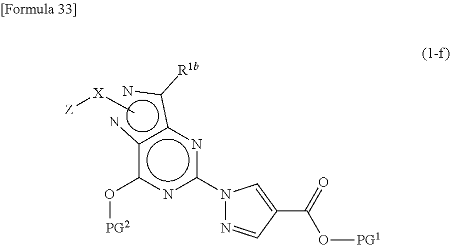

wherein R^{1b} is alkyl, fluoroalkyl or cycloalkyl, and the other symbols are as defined above,
can be prepared, for example, according to the following scheme H.

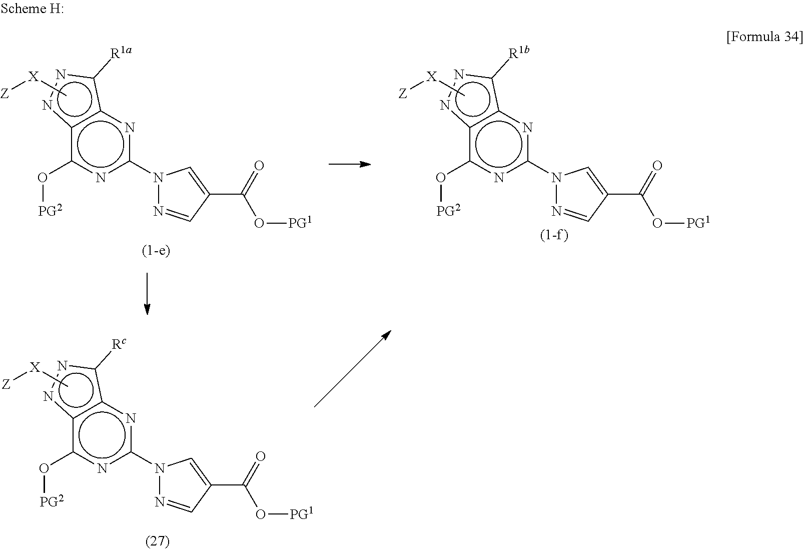

wherein R^{c} is alkenyl, and the other symbols are as defined above.

An intermediate compound (1-f) of interest is prepared by alkylation, fluoroalkylation or cycloalkylation of Compound (1-e).

Compound (1-e) is alkenylated to obtain Compound (27), which is then hydrogenated to obtain intermediate Compound (1-f) of interest wherein R^{1b} is C₂-C₆ alkyl.

The alkylation of Compound (1-e) can be carried out by reacting Compound (1-e) with the corresponding alkyl boric acid or a derivative thereof, in the same manner as the reaction of Compound (4) with Compound (5) in the above Scheme A.

The cycloalkylation of Compound (1-e) can be carried out by reacting Compound (1-e) with the corresponding cycloalkyl boric acid or a derivative thereof, in the same manner as the reaction of Compound (4) with Compound (5) in the above Scheme A.

The fluoroalkylation of Compound (1-e) can be carried out by reacting Compound (1-e) wherein R^{1a} is iodine atom with the corresponding methyl(fluorosulfonyl)difluoroacetate, potassium fluoroalkyl carboxylate or fluoroalkyl trimethylsilane, in the presence of a copper complex in the suitable solvent.

Examples for such copper complex include cuprous iodide. The solvent may be any which does not affect the reaction and include amides such as N,N-dimethylformamide, N,N-dimethylacetamide. The reaction proceeds suitably at a temperature of 50° C. to 150° C., particularly 80° C. to 120° C.

The alkenylation of Compound (1-e) can be carried out by reacting Compound (1-e) with the corresponding alkenyl boric acid or a derivative thereof, in the same manner as the reaction of Compound (4) with Compound (5) in the above Scheme A.

The hydrogenation of Compound (27) can be carried out in the presence of a catalyst under hydrogen atmosphere in a suitable solvent.

Examples for such catalyst include palladium on carbon and palladium hydroxide. The solvent may be any which does not affect the reaction and include alcohols such as methanol, ethanol, and 2-propanol. The reaction proceeds suitably at a temperature of 0° C. to 60° C., particularly 10° C. to 40° C.

For one example of the intermediate compounds of the formula (1), a compound represented by the formula (1-g):

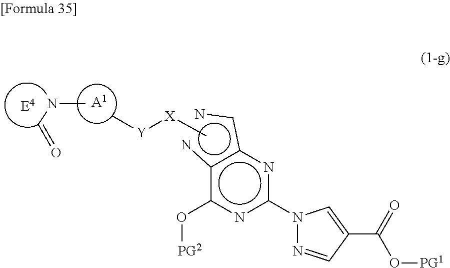

wherein,

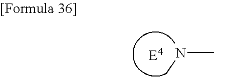

represents a nitrogen-containing non-aromatic heterocycle an optionally substituted, and the other symbols are as defined above,
can be prepared, for example, according to the following scheme J.

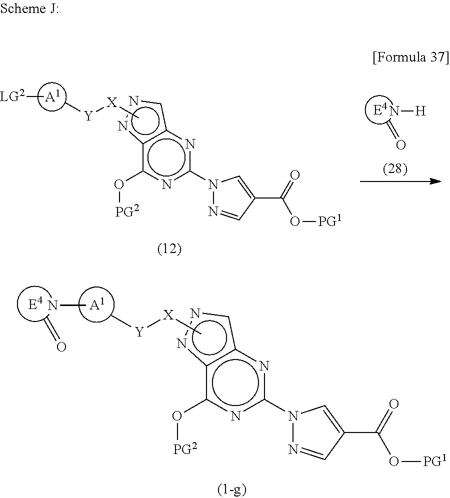

wherein the symbols are as defined above.

Compound (12) can be reacted with compound (28) to obtain intermediate Compounds (1-g) of interest.

The reaction of Compound (12) with Compound (28) can be carried out in a suitable solvent, in the presence of a palladium catalyst, a ligand and a base.

Examples for such palladium catalyst include tris(dibenzylideneacetone)dipalladium(0), palladium(II) acetate and palladium(II) chloride. Examples for such ligand include 4,5′-bis(diphenylphosphino)-9,9′-dimethylxanthene (Xantphos) and the like. Examples for such base include alkali metal carbonates such as sodium carbonate, sodium hydrogen carbonate, potassium carbonate, cesium carbonate. The solvent may be any which does not affect the reaction and include ethers such as tetrahydrofuran, 1,2-dimethoxyethane, 1,4-dioxane. The reaction proceeds suitably at a temperature of 20° C. to 120° C., particularly 50° C. to 100° C.

For one example of the intermediate compounds of the formula (1), a compound represented by the formula (1-h):

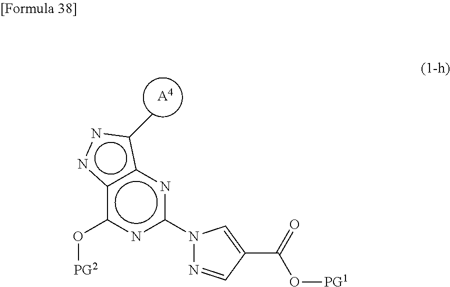

wherein,

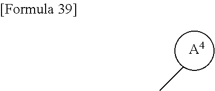

is an optionally substituted aryl or an optionally substituted cycloalkyl, and the other symbols are as defined above, can be prepared, for example, according to the following scheme K.

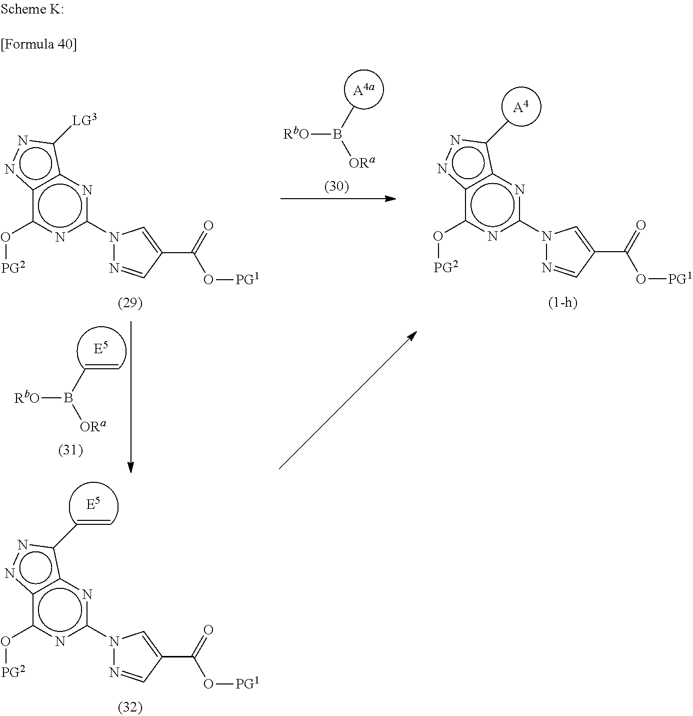

wherein
LG³ is a leaving group;

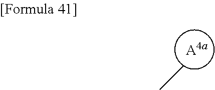

represents an optionally substituted aryl;

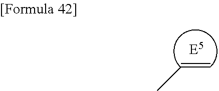

represents an optionally substituted cycloalkenyl, and
the other symbols are as defined above.

Examples for such leaving group represented by LG³ include halogen such as chlorine atom, bromine atom, iodine.

Compound (29) can be reacted with Compound (30) to obtain intermediate Compound (1-h) of interest wherein ring A′ is an optionally substituted aryl.

Compound (29) is reacted with Compound (31) to obtain Compound (32), which then can be hydrogenated to obtain intermediate Compound (1-h) of interest wherein ring A′ is cycloalkyl an optionally substituted.

The reaction of Compound (29) with Compound (30) can be carried out in the same manner as the reaction of Compound (4) with Compound (5) in the above Scheme A.

The reaction of Compound (29) with Compound (31) can be carried out in the same manner as the reaction of Compound (4) with Compound (5) in the above Scheme A.

The hydrogenation of Compound (32) may be carried out in the same manner as the reaction of Compound (27) in the above Scheme H.

For one example of the intermediate compounds of the formula (1), a compound represented by the formula (1-i):

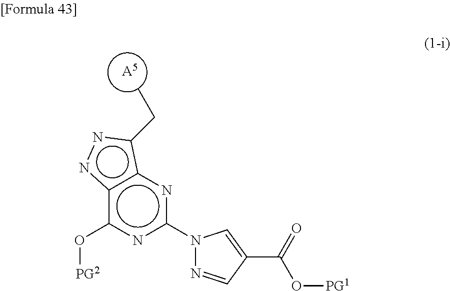

wherein,

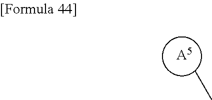

represents an optionally substituted aryl, and the other symbols are as defined above,
can be prepared, for example, according to the following scheme L.

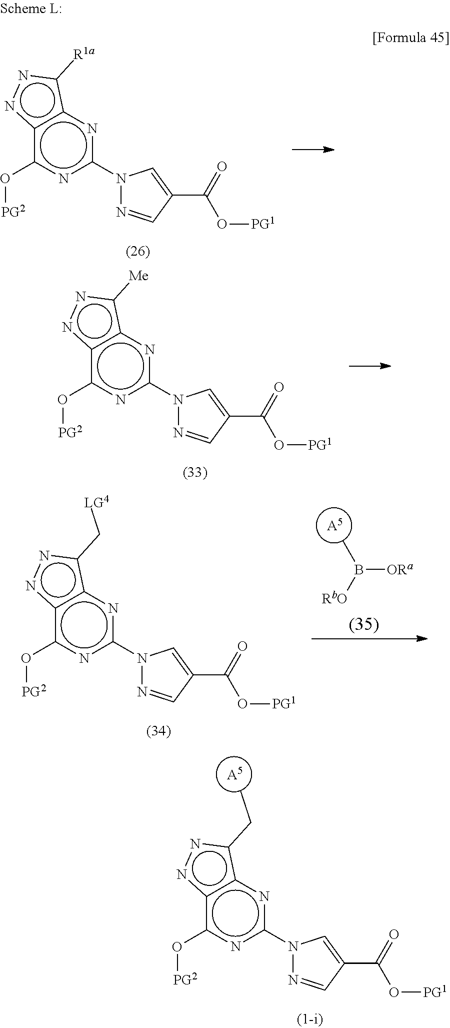

wherein Me represents methyl, LG⁴ is a leaving group, and the other symbols are as defined above.

Examples for such leaving group represented by LG⁴ include halogen such as chlorine atom, bromine atom, iodine atom.

Compound (26) is methylated to obtain Compound (33), which is then halogenated to obtain Compound (34). Then, Compound (34) can be reacted with Compound (35) to obtain intermediate Compound (1-i) of interest.

The methylation of Compound (26) can be carried out by reacting Compound (26) with methylboric acid or a derivative thereof, in the same manner as the reaction of Compound (4) with Compound (5) in the above Scheme A.

The halogenation of Compound (33) can be carried out in a solvent (e.g., carbon tetrachloride) in the presence of the corresponding halogenating agent (e.g., corresponding N-halogenosuccinimide) and an azo compound (e.g., azobisisobutyronitrile).

The reaction of Compound (34) with Compound (35) can be carried out in the same manner as the reaction of Compound (4) with Compound (5) in the above Scheme A.

The compound (I) or intermediate compound thereof, wherein the substituent group for ring A or ring A′ is an optionally substituted alkyl, can be prepared, respectively, by hydrogenating corresponding compound (I) or intermediate compound thereof having corresponding an optionally substituted alkenyl as the substituent group for ring A or ring A′.

The reaction can be carried out in the same manner as the hydrogenation of Compound (27) in the above Scheme H.

Starting materials used in the processes as described (Schemes A, B, C, D, E, F, G, H, J, K, L) is either commercially available or can be prepared readily in accordance with conventional method well known in the art.

The following examples explain the invention in more detail, but the present invention is not limited thereto.

In the following Examples, Reference Examples and Tables, “Me” means methyl, “Et” means ethyl, “*” denotes an asymmetric carbon. In addition, racemic mixtures, by dividing by chiral high performance liquid chromatography (chiral HPLC) or chiral supercritical fluid chromatography (chiral SFC), can be obtained an optically active substance. Examples of chiral HPLC column include CHIRALPAK IA, CHIRALPAK IC, CHIRALPAK ID, and CHIRALPAK IF (Daicel Chemical Industries, Ltd.), and examples of chiral SFC column include CHIRALPAK IA/SFC (Daicel Co., Ltd.). The compound with a sign “(+)”, “(+)-trans” or “(+)-cis” means that the specific rotation of the compound is plus (+), and the compound with a sign “(−)”, “(−)-trans” or “(−)-cis” means that the specific rotation of the compound is minus (−).

### EXAMPLE 1

### Preparation of 1-[1-(3,4-dichlorobenzyl)-7-hydroxy-1H-pyrazolo[4,3-d]pyrimidin-5-yl]-1H-pyrazole-4-carboxylic acid

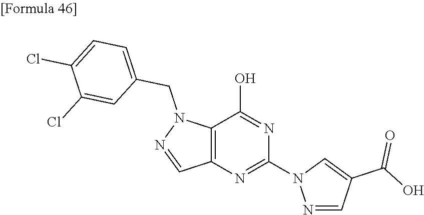 [SMILES: O=C(O)c1cnn(-c2nc(O)c3c(cnn3Cc3ccc(Cl)c(Cl)c3)n2)c1]

A solution of ethyl 1-[1-(3,4-dichloro-benzyl)-7-methoxy-1H-pyrazolo[4,3-d]pyrimidin-5-yl]ol-1H-pyrazole-4-carboxylate (237 mg), which was prepared in Reference Example 3, in 2 mol/L aqueous sodium hydroxide (5 mL), tetrahydrofuran (5 mL) and ethanol (5 mL) was stirred for 1.5 hours at 60° C. The reaction mixture was concentrated, and the resulting residue was added with water (10 mL) and 2 mol/L hydrochloric acid (5.1 mL), followed by stirring the mixture. The resulting solid was collected by filtration, washed with water and dried under reduced pressure to yield the titled compound (208 mg, 97% yield) as a colorless powder.

MS (APCI) m/z: 405/407 [M+H]⁺.

### EXAMPLE 2

### Preparation of 1-(1-{[1-(4-fluorobenzyl)piperidin-4-yl]methyl}-7-hydroxy-1H-pyrazolo[4,3-d]pyrimidin-5-yl)-1H-pyrazole-4-carboxylic acid hydrochloride

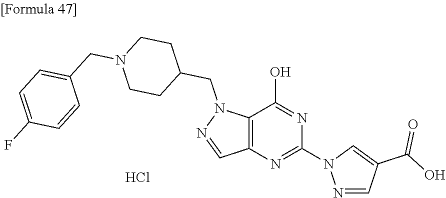 [SMILES: O=C(O)c1cnn(-c2nc(O)c3c(cnn3CC3CCN(Cc4ccc(F)cc4)CC3)n2)c1 ; Cl]

A solution of ethyl(1-{[1-(4-fluorobenzyl)piperidin-4-yl]methyl}-7-methoxy-1H-pyrazolo[4,3-d]pyrimidin-5-yl)-1H-pyrazole-4-carboxylate (49 mg), which was prepared in Reference Example 458, in 1 mol/L aqueous sodium hydroxide (0.6 mL), tetrahydrofuran (0.6 mL) and ethanol (0.6 mL) was stirred for 1.5 hours at 60° C. The reaction mixture was concentrated, and the residue was added with water (6 mL) and 1 mol/L hydrochloric acid (0.8 mL), followed by stirring the mixture. The resulting solid was collected by filtration, washed with water and dried under reduced pressure to yield the titled compound (38 mg, 78.5% yield) as a colorless powder.

MS (APCI) m/z: 452 [M+H]⁺.

### EXAMPLES 3 TO 506

The compounds listed in the following Table 1 were obtained from the corresponding starting material in the same manner as described in Example 1 or 2. A free form and a salt thereof can be converted to each other, by salt formation or desalting process as conventionally used in the art.

TABLE 1

| Example | Structure | material properties |
|---|---|---|
| 3 | 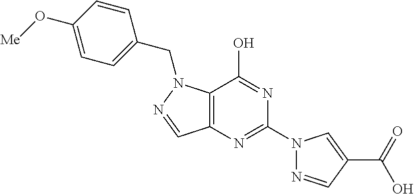 [SMILES: COc1ccc(Cn2ncc3nc(-n4cc(C(=O)O)cn4)nc(O)c32)cc1] | powder MS (APCI) m/z: 367 [M + H]⁺ |
| 4 | 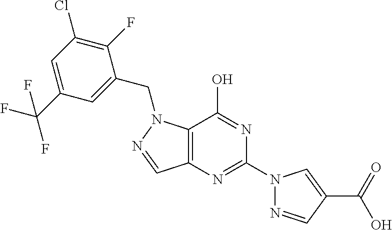 [SMILES: O=C(O)c1cnn(-c2nc(O)c3c(cnn3Cc3cc(C(F)(F)F)cc(Cl)c3F)n2)c1] | powder MS (APCI) m/z: 457/459 [M + H]⁺ |
| 5 | 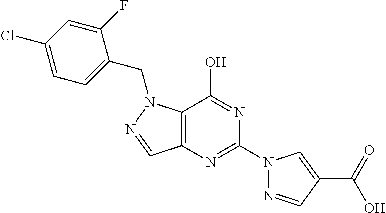 [SMILES: O=C(O)c1cnn(-c2nc(O)c3c(cnn3Cc3ccc(Cl)cc3F)n2)c1] | powder MS (APCI) m/z: 389/391 [M + H]⁺ |
| 6 | 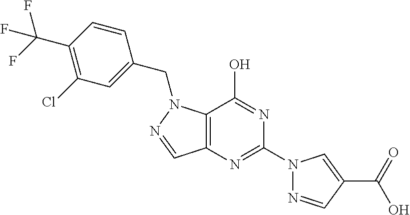 [SMILES: O=C(O)c1cnn(-c2nc(O)c3c(cnn3Cc3ccc(C(F)(F)F)c(Cl)c3)n2)c1] | powder MS (APCI) m/z: 439/441 [M + H]⁺ |
| 7 | 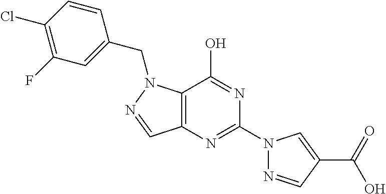 [SMILES: O=C(O)c1cnn(-c2nc(O)c3c(cnn3Cc3ccc(Cl)c(F)c3)n2)c1] | powder MS (APCI) m/z: 389/391 [M + H]⁺ |
| 8 | 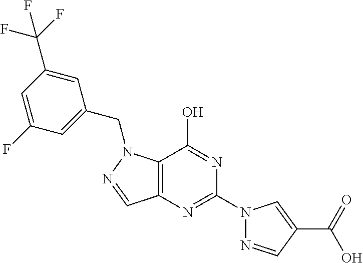 [SMILES: O=C(O)c1cnn(-c2nc(O)c3c(cnn3Cc3cc(F)cc(C(F)(F)F)c3)n2)c1] | powder MS (APCI) m/z: 423 [M + H]⁺ |
| 9 | 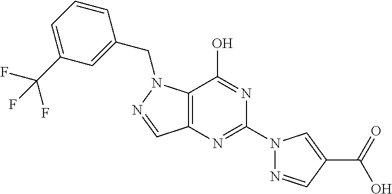 [SMILES: O=C(O)c1cnn(-c2nc(O)c3c(cnn3Cc3cccc(C(F)(F)F)c3)n2)c1] | powder MS (APCI) m/z: 405 [M + H]⁺ |
| 10 | 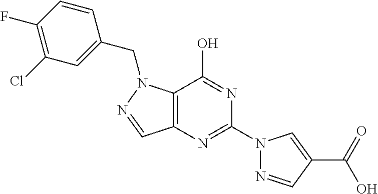 [SMILES: O=C(O)c1cnn(-c2nc(O)c3c(cnn3Cc3ccc(F)c(Cl)c3)n2)c1] | powder MS (APCI) m/z: 389/391 [M + H]⁺ |
| 11 | 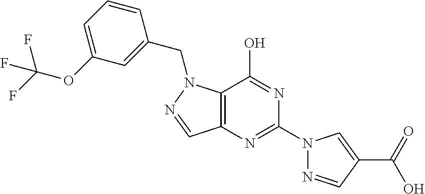 [SMILES: O=C(O)c1cnn(-c2nc(O)c3c(cnn3Cc3cccc(OC(F)(F)F)c3)n2)c1] | powder MS (APCI) m/z: 421 [M + H]⁺ |
| 12 | 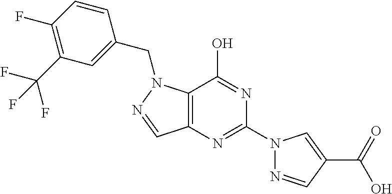 [SMILES: O=C(O)c1cnn(-c2nc(O)c3c(cnn3Cc3ccc(F)c(C(F)(F)F)c3)n2)c1] | powder MS (APCI) m/z: 423 [M + H]⁺ |
| 13 | 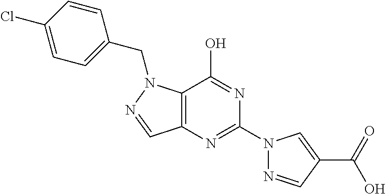 [SMILES: O=C(O)c1cnn(-c2nc(O)c3c(cnn3Cc3ccc(Cl)cc3)n2)c1] | powder MS (APCI) m/z: 371/373 [M + H]⁺ |
| 14 | 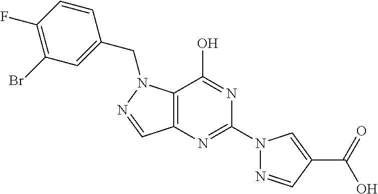 [SMILES: O=C(O)c1cnn(-c2nc(O)c3c(cnn3Cc3ccc(F)c(Br)c3)n2)c1] | powder MS (ESI) m/z: 431/433 [M − H]⁻ |
| 15 | 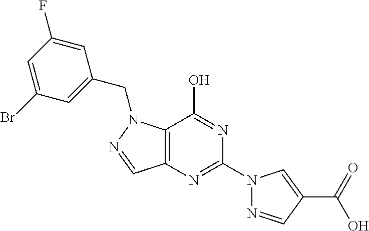 [SMILES: O=C(O)c1cnn(-c2nc(O)c3c(cnn3Cc3cc(F)cc(Br)c3)n2)c1] | powder MS (APCI) m/z: 433/435 [M + H]⁺ |
| 16 | 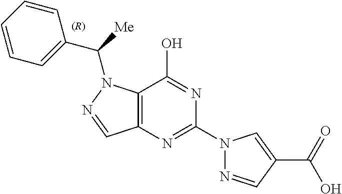 | powder MS (APCI) m/z: 351 [M + H]⁺ |
| 17 | 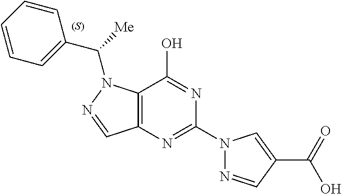 | powder MS (APCI) m/z: 351 [M + H]⁺ |
| 18 | 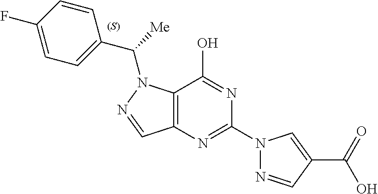 | powder MS (APCI) m/z: 369 [M + H]⁺ |
| 19 | 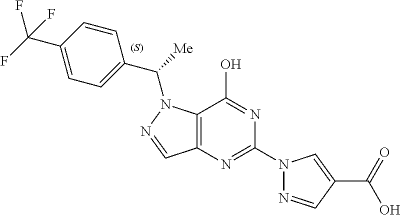 | powder MS (APCI) m/z: 419 [M + H]⁺ |
| 20 | 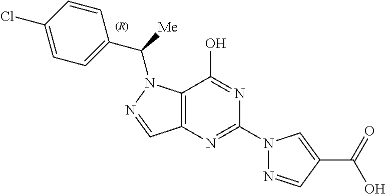 | powder MS (APCI) m/z: 385/387 [M + H]⁺ |
| 21 | 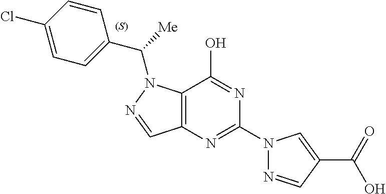 | powder MS (APCI) m/z: 385/387 [M + H]⁺ |
| 22 | 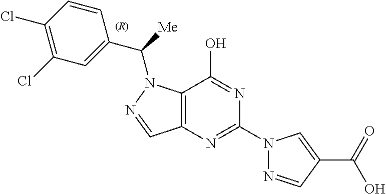 | powder MS (APCI) m/z: 419/421 [M + H]⁺ |
| 23 | 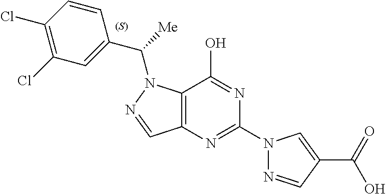 | powder MS (APCI) m/z: 419/421 [M + H]⁺ |
| 24 | 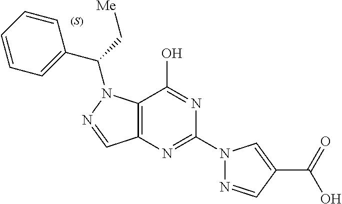 | powder MS (APCI) m/z: 365 [M + H]⁺ |
| 25 | 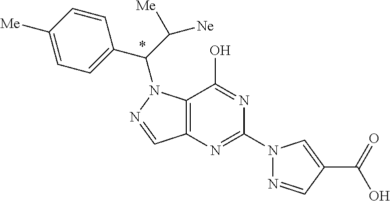 | powder MS (APCI) m/z: 393 [M + H]⁺ |
| 26 | 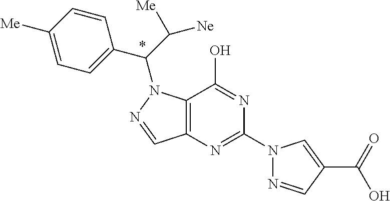 | powder MS (APCI) m/z: 393 [M + H]⁺ Enantiomer of Example 25 |
| 27 | 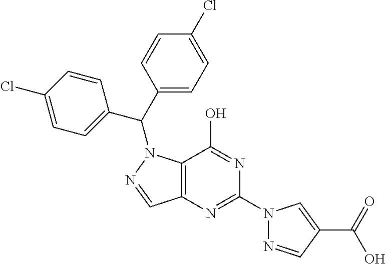 [SMILES: O=C(O)c1cnn(-c2nc(O)c3c(cnn3C(c3ccc(Cl)cc3)c3ccc(Cl)cc3)n2)c1] | powder MS (APCI) m/z: 481/483 [M + H]⁺ |
| 28 | 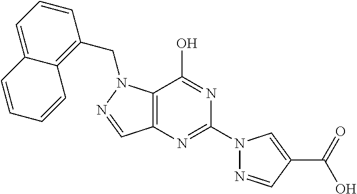 [SMILES: O=C(O)c1cnn(-c2nc(O)c3c(cnn3Cc3cccc4ccccc34)n2)c1] | powder MS (APCI) m/z: 387 [M + H]⁺ |
| 29 | 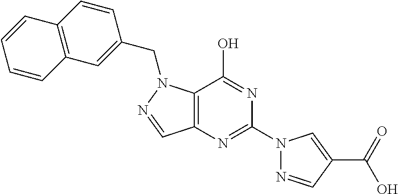 [SMILES: O=C(O)c1cnn(-c2nc(O)c3c(cnn3Cc3ccc4ccccc4c3)n2)c1] | powder MS (APCI) m/z: 387 [M + H]⁺ |
| 30 | 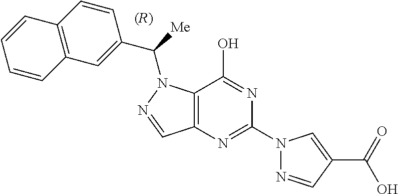 | powder MS (APCI) m/z: 401 [M + H]⁺ |
| 31 | 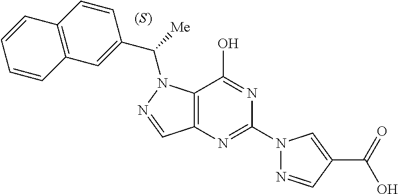 | powder MS (APCI) m/z: 401 [M + H]⁺ |
| 32 | 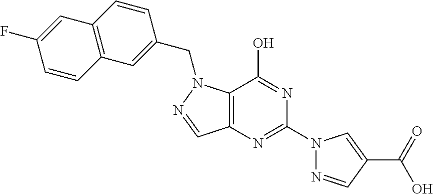 [SMILES: O=C(O)c1cnn(-c2nc(O)c3c(cnn3Cc3ccc4cc(F)ccc4c3)n2)c1] | powder MS (APCI) m/z: 405 [M + H]⁺ |
| 33 | 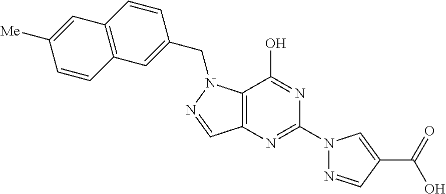 [SMILES: Cc1ccc2cc(Cn3ncc4nc(-n5cc(C(=O)O)cn5)nc(O)c43)ccc2c1] | powder MS (APCI) m/z: 401 [M + H]⁺ |
| 34 | 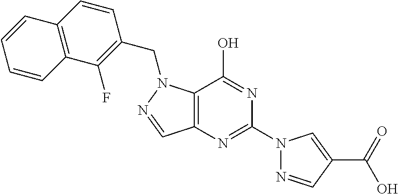 [SMILES: O=C(O)c1cnn(-c2nc(O)c3c(cnn3Cc3ccc4ccccc4c3F)n2)c1] | powder MS (APCI) m/z: 405 [M + H]⁺ |
| 35 | 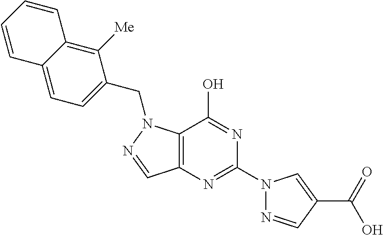 [SMILES: Cc1c(Cn2ncc3nc(-n4cc(C(=O)O)cn4)nc(O)c32)ccc2ccccc12] | powder MS (APCI) m/z: 401 [M + H]⁺ |
| 36 | 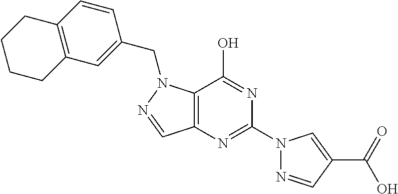 [SMILES: O=C(O)c1cnn(-c2nc(O)c3c(cnn3Cc3ccc4c(c3)CCCC4)n2)c1] | powder MS (APCI) m/z: 391 [M + H]⁺ |
| 37 | 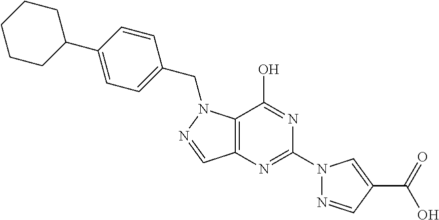 [SMILES: O=C(O)c1cnn(-c2nc(O)c3c(cnn3Cc3ccc(C4CCCCC4)cc3)n2)c1] | powder MS (APCI) m/z: 419 [M + H]⁺ |
| 38 | 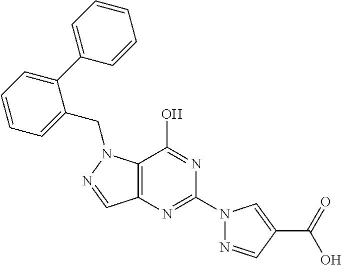 [SMILES: O=C(O)c1cnn(-c2nc(O)c3c(cnn3Cc3ccccc3-c3ccccc3)n2)c1] | powder MS (APCI) m/z: 413 [M + H]⁺ |
| 39 | 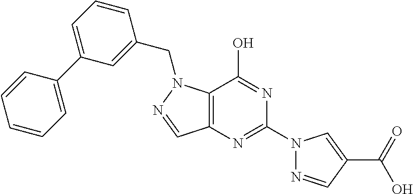 [SMILES: O=C(O)c1cnn(-c2nc(O)c3c(cnn3Cc3cccc(-c4ccccc4)c3)n2)c1] | powder MS (APCI) m/z: 413 [M + H]⁺ |
| 40 | 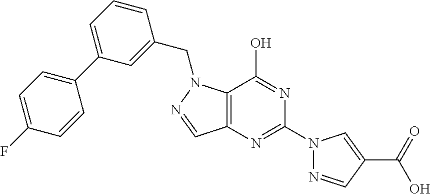 [SMILES: O=C(O)c1cnn(-c2nc(O)c3c(cnn3Cc3cccc(-c4ccc(F)cc4)c3)n2)c1] | powder MS (APCI) m/z: 431 [M + H]⁺ |
| 41 | 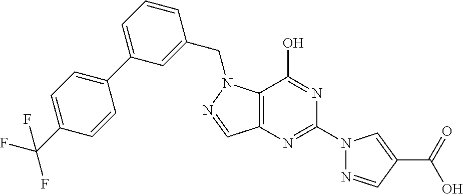 [SMILES: O=C(O)c1cnn(-c2nc(O)c3c(cnn3Cc3cccc(-c4ccc(C(F)(F)F)cc4)c3)n2)c1] | powder MS (APCI) m/z: 481 [M + H]⁺ |
| 42 | 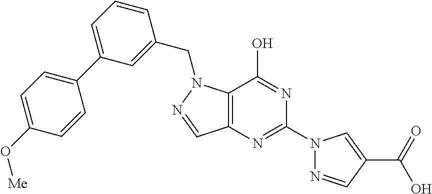 [SMILES: COc1ccc(-c2cccc(Cn3ncc4nc(-n5cc(C(=O)O)cn5)nc(O)c43)c2)cc1] | powder MS (APCI) m/z: 443 [M + H]⁺ |
| 43 | 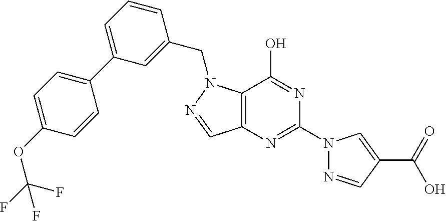 [SMILES: O=C(O)c1cnn(-c2nc(O)c3c(cnn3Cc3cccc(-c4ccc(OC(F)(F)F)cc4)c3)n2)c1] | powder MS (APCI) m/z: 497 [M + H]⁺ |
| 44 | 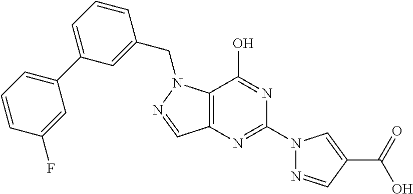 [SMILES: O=C(O)c1cnn(-c2nc(O)c3c(cnn3Cc3cccc(-c4cccc(F)c4)c3)n2)c1] | powder MS (APCI) m/z: 431 [M + H]⁺ |
| 45 | 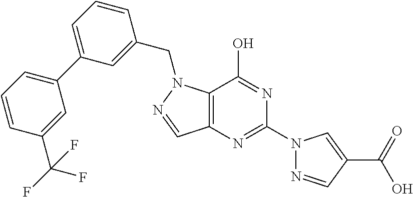 [SMILES: O=C(O)c1cnn(-c2nc(O)c3c(cnn3Cc3cccc(-c4cccc(C(F)(F)F)c4)c3)n2)c1] | powder MS (APCI) m/z: 481 [M + H]⁺ |
| 46 | 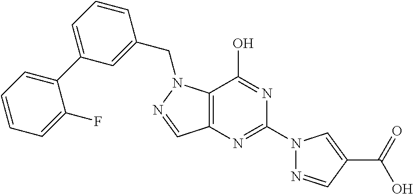 [SMILES: O=C(O)c1cnn(-c2nc(O)c3c(cnn3Cc3cccc(-c4ccccc4F)c3)n2)c1] | powder MS (APCI) m/z: 431 [M + H]⁺ |
| 47 | 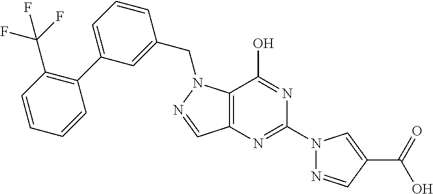 [SMILES: O=C(O)c1cnn(-c2nc(O)c3c(cnn3Cc3cccc(-c4ccccc4C(F)(F)F)c3)n2)c1] | powder MS (APCI) m/z: 481 [M + H]⁺ |
| 48 | 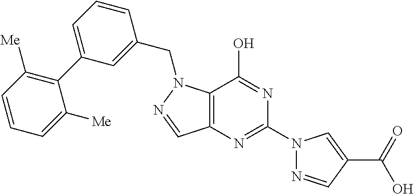 [SMILES: Cc1cccc(C)c1-c1cccc(Cn2ncc3nc(-n4cc(C(=O)O)cn4)nc(O)c32)c1] | powder MS (APCI) m/z: 441 [M + H]⁺ |
| 49 | 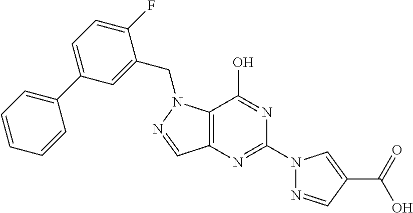 [SMILES: O=C(O)c1cnn(-c2nc(O)c3c(cnn3Cc3cc(-c4ccccc4)ccc3F)n2)c1] | powder MS (APCI) m/z: 431 [M + H]⁺ |
| 50 | 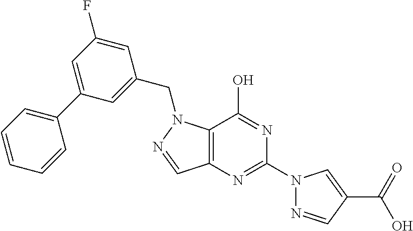 [SMILES: O=C(O)c1cnn(-c2nc(O)c3c(cnn3Cc3cc(F)cc(-c4ccccc4)c3)n2)c1] | powder MS (APCI) m/z: 431 [M + H]⁺ |
| 51 | 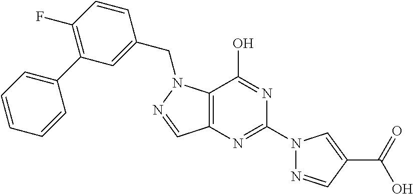 [SMILES: O=C(O)c1cnn(-c2nc(O)c3c(cnn3Cc3ccc(F)c(-c4ccccc4)c3)n2)c1] | powder MS (APCI) m/z: 431 [M + H]⁺ |
| 52 | 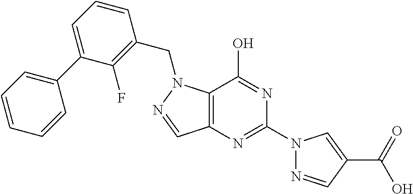 [SMILES: O=C(O)c1cnn(-c2nc(O)c3c(cnn3Cc3cccc(-c4ccccc4)c3F)n2)c1] | powder MS (APCI) m/z: 431 [M + H]⁺ |
| 53 | 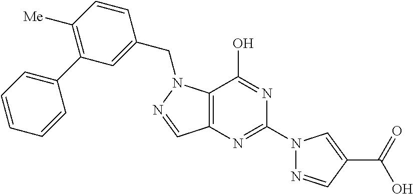 [SMILES: Cc1ccc(Cn2ncc3nc(-n4cc(C(=O)O)cn4)nc(O)c32)cc1-c1ccccc1] | powder MS (APCI) m/z: 427 [M + H]⁺ |
| 54 | 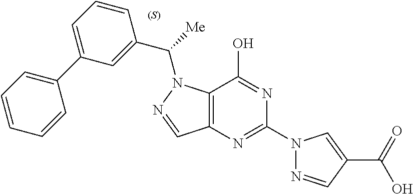 | powder MS (APCI) m/z: 427 [M + H]⁺ |
| 55 | 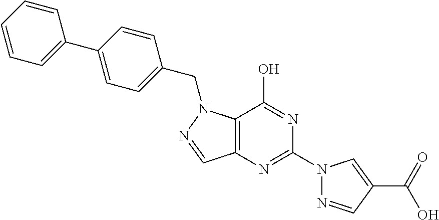 [SMILES: O=C(O)c1cnn(-c2nc(O)c3c(cnn3Cc3ccc(-c4ccccc4)cc3)n2)c1] | powder MS (APCI) m/z: 413 [M + H]⁺ |
| 56 | 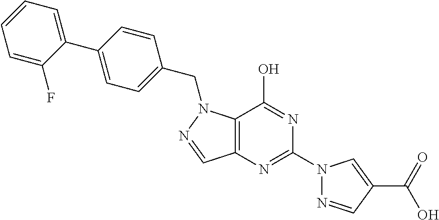 [SMILES: O=C(O)c1cnn(-c2nc(O)c3c(cnn3Cc3ccc(-c4ccccc4F)cc3)n2)c1] | powder MS (APCI) m/z: 431 [M + H]⁺ |
| 57 | 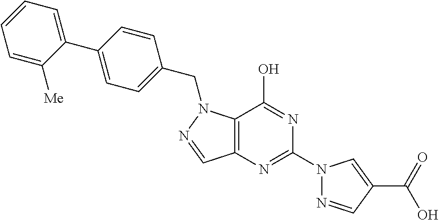 [SMILES: Cc1ccccc1-c1ccc(Cn2ncc3nc(-n4cc(C(=O)O)cn4)nc(O)c32)cc1] | powder MS (APCI) m/z: 427 [M + H]⁺ |
| 58 | 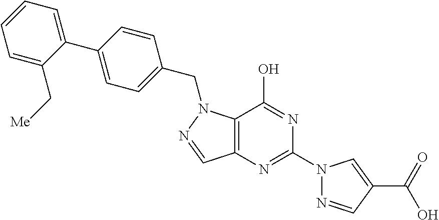 [SMILES: CCc1ccccc1-c1ccc(Cn2ncc3nc(-n4cc(C(=O)O)cn4)nc(O)c32)cc1] | powder MS (APCI) m/z: 441 [M + H]⁺ |
| 59 | 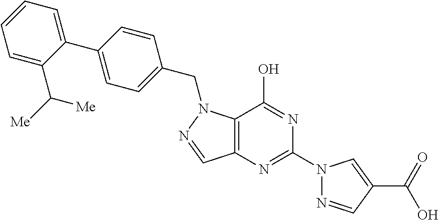 [SMILES: CC(C)c1ccccc1-c1ccc(Cn2ncc3nc(-n4cc(C(=O)O)cn4)nc(O)c32)cc1] | powder MS (APCI) m/z: 455 [M + H]⁺ |
| 60 | 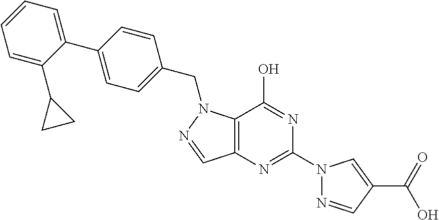 [SMILES: O=C(O)c1cnn(-c2nc(O)c3c(cnn3Cc3ccc(-c4ccccc4C4CC4)cc3)n2)c1] | powder MS (APCI) m/z: 453 [M + H]⁺ |
| 61 | 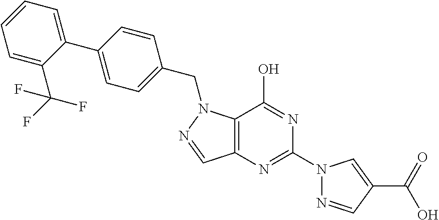 [SMILES: O=C(O)c1cnn(-c2nc(O)c3c(cnn3Cc3ccc(-c4ccccc4C(F)(F)F)cc3)n2)c1] | powder MS (APCI) m/z: 481 [M + H]⁺ |
| 62 | 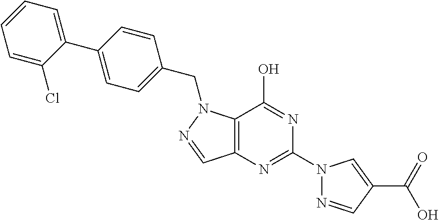 [SMILES: O=C(O)c1cnn(-c2nc(O)c3c(cnn3Cc3ccc(-c4ccccc4Cl)cc3)n2)c1] | powder MS (APCI) m/z: 447/449 [M + H]⁺ |
| 63 | 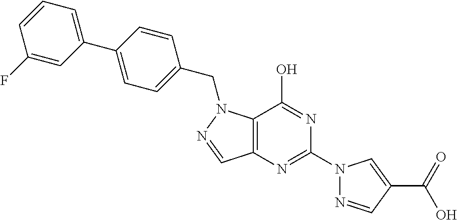 [SMILES: O=C(O)c1cnn(-c2nc(O)c3c(cnn3Cc3ccc(-c4cccc(F)c4)cc3)n2)c1] | powder MS (APCI) m/z: 431 [M + H]⁺ |
| 64 | 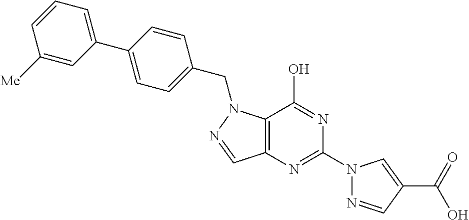 [SMILES: Cc1cccc(-c2ccc(Cn3ncc4nc(-n5cc(C(=O)O)cn5)nc(O)c43)cc2)c1] | powder MS (APCI) m/z: 427 [M + H]⁺ |
| 65 | 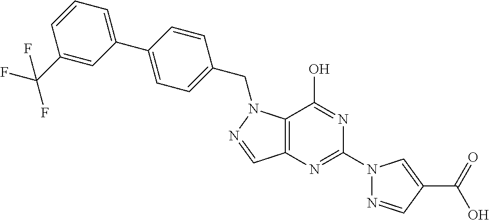 [SMILES: O=C(O)c1cnn(-c2nc(O)c3c(cnn3Cc3ccc(-c4cccc(C(F)(F)F)c4)cc3)n2)c1] | powder MS (APCI) m/z: 481 [M + H]⁺ |
| 66 | 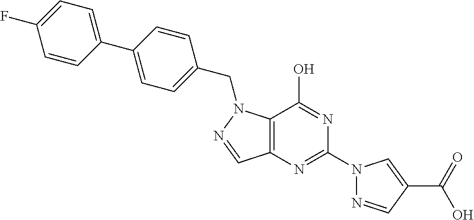 [SMILES: O=C(O)c1cnn(-c2nc(O)c3c(cnn3Cc3ccc(-c4ccc(F)cc4)cc3)n2)c1] | powder MS (APCI) m/z: 431 [M + H]⁺ |
| 67 | 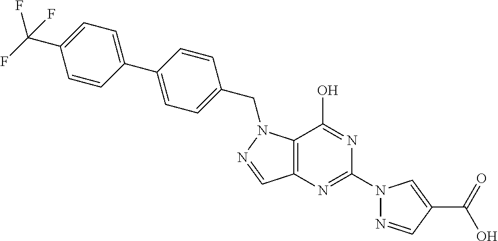 [SMILES: O=C(O)c1cnn(-c2nc(O)c3c(cnn3Cc3ccc(-c4ccc(C(F)(F)F)cc4)cc3)n2)c1] | powder MS (APCI) m/z: 481 [M + H]⁺ |
| 68 | 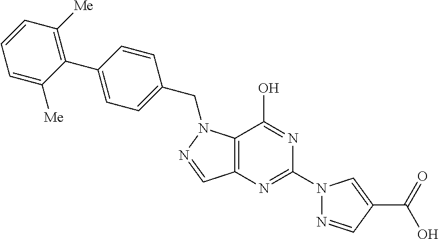 [SMILES: Cc1cccc(C)c1-c1ccc(Cn2ncc3nc(-n4cc(C(=O)O)cn4)nc(O)c32)cc1] | powder MS (APCI) m/z: 441 [M + H]⁺ |
| 69 | 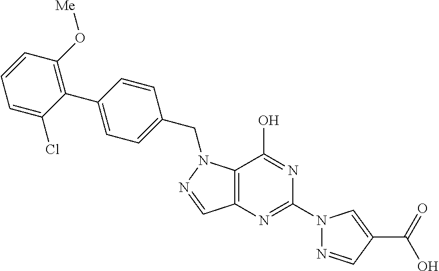 [SMILES: COc1cccc(Cl)c1-c1ccc(Cn2ncc3nc(-n4cc(C(=O)O)cn4)nc(O)c32)cc1] | powder MS (APCI) m/z: 447/479 [M + H]⁺ |
| 70 | 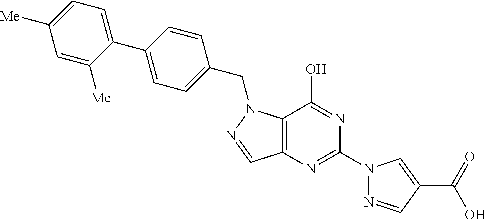 [SMILES: Cc1ccc(-c2ccc(Cn3ncc4nc(-n5cc(C(=O)O)cn5)nc(O)c43)cc2)c(C)c1] | powder MS (APCI) m/z: 441 [M + H]⁺ |
| 71 | 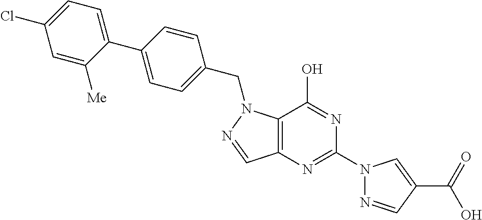 [SMILES: Cc1cc(Cl)ccc1-c1ccc(Cn2ncc3nc(-n4cc(C(=O)O)cn4)nc(O)c32)cc1] | powder MS (APCI) m/z: 461/463 [M + H]⁺ |
| 72 | 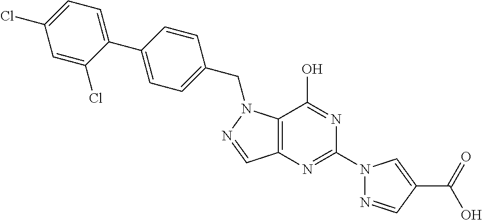 [SMILES: O=C(O)c1cnn(-c2nc(O)c3c(cnn3Cc3ccc(-c4ccc(Cl)cc4Cl)cc3)n2)c1] | powder MS (APCI) m/z: 481/483 [M + H]⁺ |
| 73 | 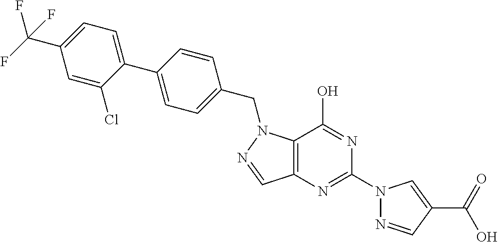 [SMILES: O=C(O)c1cnn(-c2nc(O)c3c(cnn3Cc3ccc(-c4ccc(C(F)(F)F)cc4Cl)cc3)n2)c1] | powder MS (APCI) m/z: 515/517 [M + H]⁺ |
| 74 | 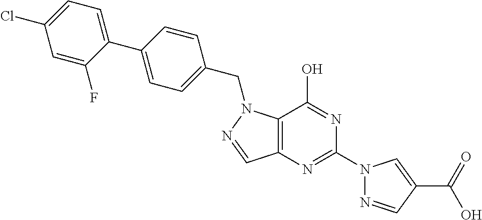 [SMILES: O=C(O)c1cnn(-c2nc(O)c3c(cnn3Cc3ccc(-c4ccc(Cl)cc4F)cc3)n2)c1] | powder MS (APCI) m/z: 465/467 [M + H]⁺ |
| 75 | 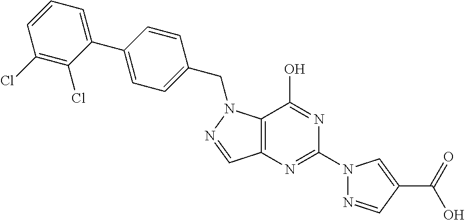 [SMILES: O=C(O)c1cnn(-c2nc(O)c3c(cnn3Cc3ccc(-c4cccc(Cl)c4Cl)cc3)n2)c1] | powder MS (APCI) m/z: 481/483 [M + H]⁺ |
| 76 | 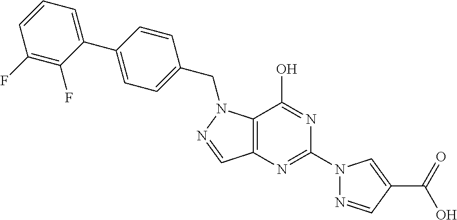 [SMILES: O=C(O)c1cnn(-c2nc(O)c3c(cnn3Cc3ccc(-c4cccc(F)c4F)cc3)n2)c1] | powder MS (APCI) m/z: 449 [M + H]⁺ |
| 77 | 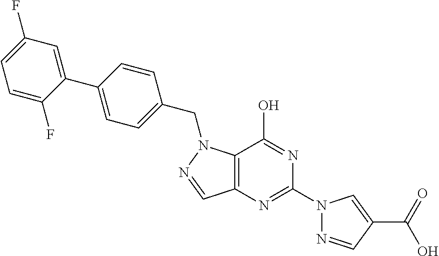 [SMILES: O=C(O)c1cnn(-c2nc(O)c3c(cnn3Cc3ccc(-c4cc(F)ccc4F)cc3)n2)c1] | powder MS (APCI) m/z: 449 [M + H]⁺ |
| 78 | 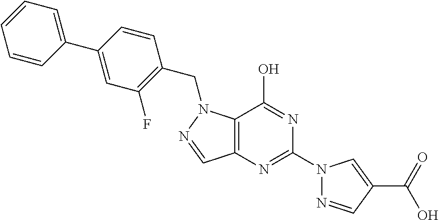 [SMILES: O=C(O)c1cnn(-c2nc(O)c3c(cnn3Cc3ccc(-c4ccccc4)cc3F)n2)c1] | powder MS (APCI) m/z: 431 [M + H]⁺ |
| 79 | 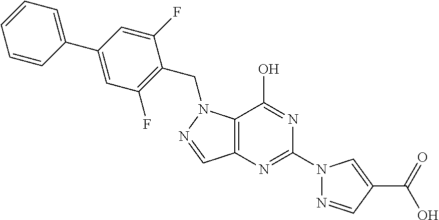 [SMILES: O=C(O)c1cnn(-c2nc(O)c3c(cnn3Cc3c(F)cc(-c4ccccc4)cc3F)n2)c1] | powder MS (APCI) m/z: 449 [M + H]⁺ |
| 80 | 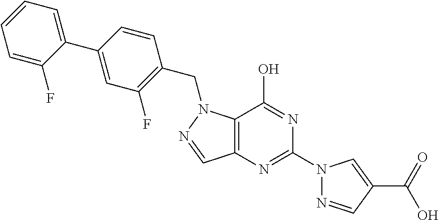 [SMILES: O=C(O)c1cnn(-c2nc(O)c3c(cnn3Cc3ccc(-c4ccccc4F)cc3F)n2)c1] | powder MS (APCI) m/z: 449 [M + H]⁺ |
| 81 | 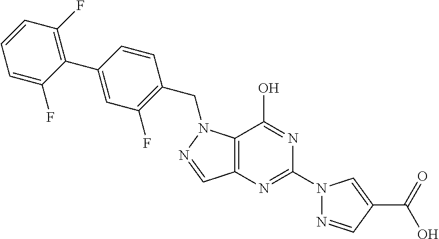 [SMILES: O=C(O)c1cnn(-c2nc(O)c3c(cnn3Cc3ccc(-c4c(F)cccc4F)cc3F)n2)c1] | powder MS (APCI) m/z: 467 [M + H]⁺ |
| 82 | 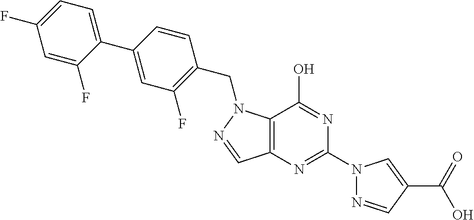 [SMILES: O=C(O)c1cnn(-c2nc(O)c3c(cnn3Cc3ccc(-c4ccc(F)cc4F)cc3F)n2)c1] | powder MS (APCI) m/z: 467 [M + H]⁺ |
| 83 | 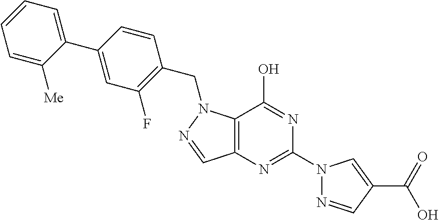 [SMILES: Cc1ccccc1-c1ccc(Cn2ncc3nc(-n4cc(C(=O)O)cn4)nc(O)c32)c(F)c1] | powder MS (APCI) m/z: 445 [M + H]⁺ |
| 84 | 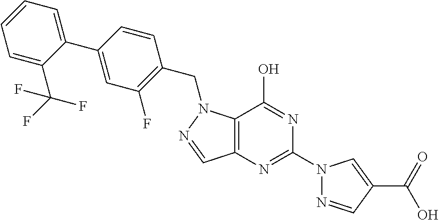 [SMILES: O=C(O)c1cnn(-c2nc(O)c3c(cnn3Cc3ccc(-c4ccccc4C(F)(F)F)cc3F)n2)c1] | powder MS (APCI) m/z: 499 [M + H]⁺ |
| 85 | 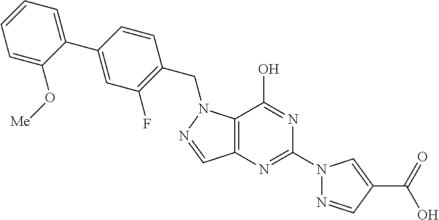 [SMILES: COc1ccccc1-c1ccc(Cn2ncc3nc(-n4cc(C(=O)O)cn4)nc(O)c32)c(F)c1] | powder MS (APCI) m/z: 461 [M + H]⁺ |
| 86 | 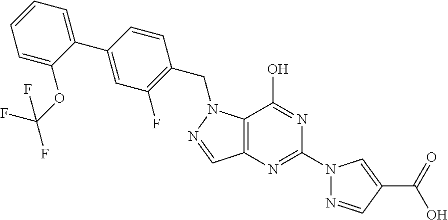 [SMILES: O=C(O)c1cnn(-c2nc(O)c3c(cnn3Cc3ccc(-c4ccccc4OC(F)(F)F)cc3F)n2)c1] | powder MS (APCI) m/z: 515 [M + H]⁺ |
| 87 | 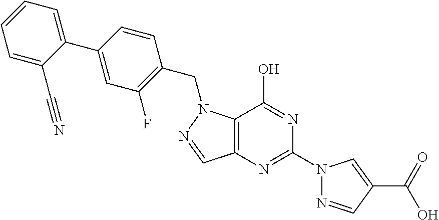 [SMILES: N#Cc1ccccc1-c1ccc(Cn2ncc3nc(-n4cc(C(=O)O)cn4)nc(O)c32)c(F)c1] | powder MS (APCI) m/z: 456 [M + H]⁺ |
| 88 | 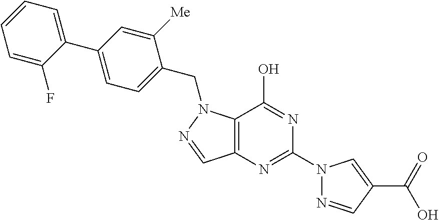 [SMILES: Cc1cc(-c2ccccc2F)ccc1Cn1ncc2nc(-n3cc(C(=O)O)cn3)nc(O)c21] | powder MS (APCI) m/z: 445 [M + H]⁺ |
| 89 | 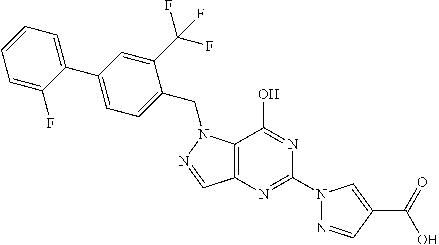 [SMILES: O=C(O)c1cnn(-c2nc(O)c3c(cnn3Cc3ccc(-c4ccccc4F)cc3C(F)(F)F)n2)c1] | powder MS (APCI) m/z: 499 [M + H]⁺ |
| 90 | 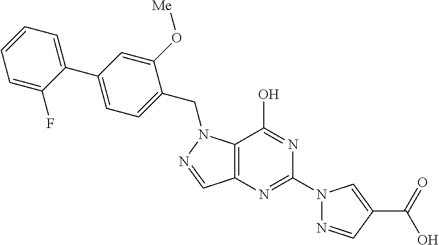 [SMILES: COc1cc(-c2ccccc2F)ccc1Cn1ncc2nc(-n3cc(C(=O)O)cn3)nc(O)c21] | powder MS (APCI) m/z: 461 [M + H]⁺ |
| 91 | 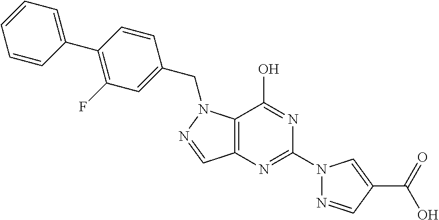 [SMILES: O=C(O)c1cnn(-c2nc(O)c3c(cnn3Cc3ccc(-c4ccccc4)c(F)c3)n2)c1] | powder MS (APCI) m/z: 431 [M + H]⁺ |
| 92 | 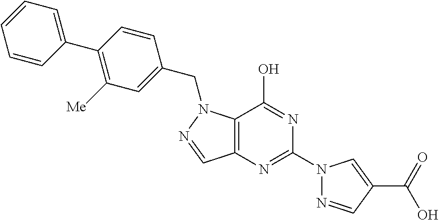 [SMILES: Cc1cc(Cn2ncc3nc(-n4cc(C(=O)O)cn4)nc(O)c32)ccc1-c1ccccc1] | powder MS (APCI) m/z: 427 [M + H]⁺ |
| 93 | 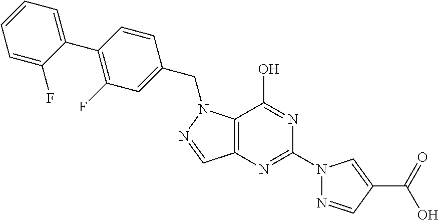 [SMILES: O=C(O)c1cnn(-c2nc(O)c3c(cnn3Cc3ccc(-c4ccccc4F)c(F)c3)n2)c1] | powder MS (APCI) m/z: 449 [M + H]⁺ |
| 94 | 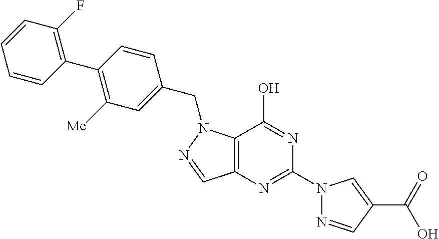 [SMILES: Cc1cc(Cn2ncc3nc(-n4cc(C(=O)O)cn4)nc(O)c32)ccc1-c1ccccc1F] | powder MS (APCI) m/z: 445 [M + H]⁺ |
| 95 | 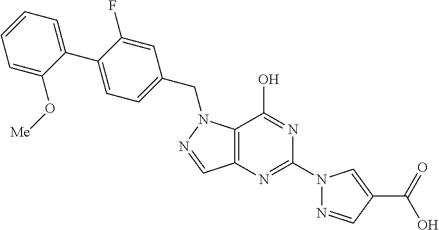 [SMILES: COc1ccccc1-c1ccc(Cn2ncc3nc(-n4cc(C(=O)O)cn4)nc(O)c32)cc1F] | powder MS (ESI) m/z: 461 [M + H]⁺ |
| 96 | 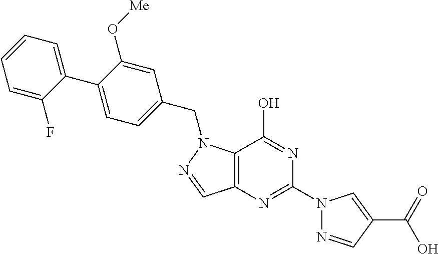 [SMILES: COc1cc(Cn2ncc3nc(-n4cc(C(=O)O)cn4)nc(O)c32)ccc1-c1ccccc1F] | powder MS (APCI) m/z: 461 [M + H]⁺ |
| 97 | 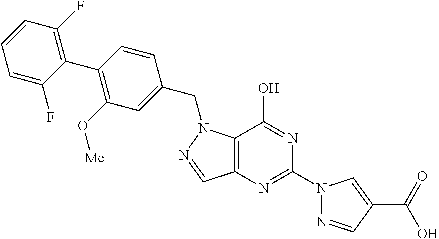 [SMILES: COc1cc(Cn2ncc3nc(-n4cc(C(=O)O)cn4)nc(O)c32)ccc1-c1c(F)cccc1F] | powder MS (ESI) m/z: 479 [M + H]⁺ |
| 98 | 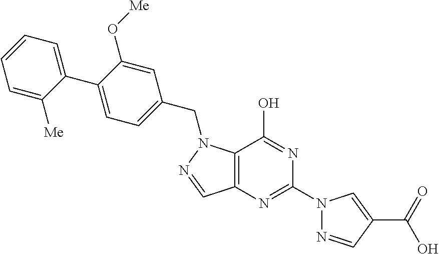 [SMILES: COc1cc(Cn2ncc3nc(-n4cc(C(=O)O)cn4)nc(O)c32)ccc1-c1ccccc1C] | powder MS (APCI) m/z: 457 [M + H]⁺ |
| 99 | 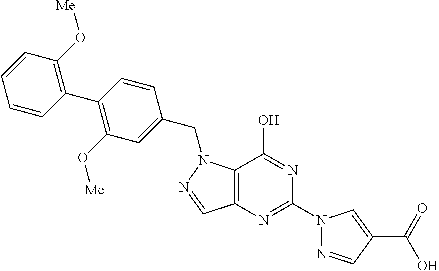 [SMILES: COc1ccccc1-c1ccc(Cn2ncc3nc(-n4cc(C(=O)O)cn4)nc(O)c32)cc1OC] | powder MS (ESI) m/z: 473 [M + H]⁺ |
| 100 | 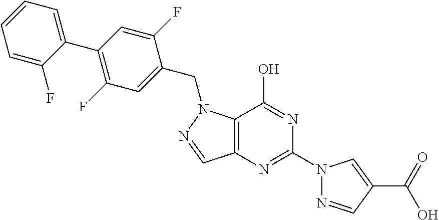 [SMILES: O=C(O)c1cnn(-c2nc(O)c3c(cnn3Cc3cc(F)c(-c4ccccc4F)cc3F)n2)c1] | powder MS (APCI) m/z: 467 [M + H]⁺ |
| 101 | 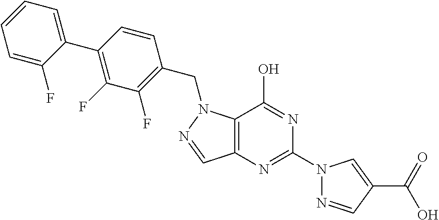 [SMILES: O=C(O)c1cnn(-c2nc(O)c3c(cnn3Cc3ccc(-c4ccccc4F)c(F)c3F)n2)c1] | powder MS (APCI) m/z: 467 [M + H]⁺ |
| 102 | 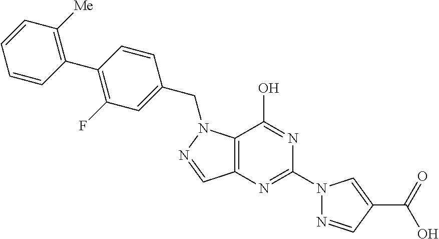 [SMILES: Cc1ccccc1-c1ccc(Cn2ncc3nc(-n4cc(C(=O)O)cn4)nc(O)c32)cc1F] | powder MS (APCI) m/z: 445 [M + H]⁺ |
| 103 | 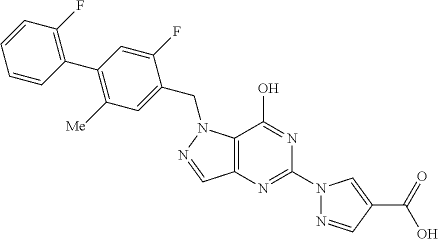 [SMILES: Cc1cc(Cn2ncc3nc(-n4cc(C(=O)O)cn4)nc(O)c32)c(F)cc1-c1ccccc1F] | powder MS (APCI) m/z: 463 [M + H]⁺ |
| 104 | 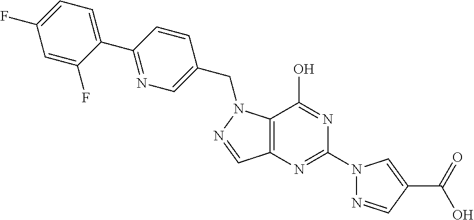 [SMILES: O=C(O)c1cnn(-c2nc(O)c3c(cnn3Cc3ccc(-c4ccc(F)cc4F)nc3)n2)c1] | powder MS (APCI) m/z: 450 [M + H]⁺ |
| 105 | 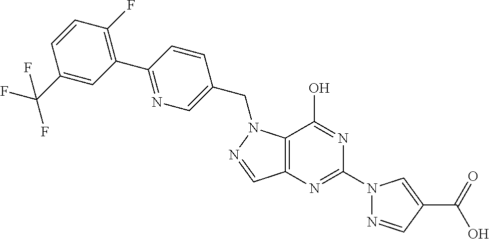 [SMILES: O=C(O)c1cnn(-c2nc(O)c3c(cnn3Cc3ccc(-c4cc(C(F)(F)F)ccc4F)nc3)n2)c1] | powder MS (APCI) m/z: 500 [M + H]⁺ |
| 106 | 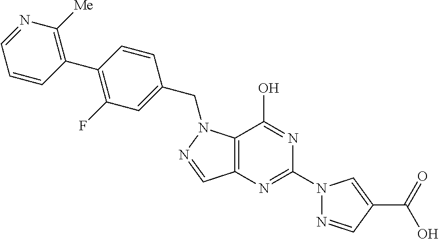 [SMILES: Cc1ncccc1-c1ccc(Cn2ncc3nc(-n4cc(C(=O)O)cn4)nc(O)c32)cc1F] | powder MS (APCI) m/z: 446 [M + H]⁺ |
| 107 | 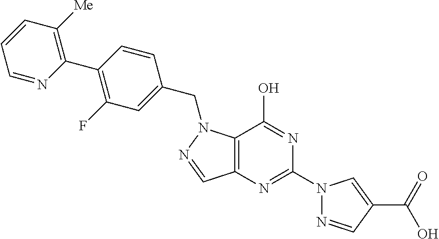 [SMILES: Cc1cccnc1-c1ccc(Cn2ncc3nc(-n4cc(C(=O)O)cn4)nc(O)c32)cc1F] | powder MS (ESI) m/z: 450 [M + H]⁺ |
| 108 | 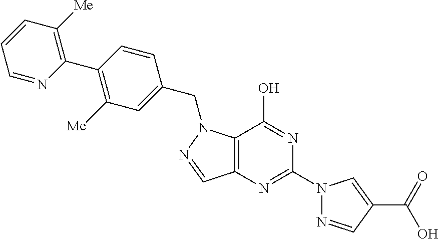 [SMILES: Cc1cc(Cn2ncc3nc(-n4cc(C(=O)O)cn4)nc(O)c32)ccc1-c1ncccc1C] | powder MS (ESI) m/z: 446 [M + H]⁺ |
| 109 | 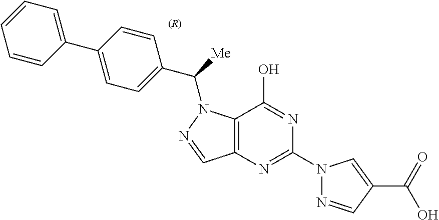 | powder MS (APCI) m/z: 427 [M + H]⁺ |
| 110 | 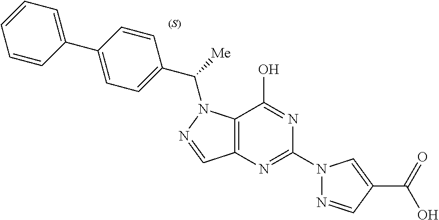 | powder MS (APCI) m/z: 427 [M + H]⁺ |
| 111 | 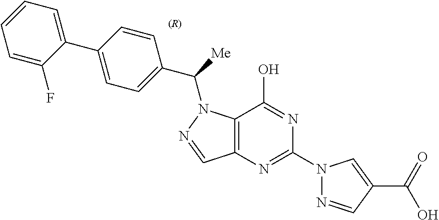 | powder MS (APCI) m/z: 445 [M + H]⁺ |
| 112 | 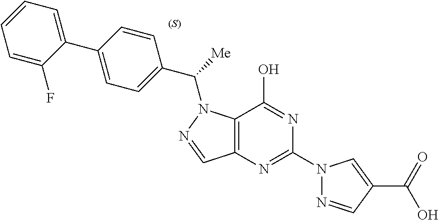 | powder MS (APCI) m/z: 445 [M + H]⁺ |
| 113 | 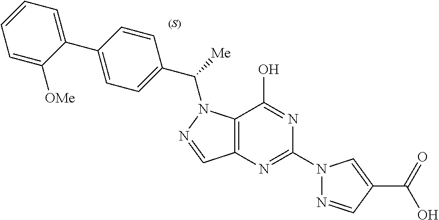 | powder MS (APCI) m/z: 457 [M + H]⁺ |
| 114 | 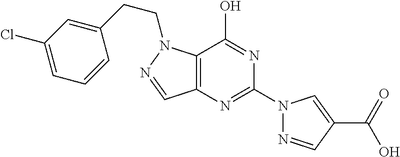 [SMILES: O=C(O)c1cnn(-c2nc(O)c3c(cnn3CCc3cccc(Cl)c3)n2)c1] | powder MS (APCI) m/z: 385/387 [M + H]⁺ |
| 115 | 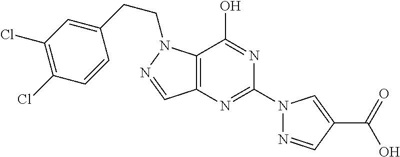 [SMILES: O=C(O)c1cnn(-c2nc(O)c3c(cnn3CCc3ccc(Cl)c(Cl)c3)n2)c1] | powder MS (APCI) m/z: 419/421 [M + H]⁺ |
| 116 | 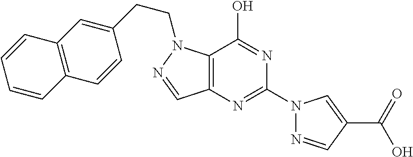 [SMILES: O=C(O)c1cnn(-c2nc(O)c3c(cnn3CCc3ccc4ccccc4c3)n2)c1] | powder MS (APCI) m/z: 401 [M + H]⁺ |
| 117 | 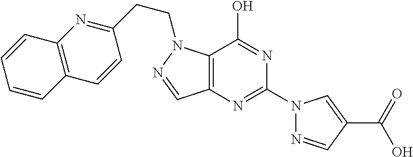 [SMILES: O=C(O)c1cnn(-c2nc(O)c3c(cnn3CCc3ccc4ccccc4n3)n2)c1] | powder MS (APCI) m/z: 402 [M + H]⁺ |
| 118 | 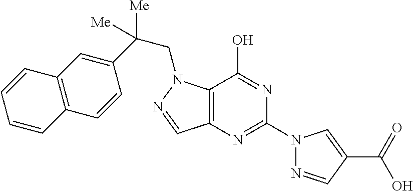 [SMILES: CC(C)(Cn1ncc2nc(-n3cc(C(=O)O)cn3)nc(O)c21)c1ccc2ccccc2c1] | powder MS (APCI) m/z: 429 [M + H]⁺ |
| 119 | 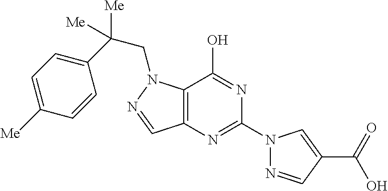 [SMILES: Cc1ccc(C(C)(C)Cn2ncc3nc(-n4cc(C(=O)O)cn4)nc(O)c32)cc1] | powder MS (APCI) m/z: 393 [M + H]⁺ |
| 120 | 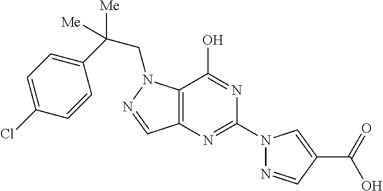 [SMILES: CC(C)(Cn1ncc2nc(-n3cc(C(=O)O)cn3)nc(O)c21)c1ccc(Cl)cc1] | powder MS (APCI) m/z: 413/415 [M + H]⁺ |
| 121 | 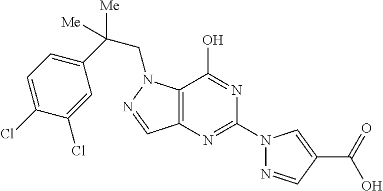 [SMILES: CC(C)(Cn1ncc2nc(-n3cc(C(=O)O)cn3)nc(O)c21)c1ccc(Cl)c(Cl)c1] | powder MS (APCI) m/z: 447/449 [M + H]⁺ |
| 122 | 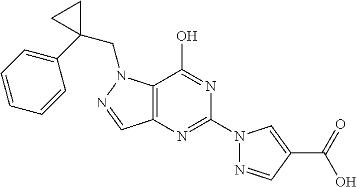 [SMILES: O=C(O)c1cnn(-c2nc(O)c3c(cnn3CC3(c4ccccc4)CC3)n2)c1] | powder MS (APCI) m/z: 377 [M + H]⁺ |
| 123 | 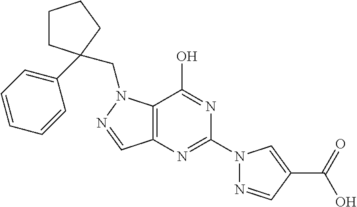 [SMILES: O=C(O)c1cnn(-c2nc(O)c3c(cnn3CC3(c4ccccc4)CCCC3)n2)c1] | powder MS (APCI) m/z: 405 [M + H]⁺ |
| 124 | 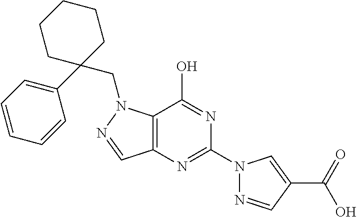 [SMILES: O=C(O)c1cnn(-c2nc(O)c3c(cnn3CC3(c4ccccc4)CCCCC3)n2)c1] | powder MS (APCI) m/z: 419 [M + H]⁺ |
| 125 | 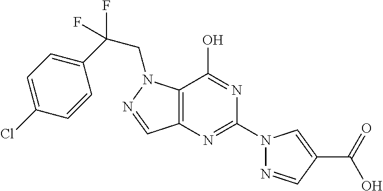 [SMILES: O=C(O)c1cnn(-c2nc(O)c3c(cnn3CC(F)(F)c3ccc(Cl)cc3)n2)c1] | powder MS (APCI) m/z: 421/423 [M + H]⁺ |
| 126 | 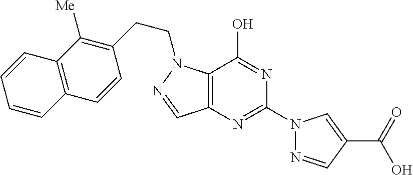 [SMILES: Cc1c(CCn2ncc3nc(-n4cc(C(=O)O)cn4)nc(O)c32)ccc2ccccc12] | powder MS (APCI) m/z: 415 [M + H]⁺ |
| 127 | 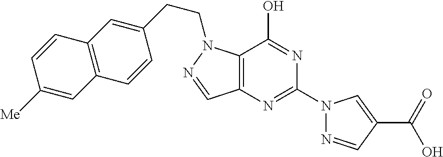 [SMILES: Cc1ccc2cc(CCn3ncc4nc(-n5cc(C(=O)O)cn5)nc(O)c43)ccc2c1] | powder MS (APCI) m/z: 415 [M + H]⁺ |
| 128 | 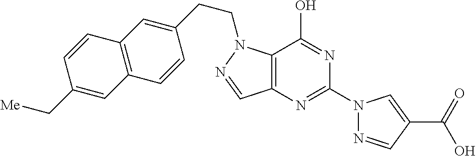 [SMILES: CCc1ccc2cc(CCn3ncc4nc(-n5cc(C(=O)O)cn5)nc(O)c43)ccc2c1] | powder MS (APCI) m/z: 429 [M + H]⁺ |
| 129 | 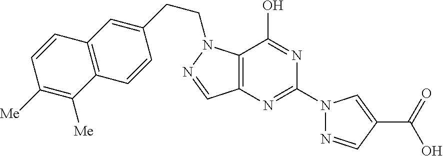 [SMILES: Cc1ccc2cc(CCn3ncc4nc(-n5cc(C(=O)O)cn5)nc(O)c43)ccc2c1C] | powder MS (ESI) m/z: 429 [M + H]⁺ |
| 130 | 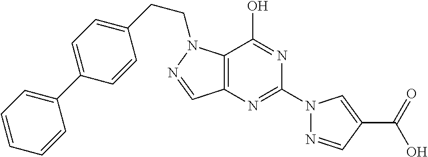 [SMILES: O=C(O)c1cnn(-c2nc(O)c3c(cnn3CCc3ccc(-c4ccccc4)cc3)n2)c1] | powder MS (APCI) m/z: 427 [M + H]⁺ |
| 131 | 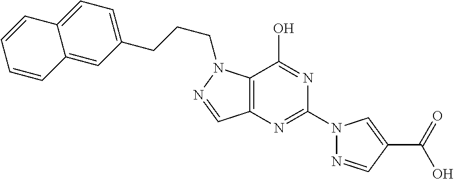 [SMILES: O=C(O)c1cnn(-c2nc(O)c3c(cnn3CCCc3ccc4ccccc4c3)n2)c1] | powder MS (APCI) m/z: 415 [M + H]⁺ |
| 132 | 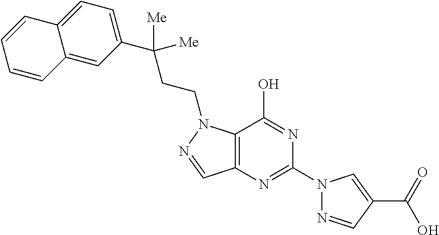 [SMILES: CC(C)(CCn1ncc2nc(-n3cc(C(=O)O)cn3)nc(O)c21)c1ccc2ccccc2c1] | powder MS (APCI) m/z: 443 [M + H]⁺ |
| 133 | 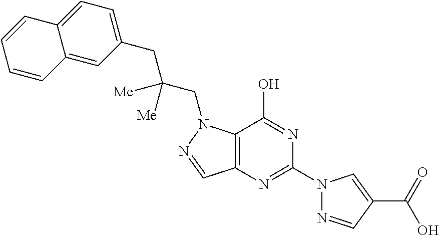 [SMILES: CC(C)(Cc1ccc2ccccc2c1)Cn1ncc2nc(-n3cc(C(=O)O)cn3)nc(O)c21] | powder MS (APCI) m/z: 443 [M + H]⁺ |
| 134 | 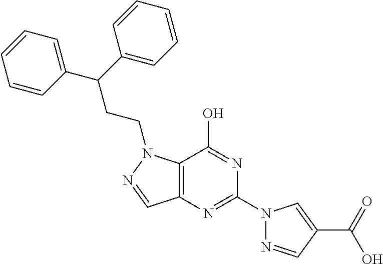 [SMILES: O=C(O)c1cnn(-c2nc(O)c3c(cnn3CCC(c3ccccc3)c3ccccc3)n2)c1] | powder MS (APCI) m/z: 441 [M + H]⁺ |
| 135 | 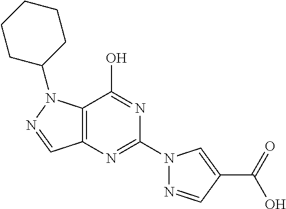 [SMILES: O=C(O)c1cnn(-c2nc(O)c3c(cnn3C3CCCCC3)n2)c1] | powder MS (APCI) m/z: 329 [M + H]⁺ |
| 136 | 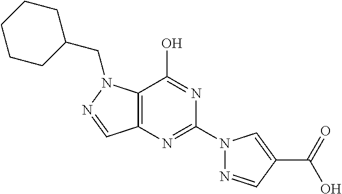 [SMILES: O=C(O)c1cnn(-c2nc(O)c3c(cnn3CC3CCCCC3)n2)c1] | powder MS (APCI) m/z: 343 [M + H]⁺ |
| 137 | 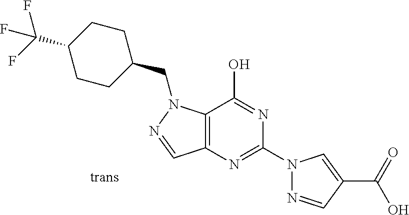 [SMILES: O=C(O)c1cnn(-c2nc(O)c3c(cnn3C[C@H]3CC[C@H](C(F)(F)F)CC3)n2)c1] | powder MS (APCI) m/z: 411 [M + H]⁺ |
| 138 | 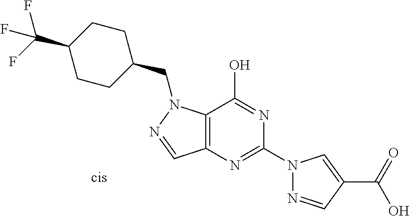 [SMILES: O=C(O)c1cnn(-c2nc(O)c3c(cnn3C[C@H]3CC[C@@H](C(F)(F)F)CC3)n2)c1] | powder MS (APCI) m/z: 411 [M + H]⁺ |
| 139 | 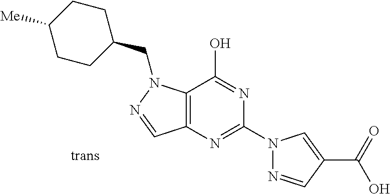 [SMILES: C[C@H]1CC[C@H](Cn2ncc3nc(-n4cc(C(=O)O)cn4)nc(O)c32)CC1] | powder MS (APCI) m/z: 357 [M + H]⁺ |
| 140 | 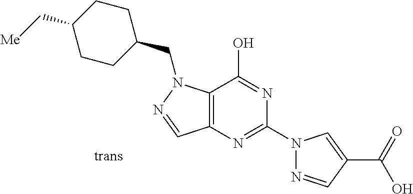 [SMILES: CC[C@H]1CC[C@H](Cn2ncc3nc(-n4cc(C(=O)O)cn4)nc(O)c32)CC1] | powder MS (APCI) m/z: 371 [M + H]⁺ |
| 141 | 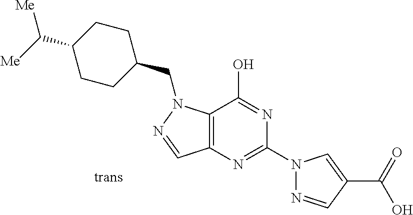 [SMILES: CC(C)[C@H]1CC[C@H](Cn2ncc3nc(-n4cc(C(=O)O)cn4)nc(O)c32)CC1] | powder MS (APCI) m/z: 385 [M + H]⁺ |
| 142 | 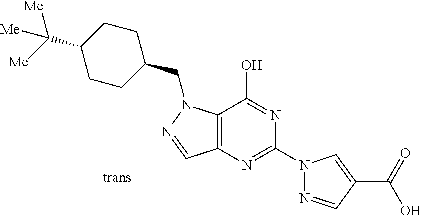 [SMILES: CC(C)(C)[C@H]1CC[C@H](Cn2ncc3nc(-n4cc(C(=O)O)cn4)nc(O)c32)CC1] | powder MS (APCI) m/z: 399 [M + H]⁺ |
| 143 | 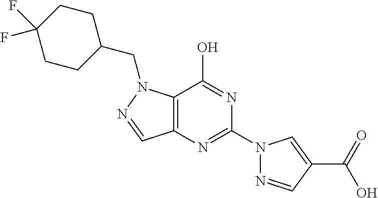 [SMILES: O=C(O)c1cnn(-c2nc(O)c3c(cnn3CC3CCC(F)(F)CC3)n2)c1] | powder MS (APCI) m/z: 379 [M + H]⁺ |
| 144 | 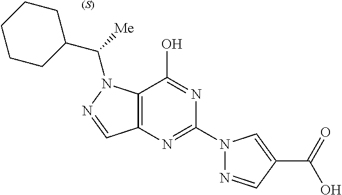 | powder MS (APCI) m/z: 357 [M + H]⁺ |
| 145 | 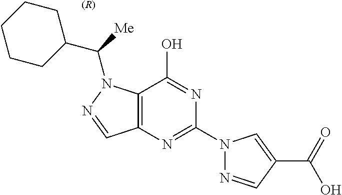 | powder MS (APCI) m/z: 357 [M + H]⁺ |
| 146 | 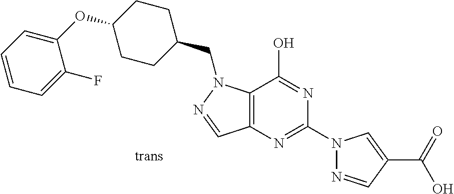 [SMILES: O=C(O)c1cnn(-c2nc(O)c3c(cnn3C[C@H]3CC[C@H](Oc4ccccc4F)CC3)n2)c1] | powder MS (APCI) m/z: 453 [M + H]⁺ |
| 147 | 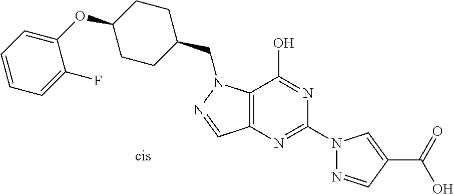 [SMILES: O=C(O)c1cnn(-c2nc(O)c3c(cnn3C[C@H]3CC[C@@H](Oc4ccccc4F)CC3)n2)c1] | powder MS (APCI) m/z: 453 [M + H]⁺ |
| 148 | 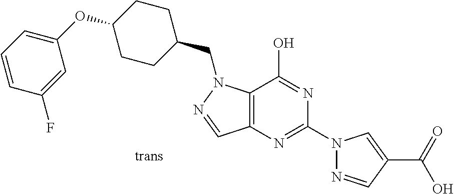 [SMILES: O=C(O)c1cnn(-c2nc(O)c3c(cnn3C[C@H]3CC[C@H](Oc4cccc(F)c4)CC3)n2)c1] | powder MS (APCI) m/z: 453 [M + H]⁺ |
| 149 | 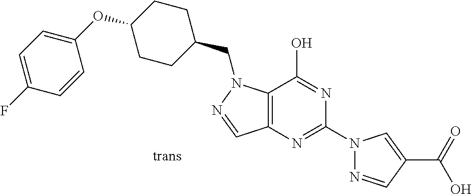 [SMILES: O=C(O)c1cnn(-c2nc(O)c3c(cnn3C[C@H]3CC[C@H](Oc4ccc(F)cc4)CC3)n2)c1] | powder MS (APCI) m/z: 453 [M + H]⁺ |
| 150 | 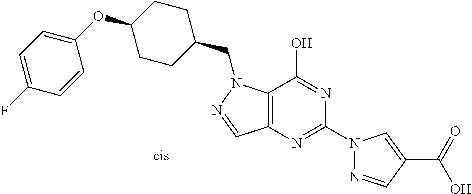 [SMILES: O=C(O)c1cnn(-c2nc(O)c3c(cnn3C[C@H]3CC[C@@H](Oc4ccc(F)cc4)CC3)n2)c1] | powder MS (APCI) m/z: 453 [M + H]⁺ |
| 151 | 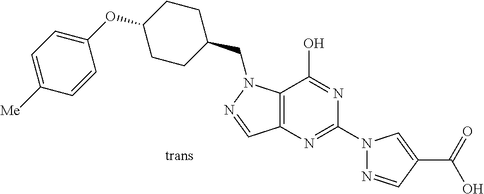 [SMILES: Cc1ccc(O[C@H]2CC[C@H](Cn3ncc4nc(-n5cc(C(=O)O)cn5)nc(O)c43)CC2)cc1] | powder MS (APCI) m/z: 449 [M + H]⁺ |
| 152 | 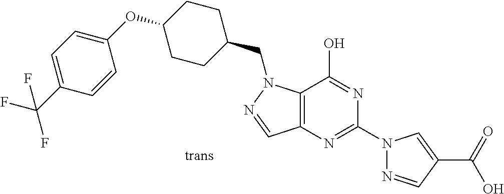 [SMILES: O=C(O)c1cnn(-c2nc(O)c3c(cnn3C[C@H]3CC[C@H](Oc4ccc(C(F)(F)F)cc4)CC3)n2)c1] | powder MS (APCI) m/z: 503 [M + H]⁺ |
| 153 | 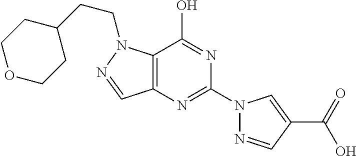 [SMILES: O=C(O)c1cnn(-c2nc(O)c3c(cnn3CCC3CCOCC3)n2)c1] | powder MS (APCI) m/z: 359 [M + H]⁺ |
| 154 | 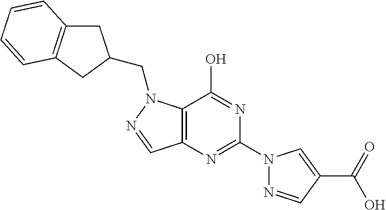 [SMILES: O=C(O)c1cnn(-c2nc(O)c3c(cnn3CC3Cc4ccccc4C3)n2)c1] | powder MS (APCI) m/z: 377 [M + H]⁺ |
| 155 | 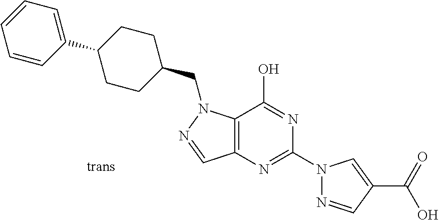 [SMILES: O=C(O)c1cnn(-c2nc(O)c3c(cnn3C[C@H]3CC[C@H](c4ccccc4)CC3)n2)c1] | powder MS (APCI) m/z: 419 [M + H]⁺ |
| 156 | 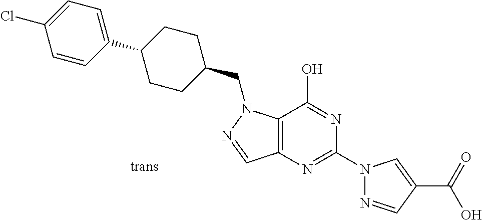 [SMILES: O=C(O)c1cnn(-c2nc(O)c3c(cnn3C[C@H]3CC[C@H](c4ccc(Cl)cc4)CC3)n2)c1] | powder MS (APCI) m/z: 453/455 [M + H]⁺ |
| 157 | 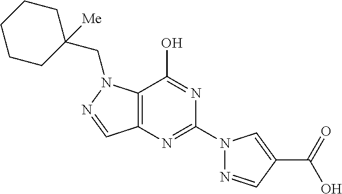 [SMILES: CC1(Cn2ncc3nc(-n4cc(C(=O)O)cn4)nc(O)c32)CCCCC1] | powder MS (APCI) m/z: 357 [M + H]⁺ |
| 158 | 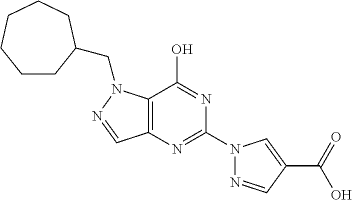 [SMILES: O=C(O)c1cnn(-c2nc(O)c3c(cnn3CC3CCCCCC3)n2)c1] | powder MS (APCI) m/z: 357 [M + H]⁺ |
| 159 | 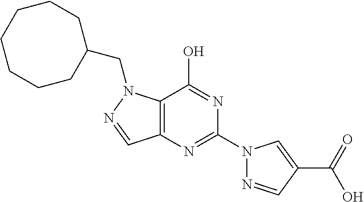 [SMILES: O=C(O)c1cnn(-c2nc(O)c3c(cnn3CC3CCCCCCC3)n2)c1] | powder MS (APCI) m/z: 371 [M + H]⁺ |
| 160 | 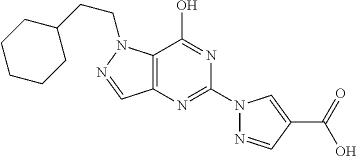 [SMILES: O=C(O)c1cnn(-c2nc(O)c3c(cnn3CCC3CCCCC3)n2)c1] | powder MS (APCI) m/z: 357 [M + H]⁺ |
| 161 | 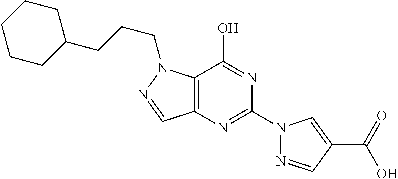 [SMILES: O=C(O)c1cnn(-c2nc(O)c3c(cnn3CCCC3CCCCC3)n2)c1] | powder MS (APCI) m/z: 371 [M + H]⁺ |
| 162 | 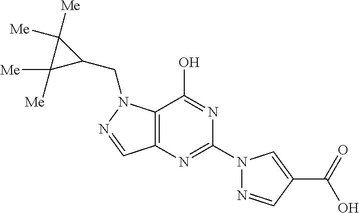 [SMILES: CC1(C)C(Cn2ncc3nc(-n4cc(C(=O)O)cn4)nc(O)c32)C1(C)C] | powder MS (APCI) m/z: 357 [M + H]⁺ |
| 163 | 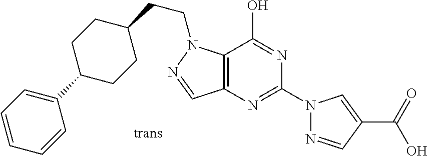 [SMILES: O=C(O)c1cnn(-c2nc(O)c3c(cnn3CC[C@H]3CC[C@H](c4ccccc4)CC3)n2)c1] | powder MS (APCI) m/z: 433 [M + H]⁺ |
| 164 | 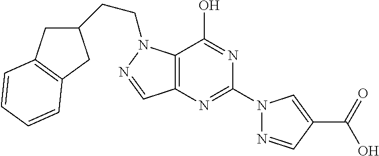 [SMILES: O=C(O)c1cnn(-c2nc(O)c3c(cnn3CCC3Cc4ccccc4C3)n2)c1] | powder MS (APCI) m/z: 391 [M + H]⁺ |
| 165 | 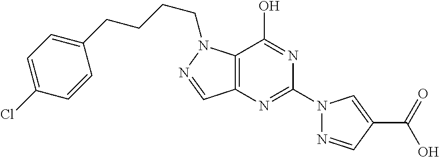 [SMILES: O=C(O)c1cnn(-c2nc(O)c3c(cnn3CCCCc3ccc(Cl)cc3)n2)c1] | powder MS (APCI) m/z: 413/415 [M + H]⁺ |
| 166 | 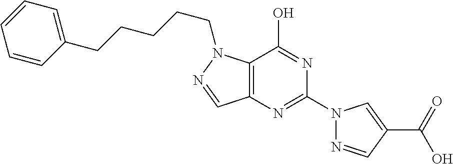 [SMILES: O=C(O)c1cnn(-c2nc(O)c3c(cnn3CCCCCc3ccccc3)n2)c1] | powder MS (APCI) m/z: 393 [M + H]⁺ |
| 167 | 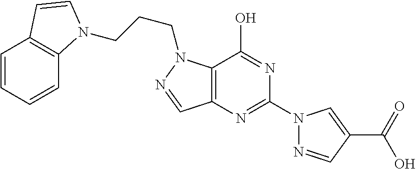 [SMILES: O=C(O)c1cnn(-c2nc(O)c3c(cnn3CCCn3ccc4ccccc43)n2)c1] | powder MS (ESI) m/z: 404 [M + H]⁺ |
| 168 | 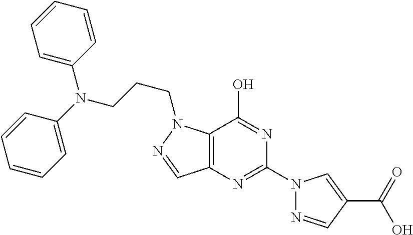 [SMILES: O=C(O)c1cnn(-c2nc(O)c3c(cnn3CCCN(c3ccccc3)c3ccccc3)n2)c1] | powder MS (ESI) m/z: 456 [M + H]⁺ |
| 169 | 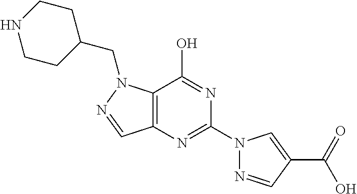 [SMILES: O=C(O)c1cnn(-c2nc(O)c3c(cnn3CC3CCNCC3)n2)c1] | powder MS (APCI) m/z: 344 [M + H]⁺ |
| 170 | 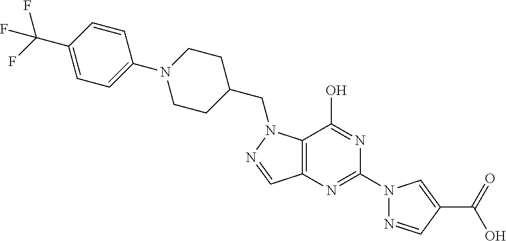 [SMILES: O=C(O)c1cnn(-c2nc(O)c3c(cnn3CC3CCN(c4ccc(C(F)(F)F)cc4)CC3)n2)c1] | powder MS (APCI) m/z: 488 [M + H]⁺ |
| 171 | 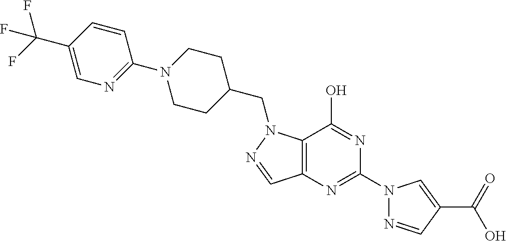 [SMILES: O=C(O)c1cnn(-c2nc(O)c3c(cnn3CC3CCN(c4ccc(C(F)(F)F)cn4)CC3)n2)c1] | powder MS (APCI) m/z: 489 [M + H]⁺ |
| 172 | 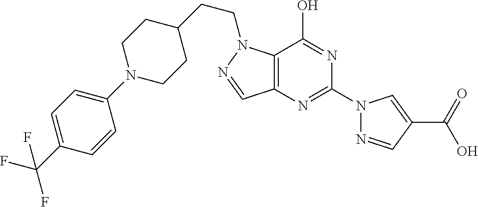 [SMILES: O=C(O)c1cnn(-c2nc(O)c3c(cnn3CCC3CCN(c4ccc(C(F)(F)F)cc4)CC3)n2)c1] | powder MS (APCI) m/z: 502 [M + H]⁺ |
| 173 | 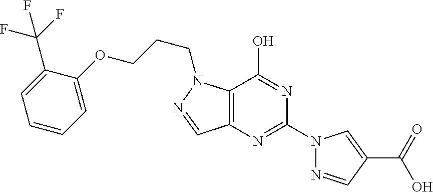 [SMILES: O=C(O)c1cnn(-c2nc(O)c3c(cnn3CCCOc3ccccc3C(F)(F)F)n2)c1] | powder MS (ESI) m/z: 449 [M + H]⁺ |
| 174 | 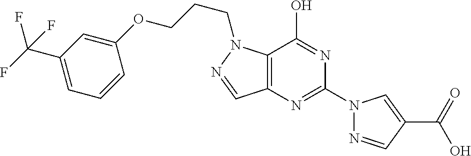 [SMILES: O=C(O)c1cnn(-c2nc(O)c3c(cnn3CCCOc3cccc(C(F)(F)F)c3)n2)c1] | powder MS (APCI) m/z: 449 [M + H]⁺ |
| 175 | 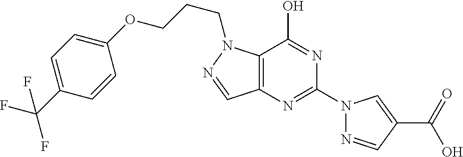 [SMILES: O=C(O)c1cnn(-c2nc(O)c3c(cnn3CCCOc3ccc(C(F)(F)F)cc3)n2)c1] | powder MS (APCI) m/z: 449 [M + H]⁺ |
| 176 | 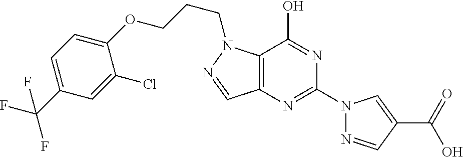 [SMILES: O=C(O)c1cnn(-c2nc(O)c3c(cnn3CCCOc3ccc(C(F)(F)F)cc3Cl)n2)c1] | powder MS (APCI) m/z: 483/485 [M + H]⁺ |
| 177 | 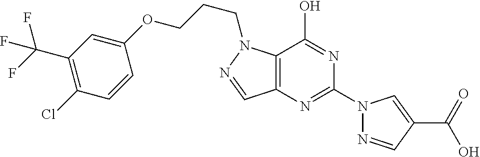 [SMILES: O=C(O)c1cnn(-c2nc(O)c3c(cnn3CCCOc3ccc(Cl)c(C(F)(F)F)c3)n2)c1] | powder MS (APCI) m/z: 483/485 [M + H]⁺ |
| 178 | 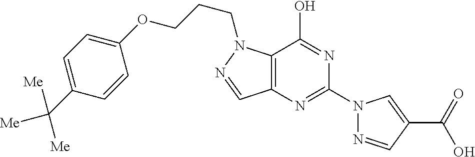 [SMILES: CC(C)(C)c1ccc(OCCCn2ncc3nc(-n4cc(C(=O)O)cn4)nc(O)c32)cc1] | powder MS (APCI) m/z: 437 [M + H]⁺ |
| 179 | 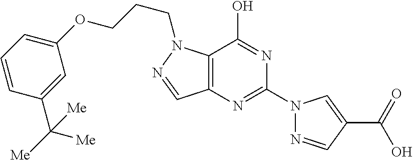 [SMILES: CC(C)(C)c1cccc(OCCCn2ncc3nc(-n4cc(C(=O)O)cn4)nc(O)c32)c1] | powder MS (APCI) m/z: 437 [M + H]⁺ |
| 180 | 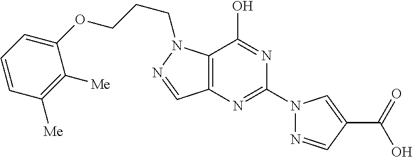 [SMILES: Cc1cccc(OCCCn2ncc3nc(-n4cc(C(=O)O)cn4)nc(O)c32)c1C] | powder MS (APCI) m/z: 409 [M + H]⁺ |
| 181 | 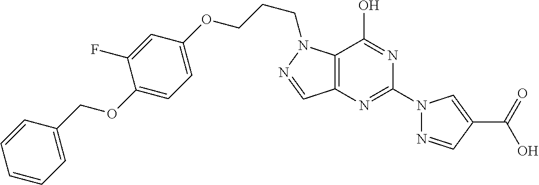 [SMILES: O=C(O)c1cnn(-c2nc(O)c3c(cnn3CCCOc3ccc(OCc4ccccc4)c(F)c3)n2)c1] | powder MS (ESI) m/z: 505 [M + H]⁺ |
| 182 | 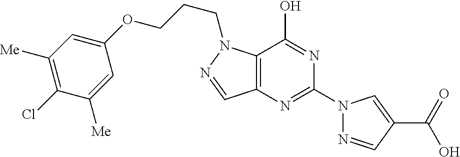 [SMILES: Cc1cc(OCCCn2ncc3nc(-n4cc(C(=O)O)cn4)nc(O)c32)cc(C)c1Cl] | powder MS (APCI) m/z: 443/445 [M + H]⁺ |
| 183 | 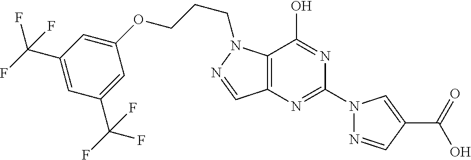 [SMILES: O=C(O)c1cnn(-c2nc(O)c3c(cnn3CCCOc3cc(C(F)(F)F)cc(C(F)(F)F)c3)n2)c1] | powder MS (APCI) m/z: 517 [M + H]⁺ |
| 184 | 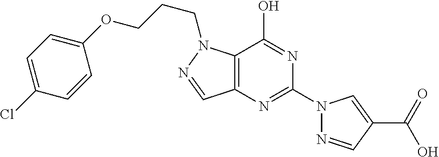 [SMILES: O=C(O)c1cnn(-c2nc(O)c3c(cnn3CCCOc3ccc(Cl)cc3)n2)c1] | powder MS (APCI) m/z: 415/417 [M + H]⁺ |
| 185 | 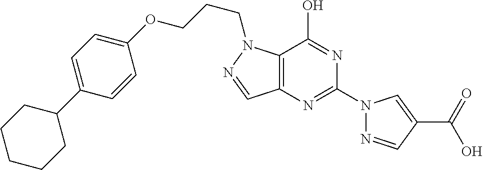 [SMILES: O=C(O)c1cnn(-c2nc(O)c3c(cnn3CCCOc3ccc(C4CCCCC4)cc3)n2)c1] | powder MS (APCI) m/z: 463 [M + H]⁺ |
| 186 | 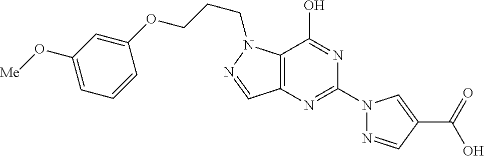 [SMILES: COc1cccc(OCCCn2ncc3nc(-n4cc(C(=O)O)cn4)nc(O)c32)c1] | powder MS (APCI) m/z: 411 [M + H]⁺ |
| 187 | 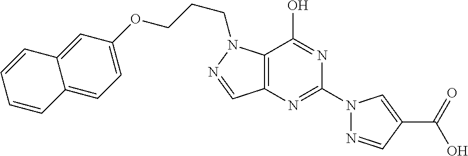 [SMILES: O=C(O)c1cnn(-c2nc(O)c3c(cnn3CCCOc3ccc4ccccc4c3)n2)c1] | powder MS (ESI) m/z: 431 [M + H]⁺ |
| 188 | 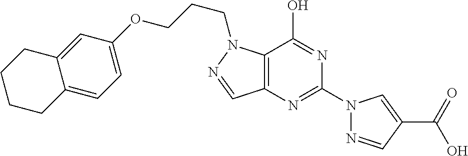 [SMILES: O=C(O)c1cnn(-c2nc(O)c3c(cnn3CCCOc3ccc4c(c3)CCCC4)n2)c1] | powder MS (APCI) m/z: 435 [M + H]⁺ |
| 189 | 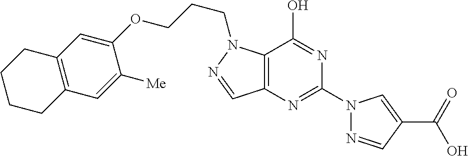 [SMILES: Cc1cc2c(cc1OCCCn1ncc3nc(-n4cc(C(=O)O)cn4)nc(O)c31)CCCC2] | powder MS (APCI) m/z: 449 [M + H]⁺ |
| 190 | 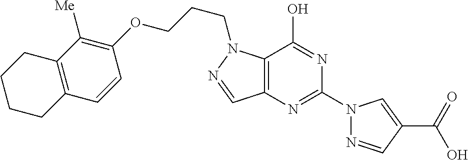 [SMILES: Cc1c(OCCCn2ncc3nc(-n4cc(C(=O)O)cn4)nc(O)c32)ccc2c1CCCC2] | powder MS (ESI) m/z: 447 [M − H]⁻ |
| 191 | 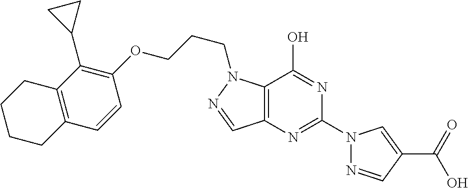 [SMILES: O=C(O)c1cnn(-c2nc(O)c3c(cnn3CCCOc3ccc4c(c3C3CC3)CCCC4)n2)c1] | powder MS (APCI) m/z: 475 [M + H]⁺ |
| 192 | 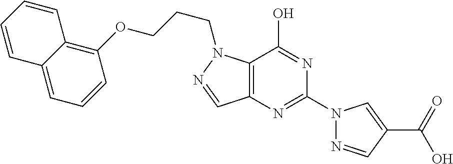 [SMILES: O=C(O)c1cnn(-c2nc(O)c3c(cnn3CCCOc3cccc4ccccc34)n2)c1] | powder MS (APCI) m/z: 431 [M + H]⁺ |
| 193 | 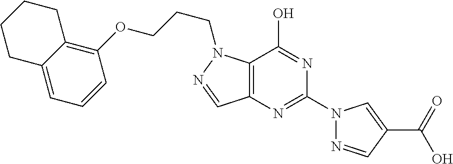 [SMILES: O=C(O)c1cnn(-c2nc(O)c3c(cnn3CCCOc3cccc4c3CCCC4)n2)c1] | powder MS (APCI) m/z: 435 [M + H]⁺ |
| 194 | 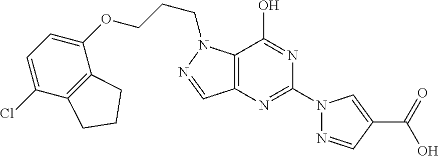 [SMILES: O=C(O)c1cnn(-c2nc(O)c3c(cnn3CCCOc3ccc(Cl)c4c3CCC4)n2)c1] | powder MS (APCI) m/z: 455/457 [M + H]⁺ |
| 195 | 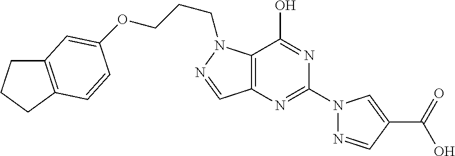 [SMILES: O=C(O)c1cnn(-c2nc(O)c3c(cnn3CCCOc3ccc4c(c3)CCC4)n2)c1] | powder MS (APCI) m/z: 421 [M + H]⁺ |
| 196 | 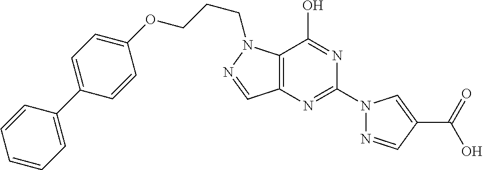 [SMILES: O=C(O)c1cnn(-c2nc(O)c3c(cnn3CCCOc3ccc(-c4ccccc4)cc3)n2)c1] | powder MS (APCI) m/z: 457 [M + H]⁺ |
| 197 | 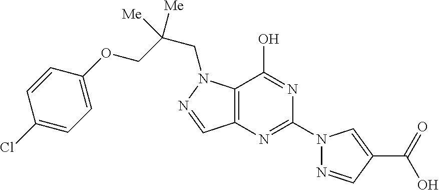 [SMILES: CC(C)(COc1ccc(Cl)cc1)Cn1ncc2nc(-n3cc(C(=O)O)cn3)nc(O)c21] | powder MS (APCI) m/z: 443/445 [M + H]⁺ |
| 198 | 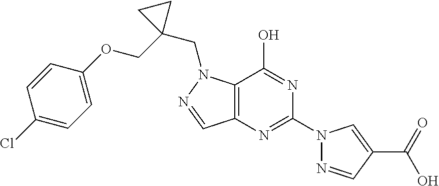 [SMILES: O=C(O)c1cnn(-c2nc(O)c3c(cnn3CC3(COc4ccc(Cl)cc4)CC3)n2)c1] | powder MS (APCI) m/z: 441/443 [M + H]⁺ |
| 199 | 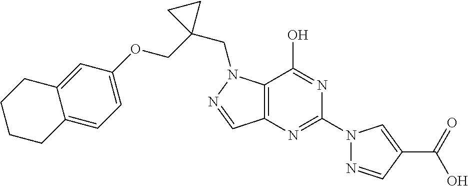 [SMILES: O=C(O)c1cnn(-c2nc(O)c3c(cnn3CC3(COc4ccc5c(c4)CCCC5)CC3)n2)c1] | powder MS (APCI) m/z: 461 [M + H]⁺ |
| 200 | 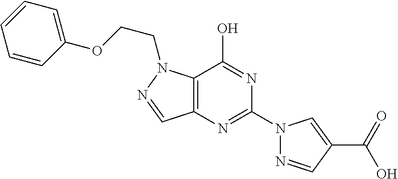 [SMILES: O=C(O)c1cnn(-c2nc(O)c3c(cnn3CCOc3ccccc3)n2)c1] | powder MS (APCI) m/z: 367 [M + H]⁺ |
| 201 | 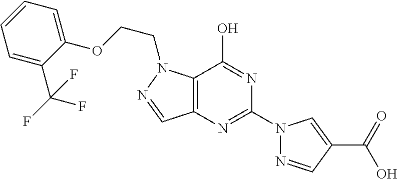 [SMILES: O=C(O)c1cnn(-c2nc(O)c3c(cnn3CCOc3ccccc3C(F)(F)F)n2)c1] | powder MS (APCI) m/z: 435 [M + H]⁺ |
| 202 | 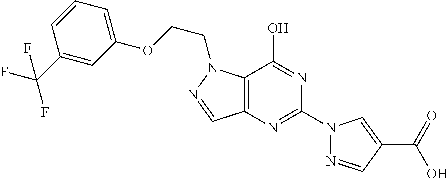 [SMILES: O=C(O)c1cnn(-c2nc(O)c3c(cnn3CCOc3cccc(C(F)(F)F)c3)n2)c1] | powder MS (APCI) m/z: 435 [M + H]⁺ |
| 203 | 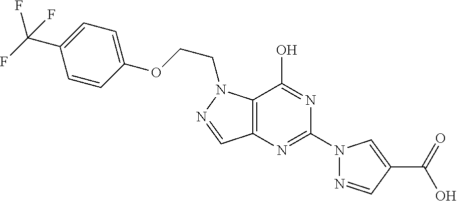 [SMILES: O=C(O)c1cnn(-c2nc(O)c3c(cnn3CCOc3ccc(C(F)(F)F)cc3)n2)c1] | powder MS (APCI) m/z: 435 [M + H]⁺ |
| 204 | 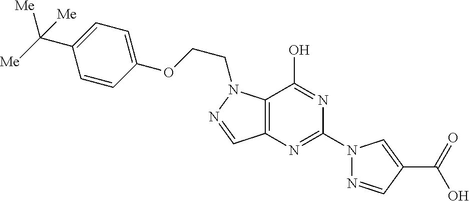 [SMILES: CC(C)(C)c1ccc(OCCn2ncc3nc(-n4cc(C(=O)O)cn4)nc(O)c32)cc1] | powder MS (APCI) m/z: 423 [M + H]⁺ |
| 205 | 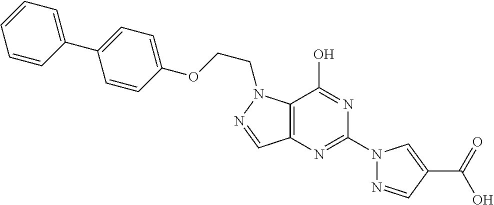 [SMILES: O=C(O)c1cnn(-c2nc(O)c3c(cnn3CCOc3ccc(-c4ccccc4)cc3)n2)c1] | powder MS (APCI) m/z: 443 [M + H]⁺ |
| 206 | 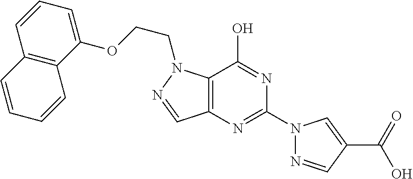 [SMILES: O=C(O)c1cnn(-c2nc(O)c3c(cnn3CCOc3cccc4ccccc34)n2)c1] | powder MS (APCI) m/z: 417 [M + H]⁺ |
| 207 | 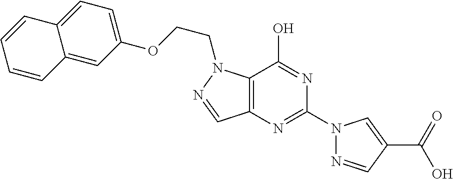 [SMILES: O=C(O)c1cnn(-c2nc(O)c3c(cnn3CCOc3ccc4ccccc4c3)n2)c1] | powder MS (APCI) m/z: 417 [M + H]⁺ |
| 208 | 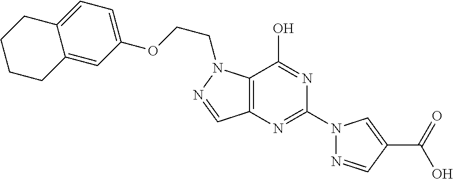 [SMILES: O=C(O)c1cnn(-c2nc(O)c3c(cnn3CCOc3ccc4c(c3)CCCC4)n2)c1] | powder MS (APCI) m/z: 421 [M + H]⁺ |
| 209 | 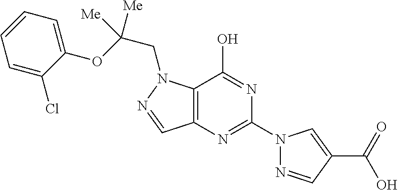 [SMILES: CC(C)(Cn1ncc2nc(-n3cc(C(=O)O)cn3)nc(O)c21)Oc1ccccc1Cl] | powder MS (APCI) m/z: 429/431 [M + H]⁺ |
| 210 | 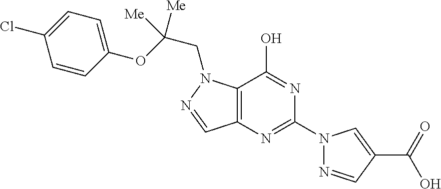 [SMILES: CC(C)(Cn1ncc2nc(-n3cc(C(=O)O)cn3)nc(O)c21)Oc1ccc(Cl)cc1] | powder MS (APCI) m/z: 428/430 [M + H]⁺ |
| 211 | 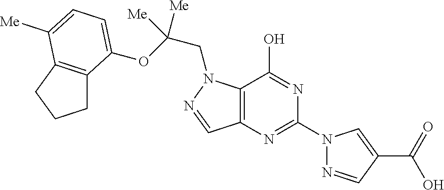 [SMILES: Cc1ccc(OC(C)(C)Cn2ncc3nc(-n4cc(C(=O)O)cn4)nc(O)c32)c2c1CCC2] | powder MS (ESI) m/z: 449 [M + H]⁺ |
| 212 | 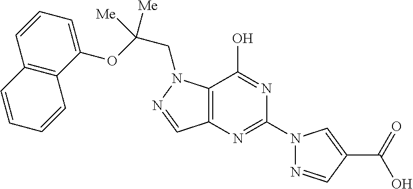 [SMILES: CC(C)(Cn1ncc2nc(-n3cc(C(=O)O)cn3)nc(O)c21)Oc1cccc2ccccc12] | powder MS (APCI) m/z: 445 [M + H]⁺ |
| 213 | 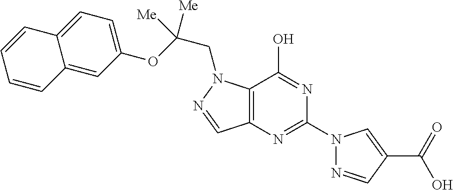 [SMILES: CC(C)(Cn1ncc2nc(-n3cc(C(=O)O)cn3)nc(O)c21)Oc1ccc2ccccc2c1] | powder MS (APCI) m/z: 445 [M + H]⁺ |
| 214 | 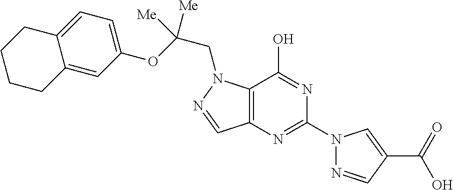 [SMILES: CC(C)(Cn1ncc2nc(-n3cc(C(=O)O)cn3)nc(O)c21)Oc1ccc2c(c1)CCCC2] | powder MS (APCI) m/z: 449 [M + H]⁺ |
| 215 | 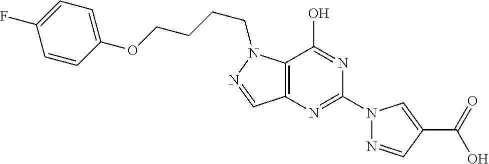 [SMILES: O=C(O)c1cnn(-c2nc(O)c3c(cnn3CCCCOc3ccc(F)cc3)n2)c1] | powder MS (APCI) m/z: 413 [M + H]⁺ |
| 216 | 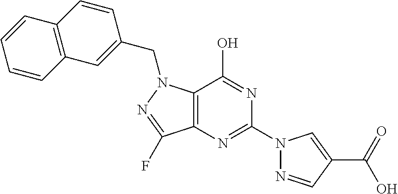 [SMILES: O=C(O)c1cnn(-c2nc(O)c3c(n2)c(F)nn3Cc2ccc3ccccc3c2)c1] | powder MS (APCI) m/z: 405 [M + H]⁺ |
| 217 | 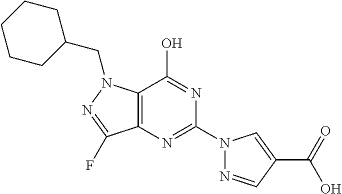 [SMILES: O=C(O)c1cnn(-c2nc(O)c3c(n2)c(F)nn3CC2CCCCC2)c1] | powder MS (APCI) m/z: 361 [M + H]⁺ |
| 218 | 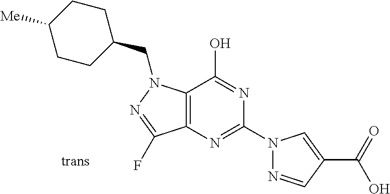 [SMILES: C[C@H]1CC[C@H](Cn2nc(F)c3nc(-n4cc(C(=O)O)cn4)nc(O)c32)CC1] | powder MS (APCI) m/z: 375 [M + H]⁺ |
| 219 | 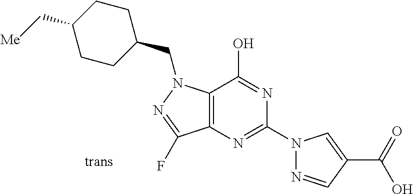 [SMILES: CC[C@H]1CC[C@H](Cn2nc(F)c3nc(-n4cc(C(=O)O)cn4)nc(O)c32)CC1] | powder MS (APCI) m/z: 389 [M + H]⁺ |
| 220 | 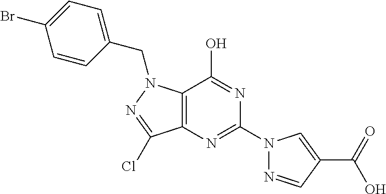 [SMILES: O=C(O)c1cnn(-c2nc(O)c3c(n2)c(Cl)nn3Cc2ccc(Br)cc2)c1] | powder MS (APCI) m/z: 449/451 [M + H]⁺ |
| 221 | 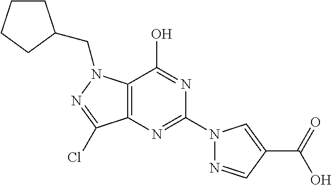 [SMILES: O=C(O)c1cnn(-c2nc(O)c3c(n2)c(Cl)nn3CC2CCCC2)c1] | powder MS (APCI) m/z: 363/365 [M + H]⁺ |
| 222 | 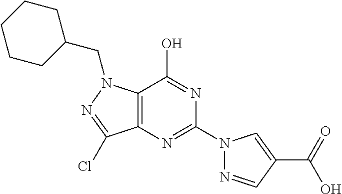 [SMILES: O=C(O)c1cnn(-c2nc(O)c3c(n2)c(Cl)nn3CC2CCCCC2)c1] | powder MS (APCI) m/z: 377/379 [M + H]⁺ |
| 223 | 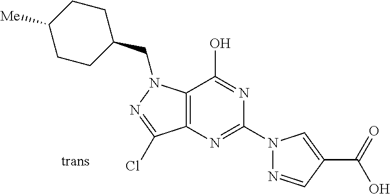 [SMILES: C[C@H]1CC[C@H](Cn2nc(Cl)c3nc(-n4cc(C(=O)O)cn4)nc(O)c32)CC1] | powder MS (APCI) m/z: 391/393 [M + H]⁺ |
| 224 | 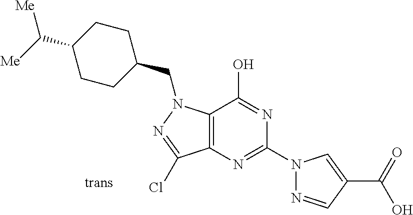 [SMILES: CC(C)[C@H]1CC[C@H](Cn2nc(Cl)c3nc(-n4cc(C(=O)O)cn4)nc(O)c32)CC1] | powder MS (APCI) m/z: 419/421 [M + H]⁺ |
| 225 | 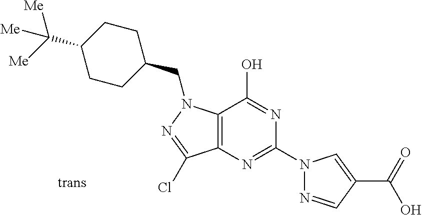 [SMILES: CC(C)(C)[C@H]1CC[C@H](Cn2nc(Cl)c3nc(-n4cc(C(=O)O)cn4)nc(O)c32)CC1] | powder MS (APCI) m/z: 433/435 [M + H]⁺ |
| 226 | 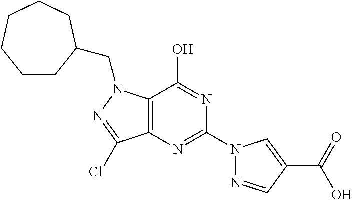 [SMILES: O=C(O)c1cnn(-c2nc(O)c3c(n2)c(Cl)nn3CC2CCCCCC2)c1] | powder MS (APCI) m/z: 391/393 [M + H]⁺ |
| 227 | 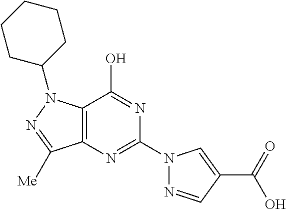 [SMILES: Cc1nn(C2CCCCC2)c2c(O)nc(-n3cc(C(=O)O)cn3)nc12] | powder MS (APCI) m/z: 343 [M + H]⁺ |
| 228 | 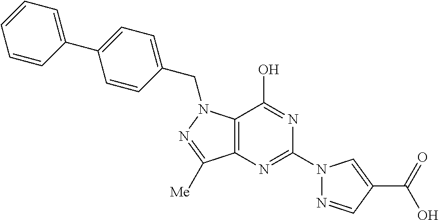 [SMILES: Cc1nn(Cc2ccc(-c3ccccc3)cc2)c2c(O)nc(-n3cc(C(=O)O)cn3)nc12] | powder MS (APCI) m/z: 427 [M + H]⁺ |
| 229 | 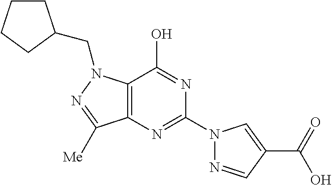 [SMILES: Cc1nn(CC2CCCC2)c2c(O)nc(-n3cc(C(=O)O)cn3)nc12] | powder MS (APCI) m/z: 343 [M + H]⁺ |
| 230 | 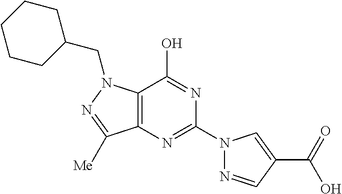 [SMILES: Cc1nn(CC2CCCCC2)c2c(O)nc(-n3cc(C(=O)O)cn3)nc12] | powder MS (APCI) m/z: 357 [M + H]⁺ |
| 231 | 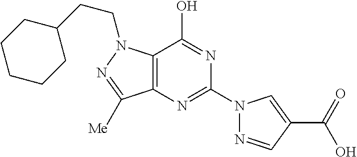 [SMILES: Cc1nn(CCC2CCCCC2)c2c(O)nc(-n3cc(C(=O)O)cn3)nc12] | powder MS (APCI) m/z: 371 [M + H]⁺ |
| 232 | 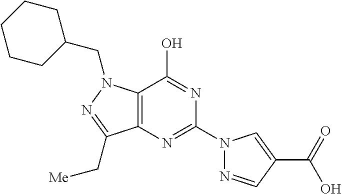 [SMILES: CCc1nn(CC2CCCCC2)c2c(O)nc(-n3cc(C(=O)O)cn3)nc12] | powder MS (APCI) m/z: 371 [M + H]⁺ |
| 233 | 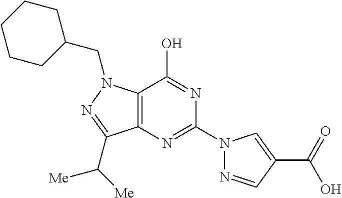 [SMILES: CC(C)c1nn(CC2CCCCC2)c2c(O)nc(-n3cc(C(=O)O)cn3)nc12] | powder MS (APCI) m/z: 385 [M + H]⁺ |
| 234 | 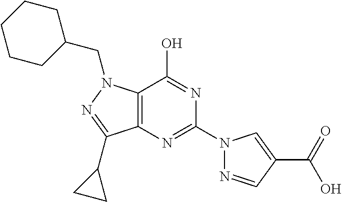 [SMILES: O=C(O)c1cnn(-c2nc(O)c3c(n2)c(C2CC2)nn3CC2CCCCC2)c1] | powder MS (APCI) m/z: 383 [M + H]⁺ |
| 235 | 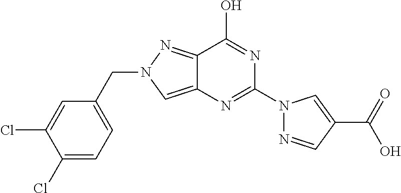 [SMILES: O=C(O)c1cnn(-c2nc(O)c3nn(Cc4ccc(Cl)c(Cl)c4)cc3n2)c1] | powder MS (APCI) m/z: 405/407 [M + H]⁺ |
| 236 | 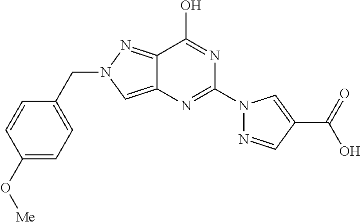 [SMILES: COc1ccc(Cn2cc3nc(-n4cc(C(=O)O)cn4)nc(O)c3n2)cc1] | powder MS (APCI) m/z: 367 [M + H]⁺ |
| 237 | 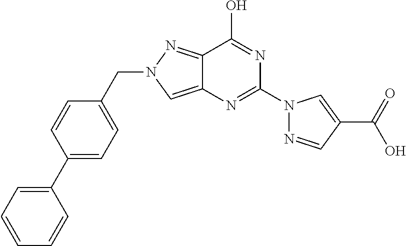 [SMILES: O=C(O)c1cnn(-c2nc(O)c3nn(Cc4ccc(-c5ccccc5)cc4)cc3n2)c1] | powder MS (APCI) m/z: 413 [M + H]⁺ |
| 238 | 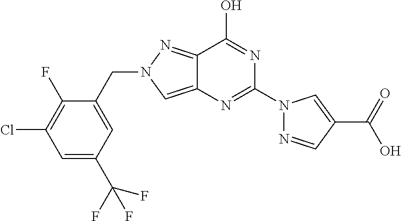 [SMILES: O=C(O)c1cnn(-c2nc(O)c3nn(Cc4cc(C(F)(F)F)cc(Cl)c4F)cc3n2)c1] | powder MS (APCI) m/z: 457/459 [M + H]⁺ |
| 239 | 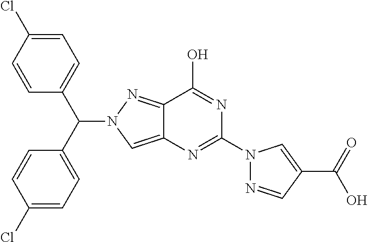 [SMILES: O=C(O)c1cnn(-c2nc(O)c3nn(C(c4ccc(Cl)cc4)c4ccc(Cl)cc4)cc3n2)c1] | powder MS (APCI) m/z: 481/483 [M + H]⁺ |
| 240 | 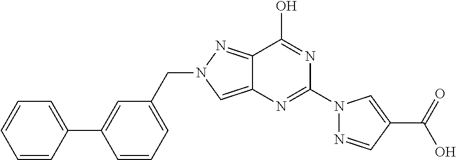 [SMILES: O=C(O)c1cnn(-c2nc(O)c3nn(Cc4cccc(-c5ccccc5)c4)cc3n2)c1] | powder MS (APCI) m/z: 413 [M + H]⁺ |
| 241 | 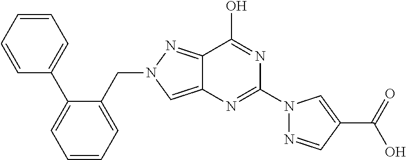 [SMILES: O=C(O)c1cnn(-c2nc(O)c3nn(Cc4ccccc4-c4ccccc4)cc3n2)c1] | powder MS (APCI) m/z: 413 [M + H]⁺ |
| 242 | 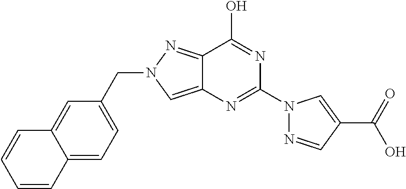 [SMILES: O=C(O)c1cnn(-c2nc(O)c3nn(Cc4ccc5ccccc5c4)cc3n2)c1] | powder MS (APCI) m/z: 387 [M + H]⁺ |
| 243 | 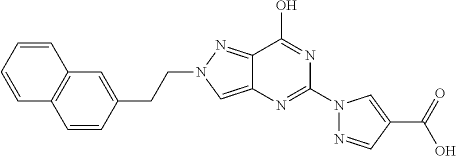 [SMILES: O=C(O)c1cnn(-c2nc(O)c3nn(CCc4ccc5ccccc5c4)cc3n2)c1] | powder MS (APCI) m/z: 401 [M + H]⁺ |
| 244 | 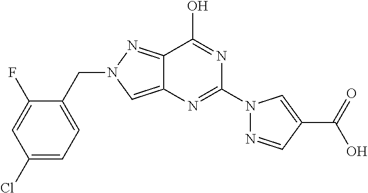 [SMILES: O=C(O)c1cnn(-c2nc(O)c3nn(Cc4ccc(Cl)cc4F)cc3n2)c1] | powder MS (APCI) m/z: 389/391 [M + H]⁺ |
| 245 | 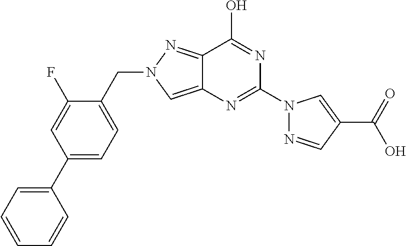 [SMILES: O=C(O)c1cnn(-c2nc(O)c3nn(Cc4ccc(-c5ccccc5)cc4F)cc3n2)c1] | powder MS (APCI) m/z: 431 [M + H]⁺ |
| 246 | 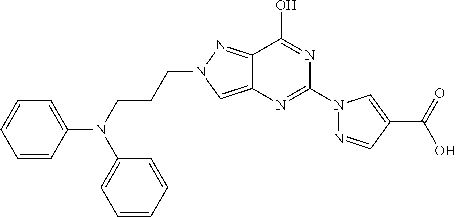 [SMILES: O=C(O)c1cnn(-c2nc(O)c3nn(CCCN(c4ccccc4)c4ccccc4)cc3n2)c1] | powder MS (ESI) m/z: 456 [M + H]⁺ |
| 247 | 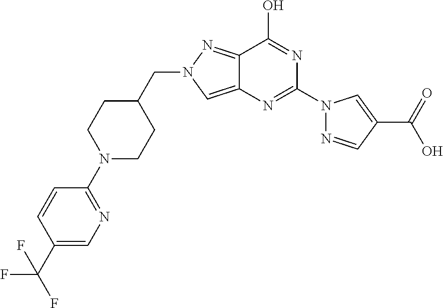 [SMILES: O=C(O)c1cnn(-c2nc(O)c3nn(CC4CCN(c5ccc(C(F)(F)F)cn5)CC4)cc3n2)c1] | powder MS (APCI) m/z: 489 [M + H]⁺ |
| 248 | 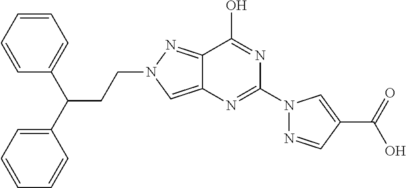 [SMILES: O=C(O)c1cnn(-c2nc(O)c3nn(CCC(c4ccccc4)c4ccccc4)cc3n2)c1] | powder MS (APCI) m/z: 441 [M + H]⁺ |
| 249 | 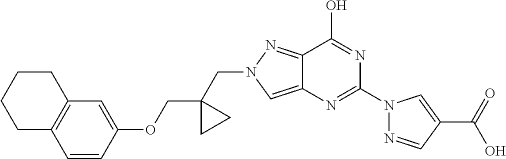 [SMILES: O=C(O)c1cnn(-c2nc(O)c3nn(CC4(COc5ccc6c(c5)CCCC6)CC4)cc3n2)c1] | powder MS (APCI) m/z: 461 [M + H]⁺ |
| 250 | 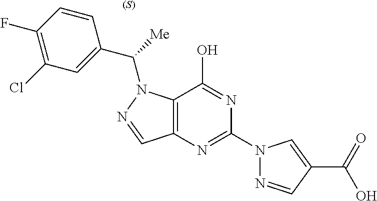 | powder MS (ESI) m/z: 401/403 [M + H]⁺ |
| 251 | 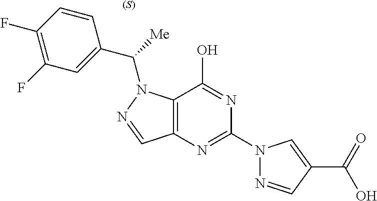 | powder MS (APCI) m/z: 387 [M + H]⁺ |
| 252 | 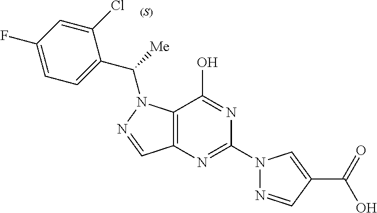 | powder MS (APCI) m/z: 403/405 [M + H]⁺ |
| 253 | 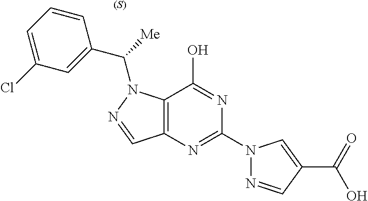 | powder MS (APCI) m/z: 385/387 [M + H]⁺ |
| 254 | 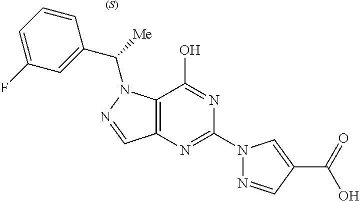 | powder MS (APCI) m/z: 369 [M + H]⁺ |
| 255 | 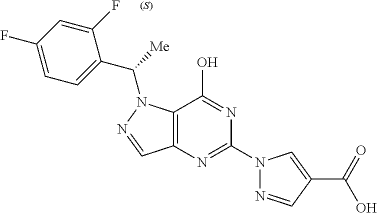 | powder MS (APCI) m/z: 387 [M + H]⁺ |
| 256 | 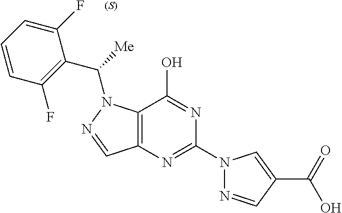 | powder MS (APCI) m/z: 387 [M + H]⁺ |
| 257 | 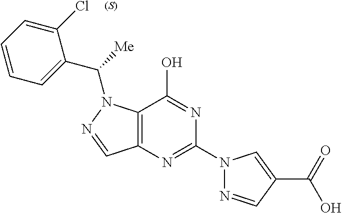 | powder MS (APCI) m/z: 385/387 [M + H]⁺ |
| 258 | 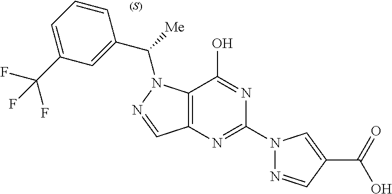 | powder MS (APCI) m/z: 419 [M + H]⁺ |
| 259 | 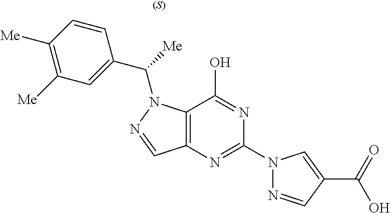 [SMILES: "(S)" ; Cc1ccc([C@H](C)n2ncc3nc(-n4cc(C(=O)O)cn4)nc(O)c32)cc1C] | powder MS (APCI) m/z: 379 [M + H]⁺ |
| 260 | 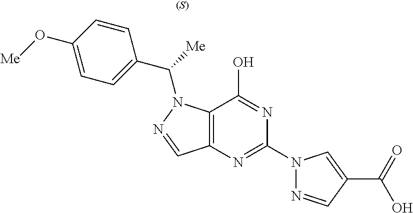 [SMILES: "(S)" ; COc1ccc([C@H](C)n2ncc3nc(-n4cc(C(=O)O)cn4)nc(O)c32)cc1] | powder MS (APCI) m/z: 381 [M + H]⁺ |
| 261 | 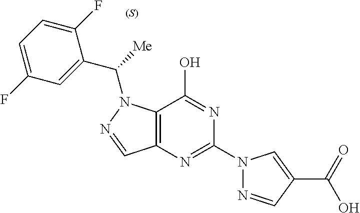 | powder MS (APCI) m/z: 387 [M + H]⁺ |
| 262 | 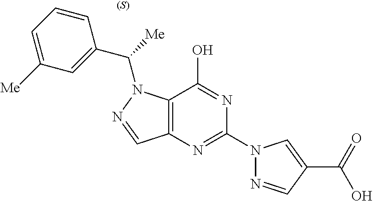 | powder MS (APCI) m/z: 365 [M + H]⁺ |
| 263 | 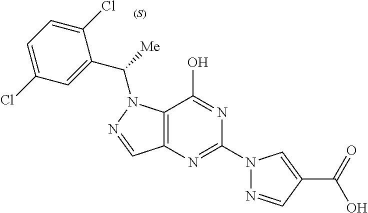 | powder MS (ESI) m/z: 419/421 [M + H]⁺ |
| 264 | 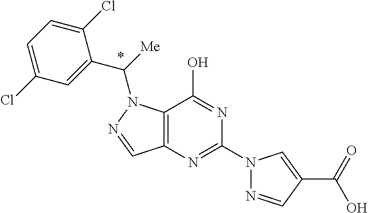 [SMILES: CC(c1cc(Cl)ccc1Cl)n1ncc2nc(-n3cc(C(=O)O)cn3)nc(O)c21] | powder MS (APCI) m/z: 419/421 [M + H]⁺ racemic mixture |
| 265 | 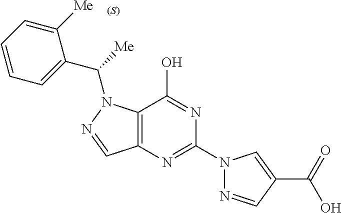 | powder MS (ESI) m/z: 365 [M + H]⁺ |
| 266 | 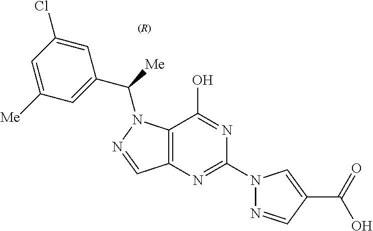 | powder MS (ESI) m/z: 399/401 [M + H]⁺ |
| 267 | 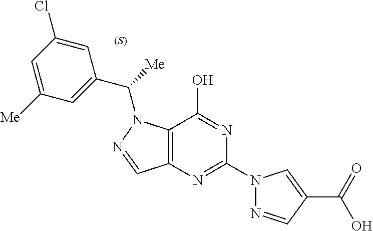 | powder MS (ESI) m/z: 399/401 [M + H]⁺ |
| 268 | 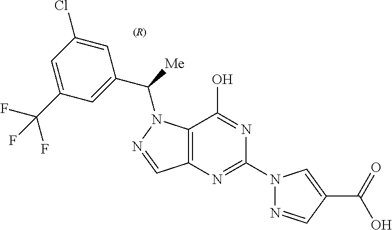 | powder MS (ESI) m/z: 451/453 [M − H]⁻ |
| 269 | 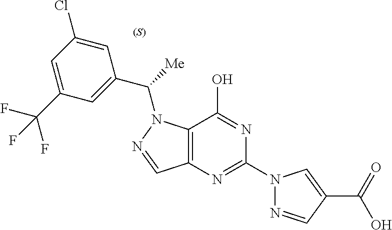 | powder MS (ESI) m/z: 451/453 [M − H]⁻ |
| 270 | 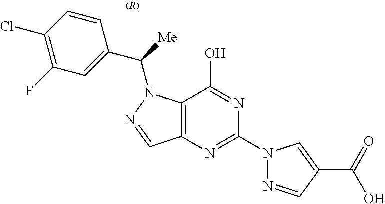 | powder MS (ESI) m/z: 403/405 [M + H]⁺ |
| 271 | 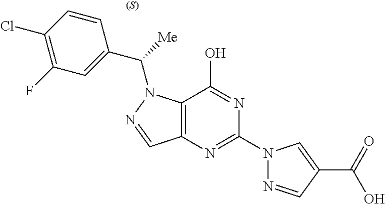 | powder MS (APCI) m/z: 403/405 [M + H]⁺ |
| 272 | 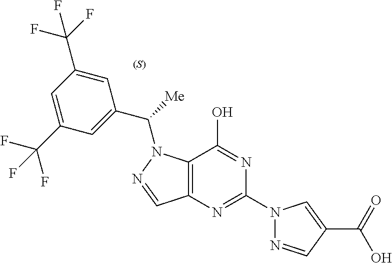 | powder MS (APCI) m/z: 487 [M + H]⁺ |
| 273 | 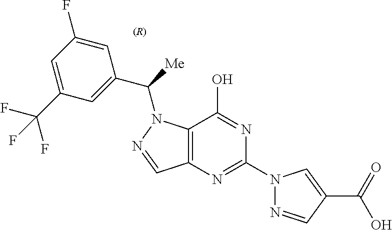 | powder MS (APCI) m/z: 437 [M + H]⁺ |
| 274 | 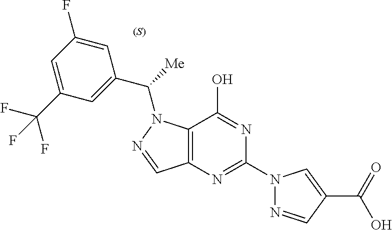 | powder MS (APCI) m/z: 437 [M + H]⁺ |
| 275 | 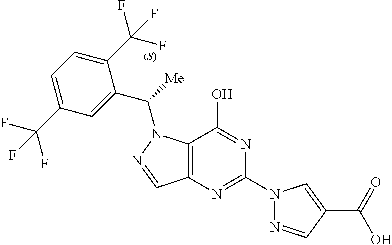 | powder MS (ESI) m/z: 487 [M + H]⁺ |
| 276 | 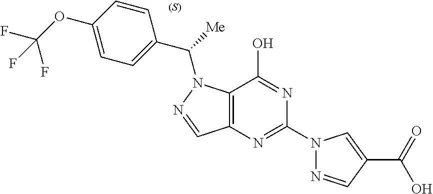 | powder MS (ESI) m/z: 435 [M + H]⁺ |
| 277 | 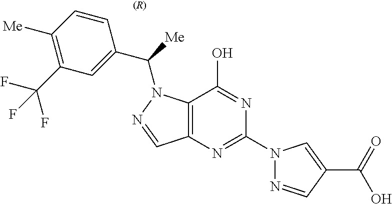 | powder MS (APCI) m/z: 433 [M + H]⁺ |
| 278 | 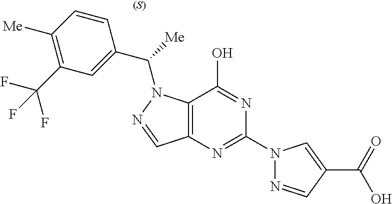 | powder MS (APCI) m/z: 433 [M + H]⁺ |
| 279 | 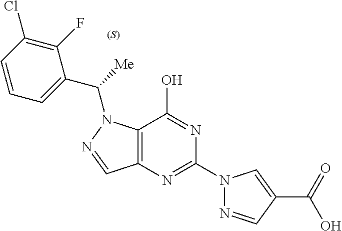 | powder MS (ESI) m/z: 401/403 [M − H]⁻ |
| 280 | 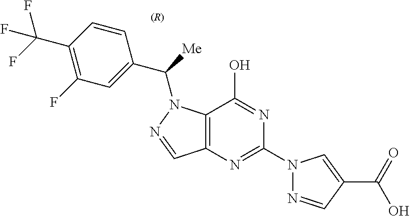 | powder MS (ESI) m/z: 435 [M − H]⁻ |
| 281 | 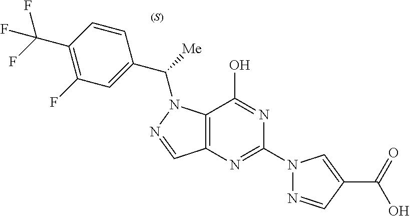 | powder MS (APCI) m/z: 437 [M + H]⁺ |
| 282 | 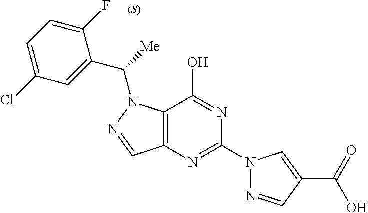 | powder MS (ESI) m/z: 403/405 [M + H]⁺ |
| 283 | 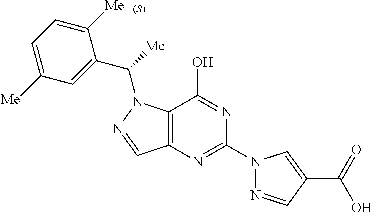 | powder MS (ESI) m/z: 379 [M + H]⁺ |
| 284 | 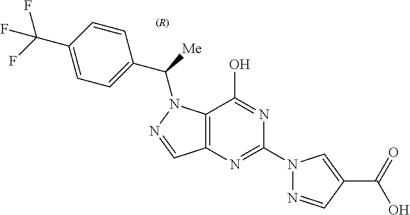 | powder MS (APCI) m/z: 419 [M + H]⁺ |
| 285 | 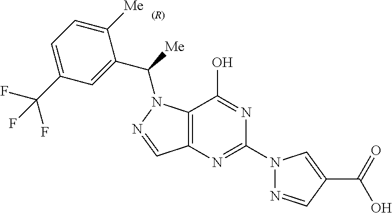 | powder MS (APCI) m/z: 433 [M + H]⁺ |
| 286 | 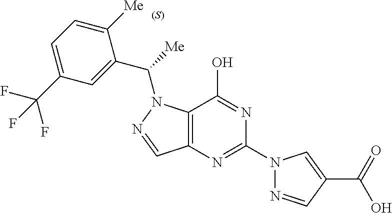 | powder MS (APCI) m/z: 433 [M + H]⁺ |
| 287 | 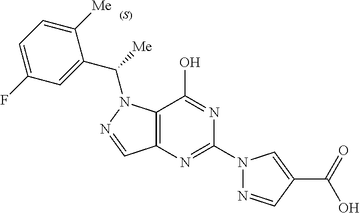 | powder MS (APCI) m/z: 383 [M + H]⁺ |
| 288 | 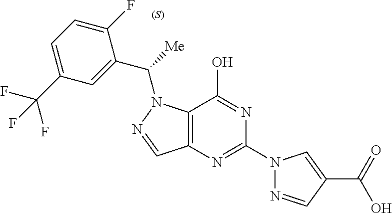 | powder MS (APCI) m/z: 437 [M + H]⁺ |
| 289 | 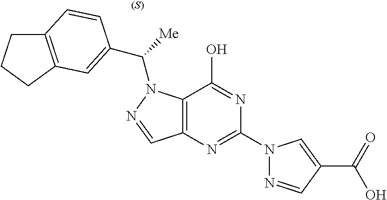 | powder MS (APCI) m/z: 391 [M + H]⁺ |
| 290 | 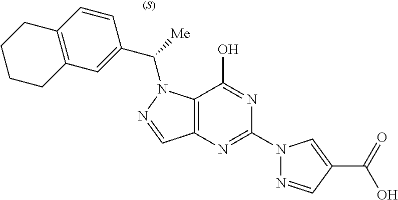 | powder MS (APCI) m/z: 405 [M + H]⁺ |
| 291 | 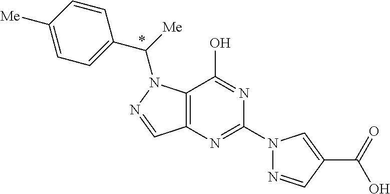 [SMILES: Cc1ccc(C(C)n2ncc3nc(-n4cc(C(=O)O)cn4)nc(O)c32)cc1] | powder MS (APCI) m/z: 365 [M + H]⁺ |
| 292 | 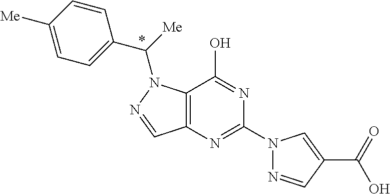 [SMILES: Cc1ccc(C(C)n2ncc3nc(-n4cc(C(=O)O)cn4)nc(O)c32)cc1] | powder MS (APCI) m/z: 365 [M + H]⁺ Enantiomer of Example 291 |
| 293 | 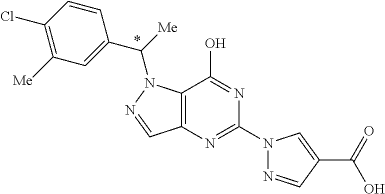 [SMILES: Cc1cc(C(C)n2ncc3nc(-n4cc(C(=O)O)cn4)nc(O)c32)ccc1Cl] | powder MS (APCI) m/z: 399/401 [M + H]⁺ |
| 294 | 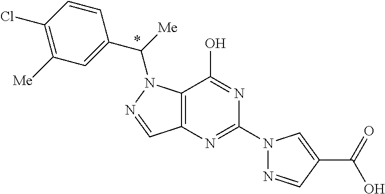 [SMILES: Cc1cc(C(C)n2ncc3nc(-n4cc(C(=O)O)cn4)nc(O)c32)ccc1Cl] | powder MS (APCI) m/z: 399/401 [M + H]⁺ Enantiomer of Example 293 |
| 295 | 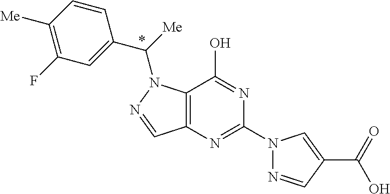 [SMILES: Cc1ccc(C(C)n2ncc3nc(-n4cc(C(=O)O)cn4)nc(O)c32)cc1F] | powder MS (APCI) m/z: 383 [M + H]⁺ |
| 296 | 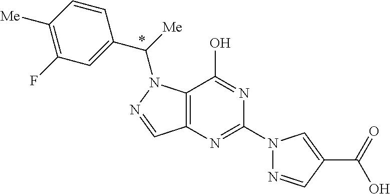 [SMILES: Cc1ccc(C(C)n2ncc3nc(-n4cc(C(=O)O)cn4)nc(O)c32)cc1F] | powder MS (APCI) m/z: 383 [M + H]⁺ Enantiomer of Example 295 |
| 297 | 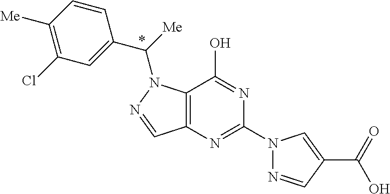 [SMILES: Cc1ccc(C(C)n2ncc3nc(-n4cc(C(=O)O)cn4)nc(O)c32)cc1Cl] | powder MS (APCI) m/z: 399/401 [M + H]⁺ |
| 298 | 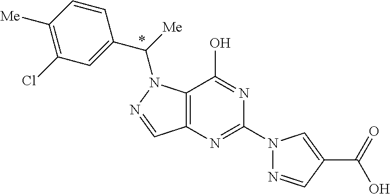 [SMILES: Cc1ccc(C(C)n2ncc3nc(-n4cc(C(=O)O)cn4)nc(O)c32)cc1Cl] | powder MS (APCI) m/z: 399/401 [M + H]⁺ Enantiomer of Example 297 |
| 299 | 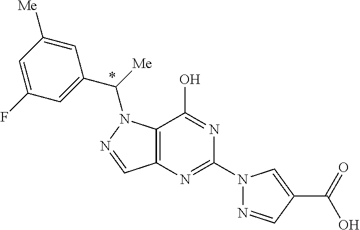 [SMILES: Cc1cc(F)cc(C(C)n2ncc3nc(-n4cc(C(=O)O)cn4)nc(O)c32)c1] | powder MS (APCI) m/z: 383 [M + H]⁺ |
| 300 | 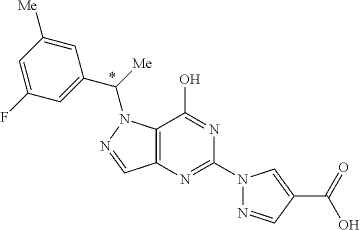 [SMILES: Cc1cc(F)cc(C(C)n2ncc3nc(-n4cc(C(=O)O)cn4)nc(O)c32)c1] | powder MS (APCI) m/z: 383 [M + H]⁺ Enantiomer of Example 299 |
| 301 | 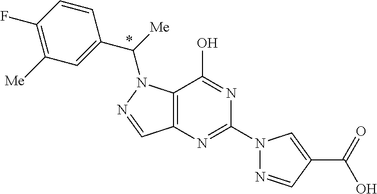 [SMILES: Cc1cc(C(C)n2ncc3nc(-n4cc(C(=O)O)cn4)nc(O)c32)ccc1F] | powder MS (APCI) m/z: 383 [M + H]⁺ |
| 302 | 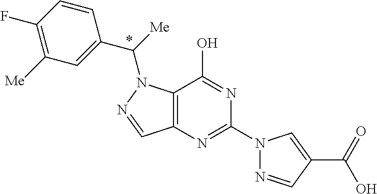 [SMILES: Cc1cc(C(C)n2ncc3nc(-n4cc(C(=O)O)cn4)nc(O)c32)ccc1F] | powder MS (APCI) m/z: 383 [M + H]⁺ Enantiomer of Example 301 |
| 303 | 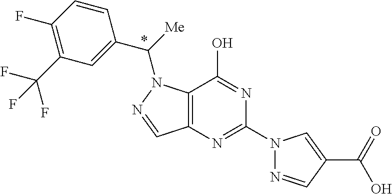 [SMILES: CC(c1ccc(F)c(C(F)(F)F)c1)n1ncc2nc(-n3cc(C(=O)O)cn3)nc(O)c21] | powder MS (APCI) m/z: 437 [M + H]⁺ |
| 304 | 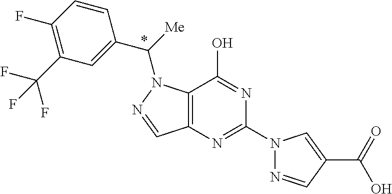 [SMILES: CC(c1ccc(F)c(C(F)(F)F)c1)n1ncc2nc(-n3cc(C(=O)O)cn3)nc(O)c21] | powder MS (APCI) m/z: 437 [M + H]⁺ Enantiomer of Example 303 |
| 305 | 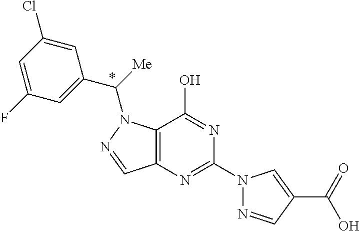 [SMILES: CC(c1cc(F)cc(Cl)c1)n1ncc2nc(-n3cc(C(=O)O)cn3)nc(O)c21] | powder MS (ESI) m/z: 401/403 [M − H]⁻ |
| 306 | 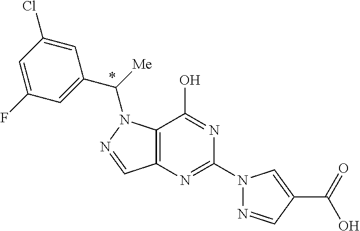 [SMILES: CC(c1cc(F)cc(Cl)c1)n1ncc2nc(-n3cc(C(=O)O)cn3)nc(O)c21] | powder MS (ESI) m/z: 401/403 [M − H]⁻ Enantiomer of Example 305 |
| 307 | 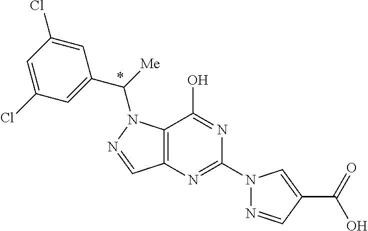 [SMILES: CC(c1cc(Cl)cc(Cl)c1)n1ncc2nc(-n3cc(C(=O)O)cn3)nc(O)c21] | powder MS (ESI) m/z: 417/419 [M − H]⁻ |
| 308 | 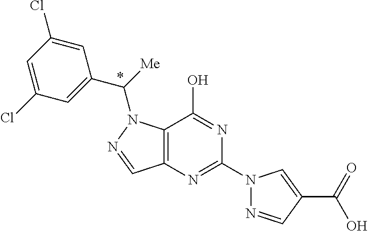 [SMILES: CC(c1cc(Cl)cc(Cl)c1)n1ncc2nc(-n3cc(C(=O)O)cn3)nc(O)c21] | powder MS (ESI) m/z: 417/419 [M − H]⁻ Enantiomer of Example 307 |
| 309 | 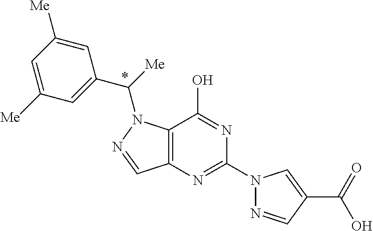 [SMILES: Cc1cc(C)cc(C(C)n2ncc3nc(-n4cc(C(=O)O)cn4)nc(O)c32)c1] | powder MS (ESI) m/z: 377 [M − H]⁻ |
| 310 | 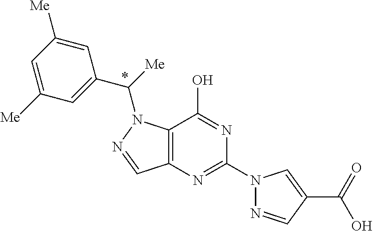 [SMILES: Cc1cc(C)cc(C(C)n2ncc3nc(-n4cc(C(=O)O)cn4)nc(O)c32)c1] | powder MS (ESI) m/z: 377 [M − H]⁻ Enantiomer of Example 309 |
| 311 | 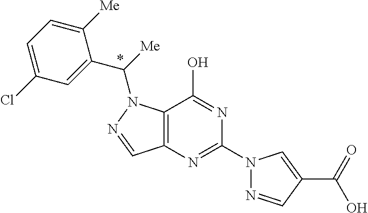 [SMILES: Cc1ccc(Cl)cc1C(C)n1ncc2nc(-n3cc(C(=O)O)cn3)nc(O)c21] | powder MS (APCI) m/z: 399/401 [M + H]⁺ |
| 312 | 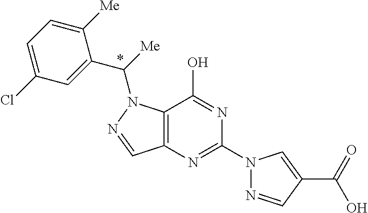 [SMILES: Cc1ccc(Cl)cc1C(C)n1ncc2nc(-n3cc(C(=O)O)cn3)nc(O)c21] | powder MS (APCI) m/z: 399/401 [M + H]⁺ Enantiomer of Example 311 |
| 313 | 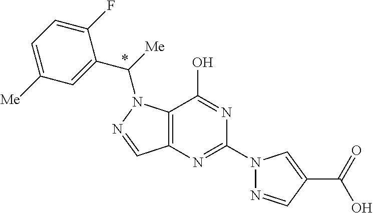 [SMILES: Cc1ccc(F)c(C(C)n2ncc3nc(-n4cc(C(=O)O)cn4)nc(O)c32)c1] | powder MS (APCI) m/z: 383 [M + H]⁺ |
| 314 | 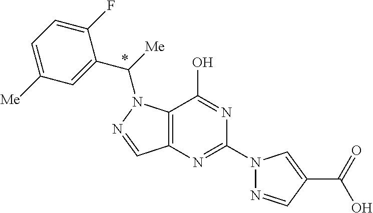 [SMILES: Cc1ccc(F)c(C(C)n2ncc3nc(-n4cc(C(=O)O)cn4)nc(O)c32)c1] | powder MS (APCI) m/z: 383 [M + H]⁺ Enantiomer of Example 313 |
| 315 | 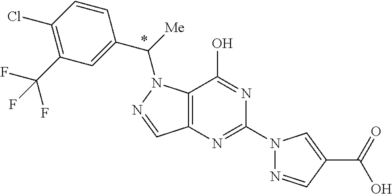 [SMILES: CC(c1ccc(Cl)c(C(F)(F)F)c1)n1ncc2nc(-n3cc(C(=O)O)cn3)nc(O)c21] | powder MS (APCI) m/z: 453/455 [M + H]⁺ |
| 316 | 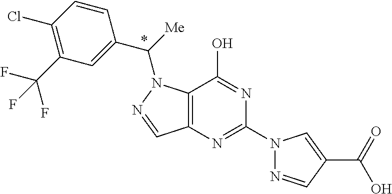 [SMILES: CC(c1ccc(Cl)c(C(F)(F)F)c1)n1ncc2nc(-n3cc(C(=O)O)cn3)nc(O)c21] | powder MS (APCI) m/z: 453/455 [M + H]⁺ Enantiomer of Example 315 |
| 317 | 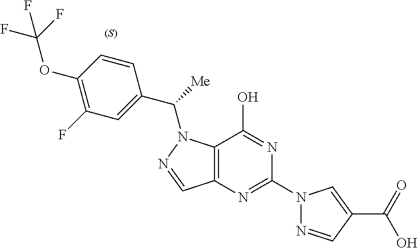 | powder MS (ESI) m/z: 453 [M + H]⁺ |
| 318 | 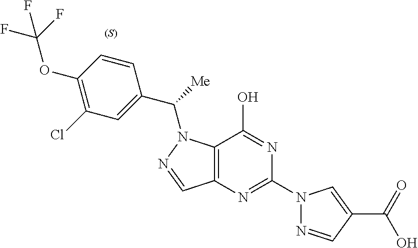 | powder MS (ESI) m/z: 469/471 [M + H]⁺ |
| 319 | 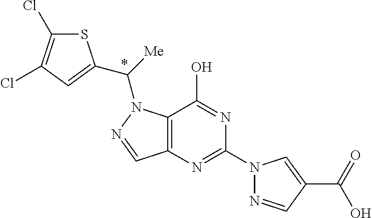 [SMILES: CC(c1cc(Cl)c(Cl)s1)n1ncc2nc(-n3cc(C(=O)O)cn3)nc(O)c21] | powder MS (ESI) m/z: 423/425 [M − H]⁻ |
| 320 | 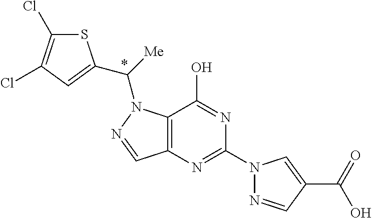 [SMILES: CC(c1cc(Cl)c(Cl)s1)n1ncc2nc(-n3cc(C(=O)O)cn3)nc(O)c21] | powder MS (ESI) m/z: 423/425 [M − H]⁻ Enantiomer of Example 319 |
| 321 | 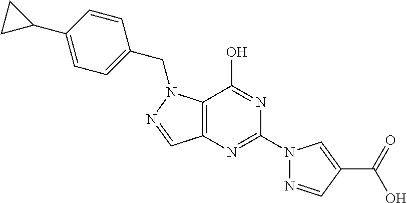 [SMILES: O=C(O)c1cnn(-c2nc(O)c3c(cnn3Cc3ccc(C4CC4)cc3)n2)c1] | powder MS (ESI) m/z: 375 [M − H]⁻ |
| 322 | 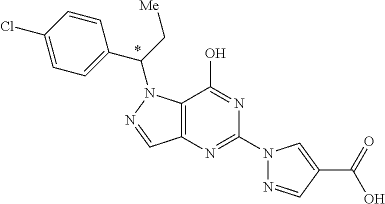 [SMILES: CCC(c1ccc(Cl)cc1)n1ncc2nc(-n3cc(C(=O)O)cn3)nc(O)c21] | powder MS (APCI) m/z: 399/401 [M + H]⁺ |
| 323 | 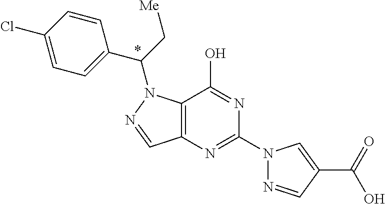 [SMILES: CCC(c1ccc(Cl)cc1)n1ncc2nc(-n3cc(C(=O)O)cn3)nc(O)c21] | powder MS (APCI) m/z: 399/401 [M + H]⁺ Enantiomer of Example 322 |
| 324 | 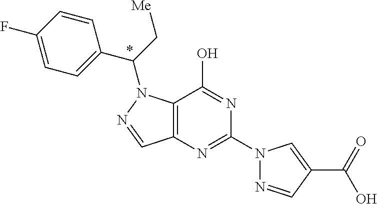 [SMILES: CCC(c1ccc(F)cc1)n1ncc2nc(-n3cc(C(=O)O)cn3)nc(O)c21] | powder MS (APCI) m/z: 383 [M + H]⁺ |
| 325 | 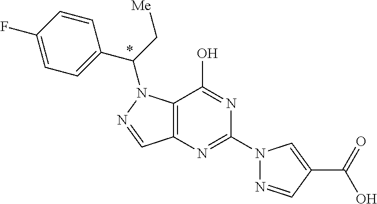 [SMILES: CCC(c1ccc(F)cc1)n1ncc2nc(-n3cc(C(=O)O)cn3)nc(O)c21] | powder MS (APCI) m/z: 383 [M + H]⁺ Enantiomer of Example 324 |
| 326 | 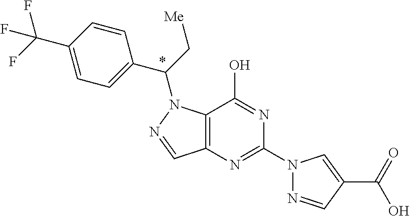 [SMILES: CCC(c1ccc(C(F)(F)F)cc1)n1ncc2nc(-n3cc(C(=O)O)cn3)nc(O)c21] | powder MS (APCI) m/z: 433 [M + H]⁺ |
| 327 | 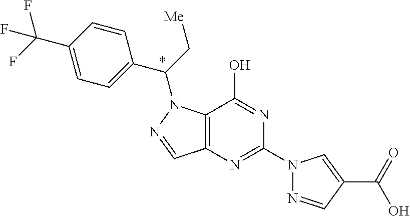 [SMILES: CCC(c1ccc(C(F)(F)F)cc1)n1ncc2nc(-n3cc(C(=O)O)cn3)nc(O)c21] | powder MS (APCI) m/z: 433 [M + H]⁺ Enantiomer of Example 326 |
| 328 | 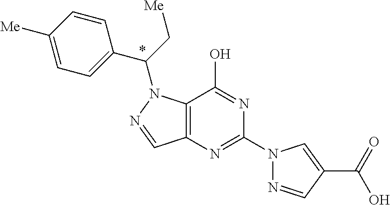 [SMILES: CCC(c1ccc(C)cc1)n1ncc2nc(-n3cc(C(=O)O)cn3)nc(O)c21] | powder MS (APCI) m/z: 379 [M + H]⁺ |
| 329 | 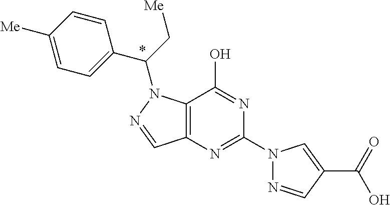 [SMILES: CCC(c1ccc(C)cc1)n1ncc2nc(-n3cc(C(=O)O)cn3)nc(O)c21] | powder MS (APCI) m/z: 379 [M + H]⁺ Enantiomer of Example 328 |
| 330 | 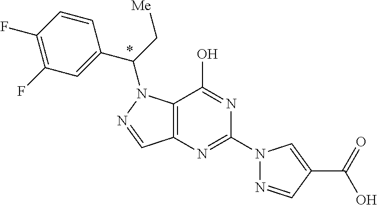 [SMILES: CCC(c1ccc(F)c(F)c1)n1ncc2nc(-n3cc(C(=O)O)cn3)nc(O)c21] | powder MS (APCI) m/z: 401 [M + H]⁺ |
| 331 | 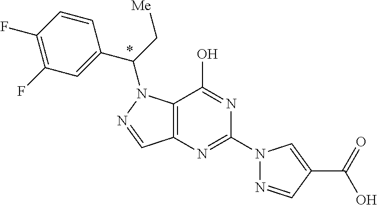 [SMILES: CCC(c1ccc(F)c(F)c1)n1ncc2nc(-n3cc(C(=O)O)cn3)nc(O)c21] | powder MS (APCI) m/z: 401 [M + H]⁺ Enantiomer of Example 330 |
| 332 | 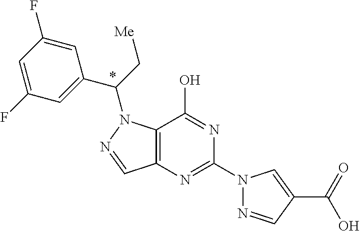 [SMILES: CCC(c1cc(F)cc(F)c1)n1ncc2nc(-n3cc(C(=O)O)cn3)nc(O)c21] | powder MS (APCI) m/z: 401 [M + H]⁺ |
| 333 | 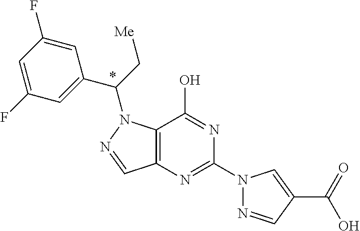 [SMILES: CCC(c1cc(F)cc(F)c1)n1ncc2nc(-n3cc(C(=O)O)cn3)nc(O)c21] | powder MS (APCI) m/z: 401 [M + H]⁺ Enantiomer of Example 332 |
| 334 | 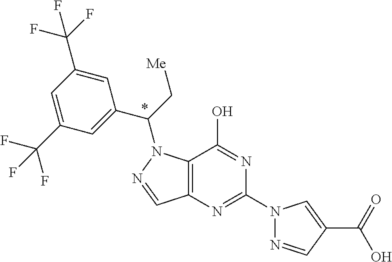 [SMILES: CCC(c1cc(C(F)(F)F)cc(C(F)(F)F)c1)n1ncc2nc(-n3cc(C(=O)O)cn3)nc(O)c21] | powder MS (APCI) m/z: 501 [M + H]⁺ |
| 335 | 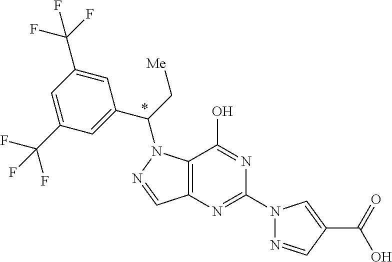 [SMILES: CCC(c1cc(C(F)(F)F)cc(C(F)(F)F)c1)n1ncc2nc(-n3cc(C(=O)O)cn3)nc(O)c21] | powder MS (APCI) m/z: 501 [M + H]⁺ Enantiomer of Example 334 |
| 336 | 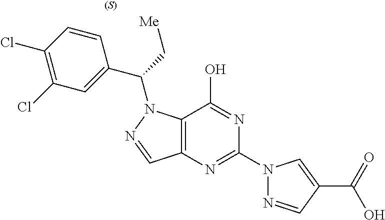 | powder MS (APCI) m/z: 433/435 [M + H]⁺ |
| 337 | 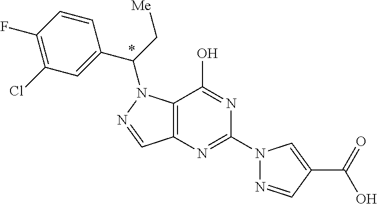 [SMILES: CCC(c1ccc(F)c(Cl)c1)n1ncc2nc(-n3cc(C(=O)O)cn3)nc(O)c21] | powder MS (APCI) m/z: 417/419 [M + H]⁺ |
| 338 | 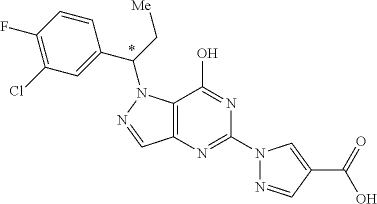 [SMILES: CCC(c1ccc(F)c(Cl)c1)n1ncc2nc(-n3cc(C(=O)O)cn3)nc(O)c21] | powder MS (APCI) m/z: 417/419 [M + H]⁺ Enantiomer of Example 337 |
| 339 | 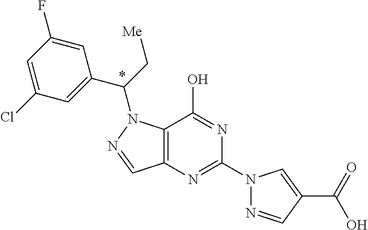 [SMILES: CCC(c1cc(F)cc(Cl)c1)n1ncc2nc(-n3cc(C(=O)O)cn3)nc(O)c21] | powder MS (APCI) m/z: 417/419 [M + H]⁺ |
| 340 | 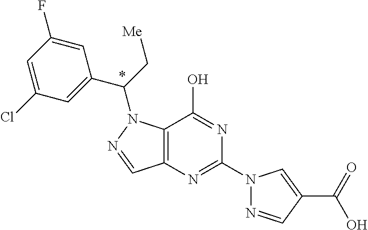 [SMILES: CCC(c1cc(F)cc(Cl)c1)n1ncc2nc(-n3cc(C(=O)O)cn3)nc(O)c21] | powder MS (APCI) m/z: 417/419 [M + H]⁺ Enantiomer of Example 339 |
| 341 | 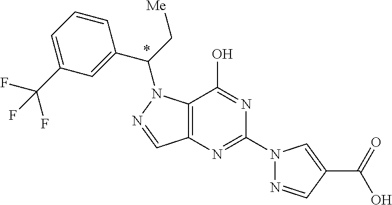 [SMILES: CCC(c1cccc(C(F)(F)F)c1)n1ncc2nc(-n3cc(C(=O)O)cn3)nc(O)c21] | powder MS (APCI) m/z: 433 [M + H]⁺ |
| 342 | 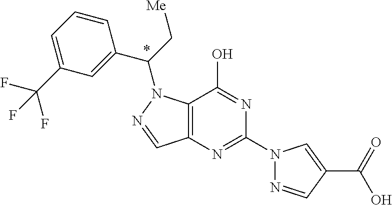 [SMILES: CCC(c1cccc(C(F)(F)F)c1)n1ncc2nc(-n3cc(C(=O)O)cn3)nc(O)c21] | powder MS (APCI) m/z: 433 [M + H]⁺ Enantiomer of Example 341 |
| 343 | 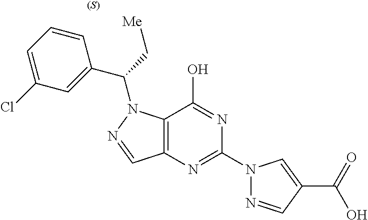 | powder MS (ESI) m/z: 397/399 [M − H]⁻ |
| 344 | 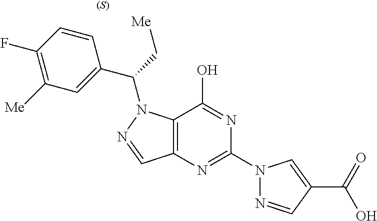 | powder MS (APCI) m/z: 397 [M + H]⁺ |
| 345 | 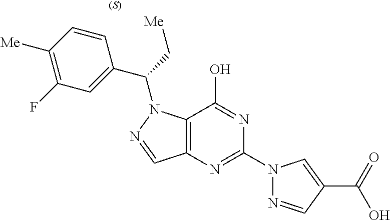 | powder MS (APCI) m/z: 397 [M + H]⁺ |
| 346 | 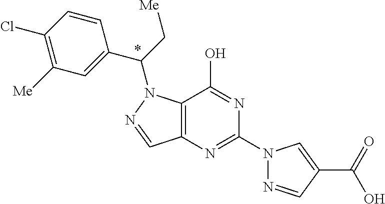 [SMILES: CCC(c1ccc(Cl)c(C)c1)n1ncc2nc(-n3cc(C(=O)O)cn3)nc(O)c21] | powder MS (APCI) m/z: 413/415 [M + H]⁺ |
| 347 | 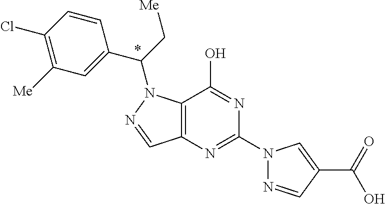 [SMILES: CCC(c1ccc(Cl)c(C)c1)n1ncc2nc(-n3cc(C(=O)O)cn3)nc(O)c21] | powder MS (APCI) m/z: 413/415 [M + H]⁺ Enantiomer of Example 346 |
| 348 | 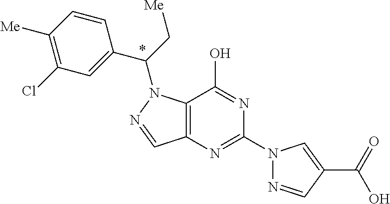 [SMILES: CCC(c1ccc(C)c(Cl)c1)n1ncc2nc(-n3cc(C(=O)O)cn3)nc(O)c21] | powder MS (APCI) m/z: 413/415 [M + H]⁺ |
| 349 | 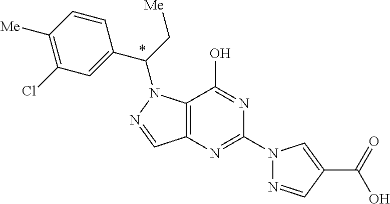 [SMILES: CCC(c1ccc(C)c(Cl)c1)n1ncc2nc(-n3cc(C(=O)O)cn3)nc(O)c21] | powder MS (APCI) m/z: 413/415 [M + H]⁺ Enantiomer of Example 348 |
| 350 | 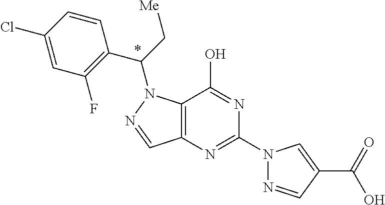 [SMILES: CCC(c1ccc(Cl)cc1F)n1ncc2nc(-n3cc(C(=O)O)cn3)nc(O)c21] | powder MS (APCI) m/z: 417/419 [M + H]⁺ |
| 351 | 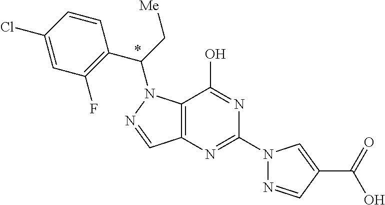 [SMILES: CCC(c1ccc(Cl)cc1F)n1ncc2nc(-n3cc(C(=O)O)cn3)nc(O)c21] | powder MS (APCI) m/z: 417/419 [M + H]⁺ Enantiomer of Example 350 |
| 352 | 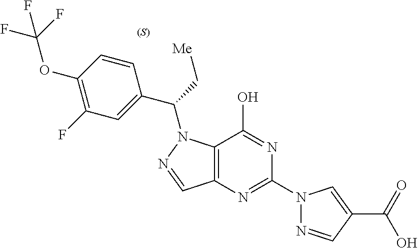 | powder MS (ESI) m/z: 467 [M + H]⁺ |
| 353 | 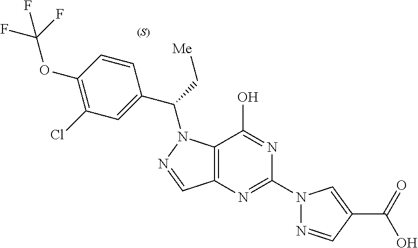 | powder MS (ESI) m/z: 483/485 [M + H]⁺ |
| 354 | 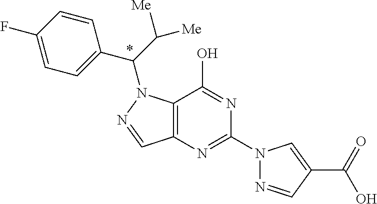 [SMILES: CC(C)C(c1ccc(F)cc1)n1ncc2nc(-n3cc(C(=O)O)cn3)nc(O)c21] | powder MS (APCI) m/z: 397 [M + H]⁺ |
| 355 | 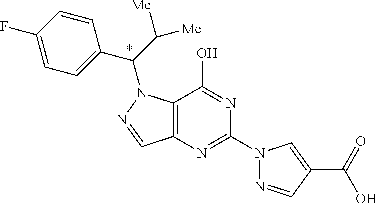 [SMILES: CC(C)C(c1ccc(F)cc1)n1ncc2nc(-n3cc(C(=O)O)cn3)nc(O)c21] | powder MS (APCI) m/z: 397 [M + H]⁺ Enantiomer of Example 354 |
| 356 | 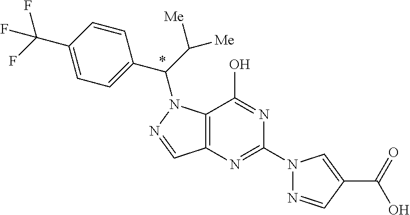 [SMILES: CC(C)C(c1ccc(C(F)(F)F)cc1)n1ncc2nc(-n3cc(C(=O)O)cn3)nc(O)c21] | powder MS (APCI) m/z: 447 [M + H]⁺ |
| 357 | 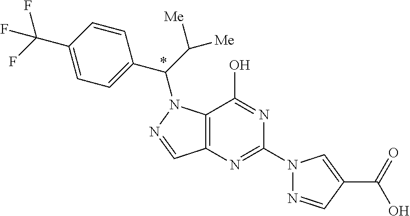 [SMILES: CC(C)C(c1ccc(C(F)(F)F)cc1)n1ncc2nc(-n3cc(C(=O)O)cn3)nc(O)c21] | powder MS (APCI) m/z: 447 [M + H]⁺ Enantiomer of Example 356 |
| 358 | 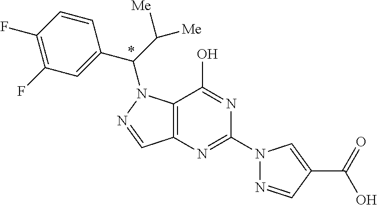 [SMILES: CC(C)C(c1ccc(F)c(F)c1)n1ncc2nc(-n3cc(C(=O)O)cn3)nc(O)c21] | powder MS (APCI) m/z: 415 [M + H]⁺ |
| 359 | 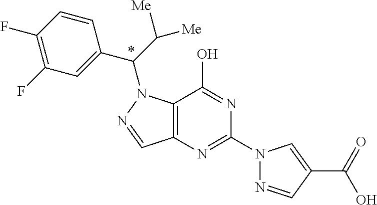 [SMILES: CC(C)C(c1ccc(F)c(F)c1)n1ncc2nc(-n3cc(C(=O)O)cn3)nc(O)c21] | powder MS (APCI) m/z: 415 [M + H]⁺ Enantiomer of Example 358 |
| 360 | 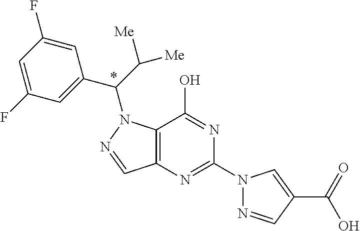 [SMILES: CC(C)C(c1cc(F)cc(F)c1)n1ncc2nc(-n3cc(C(=O)O)cn3)nc(O)c21] | powder MS (APCI) m/z: 415 [M + H]⁺ |
| 361 | 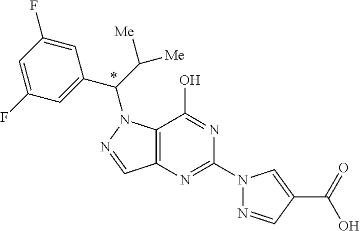 [SMILES: CC(C)C(c1cc(F)cc(F)c1)n1ncc2nc(-n3cc(C(=O)O)cn3)nc(O)c21] | powder MS (APCI) m/z: 415 [M + H]⁺ Enantiomer of Example 360 |
| 362 | 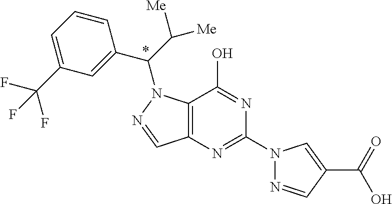 [SMILES: CC(C)C(c1cccc(C(F)(F)F)c1)n1ncc2nc(-n3cc(C(=O)O)cn3)nc(O)c21] | powder MS (APCI) m/z: 447 [M + H]⁺ |
| 363 | 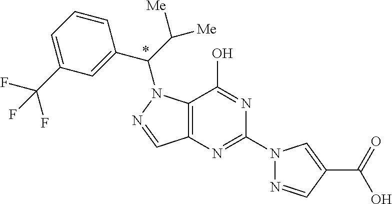 [SMILES: CC(C)C(c1cccc(C(F)(F)F)c1)n1ncc2nc(-n3cc(C(=O)O)cn3)nc(O)c21] | powder MS (APCI) m/z: 447 [M + H]⁺ Enantiomer of Example 362 |
| 364 | 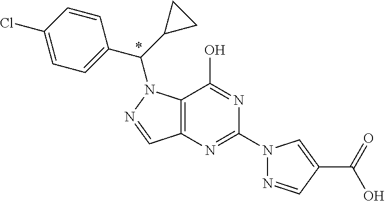 [SMILES: O=C(O)c1cnn(-c2nc(O)c3c(cnn3C(c3ccc(Cl)cc3)C3CC3)n2)c1] | powder MS (APCI) m/z: 411/413 [M + H]⁺ |
| 365 | 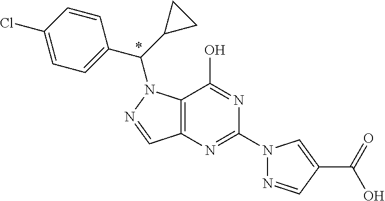 [SMILES: O=C(O)c1cnn(-c2nc(O)c3c(cnn3C(c3ccc(Cl)cc3)C3CC3)n2)c1] | powder MS (APCI) m/z: 411/413 [M + H]⁺ Enantiomer of Example 364 |
| 366 | 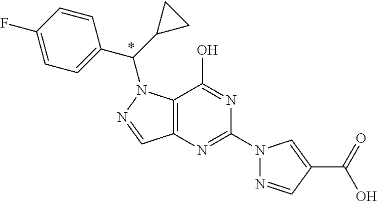 [SMILES: O=C(O)c1cnn(-c2nc(O)c3c(cnn3C(c3ccc(F)cc3)C3CC3)n2)c1] | powder MS (APCI) m/z: 395 [M + H]⁺ |
| 367 | 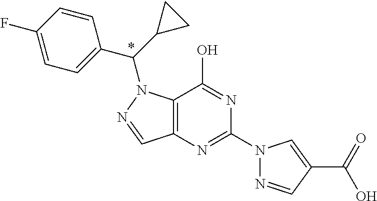 [SMILES: O=C(O)c1cnn(-c2nc(O)c3c(cnn3C(c3ccc(F)cc3)C3CC3)n2)c1] | powder MS (APCI) m/z: 395 [M + H]⁺ Enantiomer of Example 366 |
| 368 | 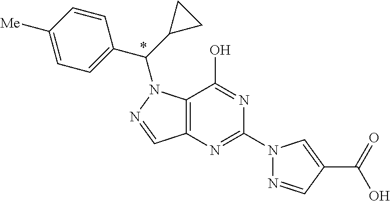 [SMILES: Cc1ccc(C(C2CC2)n2ncc3nc(-n4cc(C(=O)O)cn4)nc(O)c32)cc1] | powder MS (APCI) m/z: 391 [M + H]⁺ |
| 369 | 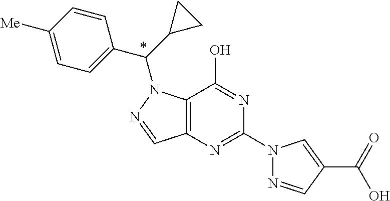 [SMILES: Cc1ccc(C(C2CC2)n2ncc3nc(-n4cc(C(=O)O)cn4)nc(O)c32)cc1] | powder MS (APCI) m/z: 391 [M + H]⁺ Enantiomer of Example 368 |
| 370 | 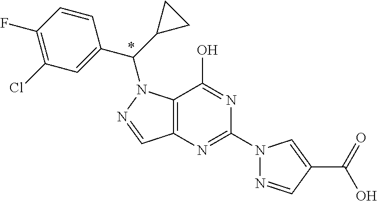 [SMILES: O=C(O)c1cnn(-c2nc(O)c3c(cnn3C(c3ccc(F)c(Cl)c3)C3CC3)n2)c1] | powder MS (APCI) m/z: 429/431 [M + H]⁺ |
| 371 | 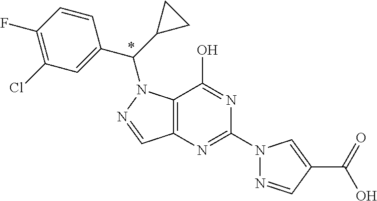 [SMILES: O=C(O)c1cnn(-c2nc(O)c3c(cnn3C(c3ccc(F)c(Cl)c3)C3CC3)n2)c1] | powder MS (APCI) m/z: 429/431 [M + H]⁺ Enantiomer of Example 370 |
| 372 | 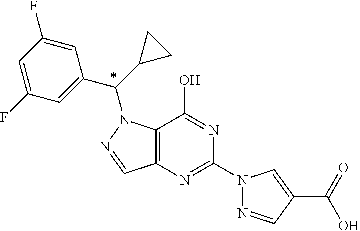 [SMILES: O=C(O)c1cnn(-c2nc(O)c3c(cnn3C(c3cc(F)cc(F)c3)C3CC3)n2)c1] | powder MS (APCI) m/z: 413 [M + H]⁺ |
| 373 | 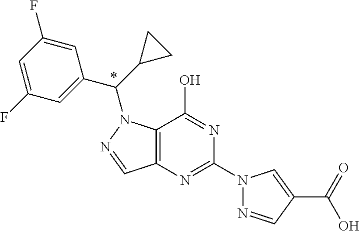 [SMILES: O=C(O)c1cnn(-c2nc(O)c3c(cnn3C(c3cc(F)cc(F)c3)C3CC3)n2)c1] | powder MS (APCI) m/z: 413 [M + H]⁺ Enantiomer of Example 372 |
| 374 | 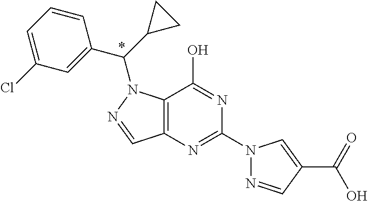 [SMILES: O=C(O)c1cnn(-c2nc(O)c3c(cnn3C(c3cccc(Cl)c3)C3CC3)n2)c1] | powder MS (APCI) m/z: 411/413 [M + H]⁺ |
| 375 | 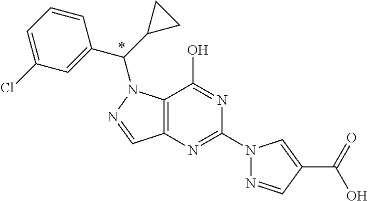 [SMILES: O=C(O)c1cnn(-c2nc(O)c3c(cnn3C(c3cccc(Cl)c3)C3CC3)n2)c1] | powder MS (APCI) m/z: 411/413 [M + H]⁺ Enantiomer of Example 374 |
| 376 | 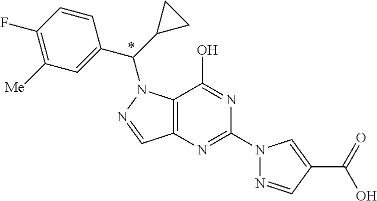 [SMILES: Cc1cc(C(C2CC2)n2ncc3nc(-n4cc(C(=O)O)cn4)nc(O)c32)ccc1F] | powder MS (APCI) m/z: 409 [M + H]⁺ |
| 377 | 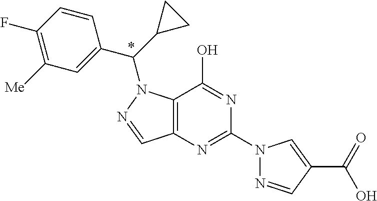 [SMILES: Cc1cc(C(C2CC2)n2ncc3nc(-n4cc(C(=O)O)cn4)nc(O)c32)ccc1F] | powder MS (APCI) m/z: 409 [M + H]⁺ Enantiomer of Example 376 |
| 378 | 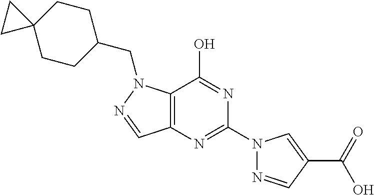 [SMILES: O=C(O)c1cnn(-c2nc(O)c3c(cnn3CC3CCC4(CC3)CC4)n2)c1] | powder MS (APCI) m/z: 369 [M + H]⁺ |
| 379 | 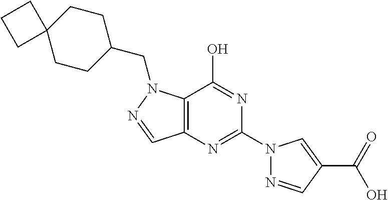 [SMILES: O=C(O)c1cnn(-c2nc(O)c3c(cnn3CC3CCC4(CCC4)CC3)n2)c1] | powder MS (APCI) m/z: 383 [M + H]⁺ |
| 380 | 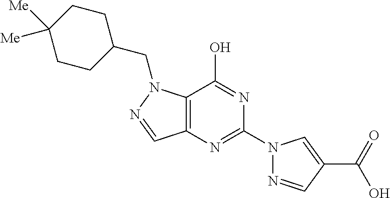 [SMILES: CC1(C)CCC(Cn2ncc3nc(-n4cc(C(=O)O)cn4)nc(O)c32)CC1] | powder MS (APCI) m/z: 371 [M + H]⁺ |
| 381 | 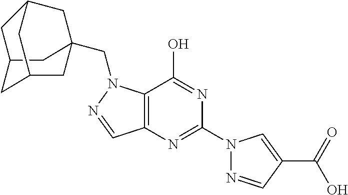 [SMILES: O=C(O)c1cnn(-c2nc(O)c3c(cnn3CC34CC5CC(CC(C5)C3)C4)n2)c1] | powder MS (ESI) m/z: 395 [M + H]⁺ |
| 382 | 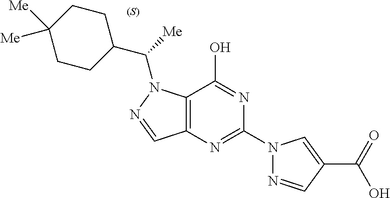 | powder MS (APCI) m/z: 385 [M + H]⁺ |
| 383 | 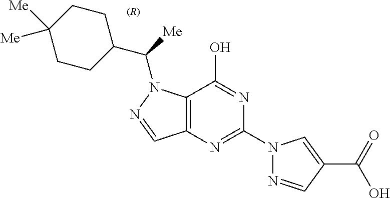 | powder MS (APCI) m/z: 385 [M + H]⁺ |
| 384 | 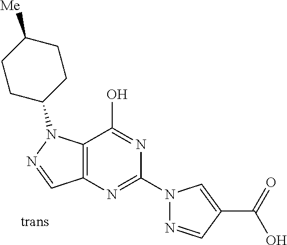 [SMILES: C[C@H]1CC[C@H](n2ncc3nc(-n4cc(C(=O)O)cn4)nc(O)c32)CC1] | powder MS (APCI) m/z: 343 [M + H]⁺ |
| 385 | 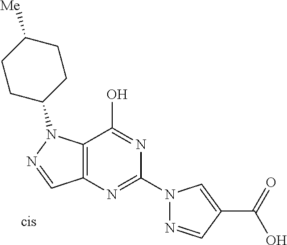 [SMILES: C[C@H]1CC[C@@H](n2ncc3nc(-n4cc(C(=O)O)cn4)nc(O)c32)CC1] | powder MS (APCI) m/z: 343 [M + H]⁺ |
| 386 | 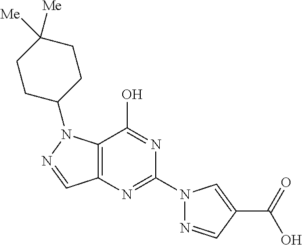 [SMILES: CC1(C)CCC(n2ncc3nc(-n4cc(C(=O)O)cn4)nc(O)c32)CC1] | powder MS (APCI) m/z: 357 [M + H]⁺ |
| 387 | 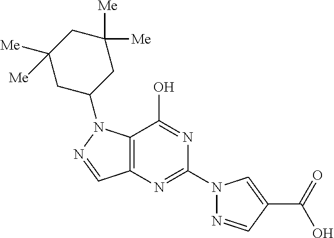 [SMILES: CC1(C)CC(n2ncc3nc(-n4cc(C(=O)O)cn4)nc(O)c32)CC(C)(C)C1] | powder MS (APCI) m/z: 385 [M + H]⁺ |
| 388 | 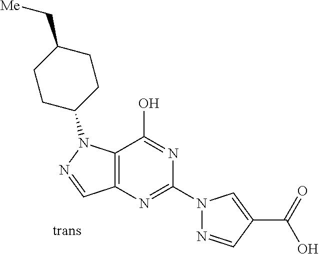 [SMILES: CC[C@H]1CC[C@H](n2ncc3nc(-n4cc(C(=O)O)cn4)nc(O)c32)CC1] | powder MS (APCI) m/z: 357 [M + H]⁺ |
| 389 | 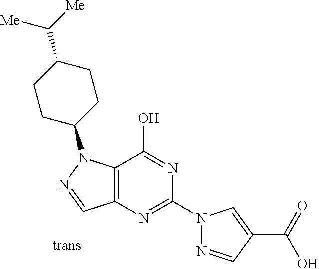 [SMILES: CC(C)[C@H]1CC[C@H](n2ncc3nc(-n4cc(C(=O)O)cn4)nc(O)c32)CC1] | powder MS (APCI) m/z: 371 [M + H]⁺ |
| 390 | 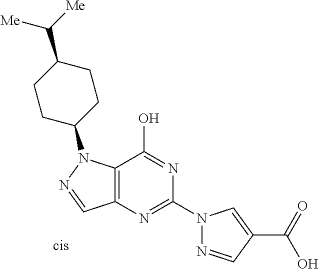 [SMILES: CC(C)[C@H]1CC[C@@H](n2ncc3nc(-n4cc(C(=O)O)cn4)nc(O)c32)CC1] | powder MS (APCI) m/z: 371 [M + H]⁺ |
| 391 | 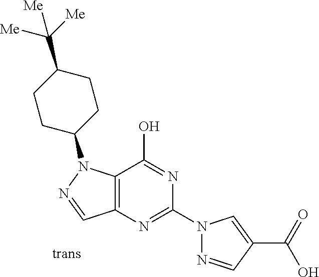 [SMILES: CC(C)(C)[C@H]1CC[C@@H](n2ncc3nc(-n4cc(C(=O)O)cn4)nc(O)c32)CC1] | powder MS (APCI) m/z: 385 [M + H]⁺ |
| 392 | 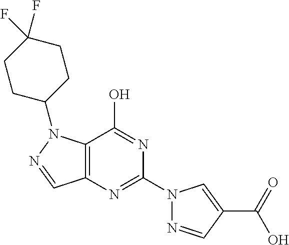 [SMILES: O=C(O)c1cnn(-c2nc(O)c3c(cnn3C3CCC(F)(F)CC3)n2)c1] | powder MS (APCI) m/z: 365 [M + H]⁺ |
| 393 | 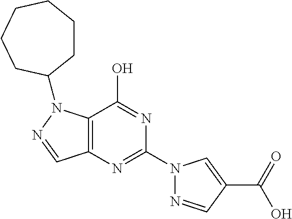 [SMILES: O=C(O)c1cnn(-c2nc(O)c3c(cnn3C3CCCCCC3)n2)c1] | powder MS (APCI) m/z: 343 [M + H]⁺ |
| 394 | 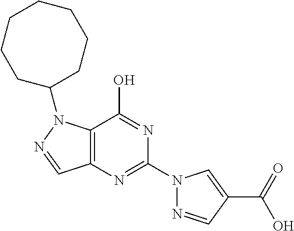 [SMILES: O=C(O)c1cnn(-c2nc(O)c3c(cnn3C3CCCCCCC3)n2)c1] | powder MS (APCI) m/z: 357 [M + H]⁺ |
| 395 | 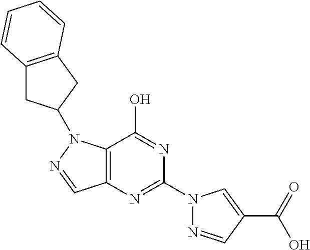 [SMILES: O=C(O)c1cnn(-c2nc(O)c3c(cnn3C3Cc4ccccc4C3)n2)c1] | powder MS (APCI) m/z: 363 [M + H]⁺ |
| 396 | 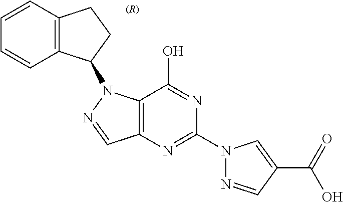 | powder MS (APCI) m/z: 363 [M + H]⁺ |
| 397 | 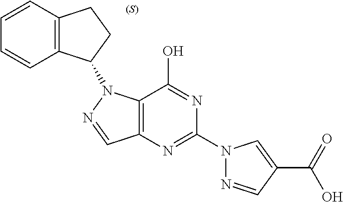 | powder MS (APCI) m/z: 363 [M + H]⁺ |
| 398 | 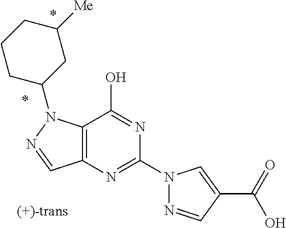 [SMILES: CC1CCCC(n2ncc3nc(-n4cc(C(=O)O)cn4)nc(O)c32)C1] | powder MS (ESI) m/z: 343 [M + H]⁺ |
| 399 | 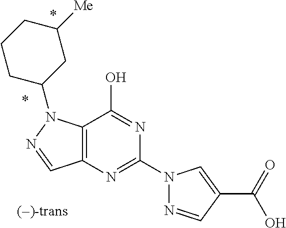 [SMILES: CC1CCCC(n2ncc3nc(-n4cc(C(=O)O)cn4)nc(O)c32)C1] | powder MS (ESI) m/z: 341 [M − H]⁻ Enantiomer of Example 398 |
| 400 | 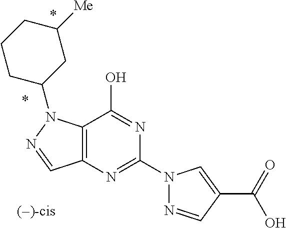 [SMILES: CC1CCCC(n2ncc3nc(-n4cc(C(=O)O)cn4)nc(O)c32)C1] | powder MS (ESI) m/z: 343 [M + H]⁺ |
| 401 | 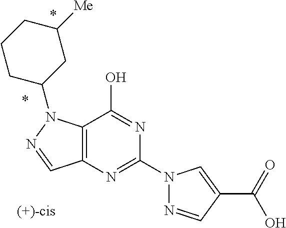 [SMILES: CC1CCCC(n2ncc3nc(-n4cc(C(=O)O)cn4)nc(O)c32)C1] | powder MS (ESI) m/z: 343 [M + H]⁺ Enantiomer of Example 400 |
| 402 | 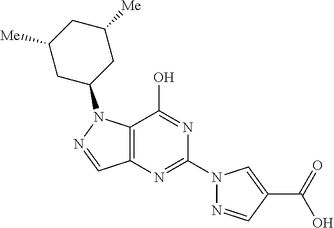 [SMILES: C[C@@H]1C[C@H](C)C[C@@H](n2ncc3nc(-n4cc(C(=O)O)cn4)nc(O)c32)C1] | powder MS (APCI) m/z: 357 [M + H]⁺ |
| 403 | 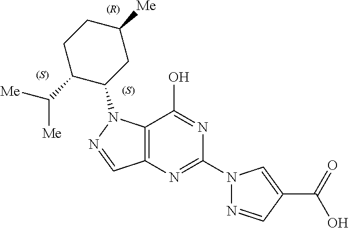 | powder MS (APCI) m/z: 385 [M + H]⁺ |
| 404 | 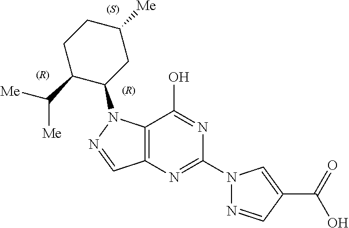 | powder MS (APCI) m/z: 385 [M + H]⁺ |
| 405 | 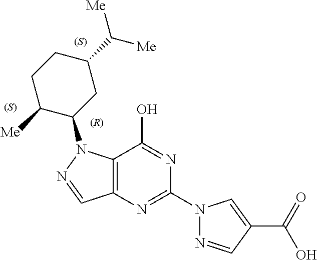 | powder MS (APCI) m/z: 385 [M + H]⁺ |
| 406 | 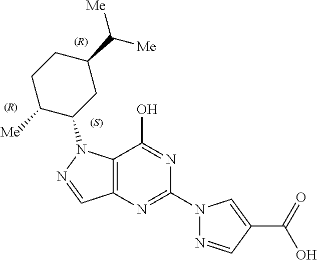 | powder MS (APCI) m/z: 385 [M + H]⁺ |
| 407 | 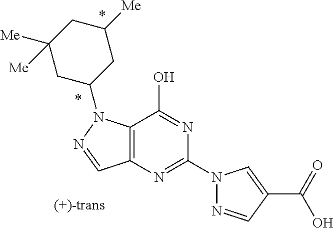 [SMILES: CC1CC(n2ncc3nc(-n4cc(C(=O)O)cn4)nc(O)c32)CC(C)(C)C1] | powder MS (ESI) m/z: 369 [M − H]⁻ |
| 408 | 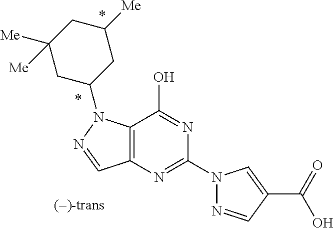 [SMILES: CC1CC(n2ncc3nc(-n4cc(C(=O)O)cn4)nc(O)c32)CC(C)(C)C1] | powder MS (ESI) m/z: 369 [M − H]⁻ Enantiomer of Example 407 |
| 409 | 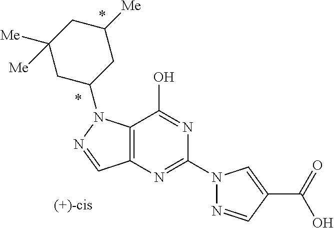 [SMILES: CC1CC(n2ncc3nc(-n4cc(C(=O)O)cn4)nc(O)c32)CC(C)(C)C1] | powder MS (ESI) m/z: 369 [M − H]⁻ |
| 410 | 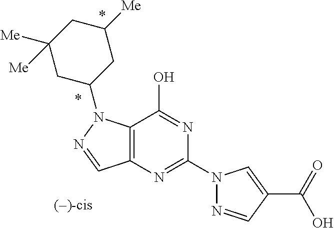 [SMILES: CC1CC(n2ncc3nc(-n4cc(C(=O)O)cn4)nc(O)c32)CC(C)(C)C1] | powder MS (ESI) m/z: 369 [M − H]⁻ Enantiomer of Example 409 |
| 411 | 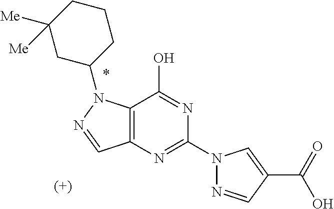 [SMILES: CC1(C)CCCC(n2ncc3nc(-n4cc(C(=O)O)cn4)nc(O)c32)C1] | powder MS (APCI) m/z: 357 [M + H]⁺ |
| 412 | 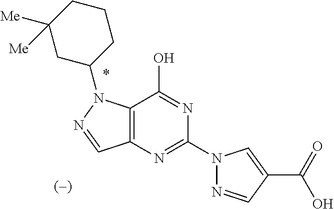 [SMILES: CC1(C)CCCC(n2ncc3nc(-n4cc(C(=O)O)cn4)nc(O)c32)C1] | powder MS (APCI) m/z: 357 [M + H]⁺ Enantiomer of Example 411 |
| 413 | 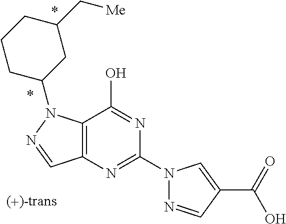 [SMILES: CCC1CCCC(n2ncc3nc(-n4cc(C(=O)O)cn4)nc(O)c32)C1] | powder MS (APCI) m/z: 357 [M + H]⁺ |
| 414 | 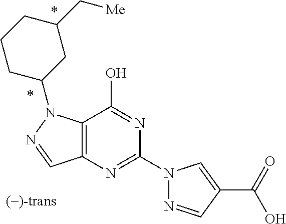 [SMILES: CCC1CCCC(n2ncc3nc(-n4cc(C(=O)O)cn4)nc(O)c32)C1] | powder MS (APCI) m/z: 357 [M + H]⁺ Enantiomer of Example 413 |
| 415 | 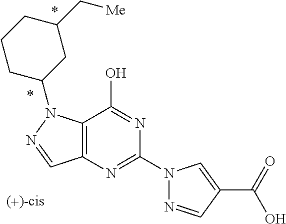 [SMILES: CCC1CCCC(n2ncc3nc(-n4cc(C(=O)O)cn4)nc(O)c32)C1] | powder MS (APCI) m/z: 357 [M + H]⁺ |
| 416 | 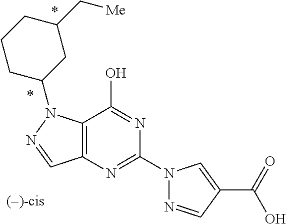 [SMILES: CCC1CCCC(n2ncc3nc(-n4cc(C(=O)O)cn4)nc(O)c32)C1] | powder MS (APCI) m/z: 357 [M + H]⁺ Enantiomer of Example 415 |
| 417 | 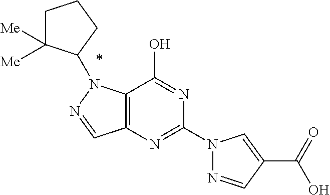 [SMILES: CC1(C)CCCC1n1ncc2nc(-n3cc(C(=O)O)cn3)nc(O)c21] | powder MS (APCI) m/z: 343 [M + H]⁺ |
| 418 | 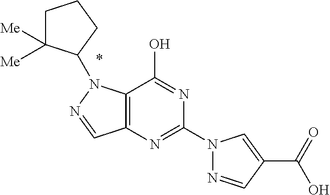 [SMILES: CC1(C)CCCC1n1ncc2nc(-n3cc(C(=O)O)cn3)nc(O)c21] | powder MS (APCI) m/z: 343 [M + H]⁺ Enantiomer of Example 417 |
| 419 | 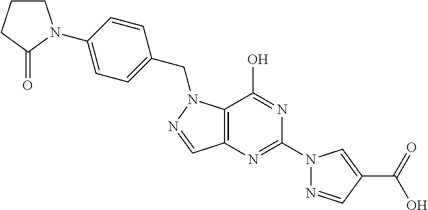 [SMILES: O=C(O)c1cnn(-c2nc(O)c3c(cnn3Cc3ccc(N4CCCC4=O)cc3)n2)c1] | powder MS (ESI) m/z: 420 [M + H]⁺ |
| 420 | 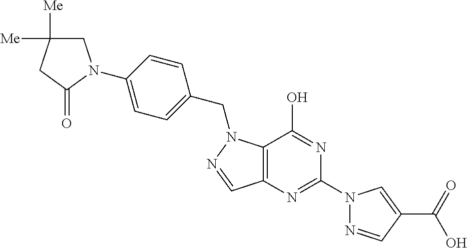 [SMILES: CC1(C)CC(=O)N(c2ccc(Cn3ncc4nc(-n5cc(C(=O)O)cn5)nc(O)c43)cc2)C1] | powder MS (ESI) m/z: 448 [M + H]⁺ |
| 421 | 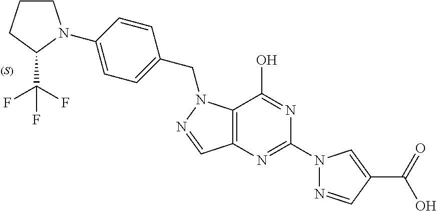 | powder MS (ESI) m/z: 473 [M + H]⁺ |
| 422 | 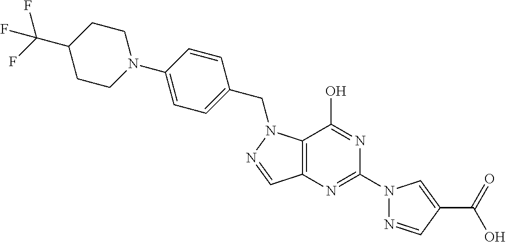 [SMILES: O=C(O)c1cnn(-c2nc(O)c3c(cnn3Cc3ccc(N4CCC(C(F)(F)F)CC4)cc3)n2)c1] | powder MS (APCI) m/z: 488 [M + H]⁺ |
| 423 | 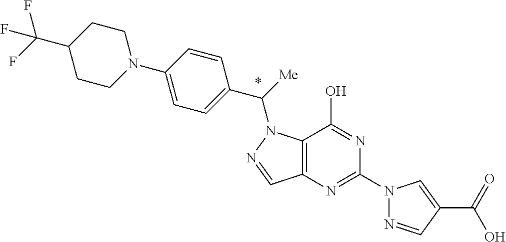 [SMILES: CC(c1ccc(N2CCC(C(F)(F)F)CC2)cc1)n1ncc2nc(-n3cc(C(=O)O)cn3)nc(O)c21] | powder MS (APCI) m/z: 502 [M + H]⁺ racemic mixture |
| 424 | 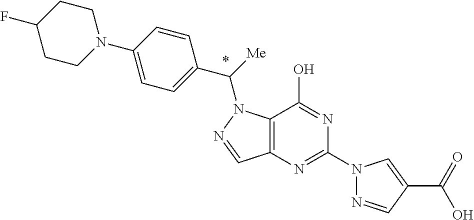 [SMILES: CC(c1ccc(N2CCC(F)CC2)cc1)n1ncc2nc(-n3cc(C(=O)O)cn3)nc(O)c21] | powder MS (APCI) m/z: 452 [M + H]⁺ racemic mixture |
| 425 | 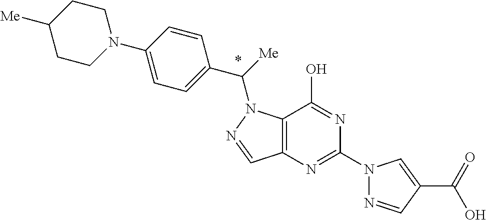 [SMILES: CC1CCN(c2ccc(C(C)n3ncc4nc(-n5cc(C(=O)O)cn5)nc(O)c43)cc2)CC1] | powder MS (APCI) m/z: 448 [M + H]⁺ racemic mixture |
| 426 | 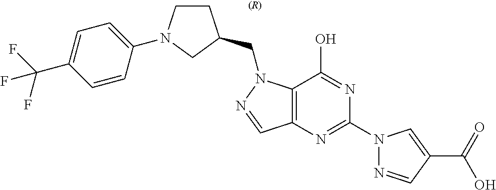 | powder MS (APCI) m/z: 474 [M + H]⁺ |
| 427 | 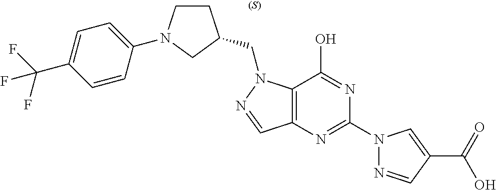 | powder MS (ESI) m/z: 474 [M + H]⁺ |
| 428 | 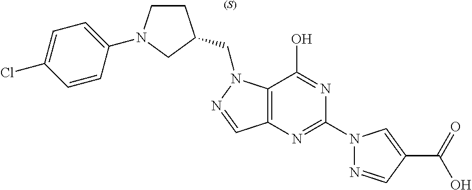 | powder MS (ESI) m/z: 438/440 [M − H]⁻ |
| 429 | 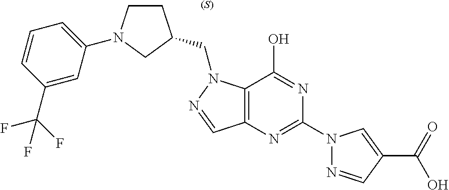 | powder MS (ESI) m/z: 474 [M + H]⁺ |
| 430 | 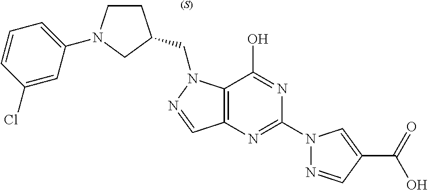 | powder MS (APCI) m/z: 440/442 [M + H]⁺ |
| 431 | 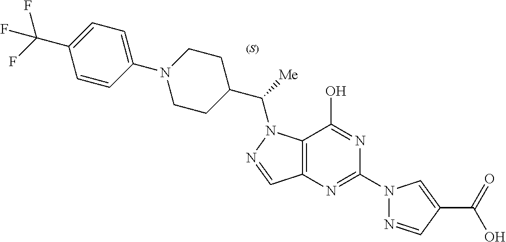 | powder MS (ESI) m/z: 502 [M + H]⁺ |
| 432 | 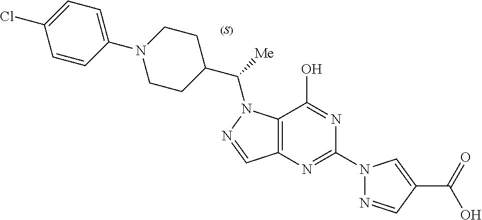 | powder MS (ESI) m/z: 468/470 [M + H]⁺ |
| 433 | 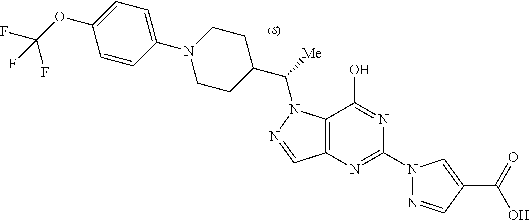 | powder MS (ESI) m/z: 518 [M + H]⁺ |
| 434 | 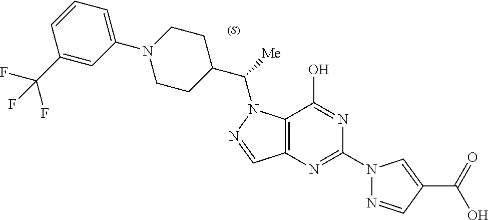 | powder MS (ESI) m/z: 502 [M + H]⁺ |
| 435 | 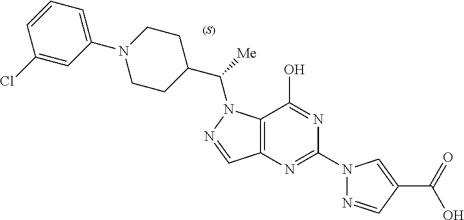 | powder MS (ESI) m/z: 468/470 [M + H]⁺ |
| 436 | 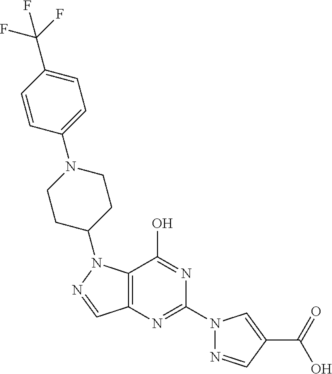 [SMILES: O=C(O)c1cnn(-c2nc(O)c3c(cnn3C3CCN(c4ccc(C(F)(F)F)cc4)CC3)n2)c1] | powder MS (ESI) m/z: 474 [M + H]⁺ |
| 437 | 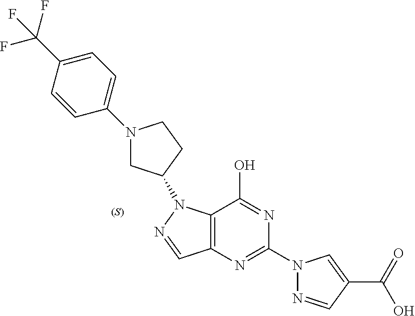 | powder MS (ESI) m/z: 460 [M + H]⁺ |
| 438 | 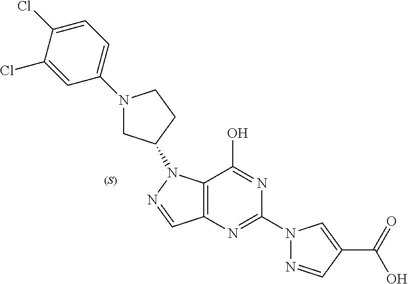 | powder MS (ESI) m/z: 460/462 [M + H]⁺ |
| 439 | 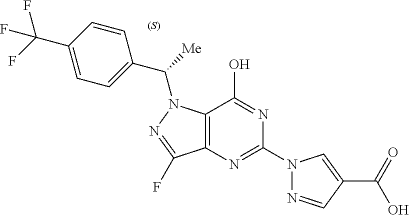 | powder MS (APCI) m/z: 437 [M + H]⁺ |
| 440 | 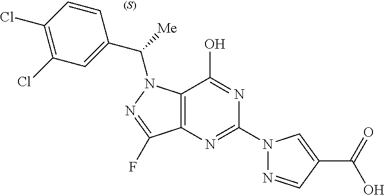 | powder MS (APCI) m/z: 437/439 [M + H]⁺ |
| 441 | 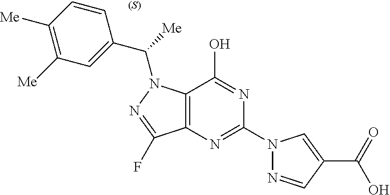 | powder MS (APCI) m/z: 397 [M + H]⁺ |
| 442 | 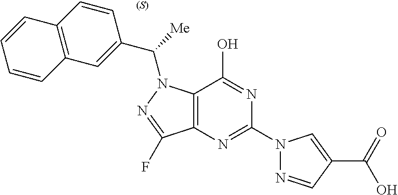 | powder MS (APCI) m/z: 419 [M + H]⁺ |
| 443 | 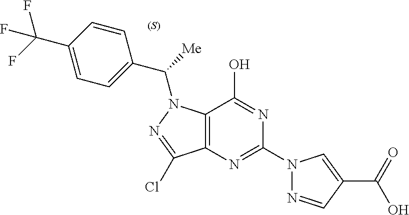 | powder MS (APCI) m/z: 453/455 [M + H]⁺ |
| 444 | 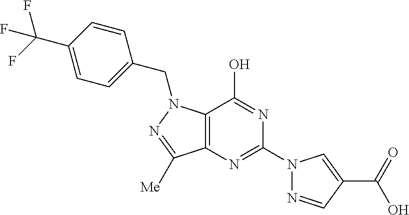 [SMILES: Cc1nn(Cc2ccc(C(F)(F)F)cc2)c2c(O)nc(-n3cc(C(=O)O)cn3)nc12] | powder MS (APCI) m/z: 419 [M + H]⁺ |
| 445 | 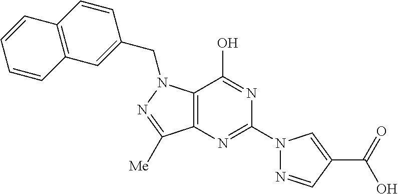 [SMILES: Cc1nn(Cc2ccc3ccccc3c2)c2c(O)nc(-n3cc(C(=O)O)cn3)nc12] | powder MS (APCI) m/z: 401 [M + H]⁺ |
| 446 | 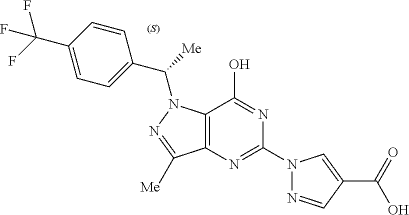 | powder MS (APCI) m/z: 433 [M + H]⁺ |
| 447 | 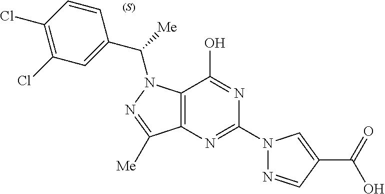 | powder MS (APCI) m/z: 433/455 [M + H]⁺ |
| 448 | 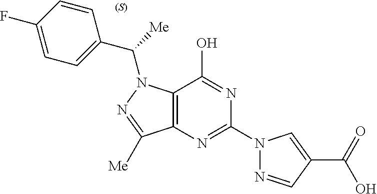 | powder MS (APCI) m/z: 383 [M + H]⁺ |
| 449 | 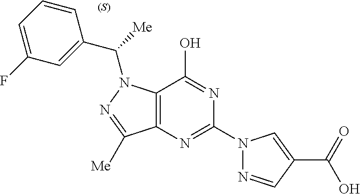 | powder MS (APCI) m/z: 383 [M + H]⁺ |
| 450 | 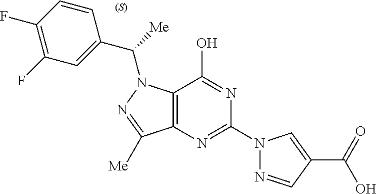 | powder MS (APCI) m/z: 401 [M + H]⁺ |
| 451 | 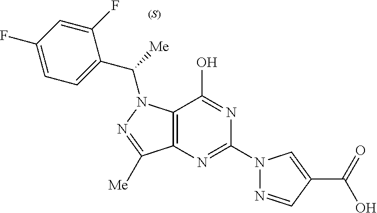 | powder MS (APCI) m/z: 401 [M + H]⁺ |
| 452 | 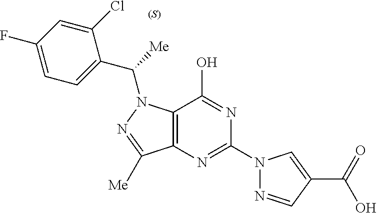 | powder MS (APCI) m/z: 417/419 [M + H]⁺ |
| 453 | 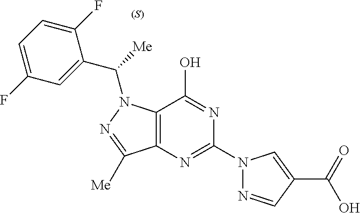 | powder MS (APCI) m/z: 401 [M + H]⁺ |
| 454 | 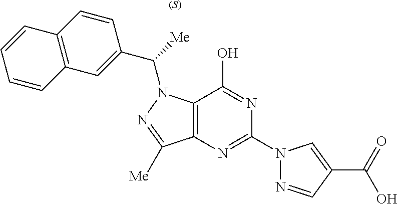 | powder MS (APCI) m/z: 415 [M + H]⁺ |
| 455 | 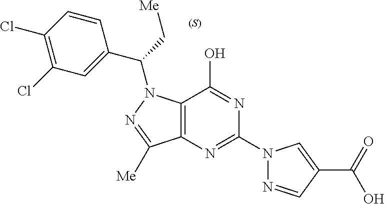 | powder MS (APCI) m/z: 447/449 [M + H]⁺ |
| 456 | 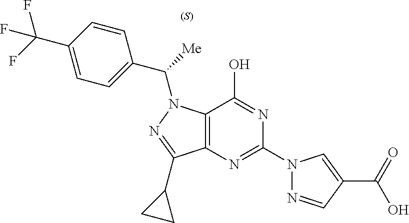 | powder MS (APCI) m/z: 459 [M + H]⁺ |
| 457 | 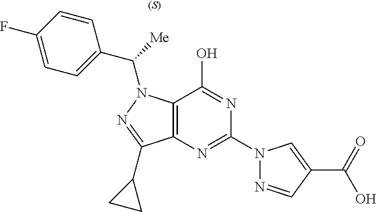 | powder MS (APCI) m/z: 409 [M + H]⁺ |
| 458 | 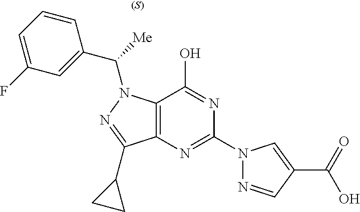 | powder MS (APCI) m/z: 409 [M + H]⁺ |
| 459 | 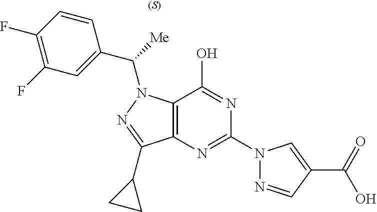 | powder MS (APCI) m/z: 427 [M + H]⁺ |
| 460 | 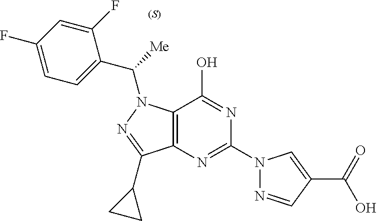 | powder MS (APCI) m/z: 427 [M + H]⁺ |
| 461 | 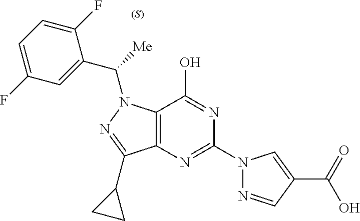 | powder MS (APCI) m/z: 427 [M + H]⁺ |
| 462 | 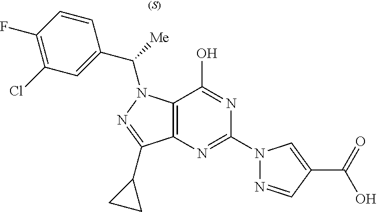 | powder MS (APCI) m/z: 443/445 [M + H]⁺ |
| 463 | 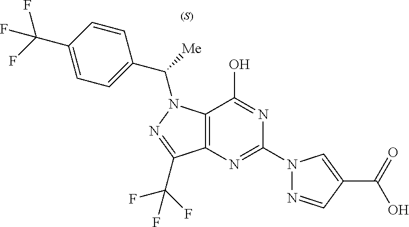 | powder MS (APCI) m/z: 487 [M + H]⁺ |
| 464 | 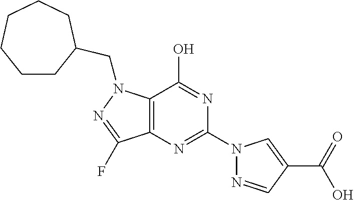 [SMILES: O=C(O)c1cnn(-c2nc(O)c3c(n2)c(F)nn3CC2CCCCCC2)c1] | powder MS (APCI) m/z: 375 [M + H]⁺ |
| 465 | 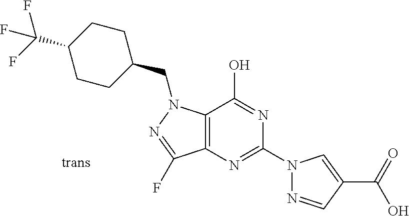 [SMILES: O=C(O)c1cnn(-c2nc(O)c3c(n2)c(F)nn3C[C@H]2CC[C@H](C(F)(F)F)CC2)c1] | powder MS (APCI) m/z: 429 [M + H]⁺ |
| 466 | 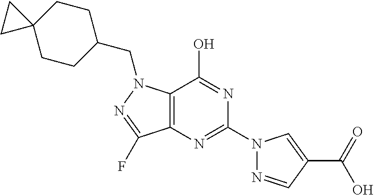 [SMILES: O=C(O)c1cnn(-c2nc(O)c3c(n2)c(F)nn3CC2CCC3(CC2)CC3)c1] | powder MS (APCI) m/z: 387 [M + H]⁺ |
| 467 | 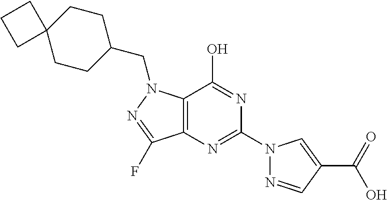 [SMILES: O=C(O)c1cnn(-c2nc(O)c3c(n2)c(F)nn3CC2CCC3(CCC3)CC2)c1] | powder MS (APCI) m/z: 401 [M + H]⁺ |
| 468 | 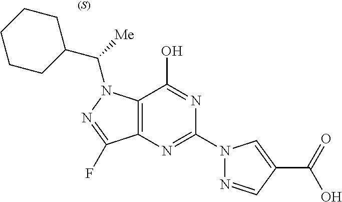 | powder MS (APCI) m/z: 375 [M + H]⁺ |
| 469 | 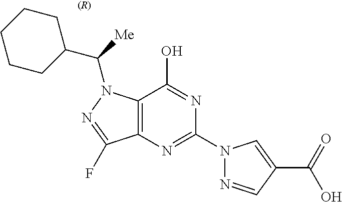 | powder MS (APCI) m/z: 375 [M + H]⁺ |
| 470 | 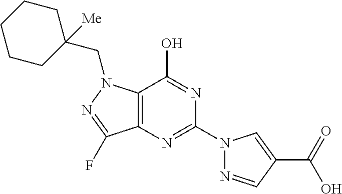 [SMILES: CC1(Cn2nc(F)c3nc(-n4cc(C(=O)O)cn4)nc(O)c32)CCCCC1] | powder MS (APCI) m/z: 375 [M + H]⁺ |
| 471 | 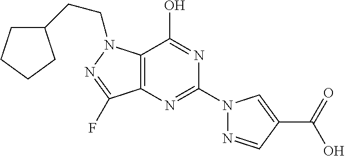 [SMILES: O=C(O)c1cnn(-c2nc(O)c3c(n2)c(F)nn3CCC2CCCC2)c1] | powder MS (APCI) m/z: 361 [M + H]⁺ |
| 472 | 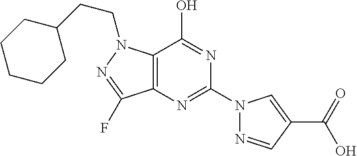 [SMILES: O=C(O)c1cnn(-c2nc(O)c3c(n2)c(F)nn3CCC2CCCCC2)c1] | powder MS (APCI) m/z: 375 [M + H]⁺ |
| 473 | 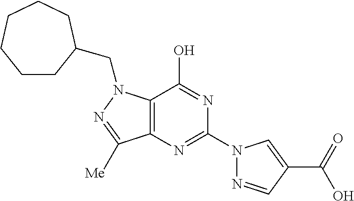 [SMILES: Cc1nn(CC2CCCCCC2)c2c(O)nc(-n3cc(C(=O)O)cn3)nc12] | powder MS (APCI) m/z: 371 [M + H]⁺ |
| 474 | 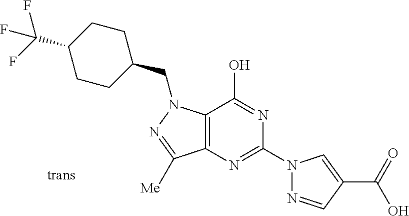 [SMILES: Cc1nn(C[C@H]2CC[C@H](C(F)(F)F)CC2)c2c(O)nc(-n3cc(C(=O)O)cn3)nc12] | powder MS (APCI) m/z: 425 [M + H]⁺ |
| 475 | 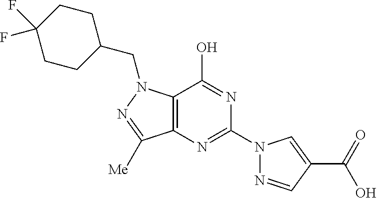 [SMILES: Cc1nn(CC2CCC(F)(F)CC2)c2c(O)nc(-n3cc(C(=O)O)cn3)nc12] | powder MS (APCI) m/z: 393 [M + H]⁺ |
| 476 | 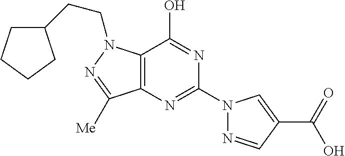 [SMILES: Cc1nn(CCC2CCCC2)c2c(O)nc(-n3cc(C(=O)O)cn3)nc12] | powder MS (APCI) m/z: 357 [M + H]⁺ |
| 477 | 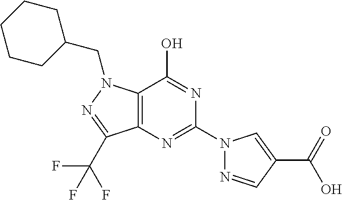 [SMILES: O=C(O)c1cnn(-c2nc(O)c3c(n2)c(C(F)(F)F)nn3CC2CCCCC2)c1] | powder MS (APCI) m/z: 411 [M + H]⁺ |
| 478 | 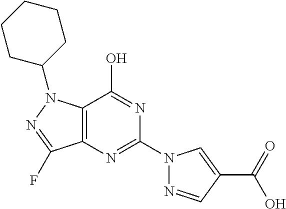 [SMILES: O=C(O)c1cnn(-c2nc(O)c3c(n2)c(F)nn3C2CCCCC2)c1] | powder MS (APCI) m/z: 347 [M + H]⁺ |
| 479 | 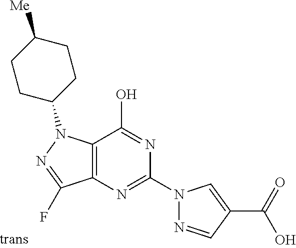 [SMILES: C[C@H]1CC[C@H](n2nc(F)c3nc(-n4cc(C(=O)O)cn4)nc(O)c32)CC1] | powder MS (APCI) m/z: 361 [M + H]⁺ |
| 480 | 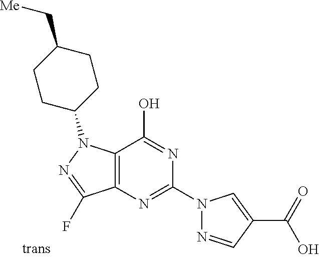 [SMILES: CC[C@H]1CC[C@H](n2nc(F)c3nc(-n4cc(C(=O)O)cn4)nc(O)c32)CC1] | powder MS (APCI) m/z: 375 [M + H]⁺ |
| 481 | 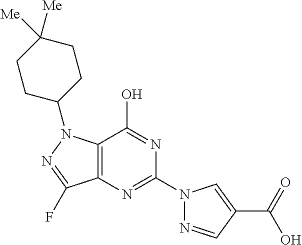 [SMILES: CC1(C)CCC(n2nc(F)c3nc(-n4cc(C(=O)O)cn4)nc(O)c32)CC1] | powder MS (APCI) m/z: 375 [M + H]⁺ |
| 482 | 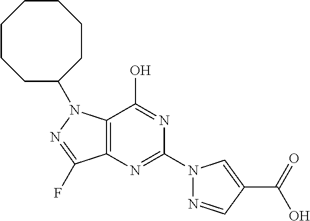 [SMILES: O=C(O)c1cnn(-c2nc(O)c3c(n2)c(F)nn3C2CCCCCCC2)c1] | powder MS (APCI) m/z: 375 [M + H]⁺ |
| 483 | 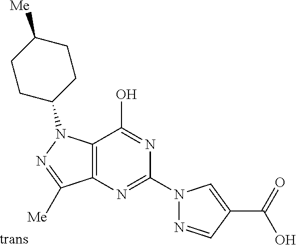 [SMILES: Cc1nn([C@H]2CC[C@H](C)CC2)c2c(O)nc(-n3cc(C(=O)O)cn3)nc12] | powder MS (APCI) m/z: 357 [M + H]⁺ |
| 484 | 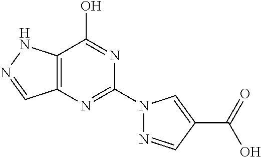 [SMILES: O=C(O)c1cnn(-c2nc(O)c3[nH]ncc3n2)c1] | powder MS (APCI) m/z: 247 [M + H]⁺ |
| 485 | 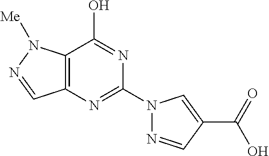 [SMILES: Cn1ncc2nc(-n3cc(C(=O)O)cn3)nc(O)c21] | powder MS (APCI) m/z: 261 [M + H]⁺ |
| 486 | 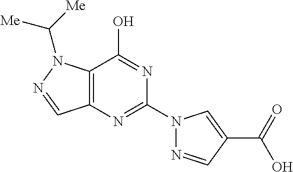 [SMILES: CC(C)n1ncc2nc(-n3cc(C(=O)O)cn3)nc(O)c21] | powder MS (APCI) m/z: 289 [M + H]⁺ |
| 487 | 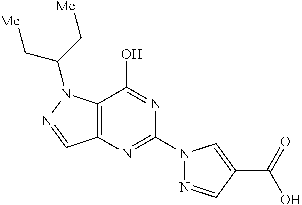 [SMILES: CCC(CC)n1ncc2nc(-n3cc(C(=O)O)cn3)nc(O)c21] | powder MS (APCI) m/z: 317 [M + H]⁺ |
| 488 | 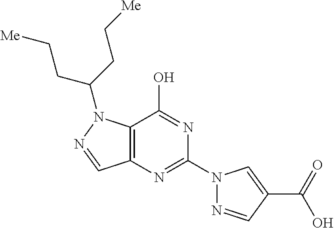 [SMILES: CCCC(CCC)n1ncc2nc(-n3cc(C(=O)O)cn3)nc(O)c21] | powder MS (APCI) m/z: 345 [M + H]⁺ |
| 489 | 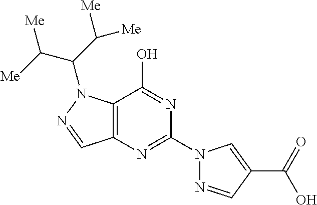 [SMILES: CC(C)C(C(C)C)n1ncc2nc(-n3cc(C(=O)O)cn3)nc(O)c21] | powder MS (APCI) m/z: 345 [M + H]⁺ |
| 490 | 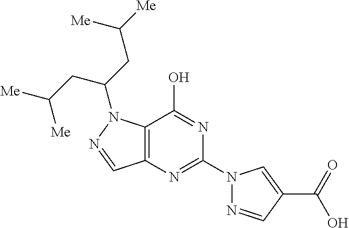 [SMILES: CC(C)CC(CC(C)C)n1ncc2nc(-n3cc(C(=O)O)cn3)nc(O)c21] | powder MS (APCI) m/z: 373 [M + H]⁺ |
| 491 | 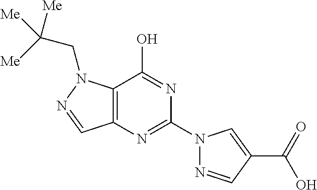 [SMILES: CC(C)(C)Cn1ncc2nc(-n3cc(C(=O)O)cn3)nc(O)c21] | powder MS (APCI) m/z: 317 [M + H]⁺ |
| 492 | 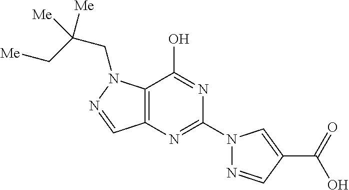 [SMILES: CCC(C)(C)Cn1ncc2nc(-n3cc(C(=O)O)cn3)nc(O)c21] | powder MS (APCI) m/z: 331 [M + H]⁺ |
| 493 | 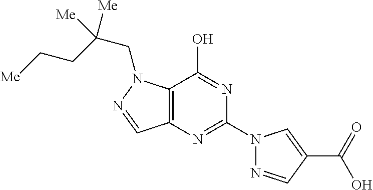 [SMILES: CCCC(C)(C)Cn1ncc2nc(-n3cc(C(=O)O)cn3)nc(O)c21] | powder MS (APCI) m/z: 345 [M + H]⁺ |
| 494 | 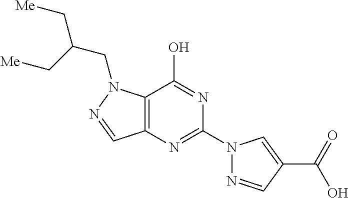 [SMILES: CCC(CC)Cn1ncc2nc(-n3cc(C(=O)O)cn3)nc(O)c21] | powder MS (APCI) m/z: 331 [M + H]⁺ |
| 495 | 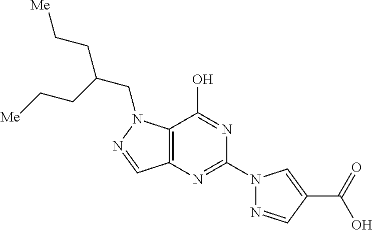 [SMILES: CCCC(CCC)Cn1ncc2nc(-n3cc(C(=O)O)cn3)nc(O)c21] | powder MS (APCI) m/z: 359 [M + H]⁺ |
| 496 | 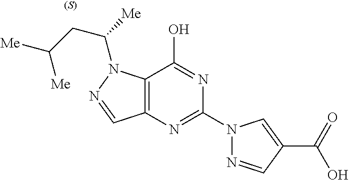 | powder MS (APCI) m/z: 331 [M + H]⁺ |
| 497 | 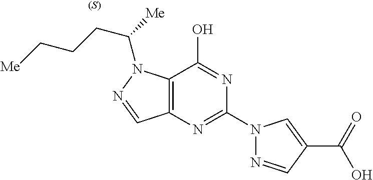 | powder MS (APCI) m/z: 331 [M + H]⁺ |
| 498 | 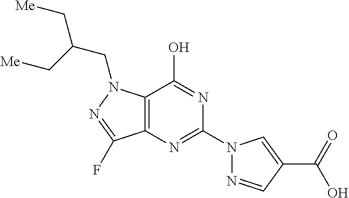 [SMILES: CCC(CC)Cn1nc(F)c2nc(-n3cc(C(=O)O)cn3)nc(O)c21] | powder MS (APCI) m/z: 349 [M + H]⁺ |
| 499 | 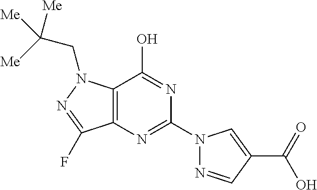 [SMILES: CC(C)(C)Cn1nc(F)c2nc(-n3cc(C(=O)O)cn3)nc(O)c21] | powder MS (APCI) m/z: 335 [M + H]⁺ |
| 500 | 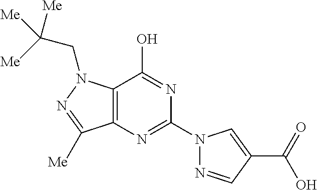 [SMILES: Cc1nn(CC(C)(C)C)c2c(O)nc(-n3cc(C(=O)O)cn3)nc12] | powder MS (APCI) m/z: 331 [M + H]⁺ |
| 501 | 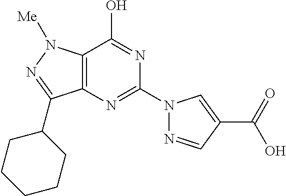 [SMILES: Cn1nc(C2CCCCC2)c2nc(-n3cc(C(=O)O)cn3)nc(O)c21] | powder MS (APCI) m/z: 343 [M + H]⁺ |
| 502 | 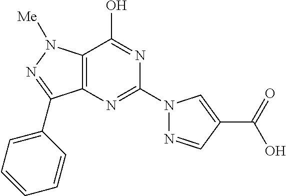 [SMILES: Cn1nc(-c2ccccc2)c2nc(-n3cc(C(=O)O)cn3)nc(O)c21] | powder MS (APCI) m/z: 337 [M + H]⁺ |
| 503 | 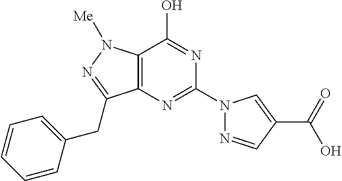 [SMILES: Cn1nc(Cc2ccccc2)c2nc(-n3cc(C(=O)O)cn3)nc(O)c21] | powder MS (APCI) m/z: 351 [M + H]⁺ |
| 504 | 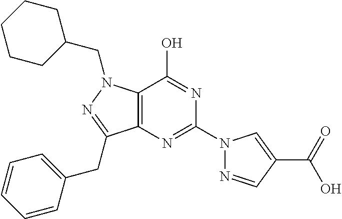 [SMILES: O=C(O)c1cnn(-c2nc(O)c3c(n2)c(Cc2ccccc2)nn3CC2CCCCC2)c1] | powder MS (APCI) m/z: 433 [M + H]⁺ |
| 505 | 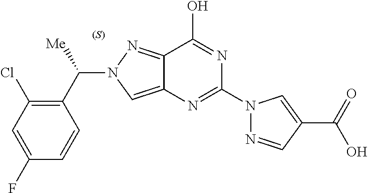 | powder MS (APCI) m/z: 403/405 [M + H]⁺ |
| 506 | 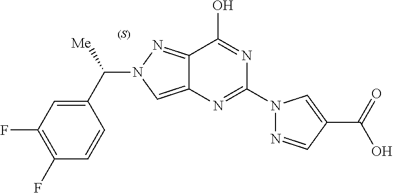 | powder MS (APCI) m/z: 401 [M + H]⁺ |

### REFERENCE EXAMPLE 1

### Preparation of ethyl 1-(7-methoxy-1H-pyrazolo[4,3-d]pyrimidin-5-yl)-1H-pyrazole-4-carboxylate

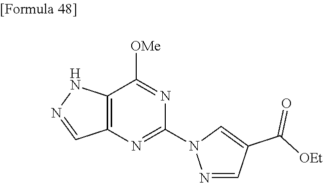 [SMILES: CCOC(=O)c1cnn(-c2nc(OC)c3[nH]ncc3n2)c1]

(1) To a solution of 5,7-dichloro-2-(4-methoxybenzyl)-2H-pyrazolo[4,3-d]pyrimidine (120 g) (see WO2006126718, Reference Examples 1 and 2) in tetrahydrofuran (480 mL) was added slowly sodium methoxide (78.6 mL, 28 wt % in methanol) under ice cooling, and the reaction mixture was stirred for 2 hours at room temperature. The reaction mixture was concentrated under reduced pressure, and the residue was added with water. The resulting crystal was collected by filtration to yield 5-chloro-7-methoxy-2-(4-methoxybenzyl)-2H-pyrazolo[4,3-d]pyrimidine (107 g, 91% yield) was obtained as colorless crystals.

MS (ESI) m/z: 305/307 [M+H]⁺.

(2) A suspension of the compound obtained in (1) (107 g), ethyl 1H-pyrazole-4-carboxylic acid (59.22 g), tripotassium phosphate (112.14 g), 2-di-t-butylphosphino-2′,4′,6′-triisopropyl biphenyl (11.22 g) and tris(dibenzylideneacetone)dipalladium(0) (8.06 g) in t-butyl alcohol (1173 mL) was stirred under nitrogen atmosphere for 4 hours at 90° C. The reaction mixture was added with water and filtered, and the resulting crystals were washed with methanol. The crystals were then dissolved in chloroform, and NH-silica gel (300 mL), silica gel (300 mL) and sodium sulfate (200 g) were added, followed by filtration to remove the insoluble material. The filtrate was concentrated under reduced pressure, the residue was added with methanol. The resulting crystals were corrected by filtration to yield ethyl 1-[7-methoxy-2-(4-methoxybenzyl)-2H-pyrazolo[4,3-d]pyrimidin-5-yl]-1H-pyrazole-4-carboxylate (99.62 g, 69% yield) as colorless crystals.

MS (ESI) m/z: 409 [M+H]⁺.

(3) A suspension of the compound obtained in (2) (99.62 g) in trifluoroacetic acid (398 mL) was stirred at 60° C. for 7 hours and then allowed to stand at room temperature for one day. The reaction mixture was concentrated under reduced pressure. The residue was suspended in chloroform and slowly added to a large amount of saturated aqueous sodium bicarbonate. The mixture was stirred, and the resulting crystals were collected by filtration, followed by washing with chloroform and water to yield the titled compound (64.11 g, 91% yield) as colorless crystals.

MS (ESI) m/z: 289 [M+H]⁺.

### REFERENCE EXAMPLE 2

### Preparation of ethyl 1-(7-methoxy-1H-pyrazolo[4,3-d]pyrimidin-5-yl)-1H-pyrazole-4-carboxylate

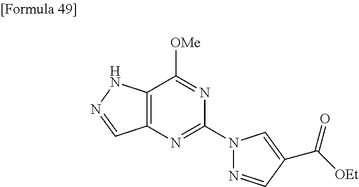 [SMILES: CCOC(=O)c1cnn(-c2nc(OC)c3[nH]ncc3n2)c1]

(1) To a solution of 5,7-dichloro-1-(4-methoxybenzyl)-1H-pyrazolo[4,3-d]pyrimidine (95 g) (cf. WO2006126718, Reference Examples 3 to 6) in tetrahydrofuran (380 mL) was added slowly sodium methoxide (65.2 mL, 28 wt % in methanol) under ice cooling, and the reaction mixture was stirred for 1.5 hours at room temperature. The reaction mixture was concentrated under reduced pressure, then added water to the residue. The resulting crystals was collected by filtration to yield 5-chloro-7-methoxy-1-(4-methoxybenzyl)-1H-pyrazolo[4,3-d]pyrimidin (94.27 g, 100% yield) as colorless crystals.

MS (APCI) m/z: 305/307 [M+H]⁺.

(2) A suspension of the compound obtained in (1) (89.27 g), ethyl 1H-pyrazole-4-carboxylate (45.16 g), tripotassium phosphate (93.3 g), 2-di-t-butylphosphino-2′,4′,6′-triisopropyl biphenyl (9.33 g) and tris(dibenzylideneacetone)dipalladium(0) (6.7 g) in t-butyl alcohol (900 mL) was stirred for 2 hours at 90° C. under nitrogen atmosphere. The reaction mixture was concentrated under reduced pressure, and the residue was added with chloroform and water. The organic layer was separated, and the aqueous layer was extracted with chloroform. The NH-silica gel (100 mL) and sodium sulfate (100 g) were added to the organic layer, and insoluble materials were removed by filtration. The filtrate was concentrated under reduced pressure, and the residue was added with methanol. The resulting crystals were collected by filtration to yield ethyl 1-[7-methoxy-1-(4-methoxybenzyl)-1H-pyrazolo[4,3-d]pyrimidin-5-yl]-1H-pyrazole-4-carboxylate (78.94 g, 66% yield) as colorless crystals.

MS (APCI) m/z: 409 [M+H]⁺.

(3) A suspension of the compound obtained in (2) (78.94 g) in trifluoroacetic acid (300 mL) was stirred for 6 hours at 60° C. and then for 5 days at room temperature. The reaction mixture was concentrated under reduced pressure, and the residue was suspended in chloroform. The suspension was added slowly to a large amount of saturated aqueous sodium bicarbonate, followed by stirring the mixture. The resulting crystals were collected by filtration and successively washed with chloroform and water to yield the titled compound (48.17 g, 86% yield) as colorless crystals.

MS (APCI) m/z: 289 [M+H]⁺.

### REFERENCE EXAMPLE 3

### Preparation of ethyl 1-[1-(3,4-dichlorobenzyl)-7-methoxy-1H-pyrazolo[4,3-d]pyrimidin-5-yl]-1H-pyrazole-4-carboxylate

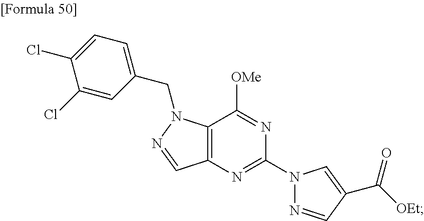 [SMILES: CCOC(=O)c1cnn(-c2nc(OC)c3c(cnn3Cc3ccc(Cl)c(Cl)c3)n2)c1]

### REFERENCE EXAMPLE 4

### Preparation of ethyl 1-[2-(3,4-dichlorobenzyl)-7-methoxy-2H-pyrazolo[4,3-d]pyrimidin-5-yl]-1H-pyrazole-4-carboxylate

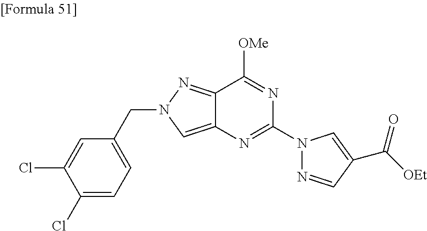 [SMILES: CCOC(=O)c1cnn(-c2nc(OC)c3nn(Cc4ccc(Cl)c(Cl)c4)cc3n2)c1]

A solution of ethyl 1-(7-methoxy-1H-pyrazolo[4,3-d]pyrimidin-5-yl)-1H-pyrazole-4-carboxylate (72 mg) prepared in Reference Example 1 or 2,4-bromomethyl-1,2-dichlorobenzene (78 mg) and potassium carbonate (86.4 mg) in acetonitrile (2 mL) was stirred for 2 hours at 80° C. After addition of chloroform and water to the reaction mixture, the organic layer was separated, and the aqueous layer was extracted with chloroform. The organic layer was concentrated under reduced pressure, and the residue was purified by silica gel column chromatography (solvent: hexane/ethyl acetate=70/30 to 40/60) to yield ethyl 1-[1-(3,4-dichlorobenzyl)-7-methoxy-1H-pyrazolo[4,3-d]pyrimidin-5-yl]-1H-pyrazole-4-carboxylate (43.2 mg, 47% yield) and ethyl 1-[2-(3,4-dichlorobenzyl)-7-methoxy-2H-pyrazolo[4,3-d]pyrimidin-5-yl]-1H-pyrazole-4-carboxylate (32 mg, 35% yield), respectively, as a colorless solid.

### Ethyl 1-[1-(3,4-dichlorobenzyl)-7-methoxy-1H-pyrazolo[4,3-d]pyrimidin-5-yl]-1H-pyrazole-4-carboxylate

MS (APCI) m/z: 447/449 [M+H]⁺.

### Ethyl 1-[2-(3,4-dichlorobenzyl)-7-methoxy-2H-pyrazolo[4,3-d]pyrimidin-5-yl]-1H-pyrazole-4-carboxylate

MS (APCI) m/z: 447/449 [M+H]⁺.

### REFERENCE EXAMPLES 5 TO 39

The compounds listed in the following Table 2 were obtained from the corresponding starting material in the same manner as described in Reference Examples 3 or 4.

TABLE 2

| Reference | | material |
|---|---|---|
| Example | Structure | properties |
| 5 | 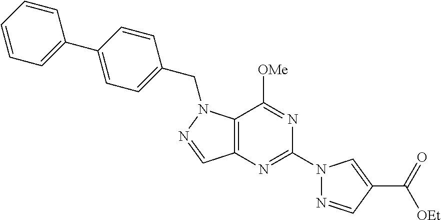 [SMILES: CCOC(=O)c1cnn(-c2nc(OC)c3c(cnn3Cc3ccc(-c4ccccc4)cc3)n2)c1] | powder MS (APCI) m/z: 455 [M + H]⁺ |
| 6 | 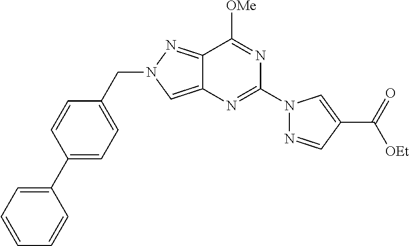 [SMILES: CCOC(=O)c1cnn(-c2nc(OC)c3nn(Cc4ccc(-c5ccccc5)cc4)cc3n2)c1] | powder MS (APCI) m/z: 455 [M + H]⁺ |
| 7 | 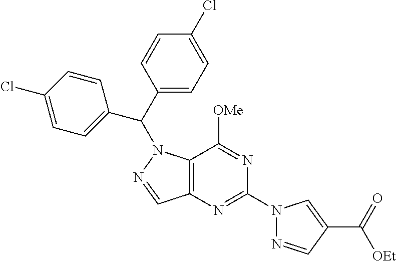 [SMILES: CCOC(=O)c1cnn(-c2nc(OC)c3c(cnn3C(c3ccc(Cl)cc3)c3ccc(Cl)cc3)n2)c1] | powder MS (APCI) m/z: 523/525 [M + H]⁺ |
| 8 | 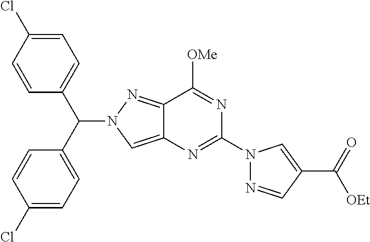 [SMILES: CCOC(=O)c1cnn(-c2nc(OC)c3nn(C(c4ccc(Cl)cc4)c4ccc(Cl)cc4)cc3n2)c1] | powder MS (APCI) m/z: 523/525 [M + H]⁺ |
| 9 | 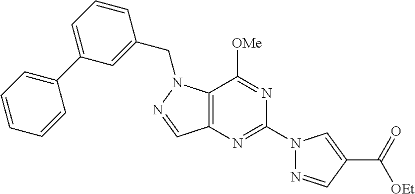 [SMILES: CCOC(=O)c1cnn(-c2nc(OC)c3c(cnn3Cc3cccc(-c4ccccc4)c3)n2)c1] | powder MS (APCI) m/z: 455 [M + H]⁺ |
| 10 | 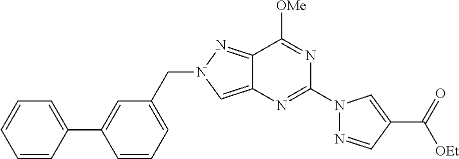 [SMILES: CCOC(=O)c1cnn(-c2nc(OC)c3nn(Cc4cccc(-c5ccccc5)c4)cc3n2)c1] | powder MS (APCI) m/z: 455 [M + H]⁺ |
| 11 | 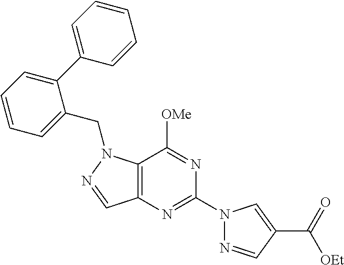 [SMILES: CCOC(=O)c1cnn(-c2nc(OC)c3c(cnn3Cc3ccccc3-c3ccccc3)n2)c1] | powder MS (APCI) m/z: 455 [M + H]⁺ |
| 12 | 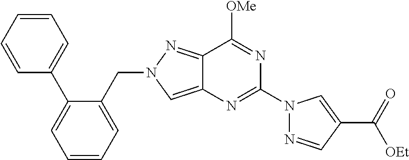 [SMILES: CCOC(=O)c1cnn(-c2nc(OC)c3nn(Cc4ccccc4-c4ccccc4)cc3n2)c1] | powder MS (APCI) m/z: 455 [M + H]⁺ |
| 13 | 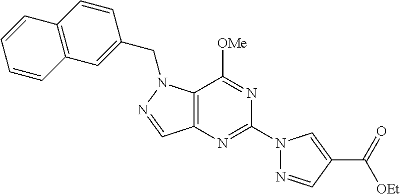 [SMILES: CCOC(=O)c1cnn(-c2nc(OC)c3c(cnn3Cc3ccc4ccccc4c3)n2)c1] | powder MS (APCI) m/z: 429 [M + H]⁺ |
| 14 | 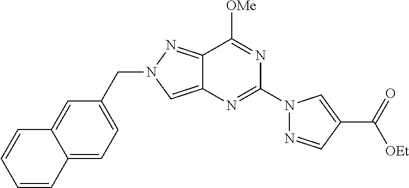 [SMILES: CCOC(=O)c1cnn(-c2nc(OC)c3nn(Cc4ccc5ccccc5c4)cc3n2)c1] | powder MS (APCI) m/z: 429 [M + H]⁺ |
| 15 | 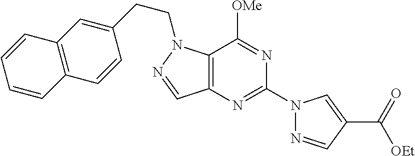 [SMILES: CCOC(=O)c1cnn(-c2nc(OC)c3c(cnn3CCc3ccc4ccccc4c3)n2)c1] | powder MS (APCI) m/z: 443 [M + H]⁺ |
| 16 | 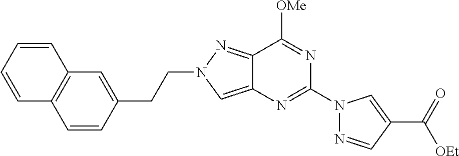 [SMILES: CCOC(=O)c1cnn(-c2nc(OC)c3nn(CCc4ccc5ccccc5c4)cc3n2)c1] | powder MS (APCI) m/z: 443 [M + H]⁺ |
| 17 | 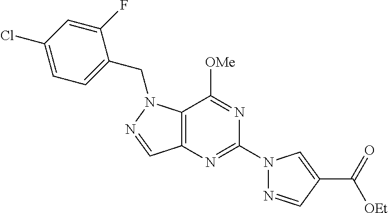 [SMILES: CCOC(=O)c1cnn(-c2nc(OC)c3c(cnn3Cc3ccc(Cl)cc3F)n2)c1] | powder MS (APCI) m/z: 431/433 [M + H]⁺ |
| 18 | 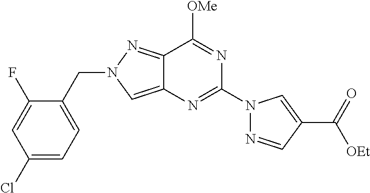 [SMILES: CCOC(=O)c1cnn(-c2nc(OC)c3nn(Cc4ccc(Cl)cc4F)cc3n2)c1] | powder MS (APCI) m/z: 431/433 [M + H]⁺ |
| 19 | 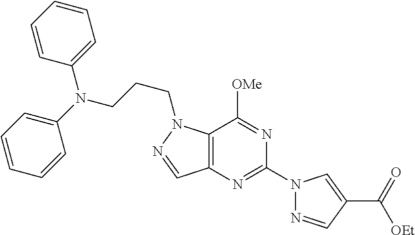 [SMILES: CCOC(=O)c1cnn(-c2nc(OC)c3c(cnn3CCCN(c3ccccc3)c3ccccc3)n2)c1] | powder MS (ESI) m/z: 498 [M + H]⁺ |
| 20 | 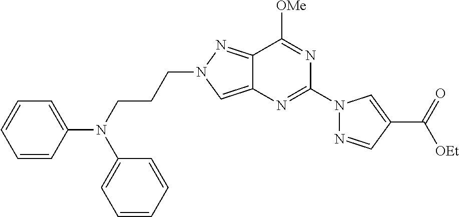 [SMILES: CCOC(=O)c1cnn(-c2nc(OC)c3nn(CCCN(c4ccccc4)c4ccccc4)cc3n2)c1] | powder MS (ESI) m/z: 498 [M + H]⁺ |
| 21 | 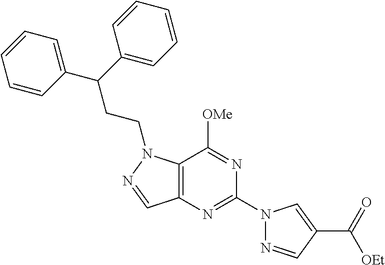 [SMILES: CCOC(=O)c1cnn(-c2nc(OC)c3c(cnn3CCC(c3ccccc3)c3ccccc3)n2)c1] | powder MS (APCI) m/z: 483 [M + H]⁺ |
| 22 | 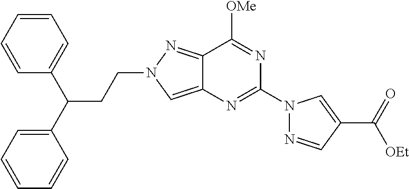 [SMILES: CCOC(=O)c1cnn(-c2nc(OC)c3nn(CCC(c4ccccc4)c4ccccc4)cc3n2)c1] | powder MS (APCI) m/z: 483 [M + H]⁺ |
| 23 | 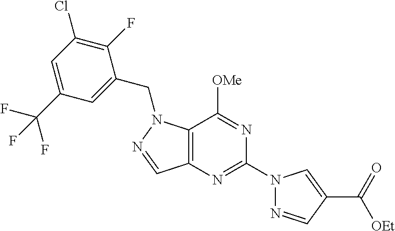 [SMILES: CCOC(=O)c1cnn(-c2nc(OC)c3c(cnn3Cc3cc(C(F)(F)F)cc(Cl)c3F)n2)c1] | powder MS (APCI) m/z: 499/501 [M + H]⁺ |
| 24 | 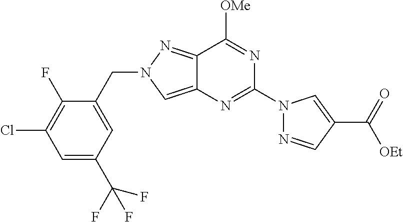 [SMILES: CCOC(=O)c1cnn(-c2nc(OC)c3nn(Cc4cc(C(F)(F)F)cc(Cl)c4F)cc3n2)c1] | powder MS (APCI) m/z: 499/501 [M + H]⁺ |
| 25 | 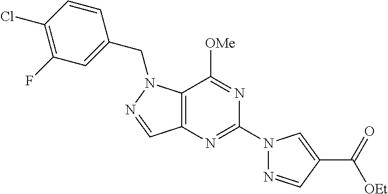 [SMILES: CCOC(=O)c1cnn(-c2nc(OC)c3c(cnn3Cc3ccc(Cl)c(F)c3)n2)c1] | powder MS (APCI) m/z: 431/433 [M + H]⁺ |
| 26 | 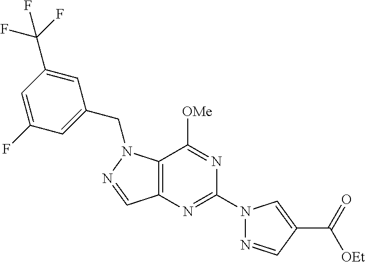 [SMILES: CCOC(=O)c1cnn(-c2nc(OC)c3c(cnn3Cc3cc(F)cc(C(F)(F)F)c3)n2)c1] | powder MS (APCI) m/z: 465 [M + H]⁺ |
| 27 | 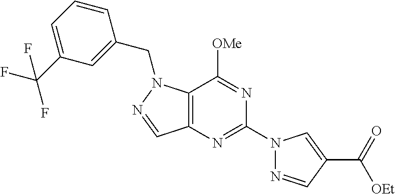 [SMILES: CCOC(=O)c1cnn(-c2nc(OC)c3c(cnn3Cc3cccc(C(F)(F)F)c3)n2)c1] | powder MS (APCI) m/z: 447 [M + H]⁺ |
| 28 | 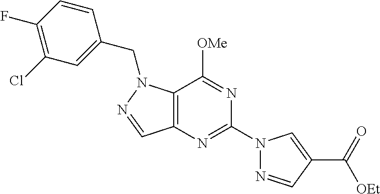 [SMILES: CCOC(=O)c1cnn(-c2nc(OC)c3c(cnn3Cc3ccc(F)c(Cl)c3)n2)c1] | powder MS (APCI) m/z: 431/433 [M + H]⁺ |
| 29 | 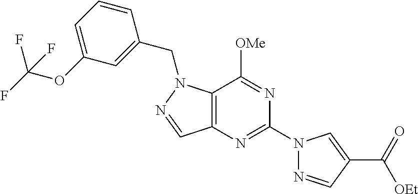 [SMILES: CCOC(=O)c1cnn(-c2nc(OC)c3c(cnn3Cc3cccc(OC(F)(F)F)c3)n2)c1] | powder MS (APCI) m/z: 463 [M + H]⁺ |
| 30 | 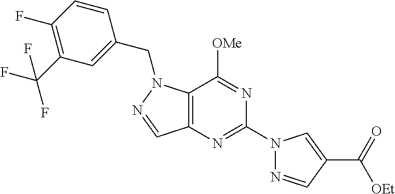 [SMILES: CCOC(=O)c1cnn(-c2nc(OC)c3c(cnn3Cc3ccc(F)c(C(F)(F)F)c3)n2)c1] | powder MS (APCI) m/z: 465 [M + H]⁺ |
| 31 | 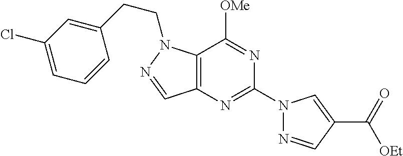 [SMILES: CCOC(=O)c1cnn(-c2nc(OC)c3c(cnn3CCc3cccc(Cl)c3)n2)c1] | powder MS (APCI) m/z: 427/429 [M + H]⁺ |
| 32 | 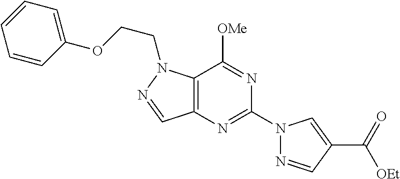 [SMILES: CCOC(=O)c1cnn(-c2nc(OC)c3c(cnn3CCOc3ccccc3)n2)c1] | powder MS (APCI) m/z: 409 [M + H]⁺ |
| 33 | 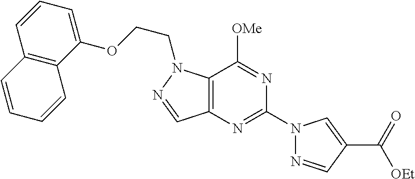 [SMILES: CCOC(=O)c1cnn(-c2nc(OC)c3c(cnn3CCOc3cccc4ccccc34)n2)c1] | powder MS (ESI) m/z: 459 [M + H]⁺ |
| 34 | 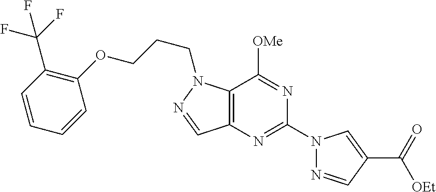 [SMILES: CCOC(=O)c1cnn(-c2nc(OC)c3c(cnn3CCCOc3ccccc3C(F)(F)F)n2)c1] | powder MS (ESI) m/z: 491 [M + H]⁺ |
| 35 | 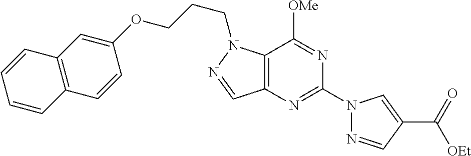 [SMILES: CCOC(=O)c1cnn(-c2nc(OC)c3c(cnn3CCCOc3ccc4ccccc4c3)n2)c1] | powder MS (ESI) m/z: 473 [M + H]⁺ |
| 36 | 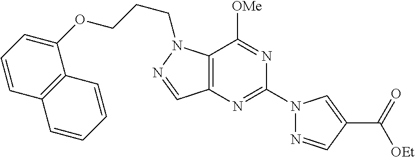 [SMILES: CCOC(=O)c1cnn(-c2nc(OC)c3c(cnn3CCCOc3cccc4ccccc34)n2)c1] | powder MS (ESI) m/z: 473 [M + H]⁺ |
| 37 | 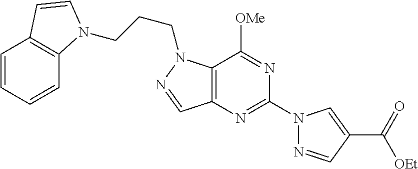 [SMILES: CCOC(=O)c1cnn(-c2nc(OC)c3c(cnn3CCCn3ccc4ccccc43)n2)c1] | powder MS (ESI) m/z: 446 [M + H]⁺ |
| 38 | 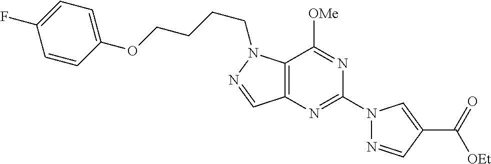 [SMILES: CCOC(=O)c1cnn(-c2nc(OC)c3c(cnn3CCCCOc3ccc(F)cc3)n2)c1] | powder MS (APCI) m/z: 455 [M + H]⁺ |
| 39 | 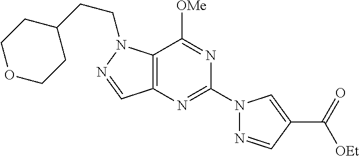 [SMILES: CCOC(=O)c1cnn(-c2nc(OC)c3c(cnn3CCC3CCOCC3)n2)c1] | powder MS (ESI) m/z: 401 [M + H]⁺ |

### REFERENCE EXAMPLE 40

### Preparation of ethyl 1-[7-methoxy-1-({1-[5-(trifluoromethyl)pyridin-2-yl]piperidin-4-yl}methyl)-1H-pyrazolo[4,3-d]pyrimidin-5-yl]-1H-pyrazole-4-carboxylate

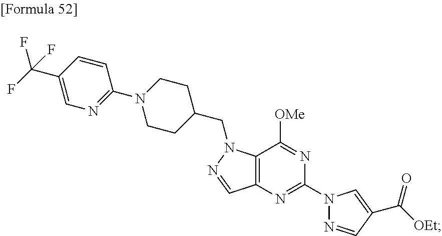 [SMILES: CCOC(=O)c1cnn(-c2nc(OC)c3c(cnn3CC3CCN(c4ccc(C(F)(F)F)cn4)CC3)n2)c1]

### REFERENCE EXAMPLE 41

### Preparation of ethyl 1-[7-methoxy-2-({1-[5-(trifluoromethyl)pyridin-2-yl]piperidin-4-yl}methyl)-2H-pyrazolo[4,3-d]pyrimidin-5-yl]-1H-pyrazole-4-carboxylate

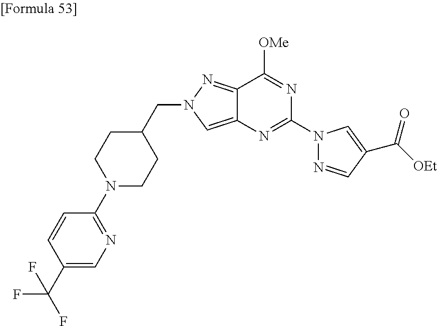 [SMILES: CCOC(=O)c1cnn(-c2nc(OC)c3nn(CC4CCN(c5ccc(C(F)(F)F)cn5)CC4)cc3n2)c1]

To a suspension of ethyl 1-(7-methoxy-1H-pyrazolo[4,3-d]pyrimidin-5-yl)-1H-pyrazole-4-carboxylate (288 mg) prepared in Reference Example 1 or 2, {1-[5-trifluoromethyl)pyridin-2-yl]piperidin-4-yl}methanol (338 mg) and triphenylphosphine (656 mg) in tetrahydrofuran (10 mL) was added diisopropyl azodicarboxylate (1.31 mL, 1.9 mol/L in toluene), and the reaction mixture was stirred at room temperature for 3.5 hours. The reaction mixture was concentrated under reduced pressure, and the resulting residue was purified by silica gel column chromatography (solvent: hexane/ethyl acetate=85/15 to 0/100) to yield ethyl 1-[7-methoxy-1-({1-[5-(trifluoromethyl)pyridin-2-yl]piperidin-4-yl}methyl)-1H-pyrazolo[4,3-d]pyrimidin-5-yl]-1H-pyrazole-4-carboxylate (327.5 mg, 62% yield) and ethyl 1-[7-methoxy-2-({1-[5-(trifluoromethyl)pyridin-2-yl]piperidin-4-yl}methyl)-2H-pyrazolo[4,3-d]pyrimidin-5-yl]-1H-pyrazole-4-carboxylate (68.6 mg, 13% yield), respectively, as a colorless solid.

### Ethyl 1-[7-methoxy-1-({1-[5-(trifluoromethyl)pyridin-2-yl]piperidin-4-yl}methyl)-1H-pyrazolo[4,3-d]pyrimidin-5-yl]-1H-pyrazole-4-carboxylate

MS (APCI) m/z: 531 [M+H]⁺.

### Ethyl 1-[7-methoxy-2-({1-[5-(trifluoromethyl)pyridin-2-yl]piperidin-4-yl}methyl)-2H-pyrazolo[4,3-d]pyrimidin-5-yl]-1H-pyrazole-4-carboxylate

MS (APCI) m/z: 531 [M+H]⁺.

### REFERENCE EXAMPLES 42 TO 355

The compounds listed in the following Table 3 were obtained from the corresponding starting material in the same manner as described in Reference Examples 40, 41. The compounds of Reference Examples 83, 84, 195 to 220, 223, 224, 226 to 239, 241 to 246, 250 to 255, 258 to 281, 302 to 305 and 311 to 322 were obtained as an optically active form by resolution of the racemic mixture using chiral HPLC (chiral HPLC column: CHIRALPAK IA, CHIRALPAK IC, CHIRALPAK ID or CHIRALPAK IF; mobile phase: a liquid mixture of three of four selected from the group consisting of hexane, methanol, ethanol, 2-propanol, tetrahydrofuran, methyl t-butyl ether and diethylamine) or chiral SFC (chiral HPLC column: CHIRALPAK IA/SFC; mobile phase: a mixture of carbon dioxide, tetrahydrofuran, ethanol and diethylamine).

TABLE 3

| Reference | | material |
|---|---|---|
| Example | Structure | properties |
| 42 | 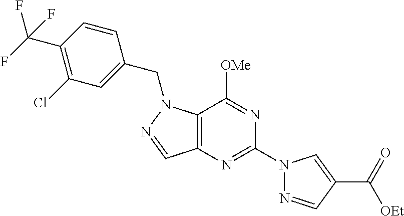 [SMILES: CCOC(=O)c1cnn(-c2nc(OC)c3c(cnn3Cc3ccc(C(F)(F)F)c(Cl)c3)n2)c1] | powder MS (APCI) m/z: 481/483 [M + H]⁺ |
| 43 | 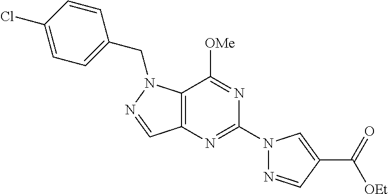 [SMILES: CCOC(=O)c1cnn(-c2nc(OC)c3c(cnn3Cc3ccc(Cl)cc3)n2)c1] | powder MS (APCI) m/z: 413/415 [M + H]⁺ |
| 44 | 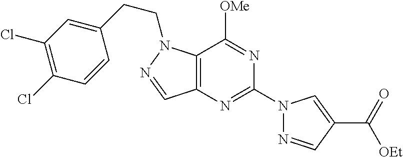 [SMILES: CCOC(=O)c1cnn(-c2nc(OC)c3c(cnn3CCc3ccc(Cl)c(Cl)c3)n2)c1] | powder MS (APCI) m/z: 461/463 [M + H]⁺ |
| 45 | 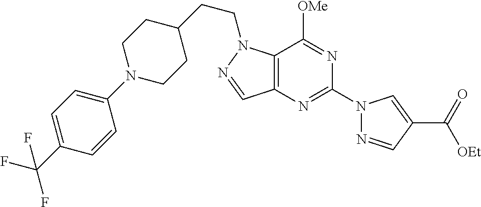 [SMILES: CCOC(=O)c1cnn(-c2nc(OC)c3c(cnn3CCC3CCN(c4ccc(C(F)(F)F)cc4)CC3)n2)c1] | powder MS (APCI) m/z: 544 [M + H]⁺ |
| 46 | 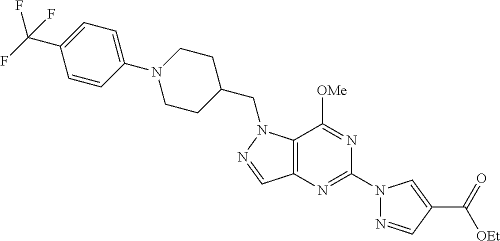 [SMILES: CCOC(=O)c1cnn(-c2nc(OC)c3c(cnn3CC3CCN(c4ccc(C(F)(F)F)cc4)CC3)n2)c1] | powder MS (APCI) m/z: 530 [M + H]⁺ |
| 47 | 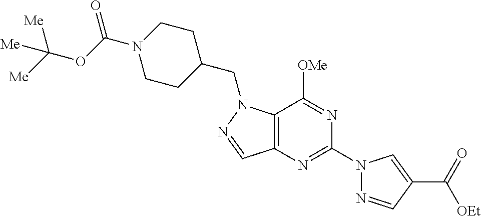 [SMILES: CCOC(=O)c1cnn(-c2nc(OC)c3c(cnn3CC3CCN(C(=O)OC(C)(C)C)CC3)n2)c1] | powder MS (APCI) m/z: 486 [M + H]⁺ |
| 48 | 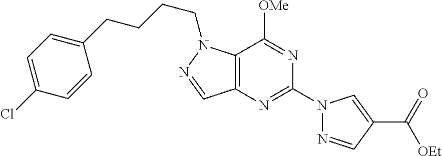 [SMILES: CCOC(=O)c1cnn(-c2nc(OC)c3c(cnn3CCCCc3ccc(Cl)cc3)n2)c1] | powder MS (APCI) m/z: 455/457 [M + H]⁺ |
| 49 | 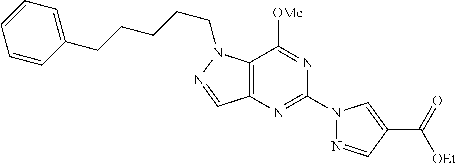 [SMILES: CCOC(=O)c1cnn(-c2nc(OC)c3c(cnn3CCCCCc3ccccc3)n2)c1] | powder MS (APCI) m/z: 435 [M + H]⁺ |
| 50 | 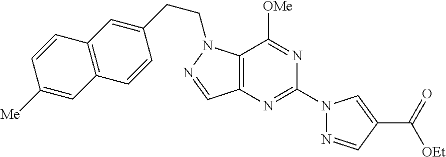 [SMILES: CCOC(=O)c1cnn(-c2nc(OC)c3c(cnn3CCc3ccc4cc(C)ccc4c3)n2)c1] | powder MS (ESI) m/z: 457 [M + H]⁺ |
| 51 | 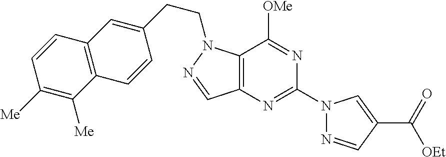 [SMILES: CCOC(=O)c1cnn(-c2nc(OC)c3c(cnn3CCc3ccc4c(C)c(C)ccc4c3)n2)c1] | powder MS (ESI) m/z: 471 [M + H]⁺ |
| 52 | 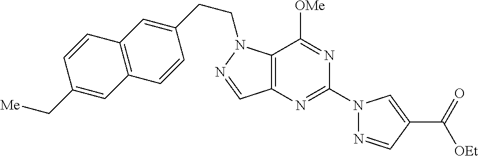 [SMILES: CCOC(=O)c1cnn(-c2nc(OC)c3c(cnn3CCc3ccc4cc(CC)ccc4c3)n2)c1] | powder MS (ESI) m/z: 471 [M + H]⁺ |
| 53 | 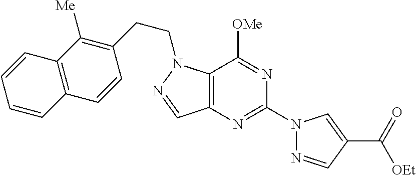 [SMILES: CCOC(=O)c1cnn(-c2nc(OC)c3c(cnn3CCc3ccc4ccccc4c3C)n2)c1] | powder MS (ESI) m/z: 457 [M + H]⁺ |
| 54 | 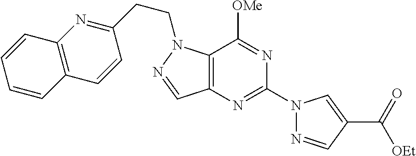 [SMILES: CCOC(=O)c1cnn(-c2nc(OC)c3c(cnn3CCc3ccc4ccccc4n3)n2)c1] | powder MS (ESI) m/z: 444 [M + H]⁺ |
| 55 | 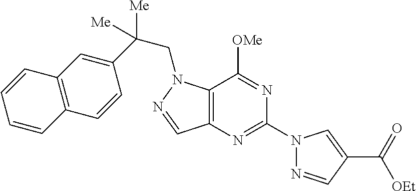 [SMILES: CCOC(=O)c1cnn(-c2nc(OC)c3c(cnn3CC(C)(C)c3ccc4ccccc4c3)n2)c1] | powder MS (ESI) m/z: 471 [M + H]⁺ |
| 56 | 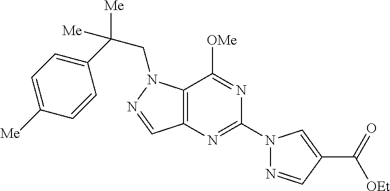 [SMILES: CCOC(=O)c1cnn(-c2nc(OC)c3c(cnn3CC(C)(C)c3ccc(C)cc3)n2)c1] | powder MS (APCI) m/z: 435 [M + H]⁺ |
| 57 | 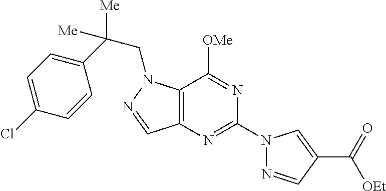 [SMILES: CCOC(=O)c1cnn(-c2nc(OC)c3c(cnn3CC(C)(C)c3ccc(Cl)cc3)n2)c1] | powder MS (APCI) m/z: 455/457 [M + H]⁺ |
| 58 | 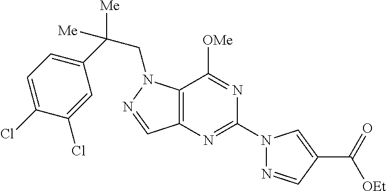 [SMILES: CCOC(=O)c1cnn(-c2nc(OC)c3c(cnn3CC(C)(C)c3ccc(Cl)c(Cl)c3)n2)c1] | powder MS (APCI) m/z: 489/491 [M + H]⁺ |
| 59 | 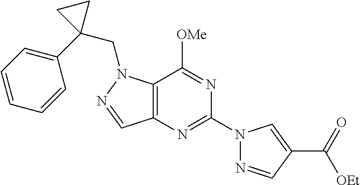 [SMILES: CCOC(=O)c1cnn(-c2nc(OC)c3c(cnn3CC3(c4ccccc4)CC3)n2)c1] | powder MS (APCI) m/z: 419 [M + H]⁺ |
| 60 | 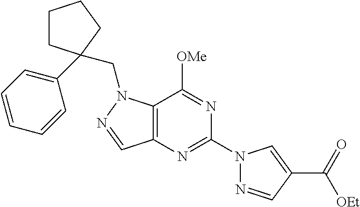 [SMILES: CCOC(=O)c1cnn(-c2nc(OC)c3c(cnn3CC3(c4ccccc4)CCCC3)n2)c1] | powder MS (APCI) m/z: 447 [M + H]⁺ |
| 61 | 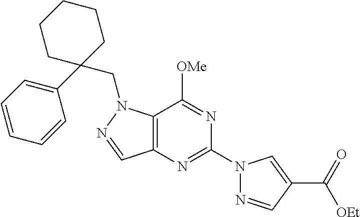 [SMILES: CCOC(=O)c1cnn(-c2nc(OC)c3c(cnn3CC3(c4ccccc4)CCCCC3)n2)c1] | powder MS (APCI) m/z: 461 [M + H]⁺ |
| 62 | 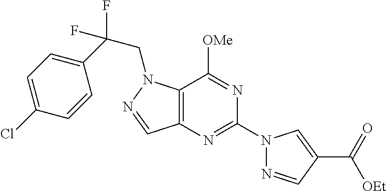 [SMILES: CCOC(=O)c1cnn(-c2nc(OC)c3c(cnn3CC(F)(F)c3ccc(Cl)cc3)n2)c1] | powder MS (APCI) m/z: 463/465 [M + H]⁺ |
| 63 | 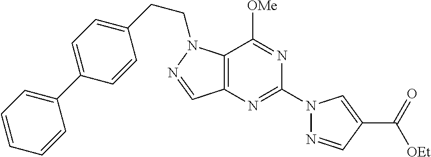 [SMILES: CCOC(=O)c1cnn(-c2nc(OC)c3c(cnn3CCc3ccc(-c4ccccc4)cc3)n2)c1] | powder MS (ESI) m/z: 469 [M + H]⁺ |
| 64 | 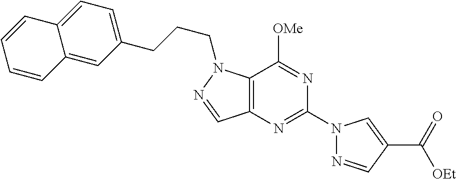 [SMILES: CCOC(=O)c1cnn(-c2nc(OC)c3c(cnn3CCCc3ccc4ccccc4c3)n2)c1] | powder MS (ESI) m/z: 457 [M + H]⁺ |
| 65 | 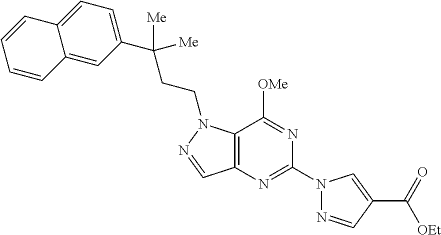 [SMILES: CCOC(=O)c1cnn(-c2nc(OC)c3c(cnn3CCC(C)(C)c3ccc4ccccc4c3)n2)c1] | powder MS (APCI) m/z: 485 [M + H]⁺ |
| 66 | 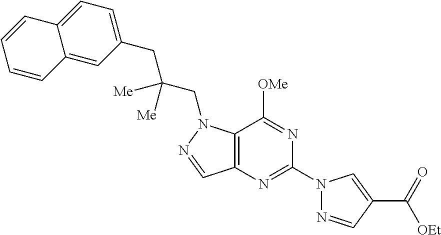 [SMILES: CCOC(=O)c1cnn(-c2nc(OC)c3c(cnn3CC(C)(C)Cc3ccc4ccccc4c3)n2)c1] | powder MS (APCI) m/z: 485 [M + H]⁺ |
| 67 | 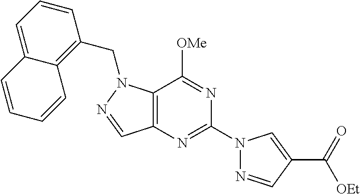 [SMILES: CCOC(=O)c1cnn(-c2nc(OC)c3c(cnn3Cc3cccc4ccccc34)n2)c1] | powder MS (APCI) m/z: 429 [M + H]⁺ |
| 68 | 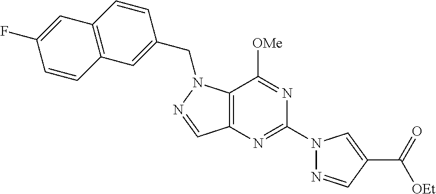 [SMILES: CCOC(=O)c1cnn(-c2nc(OC)c3c(cnn3Cc3ccc4cc(F)ccc4c3)n2)c1] | powder MS (APCI) m/z: 447 [M + H]⁺ |
| 69 | 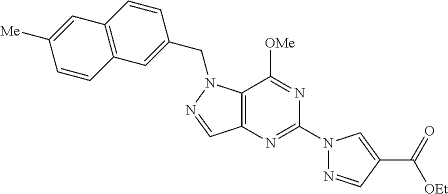 [SMILES: CCOC(=O)c1cnn(-c2nc(OC)c3c(cnn3Cc3ccc4cc(C)ccc4c3)n2)c1] | powder MS (APCI) m/z: 443 [M + H]⁺ |
| 70 | 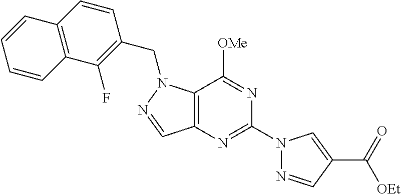 [SMILES: CCOC(=O)c1cnn(-c2nc(OC)c3c(cnn3Cc3ccc4ccccc4c3F)n2)c1] | powder MS (APCI) m/z: 447 [M + H]⁺ |
| 71 | 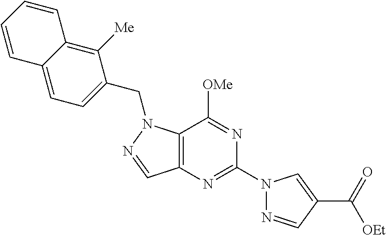 [SMILES: CCOC(=O)c1cnn(-c2nc(OC)c3c(cnn3Cc3ccc4ccccc4c3C)n2)c1] | powder MS (APCI) m/z: 443 [M + H]⁺ |
| 72 | 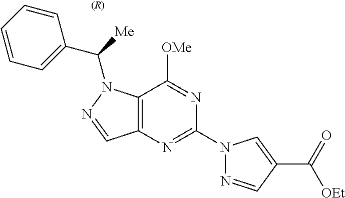 | powder MS (ESI) m/z: 393 [M + H]⁺ |
| 73 | 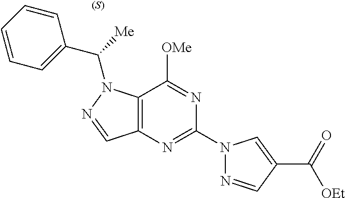 | viscous material MS (ESI) m/z: 393 [M + H]⁺ |
| 74 | 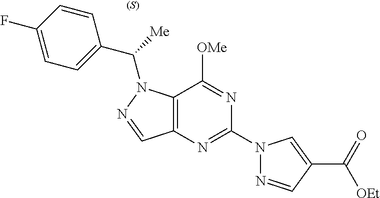 | powder MS (APCI) m/z: 411 [M + H]⁺ |
| 75 | 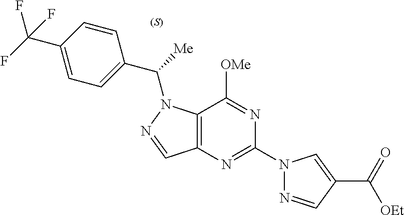 | powder MS (APCI) m/z: 461 [M + H]⁺ |
| 76 | 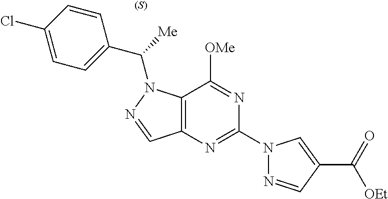 | powder MS (APCI) m/z: 427/429 [M + H]⁺ |
| 77 | 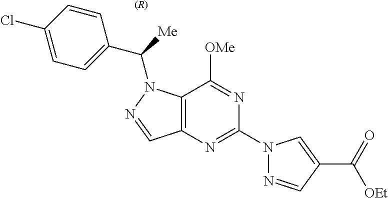 | powder MS (APCI) m/z: 427/429 [M + H]⁺ |
| 78 | 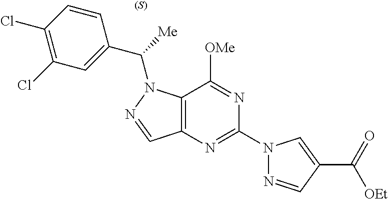 | powder MS (APCI) m/z: 461/463 [M + H]⁺ |
| 79 | 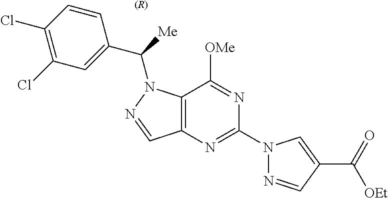 | powder MS (APCI) m/z: 461/463 [M + H]⁺ |
| 80 | 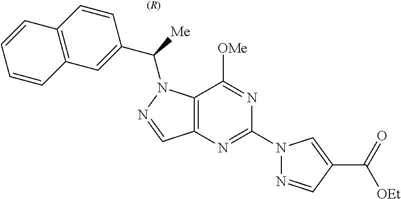 | viscous material MS (ESI) m/z: 443 [M + H]⁺ |
| 81 | 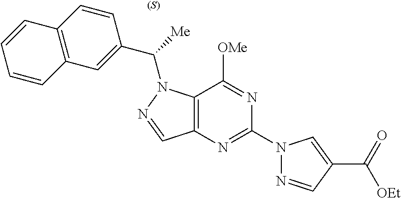 | powder MS (ESI) m/z: 443 [M + H]⁺ |
| 82 | 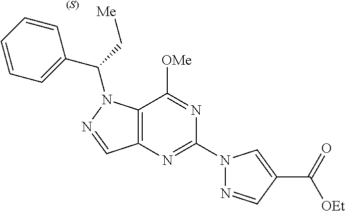 | powder MS (APCI) m/z: 407 [M + H]⁺ |
| 83 | 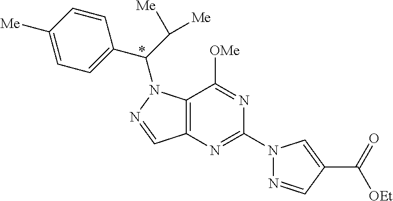 | viscous material MS (APCI) m/z: 435 [M + H]⁺ |
| 84 | 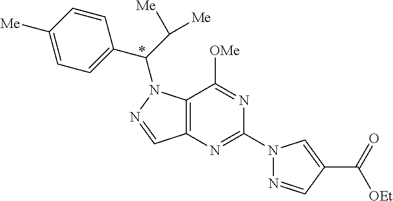 | viscous material MS (APCI) m/z: 435 [M + H]⁺ Enantiomer of Reference Example 83 |
| 85 | 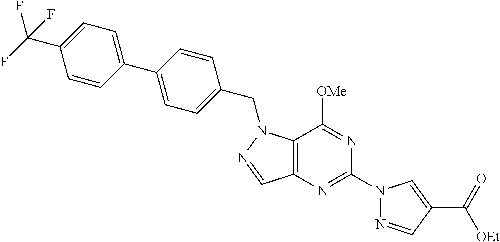 [SMILES: CCOC(=O)c1cnn(-c2nc(OC)c3c(cnn3Cc3ccc(-c4ccc(C(F)(F)F)cc4)cc3)n2)c1] | powder MS (ESI) m/z: 523 [M + H]⁺ |
| 86 | 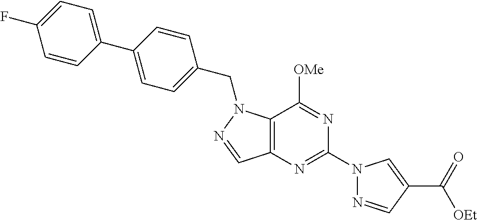 [SMILES: CCOC(=O)c1cnn(-c2nc(OC)c3c(cnn3Cc3ccc(-c4ccc(F)cc4)cc3)n2)c1] | powder MS (ESI) m/z: 473 [M + H]⁺ |
| 87 | 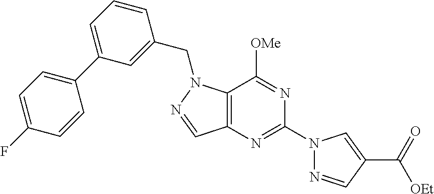 [SMILES: CCOC(=O)c1cnn(-c2nc(OC)c3c(cnn3Cc3cccc(-c4ccc(F)cc4)c3)n2)c1] | powder MS (ESI) m/z: 473 [M + H]⁺ |
| 88 | 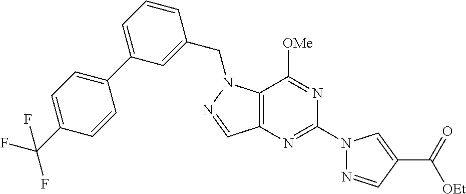 [SMILES: CCOC(=O)c1cnn(-c2nc(OC)c3c(cnn3Cc3cccc(-c4ccc(C(F)(F)F)cc4)c3)n2)c1] | powder MS (ESI) m/z: 523 [M + H]⁺ |
| 89 | 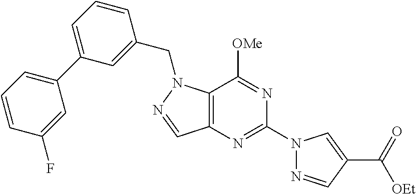 [SMILES: CCOC(=O)c1cnn(-c2nc(OC)c3c(cnn3Cc3cccc(-c4cccc(F)c4)c3)n2)c1] | powder MS (ESI) m/z: 473 [M + H]⁺ |
| 90 | 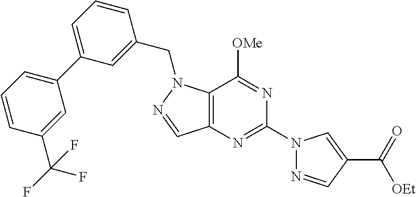 [SMILES: CCOC(=O)c1cnn(-c2nc(OC)c3c(cnn3Cc3cccc(-c4cccc(C(F)(F)F)c4)c3)n2)c1] | powder MS (ESI) m/z: 523 [M + H]⁺ |
| 91 | 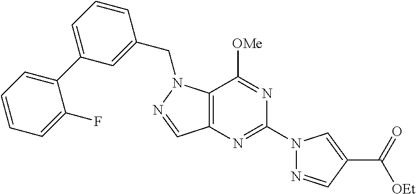 [SMILES: CCOC(=O)c1cnn(-c2nc(OC)c3c(cnn3Cc3cccc(-c4ccccc4F)c3)n2)c1] | powder MS (ESI) m/z: 473 [M + H]⁺ |
| 92 | 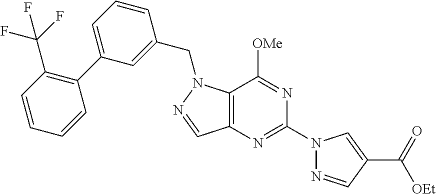 [SMILES: CCOC(=O)c1cnn(-c2nc(OC)c3c(cnn3Cc3cccc(-c4ccccc4C(F)(F)F)c3)n2)c1] | powder MS (ESI) m/z: 523 [M + H]⁺ |
| 93 | 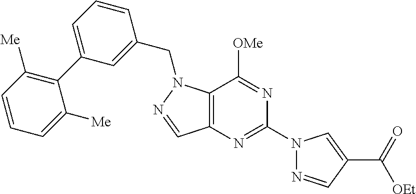 [SMILES: CCOC(=O)c1cnn(-c2nc(OC)c3c(cnn3Cc3cccc(-c4c(C)cccc4C)c3)n2)c1] | powder MS (ESI) m/z: 483 [M + H]⁺ |
| 94 | 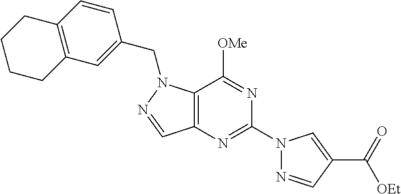 [SMILES: CCOC(=O)c1cnn(-c2nc(OC)c3c(cnn3Cc3ccc4c(c3)CCCC4)n2)c1] | powder MS (ESI) m/z: 433 [M + H]⁺ |
| 95 | 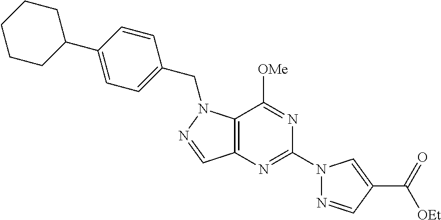 [SMILES: CCOC(=O)c1cnn(-c2nc(OC)c3c(cnn3Cc3ccc(C4CCCCC4)cc3)n2)c1] | powder MS (ESI) m/z: 461 [M + H]⁺ |
| 96 | 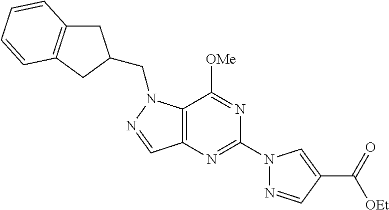 [SMILES: CCOC(=O)c1cnn(-c2nc(OC)c3c(cnn3CC3Cc4ccccc4C3)n2)c1] | powder MS (ESI) m/z: 419 [M + H]⁺ |
| 97 | 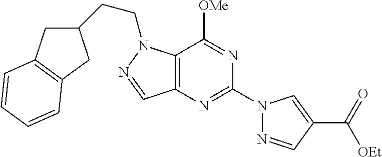 [SMILES: CCOC(=O)c1cnn(-c2nc(OC)c3c(cnn3CCC3Cc4ccccc4C3)n2)c1] | powder MS (ESI) m/z: 433 [M + H]⁺ |
| 98 | 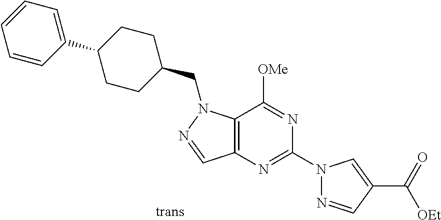 [SMILES: CCOC(=O)c1cnn(-c2nc(OC)c3c(cnn3C[C@H]3CC[C@H](c4ccccc4)CC3)n2)c1] | powder MS (ESI) m/z: 461 [M + H]⁺ |
| 99 | 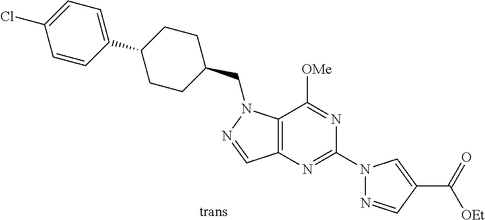 [SMILES: CCOC(=O)c1cnn(-c2nc(OC)c3c(cnn3C[C@H]3CC[C@H](c4ccc(Cl)cc4)CC3)n2)c1] | powder MS (ESI) m/z: 495/497 [M + H]⁺ |
| 100 | 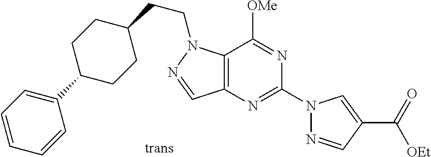 [SMILES: CCOC(=O)c1cnn(-c2nc(OC)c3c(cnn3CC[C@H]3CC[C@H](c4ccccc4)CC3)n2)c1] | powder MS (ESI) m/z: 475 [M + H]⁺ |
| 101 | 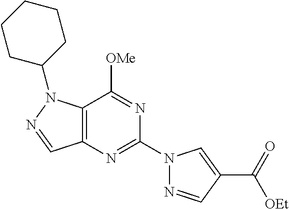 [SMILES: CCOC(=O)c1cnn(-c2nc(OC)c3c(cnn3C3CCCCC3)n2)c1] | powder MS (APCI) m/z: 371 [M + H]⁺ |
| 102 | 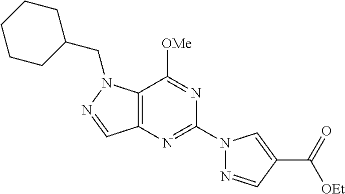 [SMILES: CCOC(=O)c1cnn(-c2nc(OC)c3c(cnn3CC3CCCCC3)n2)c1] | powder MS (APCI) m/z: 385 [M + H]⁺ |
| 103 | 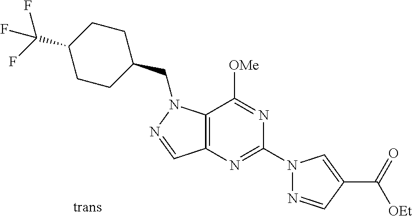 [SMILES: CCOC(=O)c1cnn(-c2nc(OC)c3c(cnn3C[C@H]3CC[C@H](C(F)(F)F)CC3)n2)c1] | powder MS (APCI) m/z: 453 [M + H]⁺ |
| 104 | 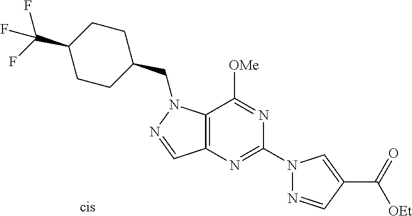 [SMILES: CCOC(=O)c1cnn(-c2nc(OC)c3c(cnn3C[C@H]3CC[C@@H](C(F)(F)F)CC3)n2)c1] | powder MS (APCI) m/z: 453 [M + H]⁺ |
| 105 | 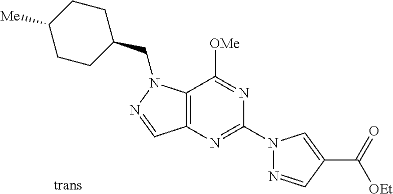 [SMILES: CCOC(=O)c1cnn(-c2nc(OC)c3c(cnn3C[C@H]3CC[C@H](C)CC3)n2)c1] | powder MS (APCI) m/z: 399 [M + H]⁺ |
| 106 | 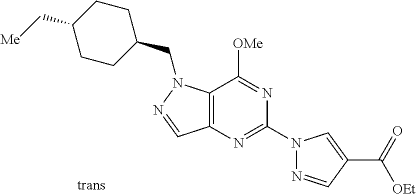 [SMILES: CCOC(=O)c1cnn(-c2nc(OC)c3c(cnn3C[C@H]3CC[C@H](CC)CC3)n2)c1] | powder MS (APCI) m/z: 413 [M + H]⁺ |
| 107 | 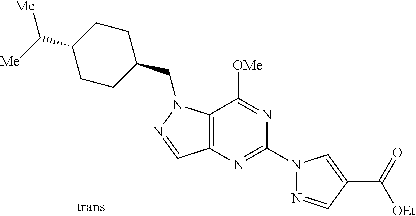 [SMILES: CCOC(=O)c1cnn(-c2nc(OC)c3c(cnn3C[C@H]3CC[C@H](C(C)C)CC3)n2)c1] | powder MS (APCI) m/z: 427 [M + H]⁺ |
| 108 | 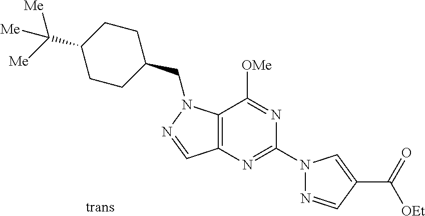 [SMILES: CCOC(=O)c1cnn(-c2nc(OC)c3c(cnn3C[C@H]3CC[C@H](C(C)(C)C)CC3)n2)c1] | powder MS (APCI) m/z: 441 [M + H]⁺ |
| 109 | 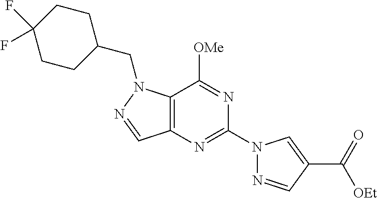 [SMILES: CCOC(=O)c1cnn(-c2nc(OC)c3c(cnn3CC3CCC(F)(F)CC3)n2)c1] | powder MS (APCI) m/z: 421 [M + H]⁺ |
| 110 | 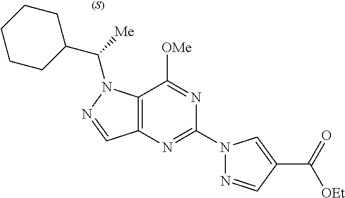 | viscous material MS (APCI) m/z: 399 [M + H]⁺ |
| 111 | 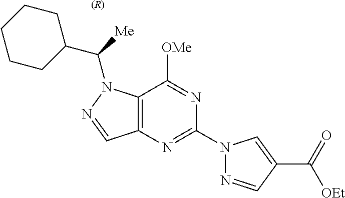 | viscous material MS (APCI) m/z: 399 [M + H]⁺ |
| 112 | 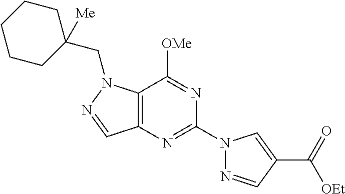 [SMILES: CCOC(=O)c1cnn(-c2nc(OC)c3c(cnn3CC3(C)CCCCC3)n2)c1] | powder MS (APCI) m/z: 399 [M + H]⁺ |
| 113 | 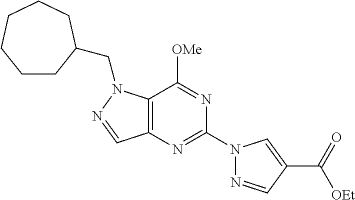 [SMILES: CCOC(=O)c1cnn(-c2nc(OC)c3c(cnn3CC3CCCCCC3)n2)c1] | powder MS (APCI) m/z: 399 [M + H]⁺ |
| 114 | 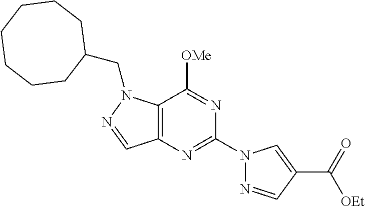 [SMILES: CCOC(=O)c1cnn(-c2nc(OC)c3c(cnn3CC3CCCCCCC3)n2)c1] | powder MS (ESI) m/z: 413 [M + H]⁺ |
| 115 | 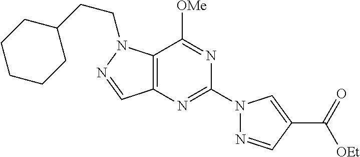 [SMILES: CCOC(=O)c1cnn(-c2nc(OC)c3c(cnn3CCC3CCCCC3)n2)c1] | powder MS (ESI) m/z: 399 [M + H]⁺ |
| 116 | 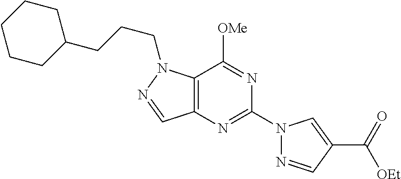 [SMILES: CCOC(=O)c1cnn(-c2nc(OC)c3c(cnn3CCCC3CCCCC3)n2)c1] | powder MS (ESI) m/z: 413 [M + H]⁺ |
| 117 | 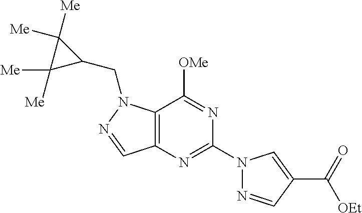 [SMILES: CCOC(=O)c1cnn(-c2nc(OC)c3c(cnn3CC3C(C)(C)C3(C)C)n2)c1] | powder MS (APCI) m/z: 399 [M + H]⁺ |
| 118 | 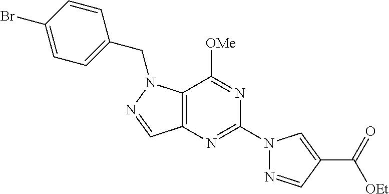 [SMILES: CCOC(=O)c1cnn(-c2nc(OC)c3c(cnn3Cc3ccc(Br)cc3)n2)c1] | powder MS (ESI) m/z: 457/459 [M + H]⁺ |
| 119 | 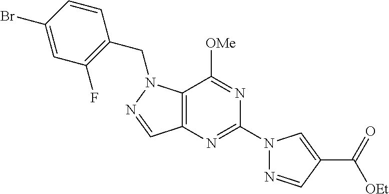 [SMILES: CCOC(=O)c1cnn(-c2nc(OC)c3c(cnn3Cc3ccc(Br)cc3F)n2)c1] | powder MS (ESI) m/z: 475/477 [M + H]⁺ |
| 120 | 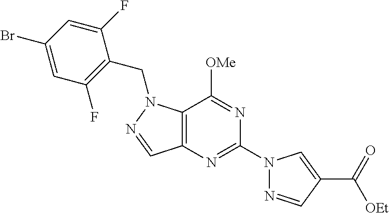 [SMILES: CCOC(=O)c1cnn(-c2nc(OC)c3c(cnn3Cc3c(F)cc(Br)cc3F)n2)c1] | powder MS (ESI) m/z: 493/495 [M + H]⁺ |
| 121 | 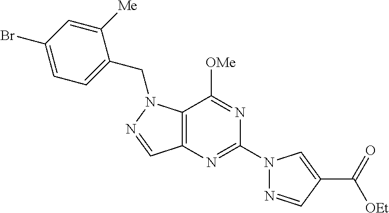 [SMILES: CCOC(=O)c1cnn(-c2nc(OC)c3c(cnn3Cc3ccc(Br)cc3C)n2)c1] | powder MS (ESI) m/z: 471/473 [M + H]⁺ |
| 122 | 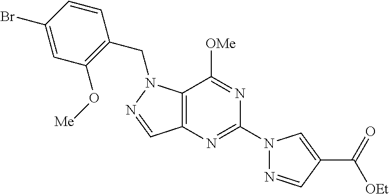 [SMILES: CCOC(=O)c1cnn(-c2nc(OC)c3c(cnn3Cc3ccc(Br)cc3OC)n2)c1] | powder MS (ESI) m/z: 487/489 [M + H]⁺ |
| 123 | 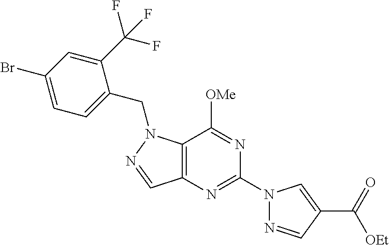 [SMILES: CCOC(=O)c1cnn(-c2nc(OC)c3c(cnn3Cc3ccc(Br)cc3C(F)(F)F)n2)c1] | powder MS (ESI) m/z: 525/527 [M + H]⁺ |
| 124 | 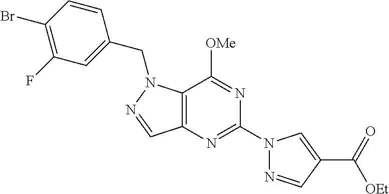 [SMILES: CCOC(=O)c1cnn(-c2nc(OC)c3c(cnn3Cc3ccc(Br)c(F)c3)n2)c1] | powder MS (APCI) m/z: 475/477 [M + H]⁺ |
| 125 | 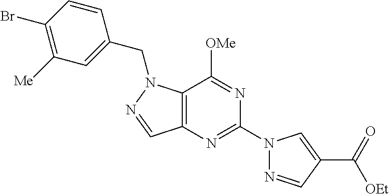 [SMILES: CCOC(=O)c1cnn(-c2nc(OC)c3c(cnn3Cc3ccc(Br)c(C)c3)n2)c1] | powder MS (APCI) m/z: 471/473 [M + H]⁺ |
| 126 | 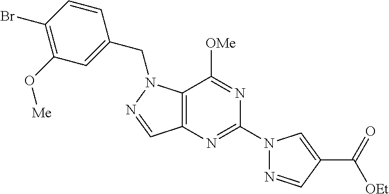 [SMILES: CCOC(=O)c1cnn(-c2nc(OC)c3c(cnn3Cc3ccc(Br)c(OC)c3)n2)c1] | powder MS (APCI) m/z: 487/489 [M + H]⁺ |
| 127 | 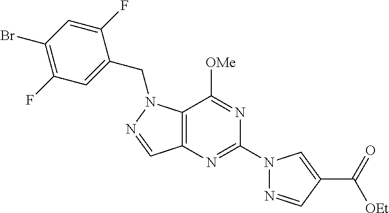 [SMILES: CCOC(=O)c1cnn(-c2nc(OC)c3c(cnn3Cc3cc(F)c(Br)cc3F)n2)c1] | powder MS (ESI) m/z: 493/495 [M + H]⁺ |
| 128 | 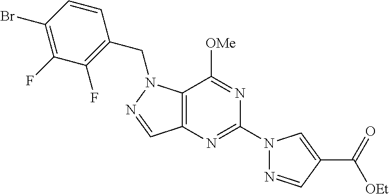 [SMILES: CCOC(=O)c1cnn(-c2nc(OC)c3c(cnn3Cc3ccc(Br)c(F)c3F)n2)c1] | powder MS (APCI) m/z: 493/495 [M + H]⁺ |
| 129 | 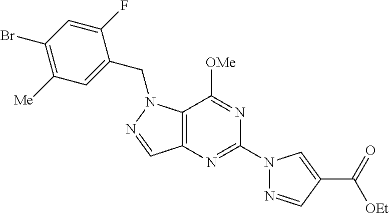 [SMILES: CCOC(=O)c1cnn(-c2nc(OC)c3c(cnn3Cc3cc(C)c(Br)cc3F)n2)c1] | powder MS (APCI) m/z: 489/491 [M + H]⁺ |
| 130 | 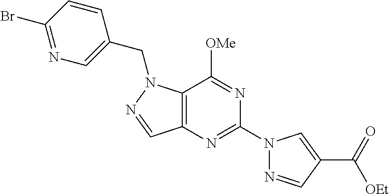 [SMILES: CCOC(=O)c1cnn(-c2nc(OC)c3c(cnn3Cc3ccc(Br)nc3)n2)c1] | powder MS (APCI) m/z: 458/460 [M + H]⁺ |
| 131 | 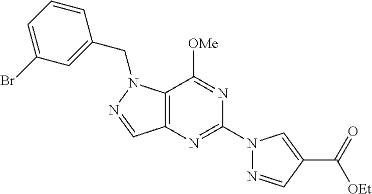 [SMILES: CCOC(=O)c1cnn(-c2nc(OC)c3c(cnn3Cc3cccc(Br)c3)n2)c1] | powder MS (ESI) m/z: 457/459 [M + H]⁺ |
| 132 | 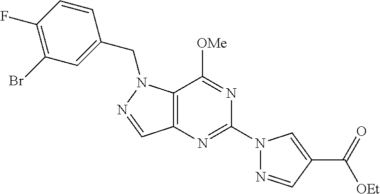 [SMILES: CCOC(=O)c1cnn(-c2nc(OC)c3c(cnn3Cc3ccc(F)c(Br)c3)n2)c1] | powder MS (ESI) m/z: 475/477 [M + H]⁺ |
| 133 | 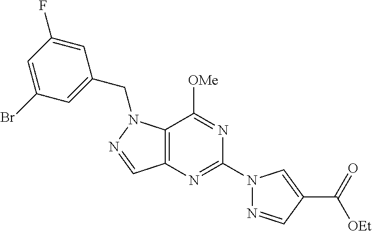 [SMILES: CCOC(=O)c1cnn(-c2nc(OC)c3c(cnn3Cc3cc(F)cc(Br)c3)n2)c1] | powder MS (ESI) m/z: 475/477 [M + H]⁺ |
| 134 | 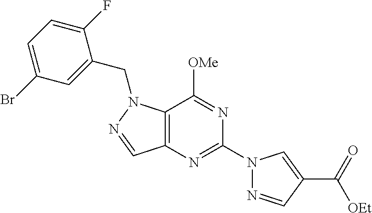 [SMILES: CCOC(=O)c1cnn(-c2nc(OC)c3c(cnn3Cc3cc(Br)ccc3F)n2)c1] | powder MS (ESI) m/z: 475/477 [M + H]⁺ |
| 135 | 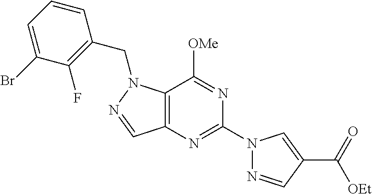 [SMILES: CCOC(=O)c1cnn(-c2nc(OC)c3c(cnn3Cc3cccc(Br)c3F)n2)c1] | powder MS (ESI) m/z: 475/477 [M + H]⁺ |
| 136 | 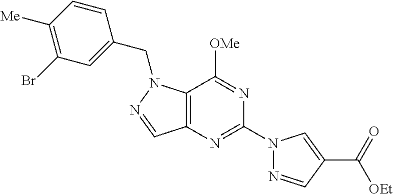 [SMILES: CCOC(=O)c1cnn(-c2nc(OC)c3c(cnn3Cc3ccc(C)c(Br)c3)n2)c1] | powder MS (ESI) m/z: 471/473 [M + H]⁺ |
| 137 | 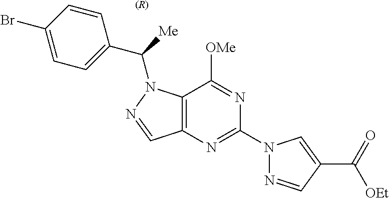 | powder MS (ESI) m/z: 471/473 [M + H]⁺ |
| 138 | 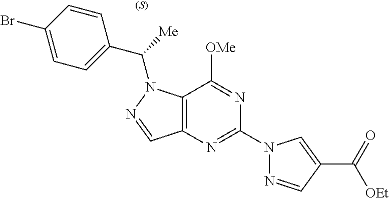 | powder MS (ESI) m/z: 471/473 [M + H]⁺ |
| 139 | 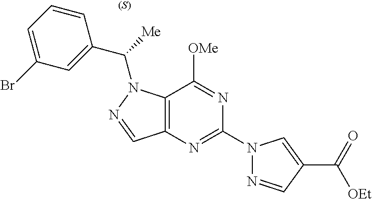 | viscous material MS (APCI) m/z: 471/473 [M + H]⁺ |
| 140 | 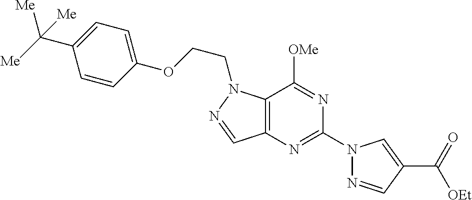 [SMILES: CCOC(=O)c1cnn(-c2nc(OC)c3c(cnn3CCOc3ccc(C(C)(C)C)cc3)n2)c1] | powder MS (ESI) m/z: 465 [M + H]⁺ |
| 141 | 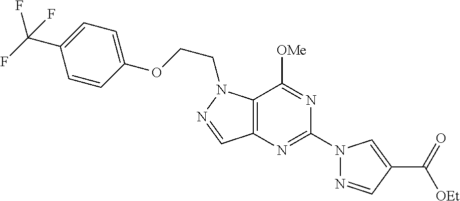 [SMILES: CCOC(=O)c1cnn(-c2nc(OC)c3c(cnn3CCOc3ccc(C(F)(F)F)cc3)n2)c1] | powder MS (ESI) m/z: 477 [M + H]⁺ |
| 142 | 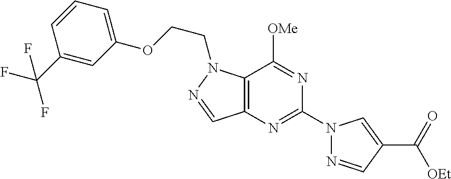 [SMILES: CCOC(=O)c1cnn(-c2nc(OC)c3c(cnn3CCOc3cccc(C(F)(F)F)c3)n2)c1] | powder MS (ESI) m/z: 477 [M + H]⁺ |
| 143 | 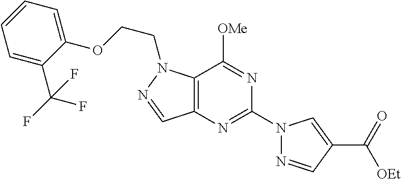 [SMILES: CCOC(=O)c1cnn(-c2nc(OC)c3c(cnn3CCOc3ccccc3C(F)(F)F)n2)c1] | powder MS (ESI) m/z: 477 [M + H]⁺ |
| 144 | 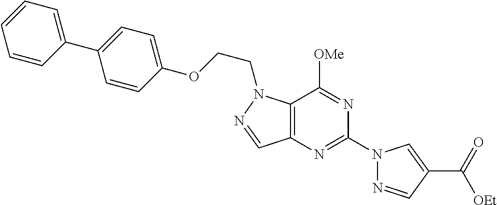 [SMILES: CCOC(=O)c1cnn(-c2nc(OC)c3c(cnn3CCOc3ccc(-c4ccccc4)cc3)n2)c1] | powder MS (ESI) m/z: 485 [M + H]⁺ |
| 145 | 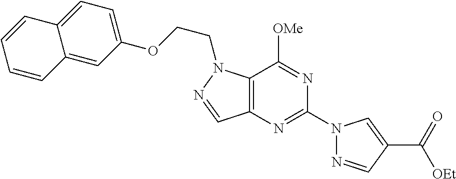 [SMILES: CCOC(=O)c1cnn(-c2nc(OC)c3c(cnn3CCOc3ccc4ccccc4c3)n2)c1] | powder MS (ESI) m/z: 459 [M + H]⁺ |
| 146 | 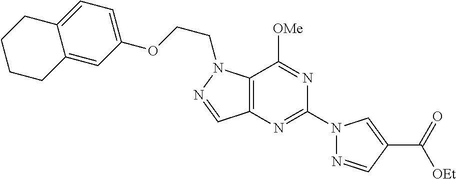 [SMILES: CCOC(=O)c1cnn(-c2nc(OC)c3c(cnn3CCOc3ccc4c(c3)CCCC4)n2)c1] | powder MS (ESI) m/z: 463 [M + H]⁺ |
| 147 | 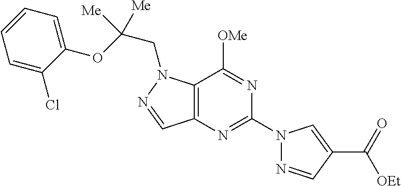 [SMILES: CCOC(=O)c1cnn(-c2nc(OC)c3c(cnn3CC(C)(C)Oc3ccccc3Cl)n2)c1] | powder MS (APCI) m/z: 471/473 [M + H]⁺ |
| 148 | 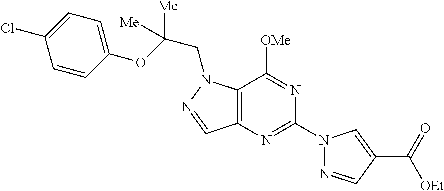 [SMILES: CCOC(=O)c1cnn(-c2nc(OC)c3c(cnn3CC(C)(C)Oc3ccc(Cl)cc3)n2)c1] | powder MS (APCI) m/z: 471/473 [M + H]⁺ |
| 149 | 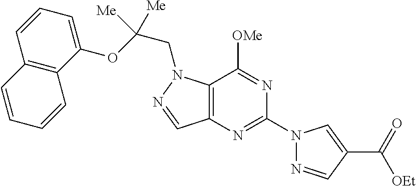 [SMILES: CCOC(=O)c1cnn(-c2nc(OC)c3c(cnn3CC(C)(C)Oc3cccc4ccccc34)n2)c1] | powder MS (APCI) m/z: 487 [M + H]⁺ |
| 150 | 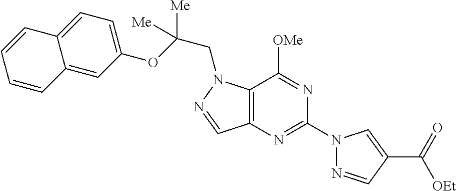 [SMILES: CCOC(=O)c1cnn(-c2nc(OC)c3c(cnn3CC(C)(C)Oc3ccc4ccccc4c3)n2)c1] | powder MS (ESI) m/z: 487 [M + H]⁺ |
| 151 | 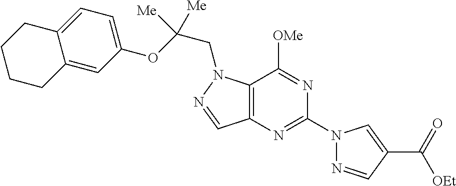 [SMILES: CCOC(=O)c1cnn(-c2nc(OC)c3c(cnn3CC(C)(C)Oc3ccc4c(c3)CCCC4)n2)c1] | powder MS (APCI) m/z: 491 [M + H]⁺ |
| 152 | 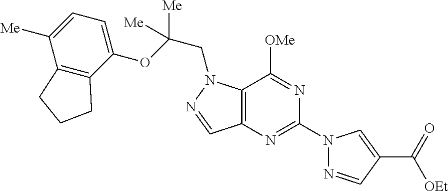 [SMILES: CCOC(=O)c1cnn(-c2nc(OC)c3c(cnn3CC(C)(C)Oc3ccc(C)c4c3CCC4)n2)c1] | powder MS (APCI) m/z: 491 [M + H]⁺ |
| 153 | 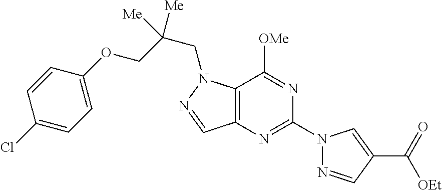 [SMILES: CCOC(=O)c1cnn(-c2nc(OC)c3c(cnn3CC(C)(C)COc3ccc(Cl)cc3)n2)c1] | viscous material MS (APCI) m/z: 485/487 [M + H]⁺ |
| 154 | 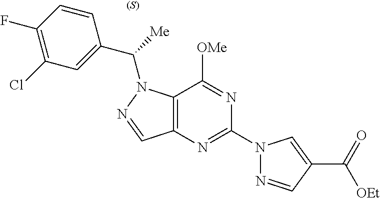 | powder MS (ESI) m/z: 445/447 [M + H]⁺ |
| 155 | 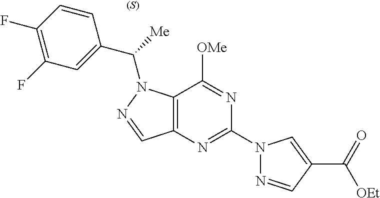 | powder MS (ESI) m/z: 429 [M + H]⁺ |
| 156 | 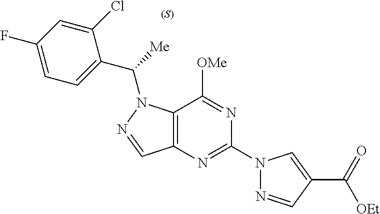 | powder MS (ESI) m/z: 445/447 [M + H]⁺ |
| 157 | 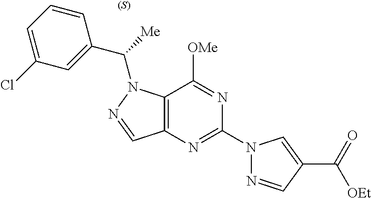 | viscous material MS (APCI) m/z: 427/429 [M + H]⁺ |
| 158 | 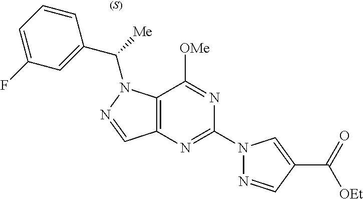 | powder MS (APCI) m/z: 411 [M + H]⁺ |
| 159 | 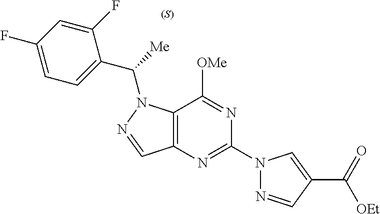 | powder MS (APCI) m/z: 429 [M + H]⁺ |
| 160 | 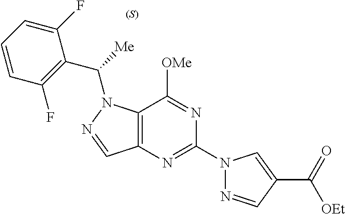 | viscous material MS (APCI) m/z: 429 [M + H]⁺ |
| 161 | 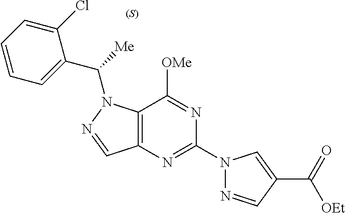 | powder MS (APCI) m/z: 427/429 [M + H]⁺ |
| 162 | 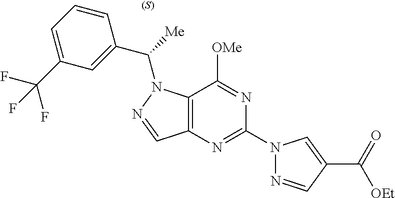 | powder MS (APCI) m/z: 461 [M + H]⁺ |
| 163 | 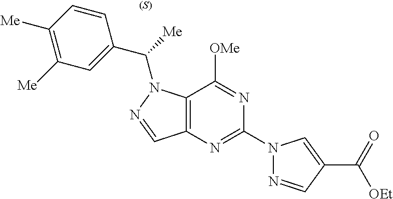 | solid MS (APCI) m/z: 421 [M + H]⁺ |
| 164 | 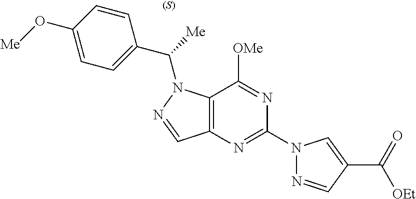 | powder MS (APCI) m/z: 423 [M + H]⁺ |
| 165 | 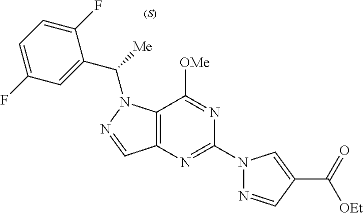 | viscous material MS (APCI) m/z: 429 [M + H]⁺ |
| 166 | 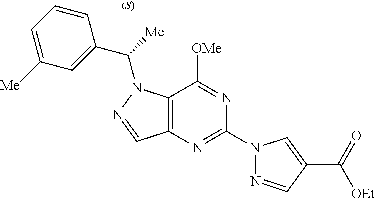 | viscous material MS (APCI) m/z: 407 [M + H]⁺ |
| 167 | 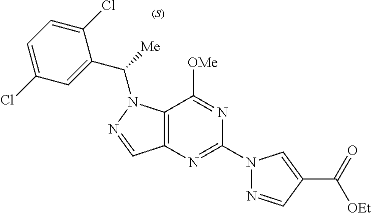 | powder MS (APCI) m/z: 461/463 [M + H]⁺ |
| 168 | 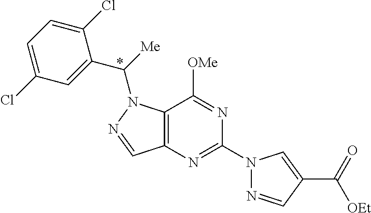 | powder MS (APCI) m/z: 461/463 [M + H]⁺ racemic mixture |
| 169 | 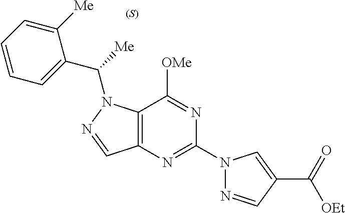 | viscous material MS (ESI) m/z: 407 [M + H]⁺ |
| 170 | 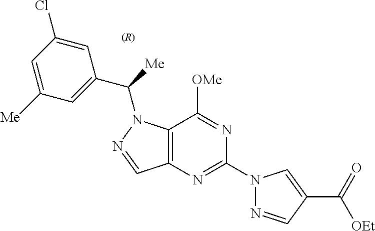 | powder MS (ESI) m/z: 441/443 [M + H]⁺ |
| 171 | 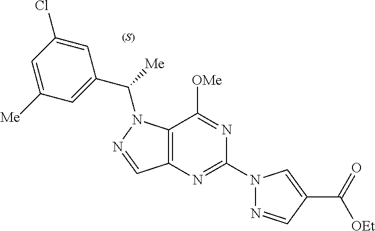 | viscous material MS (ESI) m/z: 441/443 [M + H]⁺ |
| 172 | 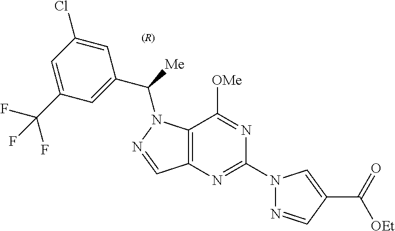 | powder MS (ESI) m/z: 495/497 [M + H]⁺ |
| 173 | 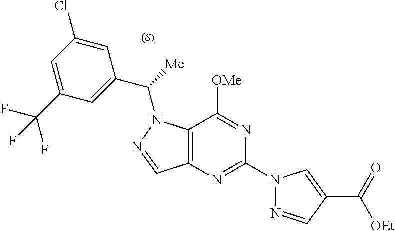 | viscous material MS (ESI) m/z: 495/497 [M + H]⁺ |
| 174 | 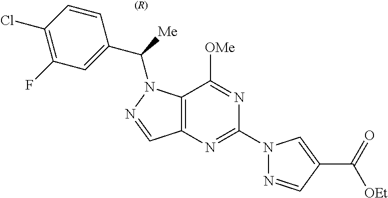 | viscous material MS (ESI) m/z: 445/447 [M + H]⁺ |
| 175 | 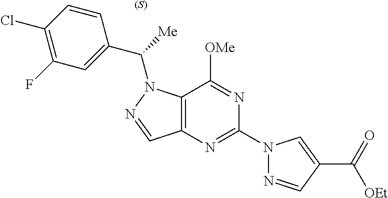 | viscous material MS (APCI) m/z: 445/447 [M + H]⁺ |
| 176 | 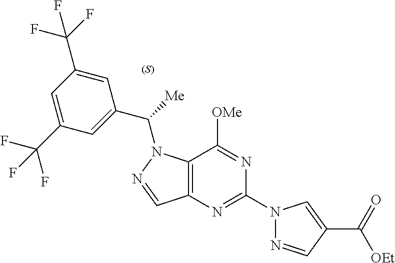 | viscous material MS (ESI) m/z: 529 [M + H]⁺ |
| 177 | 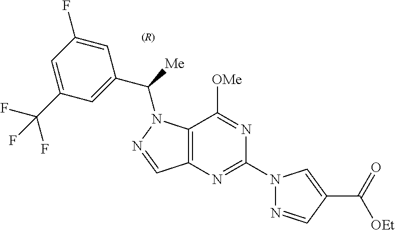 | powder MS (ESI) m/z: 479 [M + H]⁺ |
| 178 | 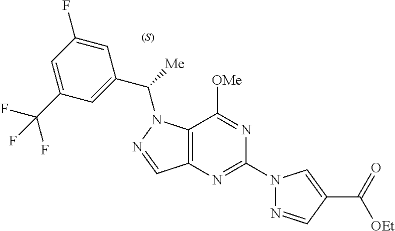 | viscous material MS (APCI) m/z: 479 [M + H]⁺ |
| 179 | 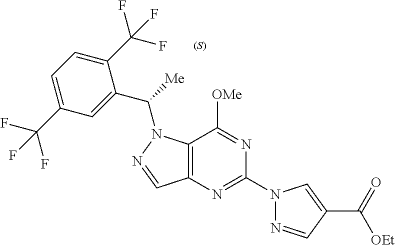 | powder MS (ESI) m/z: 529 [M + H]⁺ |
| 180 | 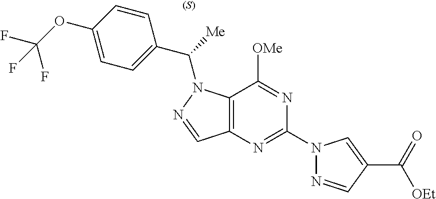 | viscous material MS (ESI) m/z: 477 [M + H]⁺ |
| 181 | 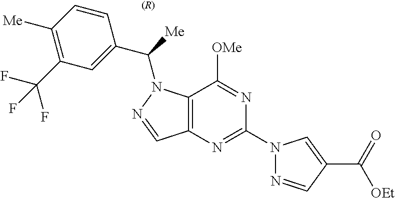 | viscous material MS (ESI) m/z: 475 [M + H]⁺ |
| 182 | 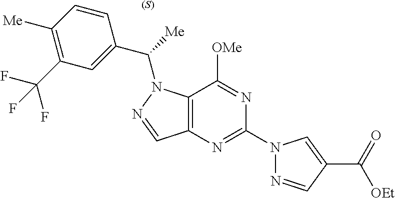 | viscous material MS (APCI) m/z: 475 [M + H]⁺ |
| 183 | 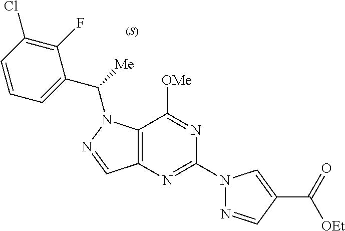 | viscous material MS (ESI) m/z: 445/447 [M + H]⁺ |
| 184 | 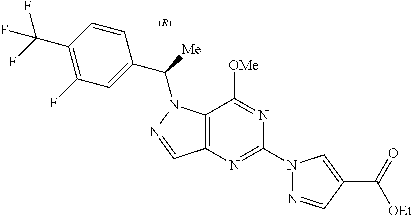 | powder MS (ESI) m/z: 479 [M + H]⁺ |
| 185 | 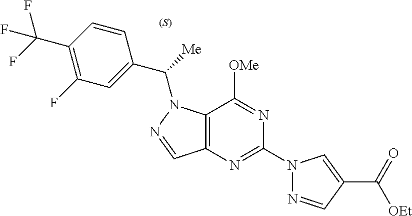 | powder MS (APCI) m/z: 479 [M + H]⁺ |
| 186 | 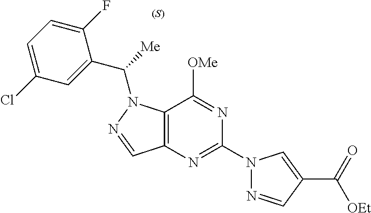 | solid MS (ESI) m/z: 445/447 [M + H]⁺ |
| 187 | 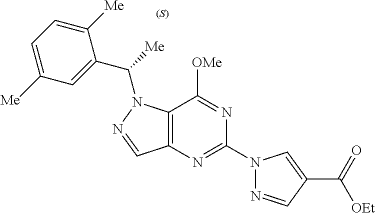 | solid MS (ESI) m/z: 421 [M + H]⁺ |
| 188 | 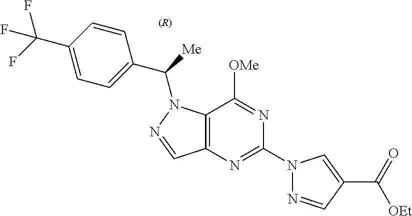 | viscous material MS (APCI) m/z: 461 [M + H]⁺ |
| 189 | 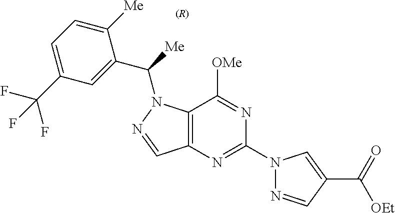 | viscous material MS (APCI) m/z: 475 [M + H]⁺ |
| 190 | 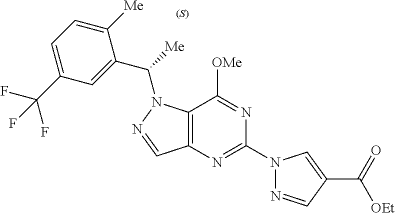 | viscous material MS (APCI) m/z: 475 [M + H]⁺ |
| 191 | 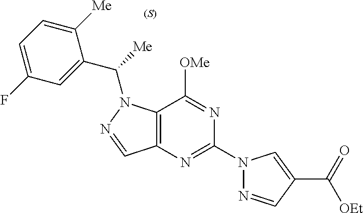 | powder MS (APCI) m/z: 425 [M + H]⁺ |
| 192 | 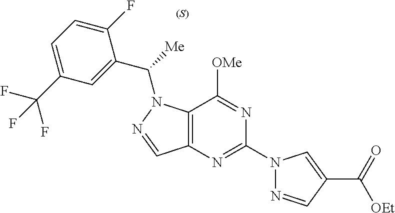 | viscous material MS (APCI) m/z: 479 [M + H]⁺ |
| 193 | 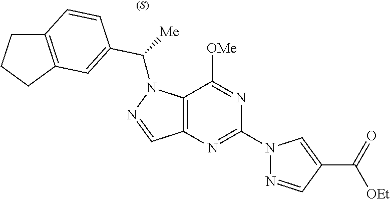 | viscous material MS (APCI) m/z: 433 [M + H]⁺ |
| 194 | 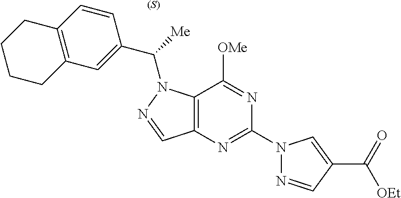 | viscous material MS (APCI) m/z: 447 [M + H]⁺ |
| 195 | 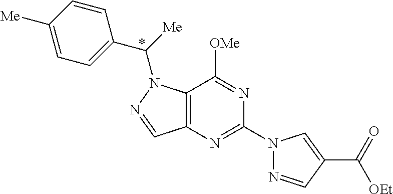 | powder MS (APCI) m/z: 407 [M + H]⁺ |
| 196 | 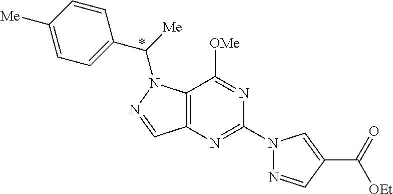 | powder MS (APCI) m/z: 407 [M + H]⁺ Enantiomer of Reference Example 195 |
| 197 | 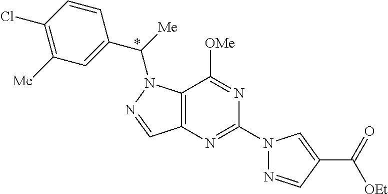 | viscous material MS (ESI) m/z: 441/443 [M + H]⁺ |
| 198 | 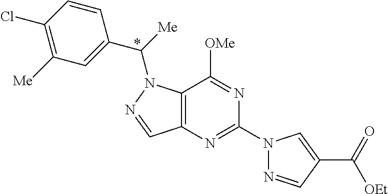 | viscous material MS (ESI) m/z: 441/443 [M + H]⁺ Enantiomer of Reference Example 197 |
| 199 | 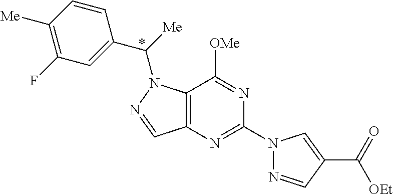 | powder MS (APCI) m/z: 425 [M + H]⁺ |
| 200 | 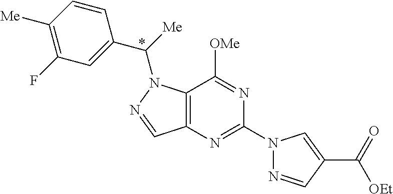 | powder MS (APCI) m/z: 425 [M + H]⁺ Enantiomer of Reference Example 199 |
| 201 | 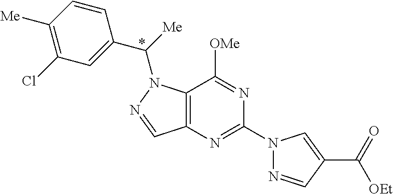 | powder MS (ESI) m/z: 441/443 [M + H]⁺ |
| 202 | 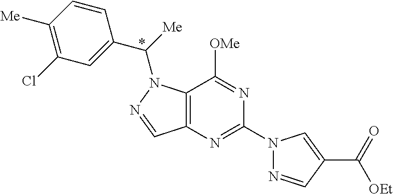 | powder MS (ESI) m/z: 441/443 [M + H]⁺ Enantiomer of Reference Example 201 |
| 203 | 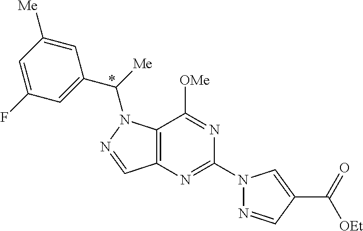 | viscous material MS (APCI) m/z: 425 [M + H]⁺ |
| 204 | 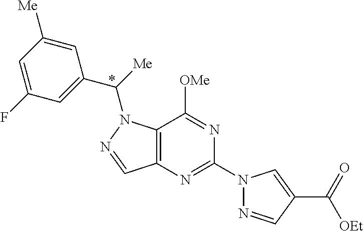 | solid MS (APCI) m/z: 425 [M + H]⁺ Enantiomer of Reference Example 203 |
| 205 | 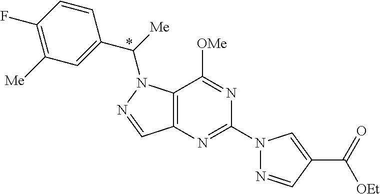 | viscous material MS (APCI) m/z: 425 [M + H]⁺ |
| 206 | 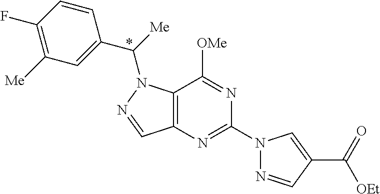 | viscous material MS (APCI) m/z: 425 [M + H]⁺ Enantiomer of Reference Example 205 |
| 207 | 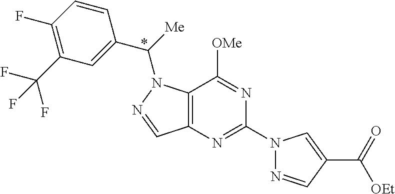 | powder MS (APCI) m/z: 479 [M + H]⁺ |
| 208 | 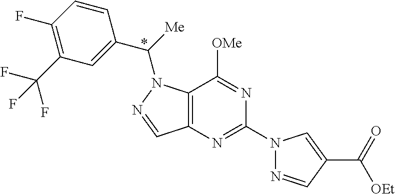 | powder MS (APCI) m/z: 479 [M + H]⁺ Enantiomer of Reference Example 207 |
| 209 | 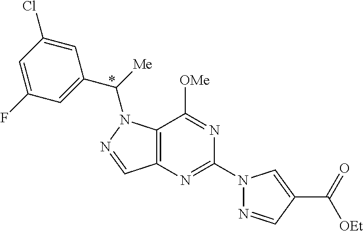 | solid MS (ESI) m/z: 445/447 [M + H]⁺ |
| 210 | 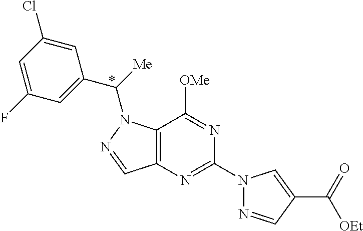 | solid MS (ESI) m/z: 445/447 [M + H]⁺ Enantiomer of Reference Example 209 |
| 211 | 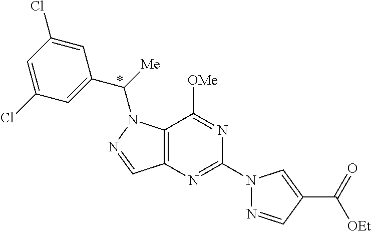 | solid MS (ESI) m/z: 461/463 [M + H]⁺ |
| 212 | 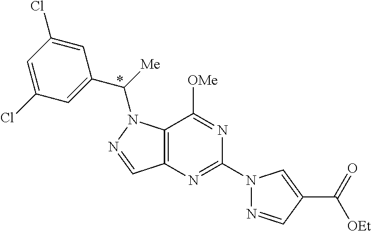 | solid MS (ESI) m/z: 461/463 [M + H]⁺ Enantiomer of Reference Example 211 |
| 213 | 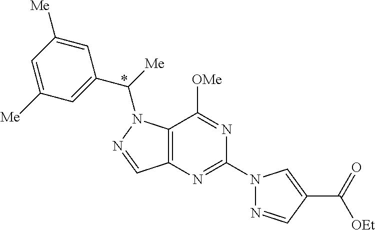 | solid MS (ESI) m/z: 421 [M + H]⁺ |
| 214 | 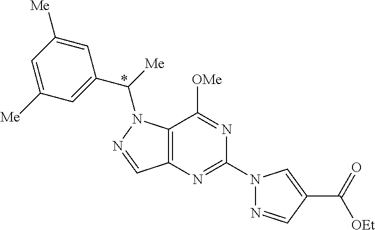 | viscous material MS (ESI) m/z: 421 [M + H]⁺ Enantiomer of Reference Example 213 |
| 215 | 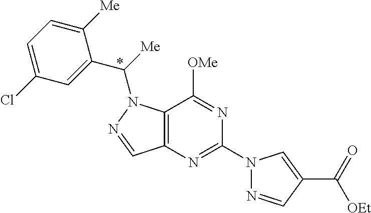 | viscous material MS (ESI) m/z: 441/443 [M + H]⁺ |
| 216 | 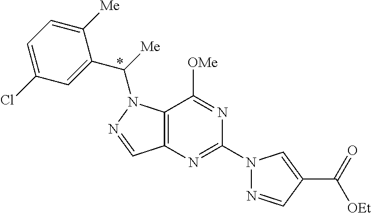 | viscous material MS (ESI) m/z: 441/443 [M + H]⁺ Enantiomer of Reference Example 215 |
| 217 | 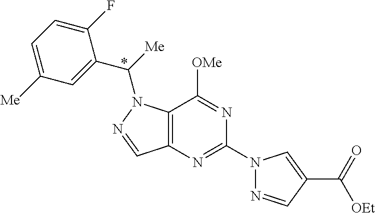 | viscous material MS (ESI) m/z: 425 [M + H]⁺ |
| 218 | 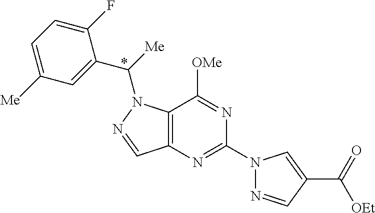 | viscous material MS (ESI) m/z: 425 [M + H]⁺ Enantiomer of Reference Example 217 |
| 219 | 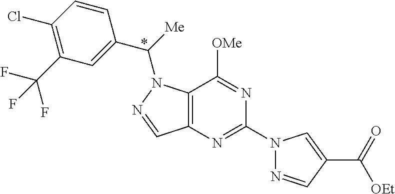 | solid MS (APCI) m/z: 495/497 [M + H]⁺ |
| 220 | 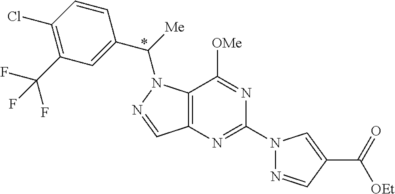 | solid MS (APCI) m/z: 495/497 [M + H]⁺ Enantiomer of Reference Example 219 |
| 221 | 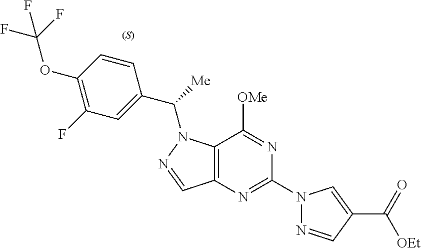 | solid MS (APCI) m/z: 495 [M + H]⁺ |
| 222 | 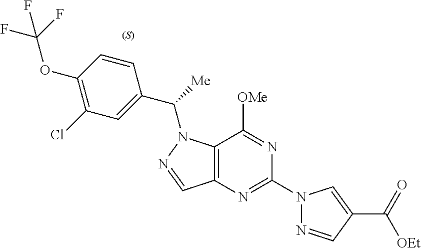 | solid MS (APCI) m/z: 511/513 [M + H]⁺ |
| 223 | 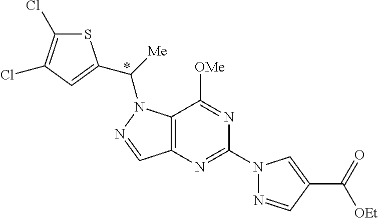 | solid MS (ESI) m/z: 467/469 [M + H]⁺ |
| 224 | 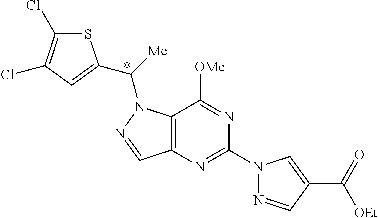 | solid MS (ESI) m/z: 467/469 [M + H]⁺ Enantiomer of Reference Example 223 |
| 225 | 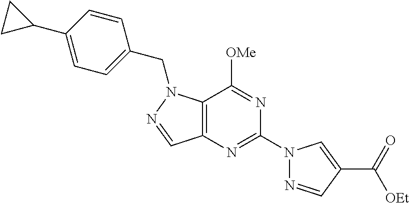 [SMILES: CCOC(=O)c1cnn(-c2nc(OC)c3c(cnn3Cc3ccc(C4CC4)cc3)n2)c1] | solid MS (ESI) m/z: 419 [M + H]⁺ |
| 226 | 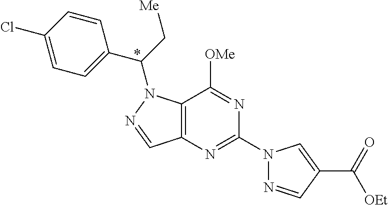 | viscous material MS (APCI) m/z: 441/443 [M + H]⁺ |
| 227 | 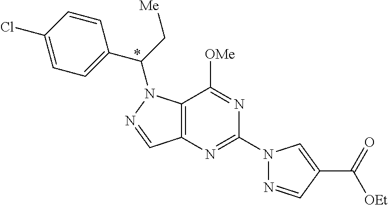 | viscous material MS (APCI) m/z: 441/443 [M + H]⁺ Enantiomer of Reference Example 226 |
| 228 | 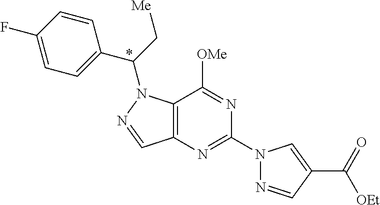 | viscous material MS (ESI) m/z: 425 [M + H]⁺ |
| 229 | 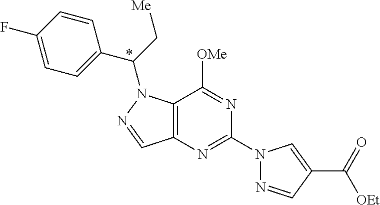 | viscous material MS (ESI) m/z: 425 [M + H]⁺ Enantiomer of Reference Example 228 |
| 230 | 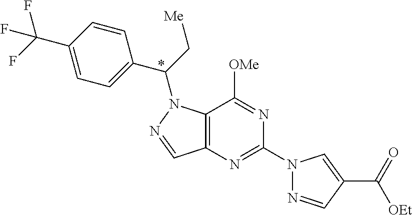 | viscous material MS (APCI) m/z: 475 [M + H]⁺ |
| 231 | 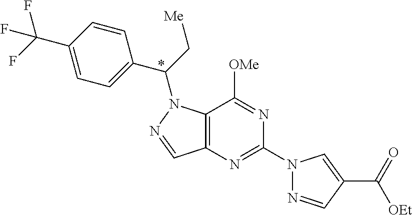 | viscous material MS (APCI) m/z: 475 [M + H]⁺ Enantiomer of Reference Example 230 |
| 232 | 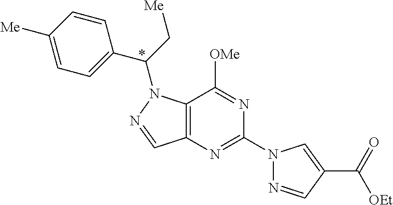 | viscous material MS (APCI) m/z: 421 [M + H]⁺ |
| 233 | 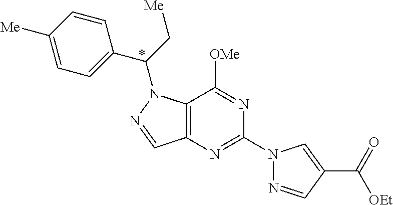 | viscous material MS (APCI) m/z: 421 [M + H]⁺ Enantiomer of Reference Example 232 |
| 234 | 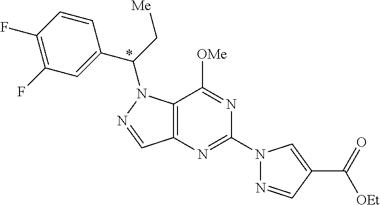 | viscous material MS (APCI) m/z: 443 [M + H]⁺ |
| 235 | 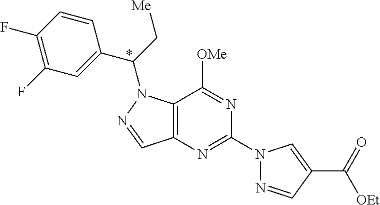 | viscous material MS (APCI) m/z: 443 [M + H]⁺ Enantiomer of Reference Example 234 |
| 236 | 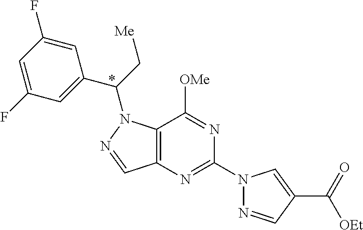 | solid MS (APCI) m/z: 443 [M + H]⁺ |
| 237 | 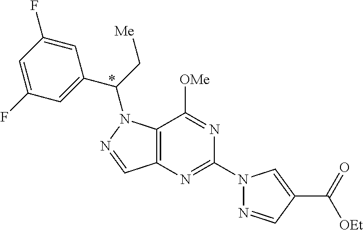 | viscous material MS (APCI) m/z: 443 [M + H]⁺ Enantiomer of Reference Example 236 |
| 238 | 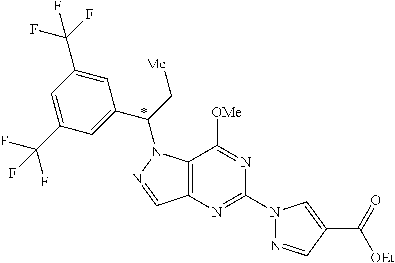 | powder MS (APCI) m/z: 543 [M + H]⁺ |
| 239 | 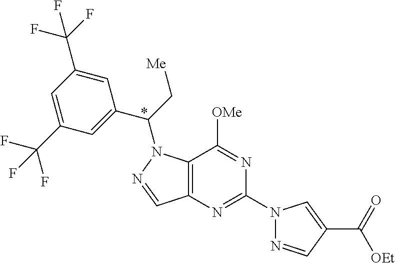 | powder MS (APCI) m/z: 543 [M + H]⁺ Enantiomer of Reference Example 238 |
| 240 | 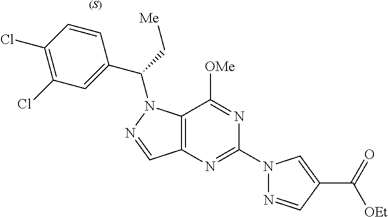 | powder MS (ESI) m/z: 475/477 [M + H]⁺ |
| 241 | 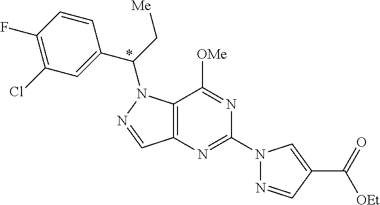 | viscous material MS (ESI) m/z: 459/461 [M + H] |
| 242 | 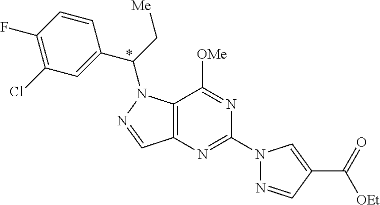 | viscous material MS (ESI) m/z: 459/461 [M + H]⁺ Enantiomer of Reference Example 241 |
| 243 | 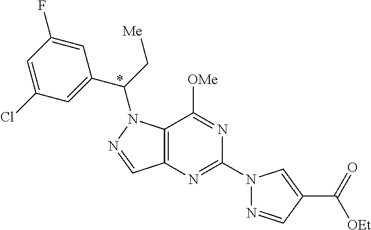 | viscous material MS (APCI) m/z: 459/461 [M + H]⁺ |
| 244 | 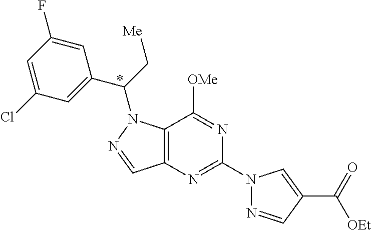 | viscous material MS (APCI) m/z: 459/461 [M + H]⁺ Enantiomer of Reference Example 243 |
| 245 | 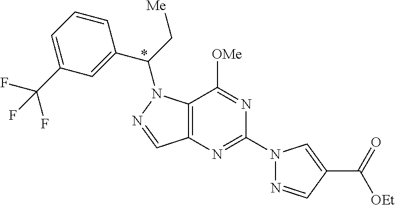 | viscous material MS (APCI) m/z: 475 [M + H]⁺ |
| 246 | 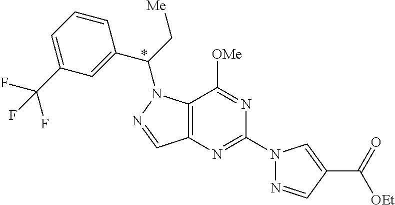 | viscous material MS (APCI) m/z: 475 [M + H]⁺ Enantiomer of Reference Example 245 |
| 247 | 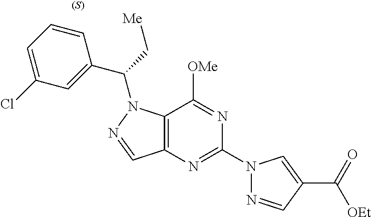 | viscous material MS (ESI) m/z: 441/443 [M + H]⁺ |
| 248 | 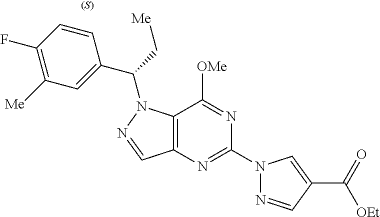 | viscous material MS (APCI) m/z: 439 [M + H]⁺ |
| 249 | 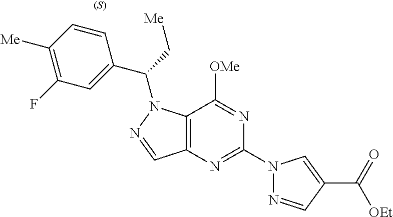 | viscous material MS (APCI) m/z: 439 [M + H]⁺ |
| 250 | 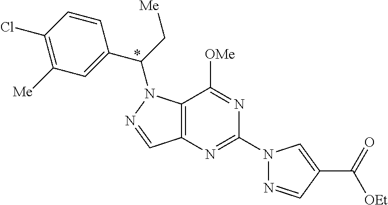 | viscous material MS (APCI) m/z: 455/457 [M + H]⁺ |
| 251 | 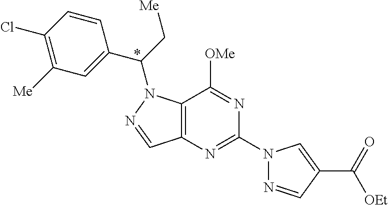 | viscous material MS (APCI) m/z: 455/457 [M + H]⁺ Enantiomer of Reference Example 250 |
| 252 | 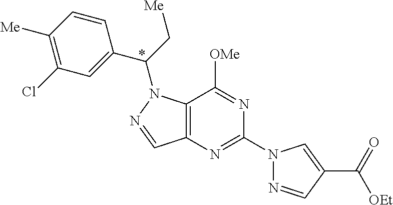 | viscous material MS (APCI) m/z: 455/457 [M + H]⁺ |
| 253 | 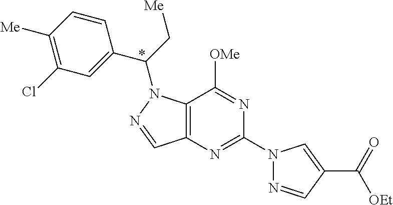 | viscous material MS (APCI) m/z: 455/457 [M + H]⁺ Enantiomer of Reference Example 252 |
| 254 | 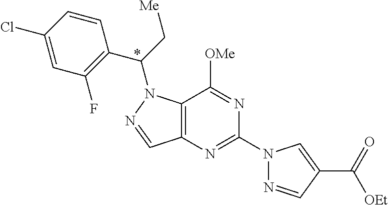 | viscous material MS (APCI) m/z: 459/461 [M + H]⁺ |
| 255 | 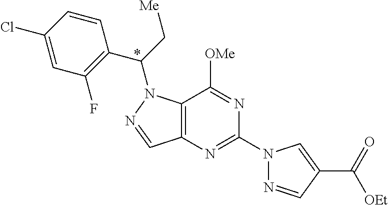 | viscous material MS (APCI) m/z: 459/461 [M + H]⁺ Enantiomer of Reference Example 254 |
| 256 | 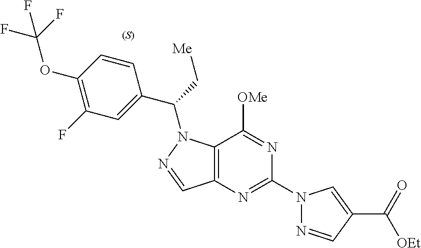 | viscous material MS (ESI) m/z: 509 [M + H]⁺ |
| 257 | 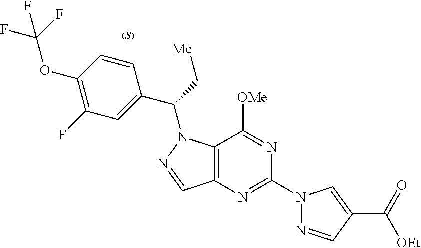 | viscous material MS (ESI) m/z: 525/527 [M + H]⁺ |
| 258 | 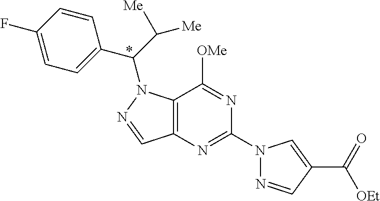 | viscous material MS (APCI) m/z: 439 [M + H]⁺ |
| 259 | 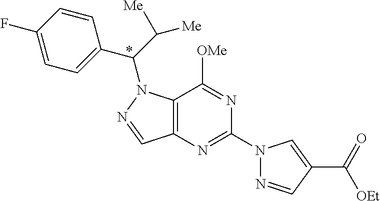 | viscous material MS (APCI) m/z: 439 [M + H]⁺ Enantiomer of Reference Example 258 |
| 260 | 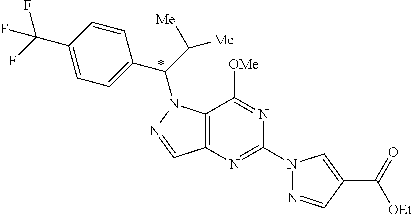 | viscous material MS (ESI) m/z: 489 [M + H]⁺ |
| 261 | 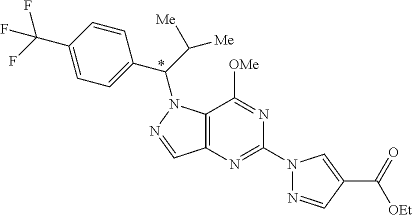 | viscous material MS (ESI) m/z: 489 [M + H]⁺ Enantiomer of Reference Example 260 |
| 262 | 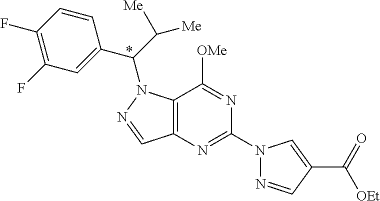 | viscous material MS (APCI) m/z: 457 [M + H]⁺ |
| 263 | 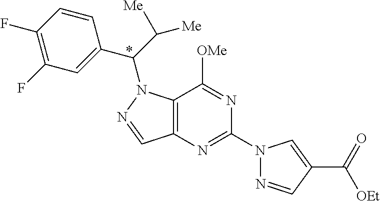 | viscous material MS (APCI) m/z: 457 [M + H]⁺ Enantiomer of Reference Example 262 |
| 264 | 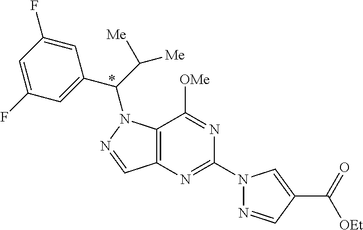 | viscous material MS (APCI) m/z: 457 [M + H]⁺ |
| 265 | 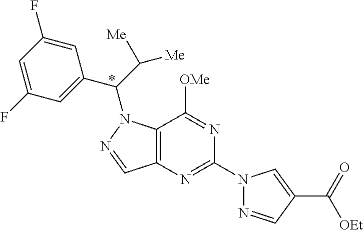 | viscous material MS (APCI) m/z: 457 [M + H]⁺ Enantiomer of Reference Example 264 |
| 266 | 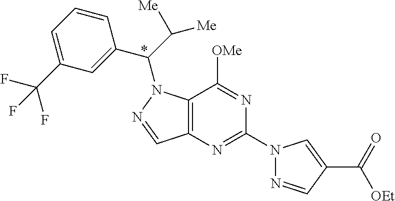 | viscous material MS (APCI) m/z: 489 [M + H]⁺ |
| 267 | 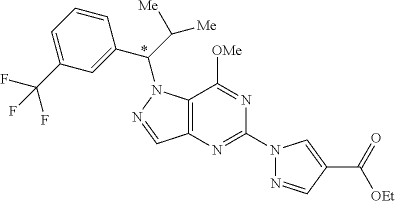 | viscous material MS (APCI) m/z: 489 [M + H]⁺ Enantiomer of Reference Example 266 |
| 268 | 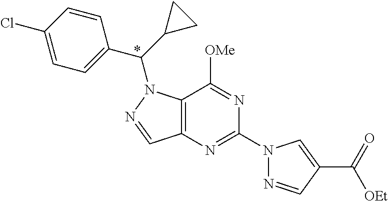 | solid MS (APCI) m/z: 453/455 [M + H]⁺ |
| 269 | 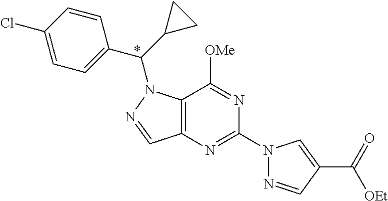 | solid MS (APCI) m/z: 453/455 [M + H]⁺ Enantiomer of Reference Example 268 |
| 270 | 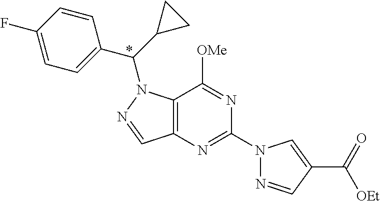 | solid MS (APCI) m/z: 437 [M + H]⁺ |
| 271 | 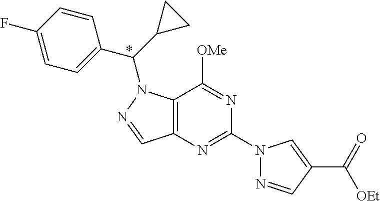 | solid MS (APCI) m/z: 437 [M + H]⁺ Enantiomer of Reference Example 270 |
| 272 | 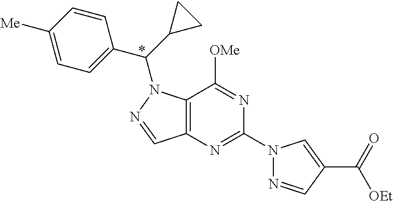 | viscous material MS (APCI) m/z: 433 [M + H]⁺ |
| 273 | 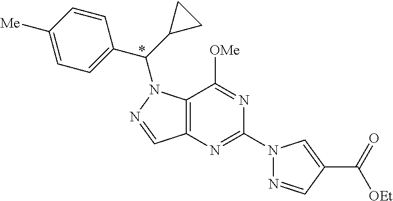 | viscous material MS (APCI) m/z: 433 [M + H]⁺ Enantiomer of Reference Example 272 |
| 274 | 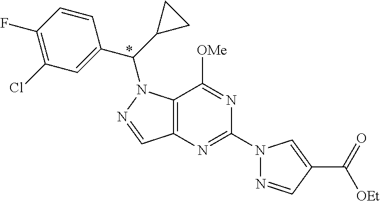 | viscous material MS (APCI) m/z: 471/473 [M + H]⁺ |
| 275 | 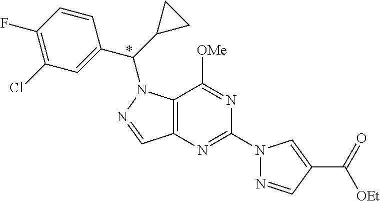 | viscous material MS (APCI) m/z: 471/473 [M + H]⁺ Enantiomer of Reference Example 274 |
| 276 | 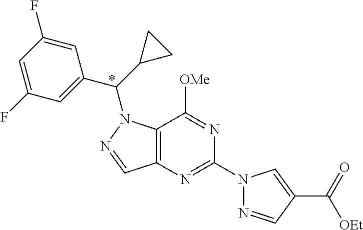 | solid MS (APCI) m/z: 455 [M + H]⁺ |
| 277 | 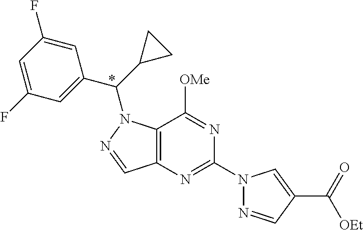 | solid MS (APCI) m/z: 455 [M + H]⁺ Enantiomer of Reference Example 276 |
| 278 | 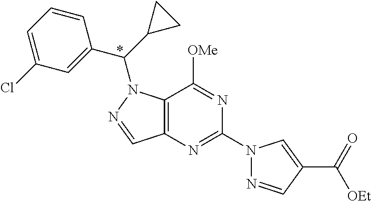 | viscous material MS (APCI) m/z: 453/455 [M + H]⁺ |
| 279 | 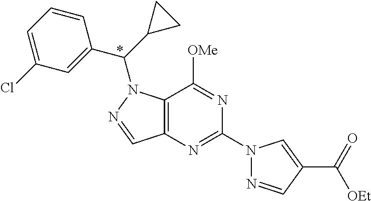 | viscous material MS (APCI) m/z: 453/455 [M + H]⁺ Enantiomer of Reference Example 278 |
| 280 | 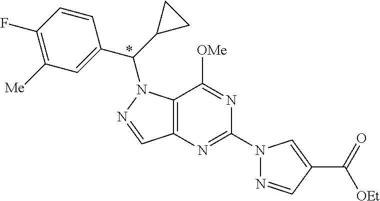 | viscous material MS (APCI) m/z: 451 [M + H]⁺ |
| 281 | 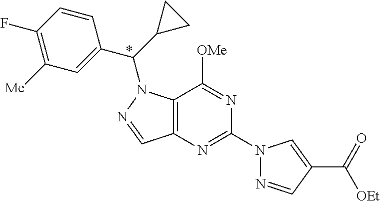 | viscous material MS (APCI) m/z: 451 [M + H]⁺ Enantiomer of Reference Example 280 |
| 282 | 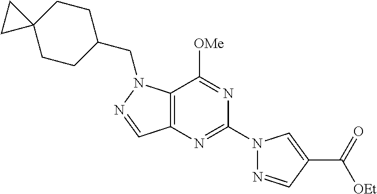 [SMILES: CCOC(=O)c1cnn(-c2nc(OC)c3c(cnn3CC3CCC4(CC3)CC4)n2)c1] | powder MS (APCI) m/z: 411 [M + H]⁺ |
| 283 | 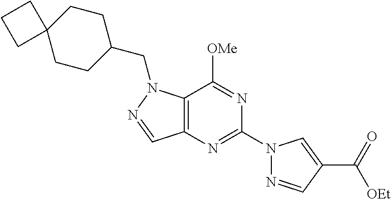 [SMILES: CCOC(=O)c1cnn(-c2nc(OC)c3c(cnn3CC3CCC4(CCC4)CC3)n2)c1] | powder MS (APCI) m/z: 425 [M + H]⁺ |
| 284 | 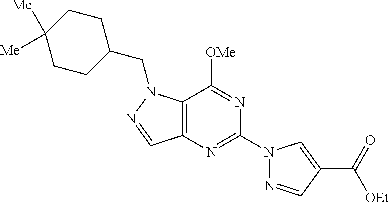 [SMILES: CCOC(=O)c1cnn(-c2nc(OC)c3c(cnn3CC3CCC(C)(C)CC3)n2)c1] | powder MS (APCI) m/z: 413 [M + H]⁺ |
| 285 | 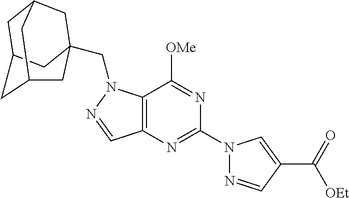 [SMILES: CCOC(=O)c1cnn(-c2nc(OC)c3c(cnn3CC34CC5CC(CC(C5)C3)C4)n2)c1] | powder MS (ESI) m/z: 437 [M + H]⁺ |
| 286 | 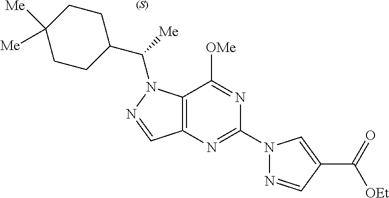 | powder MS (APCI) m/z: 427 [M + H]⁺ |
| 287 | 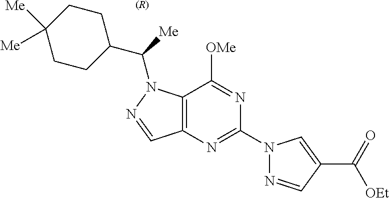 | powder MS (APCI) m/z: 427 [M + H]⁺ |
| 288 | 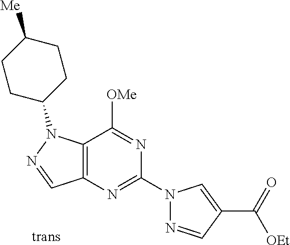 [SMILES: CCOC(=O)c1cnn(-c2nc(OC)c3c(cnn3[C@H]3CC[C@H](C)CC3)n2)c1] | powder MS (APCI) m/z: 385 [M + H]⁺ |
| 289 | 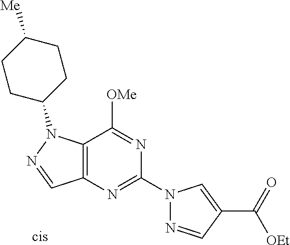 [SMILES: CCOC(=O)c1cnn(-c2nc(OC)c3c(cnn3[C@H]3CC[C@@H](C)CC3)n2)c1] | powder MS (APCI) m/z: 385 [M + H]⁺ |
| 290 | 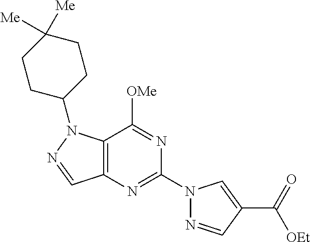 [SMILES: CCOC(=O)c1cnn(-c2nc(OC)c3c(cnn3C3CCC(C)(C)CC3)n2)c1] | powder MS (APCI) m/z: 399 [M + H]⁺ |
| 291 | 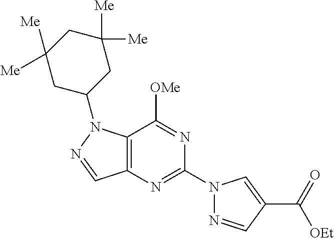 [SMILES: CCOC(=O)c1cnn(-c2nc(OC)c3c(cnn3C3CC(C)(C)CC(C)(C)C3)n2)c1] | viscous material MS (APCI) m/z: 427 [M + H]⁺ |
| 292 | 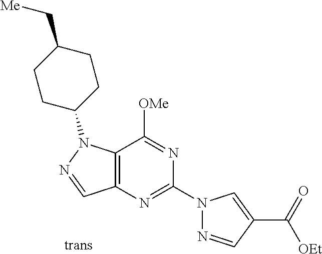 [SMILES: CCOC(=O)c1cnn(-c2nc(OC)c3c(cnn3[C@H]3CC[C@H](CC)CC3)n2)c1] | powder MS (APCI) m/z: 399 [M + H]⁺ |
| 293 | 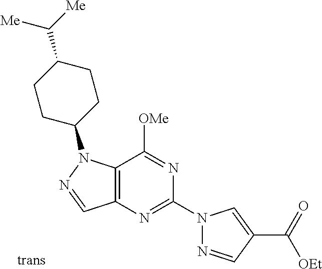 [SMILES: CCOC(=O)c1cnn(-c2nc(OC)c3c(cnn3[C@H]3CC[C@H](C(C)C)CC3)n2)c1] | powder MS (APCI) m/z: 413 [M + H]⁺ |
| 294 | 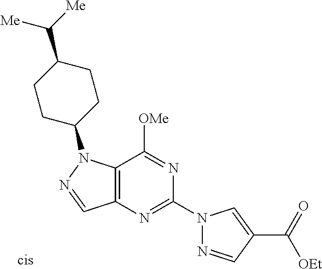 [SMILES: CCOC(=O)c1cnn(-c2nc(OC)c3c(cnn3[C@H]3CC[C@@H](C(C)C)CC3)n2)c1] | powder MS (APCI) m/z: 413 [M + H]⁺ |
| 295 | 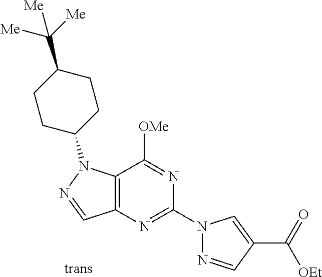 [SMILES: CCOC(=O)c1cnn(-c2nc(OC)c3c(cnn3[C@H]3CC[C@H](C(C)(C)C)CC3)n2)c1] | powder MS (APCI) m/z: 427 [M + H]⁺ |
| 296 | 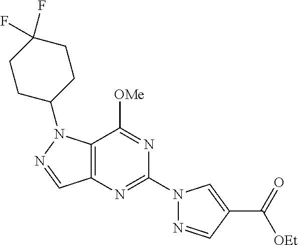 [SMILES: CCOC(=O)c1cnn(-c2nc(OC)c3c(cnn3C3CCC(F)(F)CC3)n2)c1] | powder MS (APCI) m/z: 407 [M + H]⁺ |
| 297 | 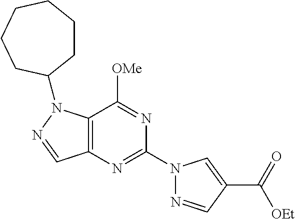 [SMILES: CCOC(=O)c1cnn(-c2nc(OC)c3c(cnn3C3CCCCCC3)n2)c1] | powder MS (APCI) m/z: 385 [M + H]⁺ |
| 298 | 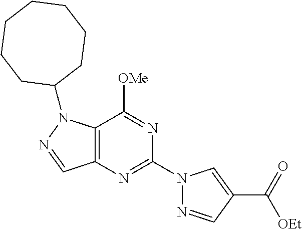 [SMILES: CCOC(=O)c1cnn(-c2nc(OC)c3c(cnn3C3CCCCCCC3)n2)c1] | powder MS (APCI) m/z: 399 [M + H]⁺ |
| 299 | 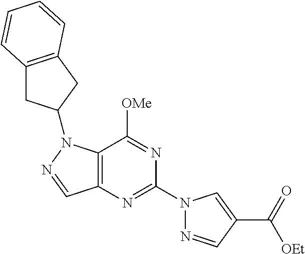 [SMILES: CCOC(=O)c1cnn(-c2nc(OC)c3c(cnn3C3Cc4ccccc4C3)n2)c1] | powder MS (APCI) m/z: 405 [M + H]⁺ |
| 300 | 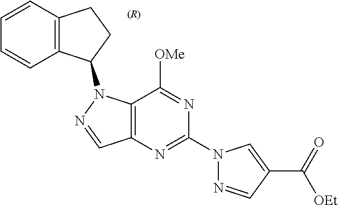 | powder MS (APCI) m/z: 405 [M + H]⁺ |
| 301 | 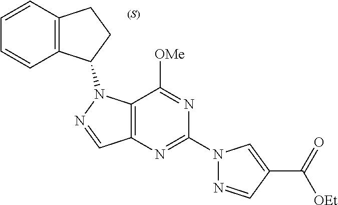 | powder MS (APCI) m/z: 405 [M + H]⁺ |
| 302 | 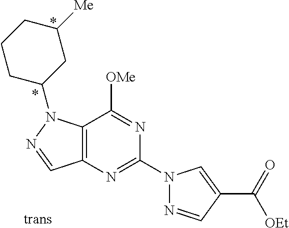 | solid MS (ESI) m/z: 385 [M + H]⁺ |
| 303 | 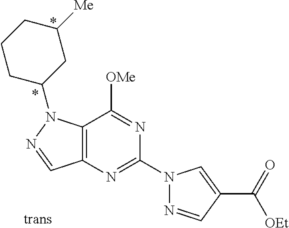 | solid MS (ESI) m/z: 385 [M + H]⁺ Enantiomer of Reference Example 302 |
| 304 | 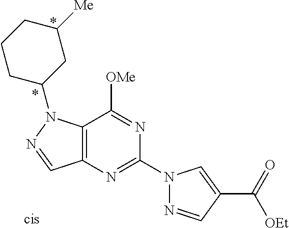 | powder MS (ESI) m/z: 385 [M + H]⁺ |
| 305 | 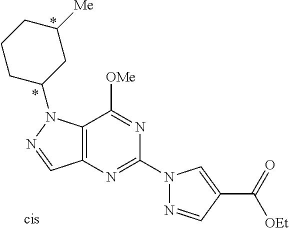 | powder MS (ESI) m/z: 385 [M + H]⁺ Enantiomer of Reference Example 304 |
| 306 | 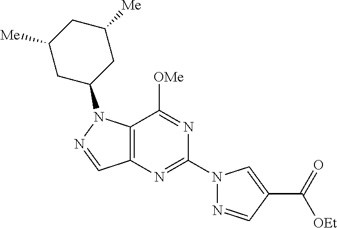 [SMILES: CCOC(=O)c1cnn(-c2nc(OC)c3c(cnn3[C@H]3C[C@H](C)C[C@H](C)C3)n2)c1] | powder MS (APCI) m/z: 399 [M + H]⁺ |
| 307 | 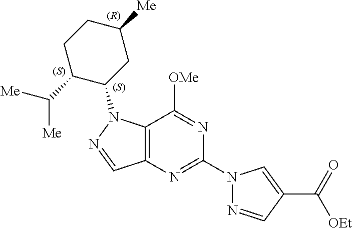 | viscous material MS (APCI) m/z: 427 [M + H]⁺ |
| 308 | 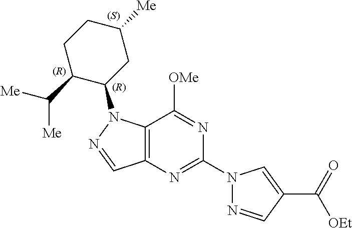 | viscous material MS (APCI) m/z: 427 [M + H]⁺ |
| 309 | 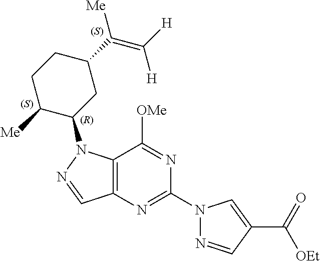 | viscous material MS (APCI) m/z: 425 [M + H]⁺ |
| 310 | 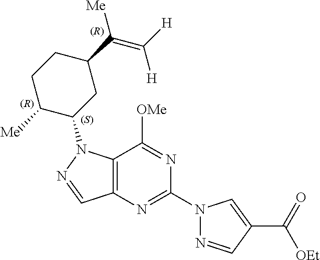 | powder MS (APCI) m/z: 425 [M + H]⁺ |
| 311 | 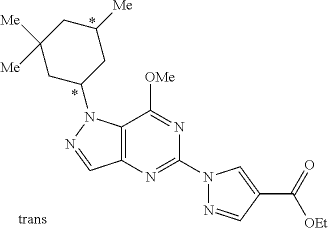 | solid MS (ESI) m/z: 413 [M + H]⁺ |
| 312 | 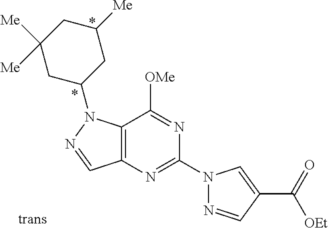 | solid MS (ESI) m/z: 413 [M + H]⁺ Enantiomer of Reference Example 311 |
| 313 | 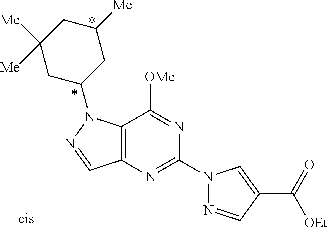 | solid MS (ESI) m/z: 413 [M + H]⁺ |
| 314 | 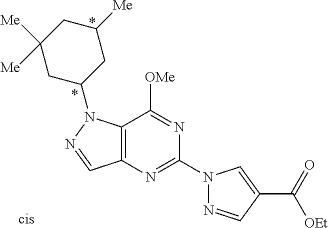 | solid MS (ESI) m/z: 413 [M + H]⁺ Enantiomer of Reference Example 313 |
| 315 | 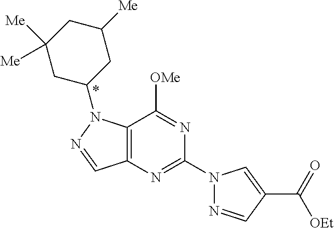 | powder MS (APCI) m/z: 399 [M + H]⁺ |
| 316 | 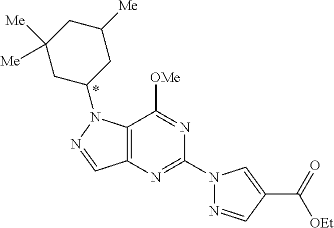 | powder MS (APCI) m/z: 399 [M + H]⁺ Enantiomer of Reference Example 315 |
| 317 | 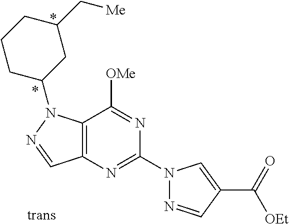 | powder MS (APCI) m/z: 399 [M + H]⁺ |
| 318 | 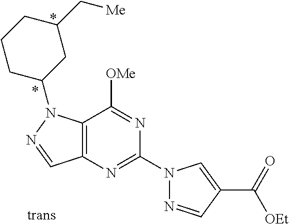 | powder MS (APCI) m/z: 399 [M + H]⁺ Enantiomer of Reference Example 317 |
| 319 | 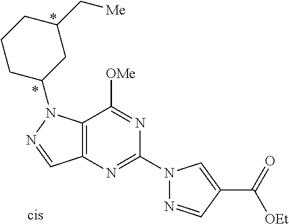 | powder MS (APCI) m/z: 399 [M + H]⁺ |
| 320 | 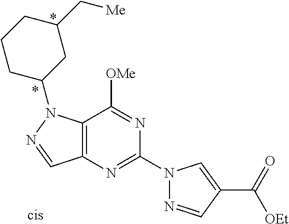 | powder MS (APCI) m/z: 399 [M + H]⁺ Enantiomer of Reference Example 319 |
| 321 | 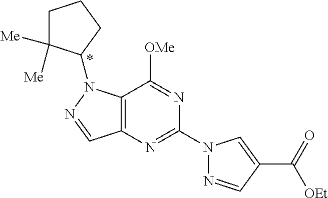 | powder MS (APCI) m/z: 385 [M + H]⁺ |
| 322 | 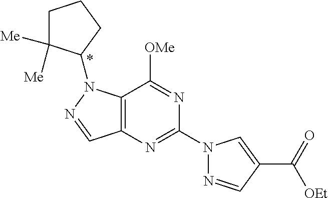 | powder MS (APCI) m/z: 385 [M + H]⁺ Enantiomer of Reference Example 321 |
| 323 | 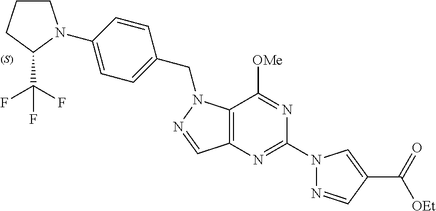 | powder MS (ESI) m/z: 516 [M + H]⁺ |
| 324 | 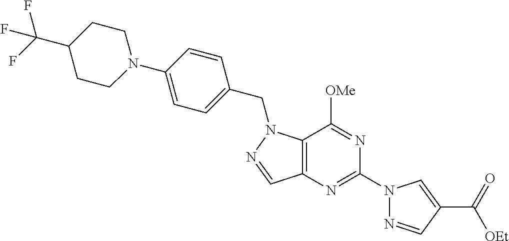 [SMILES: CCOC(=O)c1cnn(-c2nc(OC)c3c(cnn3Cc3ccc(N4CCC(C(F)(F)F)CC4)cc3)n2)c1] | powder MS (ESI) m/z: 530 [M + H]⁺ |
| 325 | 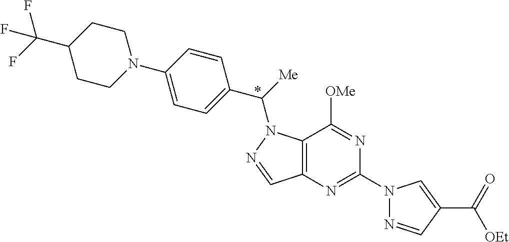 | viscous material MS (ESI) m/z: 544 [M + H]⁺ racemic mixture |
| 326 | 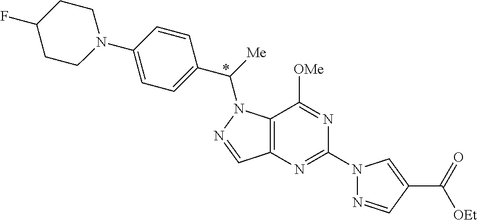 | viscous material MS (ESI) m/z: 494 [M + H]⁺ racemic mixture |
| 327 | 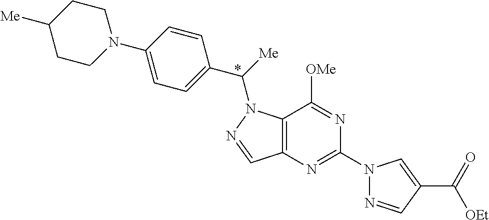 | viscous material MS (ESI) m/z: 490 [M + H]⁺ racemic mixture |
| 328 | 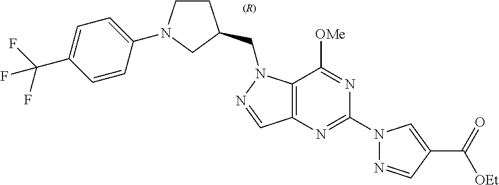 | powder MS (ESI) m/z: 516 [M + H]⁺ |
| 329 | 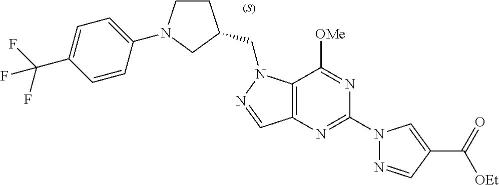 | solid MS (ESI) m/z: 516 [M + H]⁺ |
| 330 | 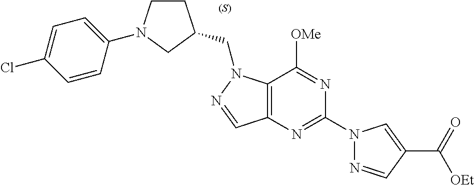 | powder MS (ESI) m/z: 482/484 [M + H]⁺ |
| 331 | 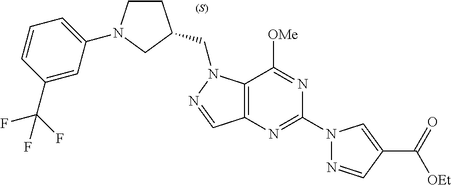 | viscous material MS (ESI) m/z: 516 [M + H]⁺ |
| 332 | 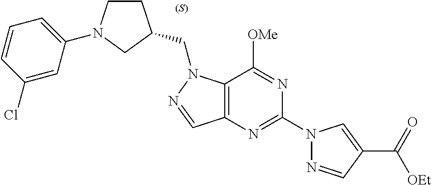 | viscous material MS (ESI) m/z: 482/484 [M + H]⁺ |
| 333 | 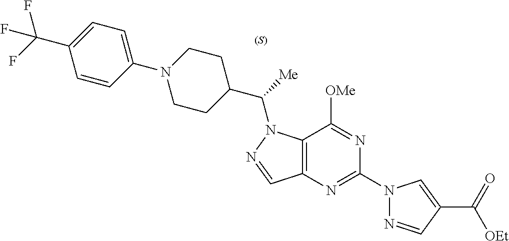 | powder MS (ESI) m/z: 544 [M + H]⁺ |
| 334 | 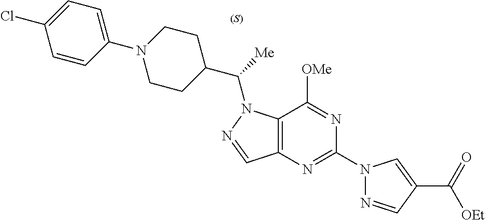 | solid MS (ESI) m/z: 510/512 [M + H]⁺ |
| 335 | 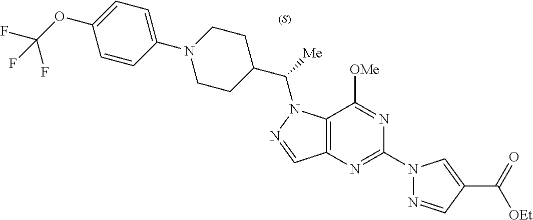 | viscous material MS (ESI) m/z: 560 [M + H]⁺ |
| 336 | 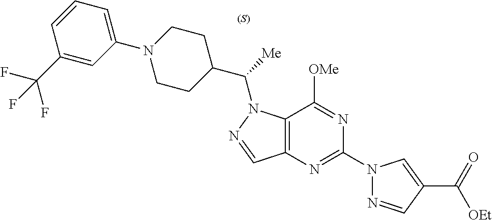 | viscous material MS (ESI) m/z: 544 [M + H]⁺ |
| 337 | 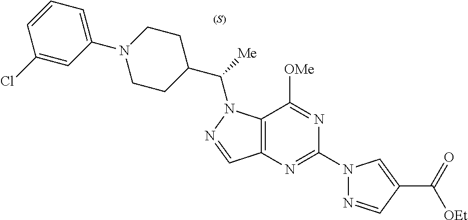 | viscous material MS (ESI) m/z: 510/512 [M + H]⁺ |
| 338 | 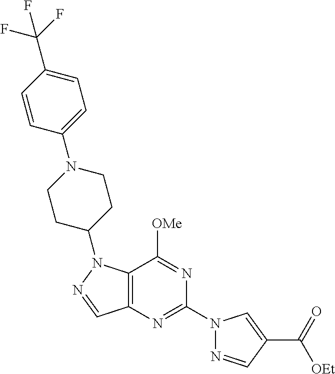 [SMILES: CCOC(=O)c1cnn(-c2nc(OC)c3c(cnn3C3CCN(c4ccc(C(F)(F)F)cc4)CC3)n2)c1] | solid MS (ESI) m/z: 516 [M + H]⁺ |
| 339 | 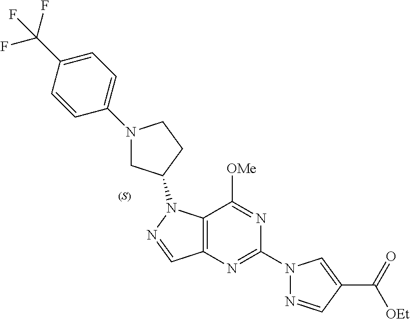 | solid MS (ESI) m/z: 502 [M + H]⁺ |
| 340 | 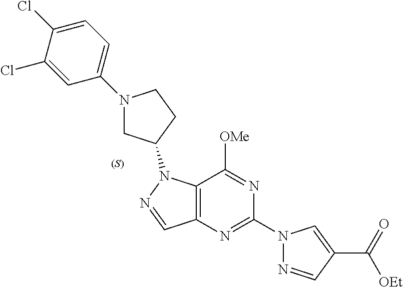 | powder MS (ESI) m/z: 502/504 [M + H]⁺ |
| 341 | 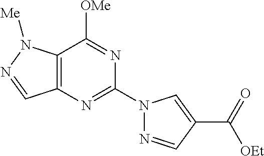 [SMILES: CCOC(=O)c1cnn(-c2nc(OC)c3c(cnn3C)n2)c1] | powder MS (APCI) m/z: 303 [M + H]⁺ |
| 342 | 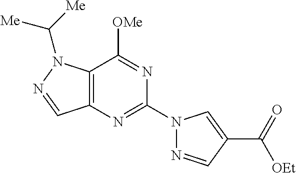 [SMILES: CCOC(=O)c1cnn(-c2nc(OC)c3c(cnn3C(C)C)n2)c1] | powder MS (APCI) m/z: 331 [M + H]⁺ |
| 343 | 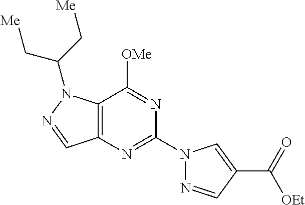 [SMILES: CCOC(=O)c1cnn(-c2nc(OC)c3c(cnn3C(CC)CC)n2)c1] | powder MS (APCI) m/z: 359 [M + H]⁺ |
| 344 | 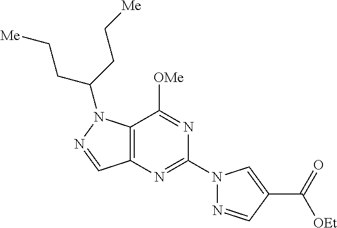 [SMILES: CCCC(CCC)n1ncc2nc(-n3cc(C(=O)OCC)cn3)nc(OC)c21] | powder MS (APCI) m/z: 387 [M + H]⁺ |
| 345 | 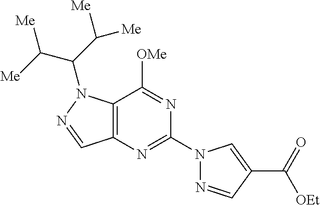 [SMILES: CCOC(=O)c1cnn(-c2nc(OC)c3c(cnn3C(C(C)C)C(C)C)n2)c1] | viscous material MS (APCI) m/z: 387 [M + H]⁺ |
| 346 | 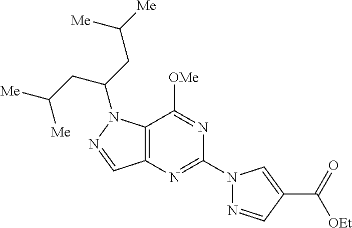 [SMILES: CCOC(=O)c1cnn(-c2nc(OC)c3c(cnn3C(CC(C)C)CC(C)C)n2)c1] | viscous material MS (APCI) m/z: 415 [M + H]⁺ |
| 347 | 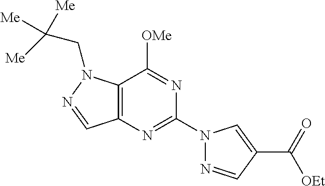 [SMILES: CCOC(=O)c1cnn(-c2nc(OC)c3c(cnn3CC(C)(C)C)n2)c1] | powder MS (APCI) m/z: 359 [M + H]⁺ |
| 348 | 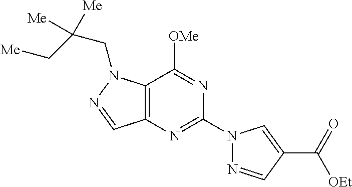 [SMILES: CCOC(=O)c1cnn(-c2nc(OC)c3c(cnn3CC(C)(C)CC)n2)c1] | powder MS (APCI) m/z: 373 [M + H]⁺ |
| 349 | 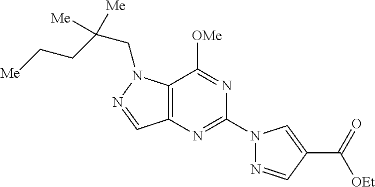 [SMILES: CCCC(C)(C)Cn1ncc2nc(-n3cc(C(=O)OCC)cn3)nc(OC)c21] | powder MS (APCI) m/z: 387 [M + H]⁺ |
| 350 | 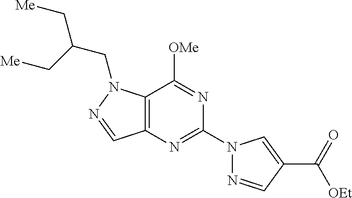 [SMILES: CCOC(=O)c1cnn(-c2nc(OC)c3c(cnn3CC(CC)CC)n2)c1] | powder MS (APCI) m/z: 373 [M + H]⁺ |
| 351 | 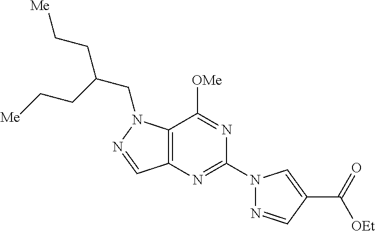 [SMILES: CCCC(CCC)Cn1ncc2nc(-n3cc(C(=O)OCC)cn3)nc(OC)c21] | powder MS (APCI) m/z: 401 [M + H]⁺ |
| 352 | 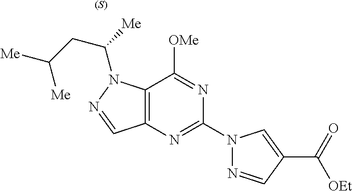 | viscous material MS (APCI) m/z: 373 [M + H]⁺ |
| 353 | 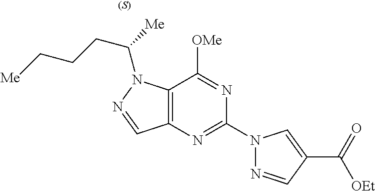 | viscous material MS (APCI) m/z: 373 [M + H]⁺ |
| 354 | 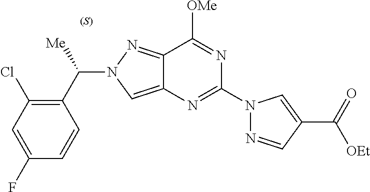 | powder MS (ESI) m/z: 445/447 [M + H]⁺ |
| 355 | 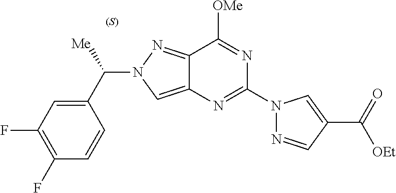 | powder MS (APCI) m/z: 443 [M + H]⁺ |

### REFERENCE EXAMPLE 356

### Preparation of ethyl 1-{2-[(3-fluorobiphenyl-4-yl)methyl]-7-methoxy-2H-pyrazolo[4,3-d]pyrimidin-5-yl}-1H-pyrazole-4-carboxylate

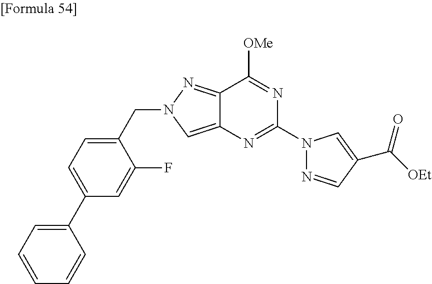 [SMILES: CCOC(=O)c1cnn(-c2nc(OC)c3nn(Cc4ccc(-c5ccccc5)cc4F)cc3n2)c1]

A suspension of ethyl 1-[2-(4-chloro-2-fluorobenzyl)-7-methoxy-2H-pyrazolo[4,3-d]pyrimidin-5-yl]-1H-pyrazole-4-carboxylate (43.7 mg) prepared in Reference Example 18, phenylboronic acid (32 mg), palladium(II) acetate (3.6 mg), tripotassium phosphate (64.3 mg) and 2-dicyclohexyl-phosphino-2′,6′-dimethoxybiphenyl (21.6 mg) in toluene (1.6 mL) was stirred under microwave irradiation for 1 hour at 100° C. and then for 30 minutes at 120° C. After addition of chloroform and water to the reaction mixture, the organic layer was separated, and the aqueous layer was extracted with chloroform. The organic layer was concentrated under reduced pressure, and the residue was purified by NH-silica gel column chromatography (solvent: hexane/ethyl acetate=70/30 to 25/75). The resulting crude product was purified by thin-layer chromatography on silica gel (solvent: chloroform/ethyl acetate=80/20) to yield the titled compound (29 mg, 61% yield) as a colorless solid.

MS (APCI) m/z: 473 [M+H]⁺.

### REFERENCE EXAMPLE 357

### Preparation of ethyl 1-{1-[(3-fluorobiphenyl-4-yl)methyl]-7-methoxy-1H-pyrazolo[4,3-d]pyrimidin-5-yl}-1H-pyrazole-4-carboxylate

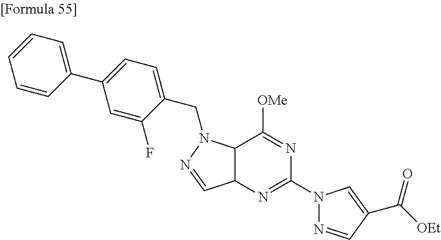 [SMILES: CCOC(=O)c1cnn(C2=NC3C=NN(Cc4ccc(-c5ccccc5)cc4F)C3C(OC)=N2)c1]

A suspension of ethyl 1-[1-(4-bromo-2-fluorobenzyl)-7-methoxy-1H-pyrazolo[4,3-d]pyrimidin-5-yl]-1H-pyrazole-4-carboxylate prepared in Reference Example 119 (356 mg), phenylboronic acid (183 mg), palladium (II) acetate (8.4 mg), tripotassium phosphate (477 mg) and 2-dicyclohexyl-phosphino-2′,6′-dimethoxybipheny (130.8 mg) in toluene (7 mL) was stirred for 19.5 hours at 100° C. After adding water to the reaction mixture, the organic layer was separated, and the aqueous layer was extracted with ethyl acetate. The organic layer was concentrated under reduced pressure, and the residue was purified by silica gel column chromatography (solvent: hexane/ethyl acetate=80/20 to 50/50) to yield the titled compound (258.7 mg, 73.1% yield) as a colorless solid.

MS (APCI) m/z: 473 [M+H]⁺.

### REFERENCE EXAMPLES 358 TO 421

The compounds listed in the following Table 4 were obtained from the corresponding starting material in the same manner as described in Reference Examples 356 or 357.

TABLE 4

| Reference | | material |
|---|---|---|
| Example | Structure | properties |
| 358 | 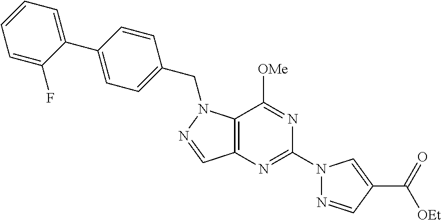 [SMILES: CCOC(=O)c1cnn(-c2nc(OC)c3c(cnn3Cc3ccc(-c4ccccc4F)cc3)n2)c1] | powder MS (ESI) m/z: 473 [M + H]⁺ |
| 359 | 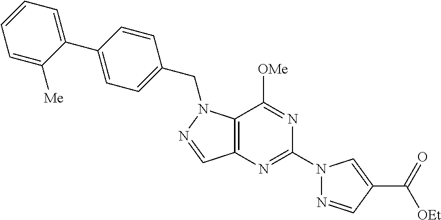 [SMILES: CCOC(=O)c1cnn(-c2nc(OC)c3c(cnn3Cc3ccc(-c4ccccc4C)cc3)n2)c1] | powder MS (ESI) m/z: 469 [M + H]⁺ |
| 360 | 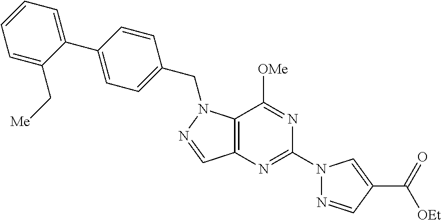 [SMILES: CCOC(=O)c1cnn(-c2nc(OC)c3c(cnn3Cc3ccc(-c4ccccc4CC)cc3)n2)c1] | powder MS (APCI) m/z: 483 [M + H]⁺ |
| 361 | 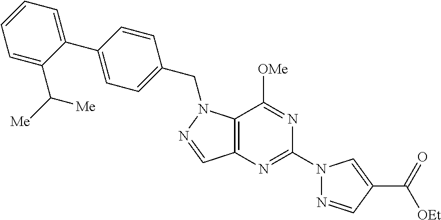 [SMILES: CCOC(=O)c1cnn(-c2nc(OC)c3c(cnn3Cc3ccc(-c4ccccc4C(C)C)cc3)n2)c1] | powder MS (APCI) m/z: 497 [M + H]⁺ |
| 362 | 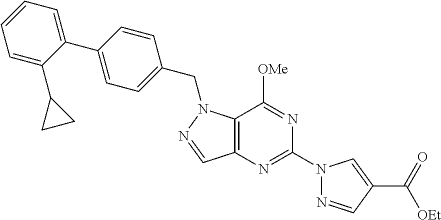 [SMILES: CCOC(=O)c1cnn(-c2nc(OC)c3c(cnn3Cc3ccc(-c4ccccc4C4CC4)cc3)n2)c1] | powder MS (APCI) m/z: 495 [M + H]⁺ |
| 363 | 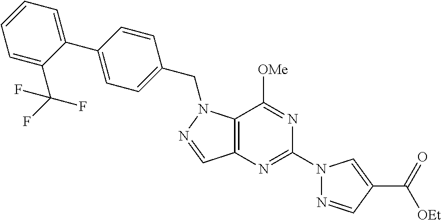 [SMILES: CCOC(=O)c1cnn(-c2nc(OC)c3c(cnn3Cc3ccc(-c4ccccc4C(F)(F)F)cc3)n2)c1] | powder MS (ESI) m/z: 523 [M + H]⁺ |
| 364 | 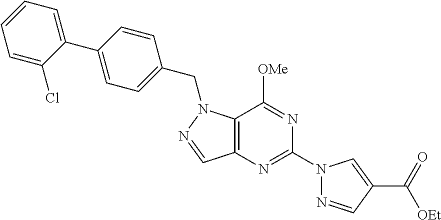 [SMILES: CCOC(=O)c1cnn(-c2nc(OC)c3c(cnn3Cc3ccc(-c4ccccc4Cl)cc3)n2)c1] | powder MS (ESI) m/z: 489/491 [M + H]⁺ |
| 365 | 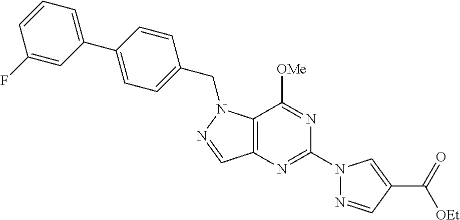 [SMILES: CCOC(=O)c1cnn(-c2nc(OC)c3c(cnn3Cc3ccc(-c4cccc(F)c4)cc3)n2)c1] | powder MS (ESI) m/z: 473 [M + H]⁺ |
| 366 | 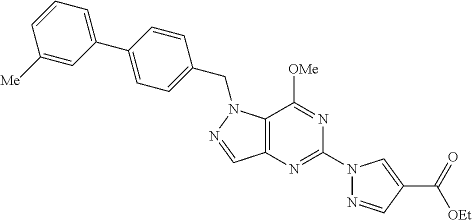 [SMILES: CCOC(=O)c1cnn(-c2nc(OC)c3c(cnn3Cc3ccc(-c4cccc(C)c4)cc3)n2)c1] | powder MS (ESI) m/z: 469 [M + H]⁺ |
| 367 | 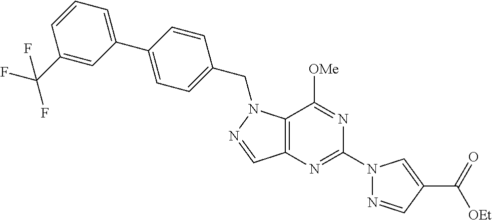 [SMILES: CCOC(=O)c1cnn(-c2nc(OC)c3c(cnn3Cc3ccc(-c4cccc(C(F)(F)F)c4)cc3)n2)c1] | powder MS (ESI) m/z: 523 [M + H]⁺ |
| 368 | 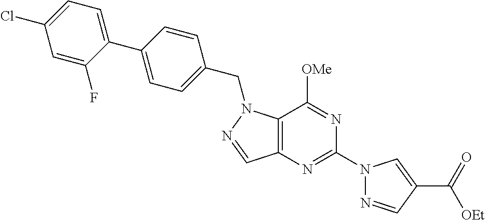 [SMILES: CCOC(=O)c1cnn(-c2nc(OC)c3c(cnn3Cc3ccc(-c4ccc(Cl)cc4F)cc3)n2)c1] | powder MS (ESI) m/z: 507/509 [M + H]⁺ |
| 369 | 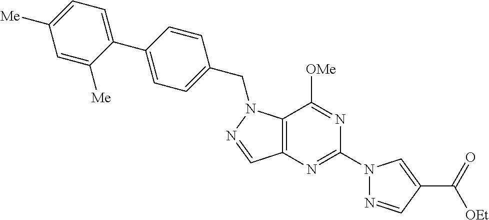 [SMILES: CCOC(=O)c1cnn(-c2nc(OC)c3c(cnn3Cc3ccc(-c4ccc(C)cc4C)cc3)n2)c1] | powder MS (APCI) m/z: 483 [M + H]⁺ |
| 370 | 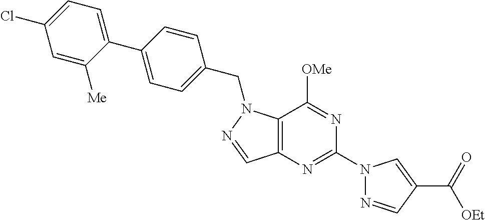 [SMILES: CCOC(=O)c1cnn(-c2nc(OC)c3c(cnn3Cc3ccc(-c4ccc(Cl)cc4C)cc3)n2)c1] | powder MS (APCI) m/z: 503/505 [M + H]⁺ |
| 371 | 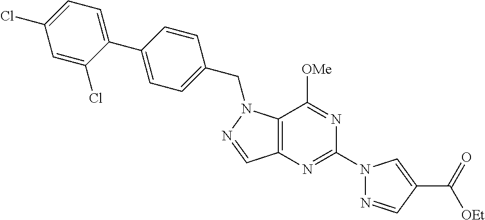 [SMILES: CCOC(=O)c1cnn(-c2nc(OC)c3c(cnn3Cc3ccc(-c4ccc(Cl)cc4Cl)cc3)n2)c1] | powder MS (APCI) m/z: 523/525 [M + H]⁺ |
| 372 | 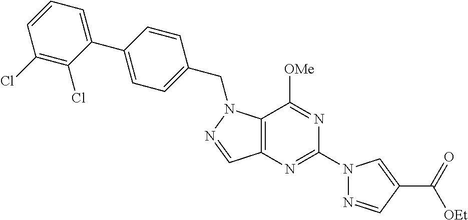 [SMILES: CCOC(=O)c1cnn(-c2nc(OC)c3c(cnn3Cc3ccc(-c4cccc(Cl)c4Cl)cc3)n2)c1] | powder MS (ESI) m/z: 523/525 [M + H]⁺ |
| 373 | 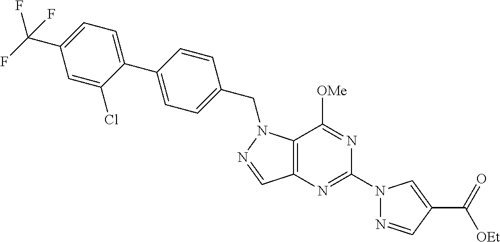 [SMILES: CCOC(=O)c1cnn(-c2nc(OC)c3c(cnn3Cc3ccc(-c4ccc(C(F)(F)F)cc4Cl)cc3)n2)c1] | powder MS (ESI) m/z: 557/559 [M + H]⁺ |
| 374 | 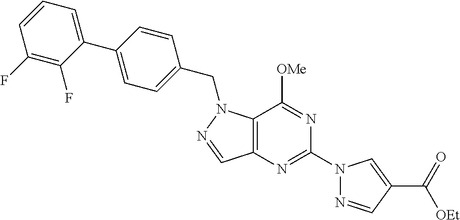 [SMILES: CCOC(=O)c1cnn(-c2nc(OC)c3c(cnn3Cc3ccc(-c4cccc(F)c4F)cc3)n2)c1] | powder MS (APCI) m/z: 491 [M + H]⁺ |
| 375 | 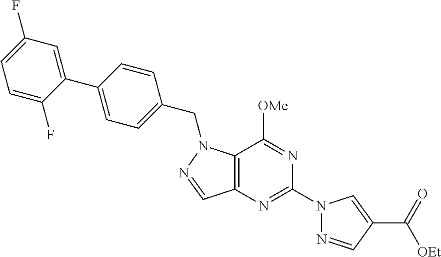 [SMILES: CCOC(=O)c1cnn(-c2nc(OC)c3c(cnn3Cc3ccc(-c4cc(F)ccc4F)cc3)n2)c1] | powder MS (ESI) m/z: 491 [M + H]⁺ |
| 376 | 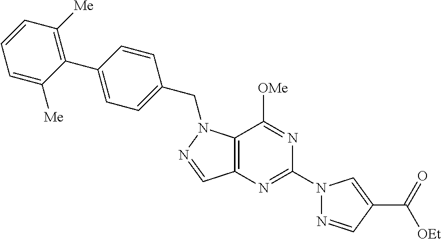 [SMILES: CCOC(=O)c1cnn(-c2nc(OC)c3c(cnn3Cc3ccc(-c4c(C)cccc4C)cc3)n2)c1] | powder MS (ESI) m/z: 483 [M + H]⁺ |
| 377 | 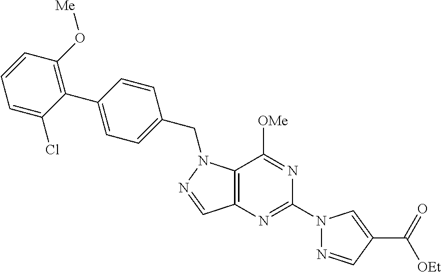 [SMILES: CCOC(=O)c1cnn(-c2nc(OC)c3c(cnn3Cc3ccc(-c4c(Cl)cccc4OC)cc3)n2)c1] | powder MS (APCI) m/z: 519/521 [M + H]⁺ |
| 378 | 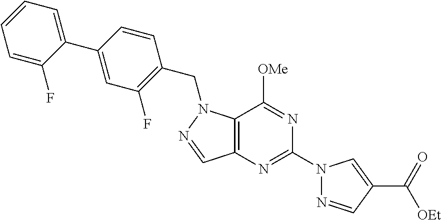 [SMILES: CCOC(=O)c1cnn(-c2nc(OC)c3c(cnn3Cc3ccc(-c4ccccc4F)cc3F)n2)c1] | powder MS (ESI) m/z: 491 [M + H]⁺ |
| 379 | 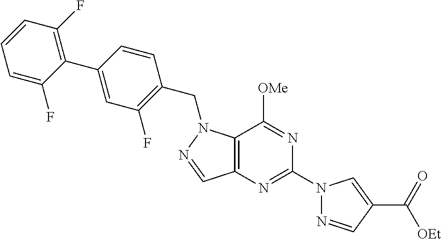 [SMILES: CCOC(=O)c1cnn(-c2nc(OC)c3c(cnn3Cc3ccc(-c4c(F)cccc4F)cc3F)n2)c1] | powder MS (ESI) m/z: 509 [M + H]⁺ |
| 380 | 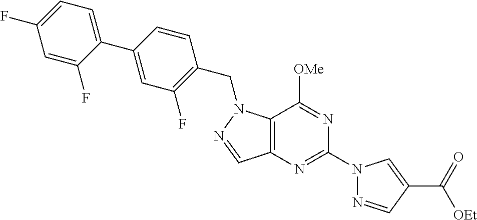 [SMILES: CCOC(=O)c1cnn(-c2nc(OC)c3c(cnn3Cc3ccc(-c4ccc(F)cc4F)cc3F)n2)c1] | powder MS (APCI) m/z: 509 [M + H]⁺ |
| 381 | 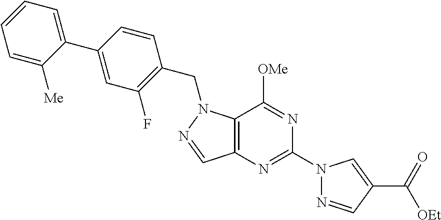 [SMILES: CCOC(=O)c1cnn(-c2nc(OC)c3c(cnn3Cc3ccc(-c4ccccc4C)cc3F)n2)c1] | powder MS (ESI) m/z: 487 [M + H]⁺ |
| 382 | 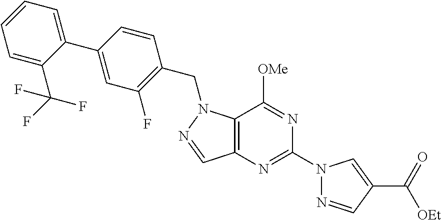 [SMILES: CCOC(=O)c1cnn(-c2nc(OC)c3c(cnn3Cc3ccc(-c4ccccc4C(F)(F)F)cc3F)n2)c1] | powder MS (ESI) m/z: 541 [M + H]⁺ |
| 383 | 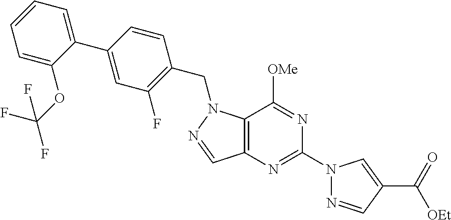 [SMILES: CCOC(=O)c1cnn(-c2nc(OC)c3c(cnn3Cc3ccc(-c4ccccc4OC(F)(F)F)cc3F)n2)c1] | powder MS (ESI) m/z: 557 [M + H]⁺ |
| 384 | 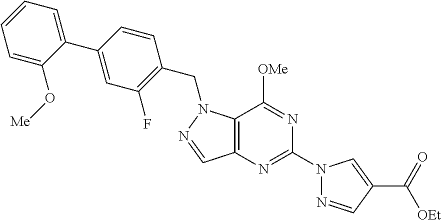 [SMILES: CCOC(=O)c1cnn(-c2nc(OC)c3c(cnn3Cc3ccc(-c4ccccc4OC)cc3F)n2)c1] | powder MS (ESI) m/z: 503 [M + H]⁺ |
| 385 | 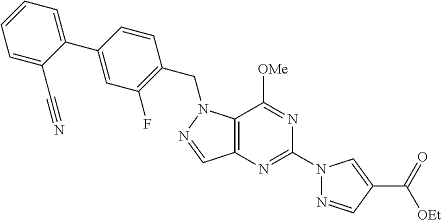 [SMILES: CCOC(=O)c1cnn(-c2nc(OC)c3c(cnn3Cc3ccc(-c4ccccc4C#N)cc3F)n2)c1] | powder MS (ESI) m/z: 498 [M + H]⁺ |
| 386 | 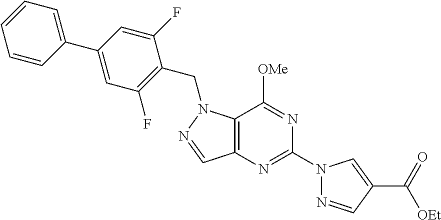 [SMILES: CCOC(=O)c1cnn(-c2nc(OC)c3c(cnn3Cc3c(F)cc(-c4ccccc4)cc3F)n2)c1] | powder MS (ESI) m/z: 491 [M + H]⁺ |
| 387 | 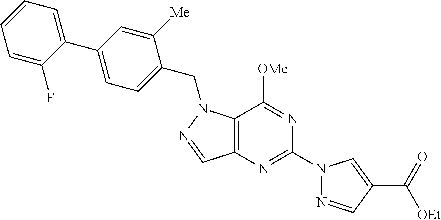 [SMILES: CCOC(=O)c1cnn(-c2nc(OC)c3c(cnn3Cc3ccc(-c4ccccc4F)cc3C)n2)c1] | powder MS (ESI) m/z: 487 [M + H]⁺ |
| 388 | 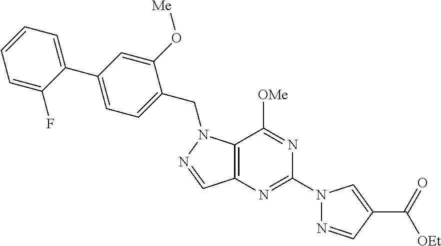 [SMILES: CCOC(=O)c1cnn(-c2nc(OC)c3c(cnn3Cc3ccc(-c4ccccc4F)cc3OC)n2)c1] | powder MS (ESI) m/z: 503 [M + H]⁺ |
| 389 | 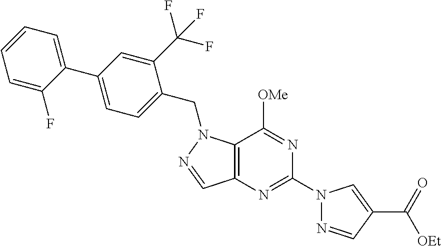 [SMILES: CCOC(=O)c1cnn(-c2nc(OC)c3c(cnn3Cc3ccc(-c4ccccc4F)cc3C(F)(F)F)n2)c1] | powder MS (ESI) m/z: 541 [M + H]⁺ |
| 390 | 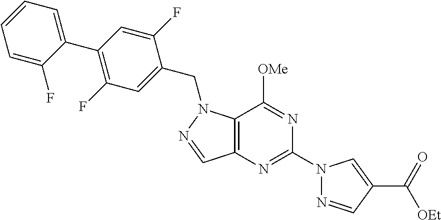 [SMILES: CCOC(=O)c1cnn(-c2nc(OC)c3c(cnn3Cc3cc(F)c(-c4ccccc4F)cc3F)n2)c1] | powder MS (ESI) m/z: 509 [M + H]⁺ |
| 391 | 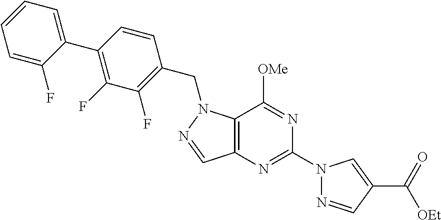 [SMILES: CCOC(=O)c1cnn(-c2nc(OC)c3c(cnn3Cc3ccc(-c4ccccc4F)c(F)c3F)n2)c1] | powder MS (APCI) m/z: 509 [M + H]⁺ |
| 392 | 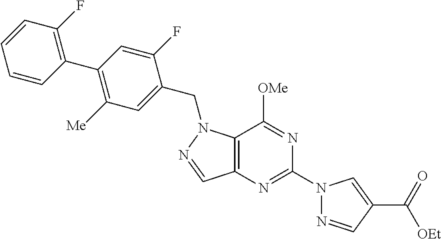 [SMILES: CCOC(=O)c1cnn(-c2nc(OC)c3c(cnn3Cc3cc(C)c(-c4ccccc4F)cc3F)n2)c1] | powder MS (APCI) m/z: 505 [M + H]⁺ |
| 393 | 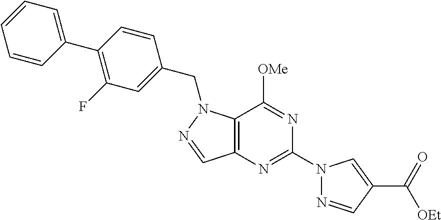 [SMILES: CCOC(=O)c1cnn(-c2nc(OC)c3c(cnn3Cc3ccc(-c4ccccc4)c(F)c3)n2)c1] | powder MS (APCI) m/z: 473 [M + H]⁺ |
| 394 | 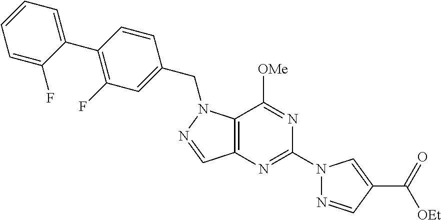 [SMILES: CCOC(=O)c1cnn(-c2nc(OC)c3c(cnn3Cc3ccc(-c4ccccc4F)c(F)c3)n2)c1] | powder MS (APCI) m/z: 491 [M + H]⁺ |
| 395 | 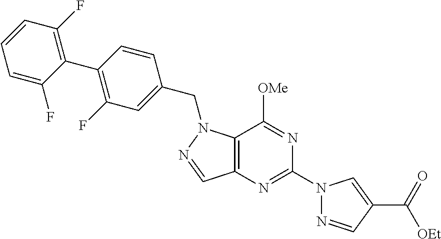 [SMILES: CCOC(=O)c1cnn(-c2nc(OC)c3c(cnn3Cc3ccc(-c4c(F)cccc4F)c(F)c3)n2)c1] | powder MS (APCI) m/z: 509 [M + H]⁺ |
| 396 | 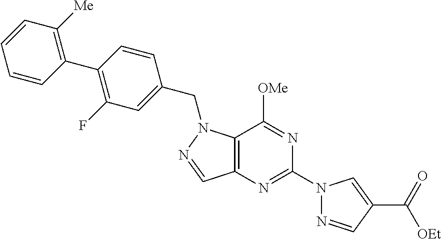 [SMILES: CCOC(=O)c1cnn(-c2nc(OC)c3c(cnn3Cc3ccc(-c4ccccc4C)c(F)c3)n2)c1] | powder MS (APCI) m/z: 487 [M + H]⁺ |
| 397 | 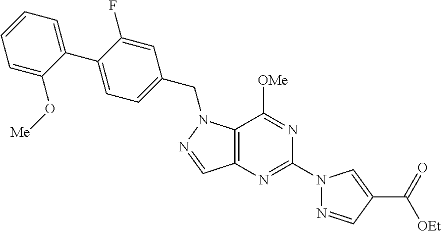 [SMILES: CCOC(=O)c1cnn(-c2nc(OC)c3c(cnn3Cc3ccc(-c4ccccc4OC)c(F)c3)n2)c1] | powder MS (ESI) m/z: 503 [M + H]⁺ |
| 398 | 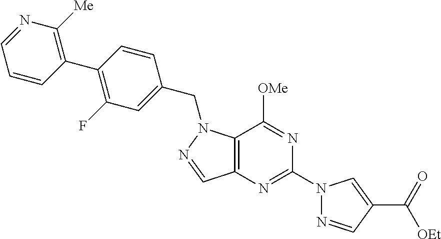 [SMILES: CCOC(=O)c1cnn(-c2nc(OC)c3c(cnn3Cc3ccc(-c4cccnc4C)c(F)c3)n2)c1] | powder MS (APCI) m/z: 488 [M + H]⁺ |
| 399 | 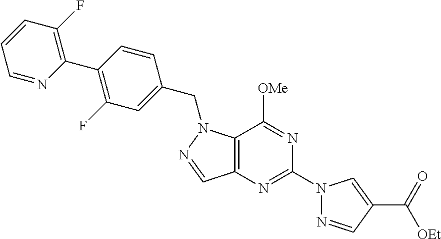 [SMILES: CCOC(=O)c1cnn(-c2nc(OC)c3c(cnn3Cc3ccc(-c4ncccc4F)c(F)c3)n2)c1] | powder MS (ESI) m/z: 492 [M + H]⁺ |
| 400 | 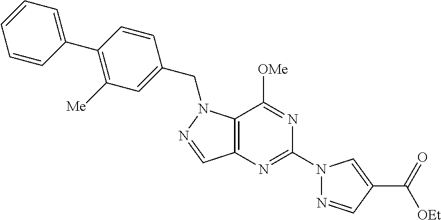 [SMILES: CCOC(=O)c1cnn(-c2nc(OC)c3c(cnn3Cc3ccc(-c4ccccc4)c(C)c3)n2)c1] | powder MS (APCI) m/z: 469 [M + H]⁺ |
| 401 | 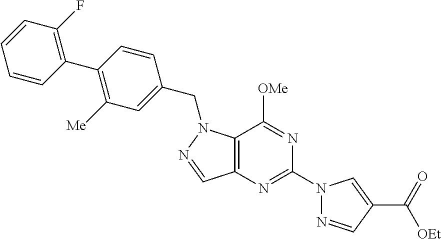 [SMILES: CCOC(=O)c1cnn(-c2nc(OC)c3c(cnn3Cc3ccc(-c4ccccc4F)c(C)c3)n2)c1] | powder MS (APCI) m/z: 487 [M + H]⁺ |
| 402 | 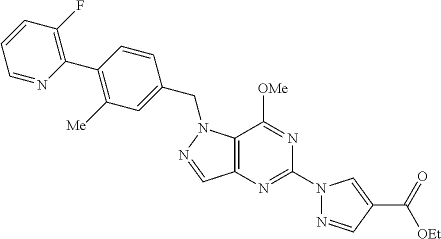 [SMILES: CCOC(=O)c1cnn(-c2nc(OC)c3c(cnn3Cc3ccc(-c4ncccc4F)c(C)c3)n2)c1] | powder MS (ESI) m/z: 488 [M + H]⁺ |
| 403 | 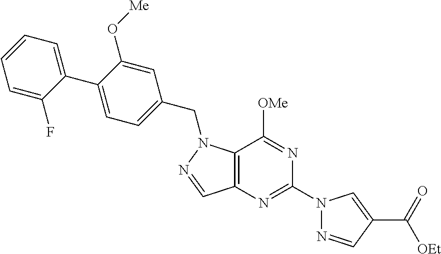 [SMILES: CCOC(=O)c1cnn(-c2nc(OC)c3c(cnn3Cc3ccc(-c4ccccc4F)c(OC)c3)n2)c1] | powder MS (APCI) m/z: 503 [M + H]⁺ |
| 404 | 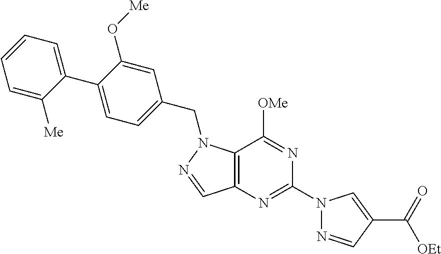 [SMILES: CCOC(=O)c1cnn(-c2nc(OC)c3c(cnn3Cc3ccc(-c4ccccc4C)c(OC)c3)n2)c1] | powder MS (APCI) m/z: 499 [M + H]⁺ |
| 405 | 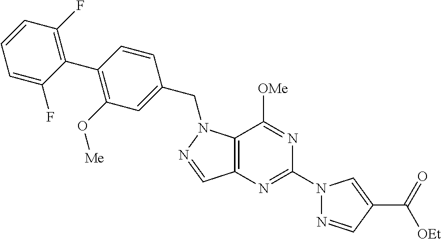 [SMILES: CCOC(=O)c1cnn(-c2nc(OC)c3c(cnn3Cc3ccc(-c4c(F)cccc4F)c(OC)c3)n2)c1] | powder MS (ESI) m/z: 521 [M + H]⁺ |
| 406 | 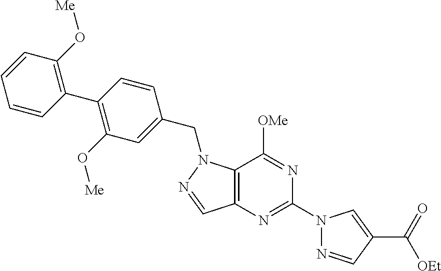 [SMILES: CCOC(=O)c1cnn(-c2nc(OC)c3c(cnn3Cc3ccc(-c4ccccc4OC)c(OC)c3)n2)c1] | powder MS (ESI) m/z: 515 [M + H]⁺ |
| 407 | 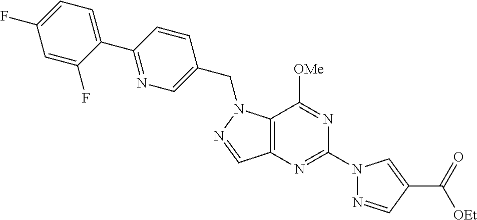 [SMILES: CCOC(=O)c1cnn(-c2nc(OC)c3c(cnn3Cc3ccc(-c4ccc(F)cc4F)nc3)n2)c1] | powder MS (APCI) m/z: 492 [M + H]⁺ |
| 408 | 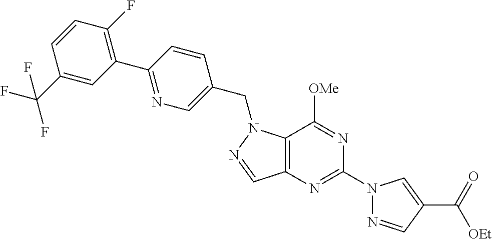 [SMILES: CCOC(=O)c1cnn(-c2nc(OC)c3c(cnn3Cc3ccc(-c4cc(C(F)(F)F)ccc4F)nc3)n2)c1] | powder MS (APCI) m/z: 542 [M + H]⁺ |
| 409 | 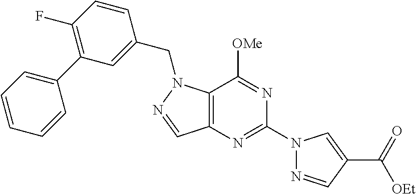 [SMILES: CCOC(=O)c1cnn(-c2nc(OC)c3c(cnn3Cc3ccc(F)c(-c4ccccc4)c3)n2)c1] | powder MS (ESI) m/z: 473 [M + H]⁺ |
| 410 | 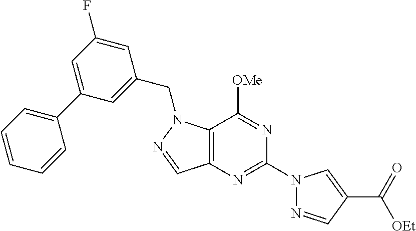 [SMILES: CCOC(=O)c1cnn(-c2nc(OC)c3c(cnn3Cc3cc(F)cc(-c4ccccc4)c3)n2)c1] | powder MS (ESI) m/z: 473 [M + H]⁺ |
| 411 | 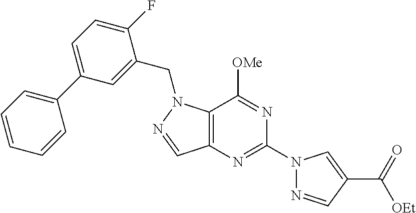 [SMILES: CCOC(=O)c1cnn(-c2nc(OC)c3c(cnn3Cc3cc(-c4ccccc4)ccc3F)n2)c1] | powder MS (ESI) m/z: 473 [M + H]⁺ |
| 412 | 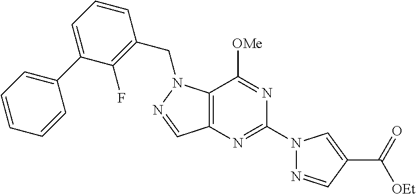 [SMILES: CCOC(=O)c1cnn(-c2nc(OC)c3c(cnn3Cc3cccc(-c4ccccc4)c3F)n2)c1] | powder MS (ESI) m/z: 473 [M + H]⁺ |
| 413 | 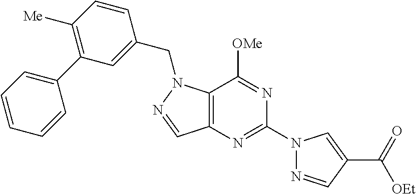 [SMILES: CCOC(=O)c1cnn(-c2nc(OC)c3c(cnn3Cc3ccc(C)c(-c4ccccc4)c3)n2)c1] | powder MS (ESI) m/z: 469 [M + H]⁺ |
| 414 | 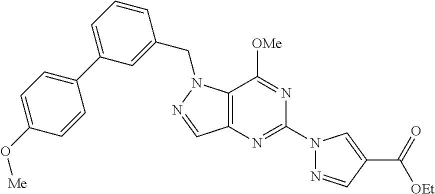 [SMILES: CCOC(=O)c1cnn(-c2nc(OC)c3c(cnn3Cc3cccc(-c4ccc(OC)cc4)c3)n2)c1] | powder MS (ESI) m/z: 485 [M + H]⁺ |
| 415 | 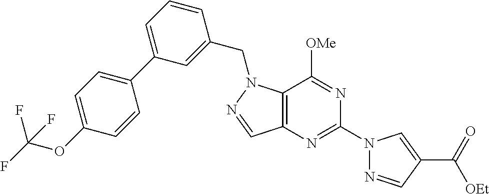 [SMILES: CCOC(=O)c1cnn(-c2nc(OC)c3c(cnn3Cc3cccc(-c4ccc(OC(F)(F)F)cc4)c3)n2)c1] | powder MS (ESI) m/z: 539 [M + H]⁺ |
| 416 | 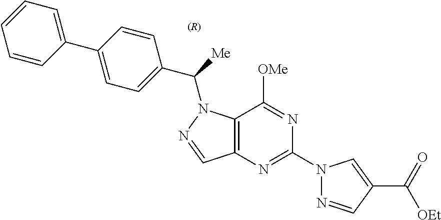 | viscous material MS (ESI) m/z: 469 [M + H]⁺ |
| 417 | 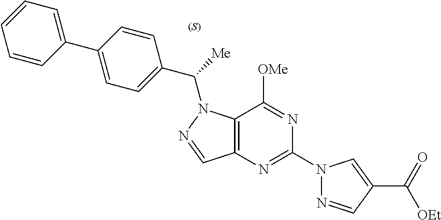 | viscous material MS (ESI) m/z: 469 [M + H]⁺ |
| 418 | 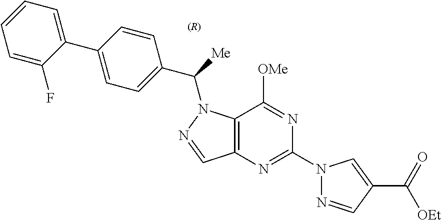 | viscous material MS (APCI) m/z: 487 [M + H]⁺ |
| 419 | 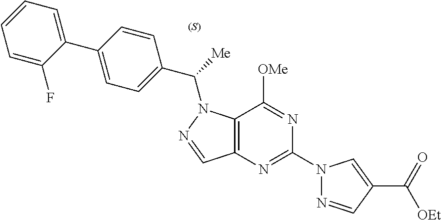 | powder MS (APCI) m/z: 487 [M + H]⁺ |
| 420 | 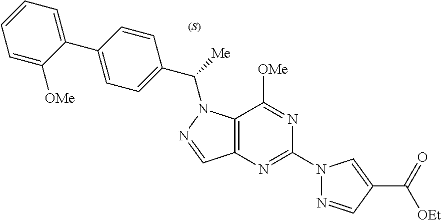 | powder MS (APCI) m/z: 499 [M + H]⁺ |
| 421 | 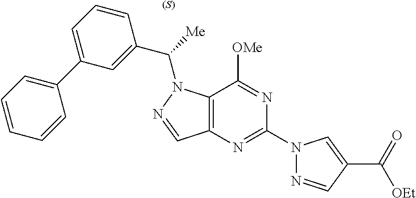 | viscous material MS (APCI) m/z: 469 [M + H]⁺ |

### REFERENCE EXAMPLE 422

### Preparation of ethyl 1-[1-(3-hydroxypropyl)-7-methoxy-1H-pyrazolo[4,3-d]pyrimidin-5-yl]-1H-pyrazole-4-carboxylate

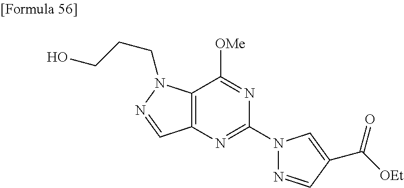 [SMILES: CCOC(=O)c1cnn(-c2nc(OC)c3c(cnn3CCCO)n2)c1]

(1) To a suspension of ethyl 1-(7-methoxy-1H-pyrazolo[4,3-d]pyrimidin-5-yl)-1H-pyrazole-4-carboxylate prepared in Reference Example 1 or 2 (1.01 g), 3-(t-butyldimethylsiloxy)propanol (1.12 mL) and triphenylphosphine (1.84 g) in tetrahydrofuran (10 mL) was added diisopropyl azodicarboxylate (3.69 mL, 1.9 mol/L in toluene), and the reaction mixture was stirred overnight at room temperature. The reaction mixture was concentrated under reduced pressure, and the residue was purified by silica gel column chromatography (solvent: hexane/ethyl acetate=95/5 to 50/50) to yield a crude ethyl 1-[1-(3-{[t-butyl(dimethyl)silyl]oxy}propyl)-7-methoxy-1H-pyrazolo[4,3-d]pyrimidin-5-yl]-1H-pyrazole-4-carboxylate (1.98 g) as a colorless powder.

MS (ESI) m/z: 461 [M+H]⁺

(2) To a solution of the crude product obtained in (1) (1.97 g) in chloroform (5 mL) was added hydrogen chloride (10 mL, 4 mol/L in 1,4-dioxane), and the reaction mixture was stirred for 30 minutes at room temperature. The reaction mixture was concentrated under reduced pressure, and the residue was added with saturated sodium bicarbonate aqueous solution, followed by extracting three times the mixture with chloroform. The organic layer was concentrated under reduced pressure, and the residue was purified by silica gel column chromatography (solvent: chloroform/methanol=100/0 to 95/5) to yield the titled compound (716 mg, 59% yield in two steps) as a colorless solid.

MS (ESI) m/z: 347 [M+H]⁺.

### REFERENCE EXAMPLE 423

### Preparation of ethyl 1-{1-[3-(biphenyl-4-yloxy)propyl]-7-methoxy-1H-pyrazolo[4,3-d]pyrimidin-5-yl}-1H-pyrazole-4-carboxylate

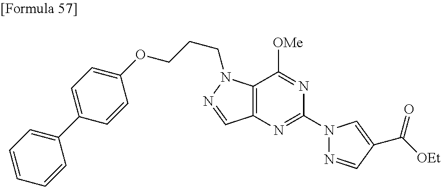 [SMILES: CCOC(=O)c1cnn(-c2nc(OC)c3c(cnn3CCCOc3ccc(-c4ccccc4)cc3)n2)c1]

To a suspension of ethyl 1-[1-(3-hydroxypropyl)-7-methoxy-1H-pyrazolo[4,3-d]pyrimidin-5-yl]-1H-pyrazole-4-carboxylate prepared in Reference Example 422 (70 mg), 4-phenylphenol (52 mg) and triphenylphosphine (106 mg) in tetrahydrofuran (2 mL) was added diisopropyl azodicarboxylate (213 μL, 1.9 mol/L in toluene), and the reaction mixture was stirred at room temperature for 1.5 hours. The reaction mixture was concentrated under reduced pressure, and the residue was purified by silica gel column chromatography (solvent: hexane/ethyl acetate=90/10 to 50/50) to yield the titled compound (101 mg, 100% yield) as a colorless powder.

MS (ESI) m/z: 499 [M+H]⁺.

### REFERENCE EXAMPLE 424 to 431

The compounds listed in the following Table 5 were obtained from the corresponding starting material in the same manner as described in Reference Examples 423.

TABLE 5

| Reference | | material |
|---|---|---|
| Example | Structure | properties |
| 424 | 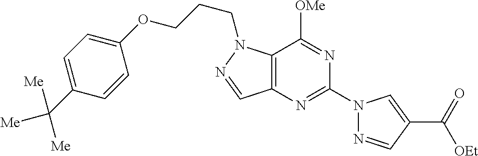 [SMILES: CCOC(=O)c1cnn(-c2nc(OC)c3c(cnn3CCCOc3ccc(C(C)(C)C)cc3)n2)c1] | powder MS (ESI) m/z: 479 [M + H]⁺ |
| 425 | 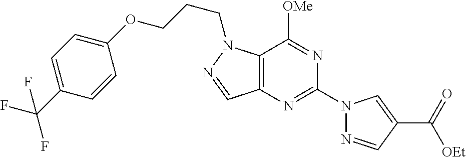 [SMILES: CCOC(=O)c1cnn(-c2nc(OC)c3c(cnn3CCCOc3ccc(C(F)(F)F)cc3)n2)c1] | powder MS (ESI) m/z: 491 [M + H]⁺ |
| 426 | 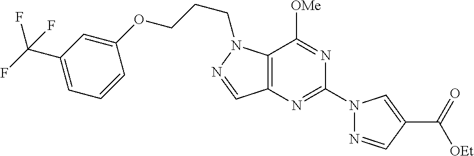 [SMILES: CCOC(=O)c1cnn(-c2nc(OC)c3c(cnn3CCCOc3cccc(C(F)(F)F)c3)n2)c1] | powder MS (ESI) m/z: 491 [M + H]⁺ |
| 427 | 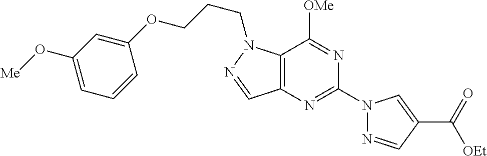 [SMILES: CCOC(=O)c1cnn(-c2nc(OC)c3c(cnn3CCCOc3cccc(OC)c3)n2)c1] | powder MS (APCI) m/z: 453 [M + H]⁺ |
| 428 | 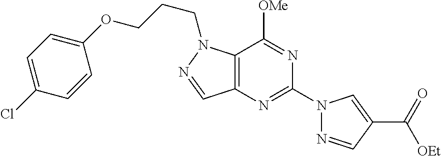 [SMILES: CCOC(=O)c1cnn(-c2nc(OC)c3c(cnn3CCCOc3ccc(Cl)cc3)n2)c1] | powder MS (APCI) m/z: 457/459 [M + H]⁺ |
| 429 | 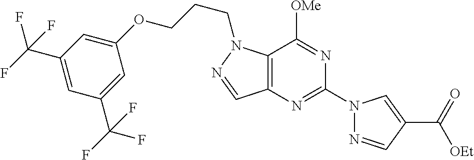 [SMILES: CCOC(=O)c1cnn(-c2nc(OC)c3c(cnn3CCCOc3cc(C(F)(F)F)cc(C(F)(F)F)c3)n2)c1] | powder MS (APCI) m/z: 559 [M + H]⁺ |
| 430 | 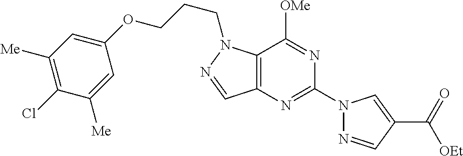 [SMILES: CCOC(=O)c1cnn(-c2nc(OC)c3c(cnn3CCCOc3cc(C)c(Cl)c(C)c3)n2)c1] | powder MS (APCI) m/z: 485/487 [M + H]⁺ |
| 431 | 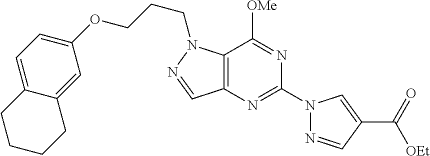 [SMILES: CCOC(=O)c1cnn(-c2nc(OC)c3c(cnn3CCCOc3ccc4c(c3)CCCC4)n2)c1] | powder MS (ESI) m/z: 477 [M + H]⁺ |

### REFERENCE EXAMPLE 432

### Preparation of ethyl 1-{1-[3-(4-cyclohexylphenoxyl)propyl]-7-methoxy-1H-pyrazolo[4,3-d]pyrimidin-5-yl}-1H-pyrazole-4-carboxylate

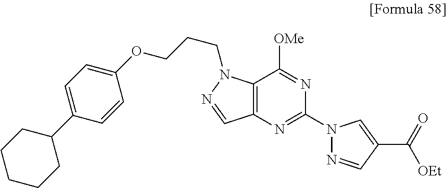 [SMILES: CCOC(=O)c1cnn(-c2nc(OC)c3c(cnn3CCCOc3ccc(C4CCCCC4)cc3)n2)c1]

To a suspension of ethyl 1-[1-(3-hydroxypropyl)-7-methoxy-1H-pyrazolo[4,3-d]pyrimidin-5-yl]-1H-pyrazole-4-carboxylate prepared in Reference Example 422 (50 mg), 4-cyclohexylphenol (38 mg) and triphenylphosphine (76 mg) in tetrahydrofuran (2 mL) was added 1,1′-azobis(N,N-dimethylformamide) (50 mg), and the reaction mixture was stirred for 2 hours at 60° C. The reaction mixture was concentrated under reduced pressure, and the residue was purified by silica gel column chromatography (solvent: hexane/ethyl acetate=80/20 to 60/40) to yield the titled compound (70 mg, 96% yield) as a colorless solid.

MS (APCI) m/z: 505 [M+H]⁺.

### REFERENCE EXAMPLES 433 TO 443

The compounds listed in the following Table 6 were obtained from the corresponding starting material in the same manner as described in Reference Example 432.

TABLE 6

| Reference | | material |
|---|---|---|
| Example | Structure | properties |
| 433 | 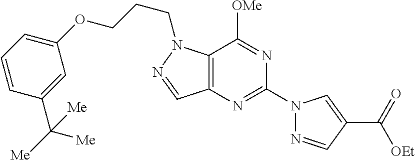 [SMILES: CCOC(=O)c1cnn(-c2nc(OC)c3c(cnn3CCCOc3cccc(C(C)(C)C)c3)n2)c1] | powder MS (APCI) m/z: 479 [M + H]⁺ |
| 434 | 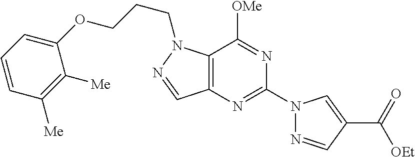 [SMILES: CCOC(=O)c1cnn(-c2nc(OC)c3c(cnn3CCCOc3cccc(C)c3C)n2)c1] | powder MS (APCI) m/z: 451 [M + H]⁺ |
| 435 | 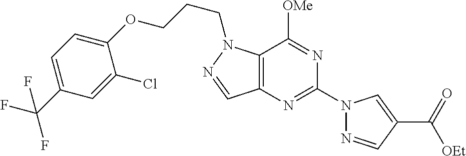 [SMILES: CCOC(=O)c1cnn(-c2nc(OC)c3c(cnn3CCCOc3ccc(C(F)(F)F)cc3Cl)n2)c1] | powder MS (APCI) m/z: 525/527 [M + H]⁺ |
| 436 | 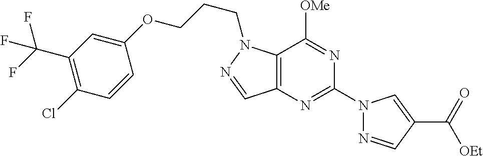 [SMILES: CCOC(=O)c1cnn(-c2nc(OC)c3c(cnn3CCCOc3ccc(Cl)c(C(F)(F)F)c3)n2)c1] | powder MS (APCI) m/z: 525/527 [M + H]⁺ |
| 437 | 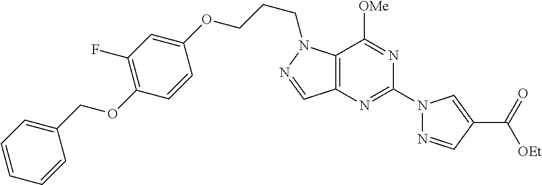 [SMILES: CCOC(=O)c1cnn(-c2nc(OC)c3c(cnn3CCCOc3ccc(OCc4ccccc4)c(F)c3)n2)c1] | powder MS (ESI) m/z: 547 [M + H]⁺ |
| 438 | 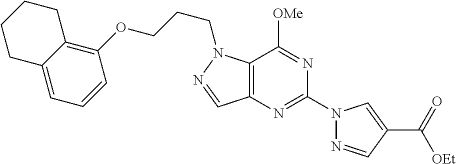 [SMILES: CCOC(=O)c1cnn(-c2nc(OC)c3c(cnn3CCCOc3cccc4c3CCCC4)n2)c1] | powder MS (APCI) m/z: 477 [M + H]⁺ |
| 439 | 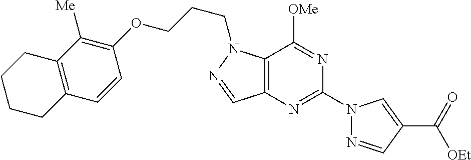 [SMILES: CCOC(=O)c1cnn(-c2nc(OC)c3c(cnn3CCCOc3ccc4c(c3C)CCCC4)n2)c1] | powder MS (APCI) m/z: 491 [M + H]⁺ |
| 440 | 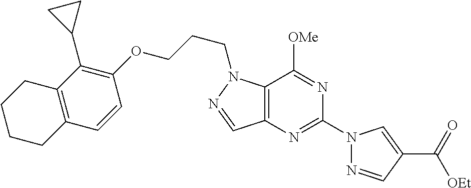 [SMILES: CCOC(=O)c1cnn(-c2nc(OC)c3c(cnn3CCCOc3ccc4c(c3C3CC3)CCCC4)n2)c1] | powder MS (APCI) m/z: 517 [M + H]⁺ |
| 441 | 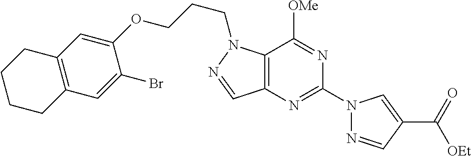 [SMILES: CCOC(=O)c1cnn(-c2nc(OC)c3c(cnn3CCCOc3cc4c(cc3Br)CCCC4)n2)c1] | powder MS (APCI) m/z: 555/557 [M + H]⁺ |
| 442 | 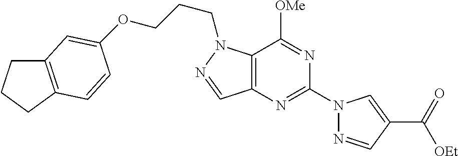 [SMILES: CCOC(=O)c1cnn(-c2nc(OC)c3c(cnn3CCCOc3ccc4c(c3)CCC4)n2)c1] | powder MS (APCI) m/z: 463 [M + H]⁺ |
| 443 | 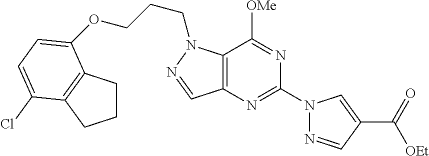 [SMILES: CCOC(=O)c1cnn(-c2nc(OC)c3c(cnn3CCCOc3ccc(Cl)c4c3CCC4)n2)c1] | powder MS (APCI) m/z: 497/499 [M + H]⁺ |

### REFERENCE EXAMPLE 444

### Preparation of ethyl 1-{1-[(cis-4-hydroxycyclohexyl)methyl]-7-methoxy-1H-pyrazolo[4,3-d]pyrimidin-5-yl}-1H-pyrazole-4-carboxylate

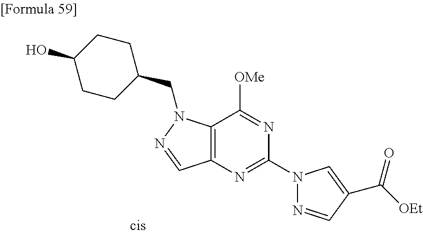 [SMILES: CCOC(=O)c1cnn(-c2nc(OC)c3c(cnn3C[C@H]3CC[C@@H](O)CC3)n2)c1]

(1) To a suspension of ethyl 1-(7-methoxy-1H-pyrazolo[4,3-d]pyrimidin-5-yl)-1H-pyrazole-4-carboxylate prepared in Reference Example 1 or 2 (1.04 g), (cis-4-{[t-butyl(dimethyl)silyl]oxy}cyclohexyl)methanol (1.32 g) and triphenylphosphine (1.89 g) in tetrahydrofuran (20 mL) was added diisopropyl azodicarboxylate (3.80 mL, 1.9 mol/L in toluene), and the reaction mixture was stirred for 1.5 hours at room temperature. The reaction mixture was concentrated under reduced pressure, and the residue was purified by silica gel column chromatography (solvent: hexane/ethyl acetate=90/10 to 70/30) to yield a crude ethyl 1-{1-[(cis-4-{[t-butyl(dimethyl)silyl]oxy}cyclohexyl)methyl]-7-methoxy-1H-pyrazolo[4,3-d]pyrimidin-5-yl}-1H-pyrazole-4-carboxylate (1.31 g) as a pale yellow powder.

MS (APCI) m/z: 515 [M+H]⁺

(2) To a solution of the crude product obtained in (1) (1.30 g) in chloroform (5 mL) was added hydrogen chloride (10 mL, 4 mol/L in 1,4-dioxane), and the reaction mixture was stirred for 30 minutes at room temperature. The reaction mixture was concentrated under reduced pressure, the residue was added with saturated sodium bicarbonate aqueous solution, followed by extracting the mixture three times with chloroform. The organic layer was concentrated under reduced pressure, and the residue was purified by silica gel column chromatography (solvent: chloroform/methanol=100/0 to 97/3) to yield the titled compound (815 mg, 56% yield in two steps) as a colorless solid.

MS (APCI) m/z: 401 [M+H]⁺.

### REFERENCE EXAMPLE 445 to 447

The compounds listed in the following Table 7 were obtained from the corresponding starting material in the same manner as described in Reference Example 444.

TABLE 7

| Reference | | material |
|---|---|---|
| Example | Structure | properties |
| 445 | 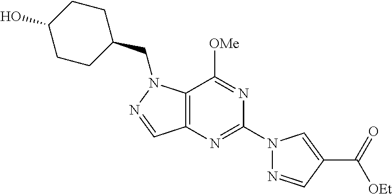 [SMILES: CCOC(=O)c1cnn(-c2nc(OC)c3c(cnn3C[C@H]3CC[C@H](O)CC3)n2)c1] | powder MS (APCI) m/z: 401 [M + H]⁺ |
| 446 | 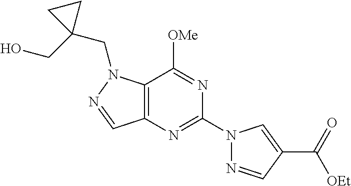 [SMILES: CCOC(=O)c1cnn(-c2nc(OC)c3c(cnn3CC3(CO)CC3)n2)c1] | powder MS (APCI) m/z: 373 [M + H]⁺ |
| 447 | 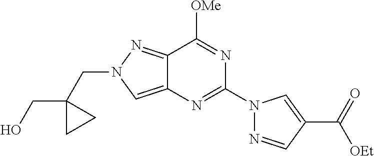 [SMILES: CCOC(=O)c1cnn(-c2nc(OC)c3nn(CC4(CO)CC4)cc3n2)c1] | powder MS (APCI) m/z: 373 [M + H]⁺ |

### REFERENCE EXAMPLE 448

### Preparation of ethyl 1-(7-methoxy-1-{[trans-4-(4-methylphenoxyl)cyclohexyl]methyl}-1H-pyrazolo[4,3-d]pyrimidin-5-yl)-1H-pyrazole-4-carboxylate

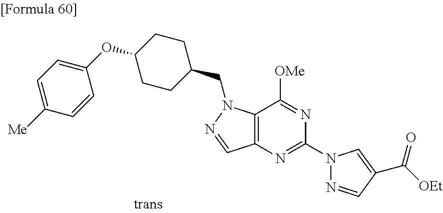 [SMILES: CCOC(=O)c1cnn(-c2nc(OC)c3c(cnn3C[C@H]3CC[C@H](Oc4ccc(C)cc4)CC3)n2)c1]

To a suspension of ethyl 1-{1-[(cis-4-hydroxycyclohexyl)methyl]-7-methoxy-1H-pyrazolo[4,3-d]pyrimidin-5-yl}-1H-pyrazole-4-carboxylate prepared in Reference Example 444 (103 mg), 4-methylphenol (42 mg) and triphenylphosphine (135 mg) in tetrahydrofuran (2 mL) was added diisopropyl azodicarboxylate (271 μL, 1.9 mol/L in toluene), and the reaction mixture was stirred for 1.7 hours at room temperature. The reaction mixture was concentrated under reduced pressure, and the residue was purified by silica gel column chromatography (solvent: hexane/ethyl acetate=90/10 to 50/50) to yield the titled compound (51 mg, 40% yield) as a colorless powder.

MS (APCI) m/z: 491 [M+H]⁺.

### REFERENCE EXAMPLES 449 TO 454

The compounds listed in the following Table 8 were obtained from the corresponding starting material in the same manner as described in Reference Example 448.

TABLE 8

| Reference | | material |
|---|---|---|
| Example | Structure | properties |
| 449 | 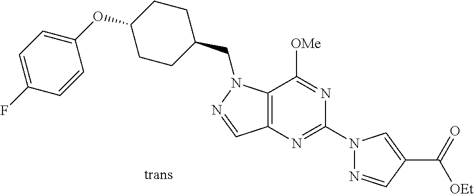 [SMILES: CCOC(=O)c1cnn(-c2nc(OC)c3c(cnn3C[C@H]3CC[C@H](Oc4ccc(F)cc4)CC3)n2)c1] | powder MS (ESI) m/z: 495 [M + H]⁺ |
| 450 | 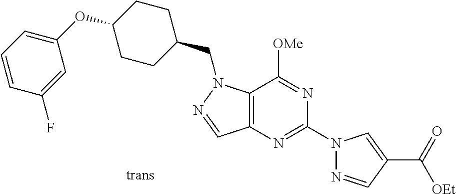 [SMILES: CCOC(=O)c1cnn(-c2nc(OC)c3c(cnn3C[C@H]3CC[C@H](Oc4cccc(F)c4)CC3)n2)c1] | powder MS (APCI) m/z: 495 [M + H]⁺ |
| 451 | 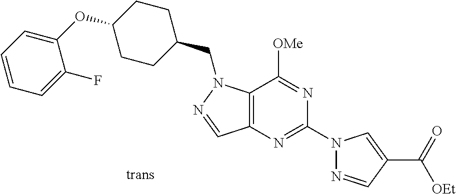 [SMILES: CCOC(=O)c1cnn(-c2nc(OC)c3c(cnn3C[C@H]3CC[C@H](Oc4ccccc4F)CC3)n2)c1] | powder MS (APCI) m/z: 495 [M + H]⁺ |
| 452 | 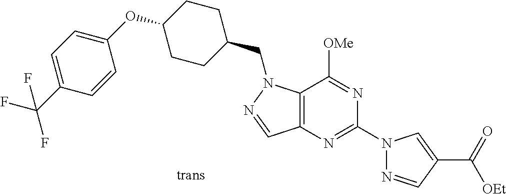 [SMILES: CCOC(=O)c1cnn(-c2nc(OC)c3c(cnn3C[C@H]3CC[C@H](Oc4ccc(C(F)(F)F)cc4)CC3)n2)c1] | powder MS (APCI) m/z: 545 [M + H]⁺ |
| 453 | 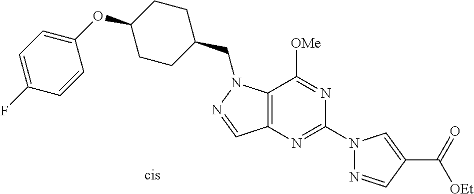 [SMILES: CCOC(=O)c1cnn(-c2nc(OC)c3c(cnn3C[C@H]3CC[C@@H](Oc4ccc(F)cc4)CC3)n2)c1] | powder MS (ESI) m/z: 495 [M + H]⁺ |
| 454 | 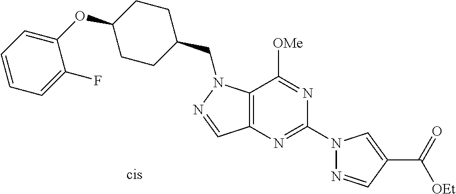 [SMILES: CCOC(=O)c1cnn(-c2nc(OC)c3c(cnn3C[C@H]3CC[C@@H](Oc4ccccc4F)CC3)n2)c1] | powder MS (APCI) m/z: 495 [M + H]⁺ |

### REFERENCE EXAMPLE 455

### Preparation of ethyl 1-[1-({1-[(4-chlorophenoxy)methyl]cyclopropyl}methyl)-7-methoxy-1H-pyrazolo[4,3-d]pyrimidin-5-yl]-1H-pyrazole-4-carboxylate

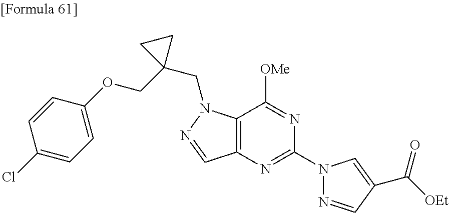 [SMILES: CCOC(=O)c1cnn(-c2nc(OC)c3c(cnn3CC3(COc4ccc(Cl)cc4)CC3)n2)c1]

Ethyl 1-(1-{[1-(hydroxymethyl)cyclopropyl]methyl}-7-methoxy-1H-pyrazolo[4,3-d]pyrimidin-5-yl)-1H-pyrazole-4-carboxylate prepared in Reference Example 446 was reacted with 4-chlorophenol in the same manner as described in Reference Example 432 to yield the titled compound as a powder.

MS (APCI) m/z: 483/485 [M+H]⁺.

### REFERENCE EXAMPLES 456, 457

The compounds listed in the following Table 9 were obtained from the corresponding starting material in the same manner as described in Reference Example 455.

TABLE 9

| Reference | | material |
|---|---|---|
| Example | Structure | properties |
| 456 | 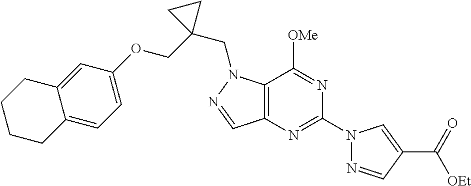 [SMILES: CCOC(=O)c1cnn(-c2nc(OC)c3c(cnn3CC3(COc4ccc5c(c4)CCCC5)CC3)n2)c1] | powder MS (APCI) m/z: 503 [M + H]⁺ |
| 457 | 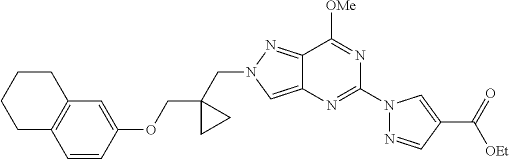 [SMILES: CCOC(=O)c1cnn(-c2nc(OC)c3nn(CC4(COc5ccc6c(c5)CCCC6)CC4)cc3n2)c1] | powder MS (APCI) m/z: 503 [M + H]⁺ |

### REFERENCE EXAMPLE 458

### Preparation of ethyl 1-(1-{[1-(4-fluorobenzyl)piperidin-4-yl]methyl}-7-methoxy-1H-pyrazolo[4,3-d]pyrimidin-5-yl)-1H-pyrazole-4-carboxylate

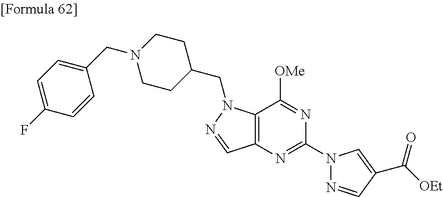 [SMILES: CCOC(=O)c1cnn(-c2nc(OC)c3c(cnn3CC3CCN(Cc4ccc(F)cc4)CC3)n2)c1]

(1) To a suspension of t-butyl 4-({5-[4-(ethoxycarbonyl)-1H-pyrazol-1-yl]-7-methoxy-1H-pyrazolo[4,3-d]pyrimidin-1-yl}methyl)piperidine-1-carboxylate prepared in Reference Example 47 (892 mg) in tetrahydrofuran (10 mL) and 1,4-dioxane (20 mL), hydrogen chloride (13.5 mL, 4 mol/L in ethyl acetate), and the reaction mixture was stirred at room temperature for 25 hours. Ethyl acetate (30 mL) was added to the reaction mixture, and the resulting solid was collected by filtration and washed with ethyl acetate, followed dryness under reduced pressure to yield ethyl 1-[7-methoxy-1-(piperidin-4-ylmethyl)-1H-pyrazolo[4,3-d]pyrimidin-5-yl]-1H-pyrazole-4-carboxylate hydrochloride (778 mg, 100% yield) as a colorless powder.

MS (APCI) m/z: 386 [M+H]⁺.

(2) To a suspension of the compound obtained in (1) (105 mg) and 4-fluorobenzaldehyde (46.6 mg) in dichloromethane (2.5 mL) was added sodium triacetoxyborohydride (79.5 mg), and the reaction mixture was stirred for 3 hours at room temperature. Additional 4-fluorobenzaldehyde (93.2 mg) and sodium triacetoxyborohydride (318 mg) were added to the reaction mixture, and the reaction mixture was stirred at room temperature for 15.5 hours. After saturated aqueous sodium bicarbonate was added to the reaction mixture, the organic layer was separated, and the aqueous layer was extracted with chloroform. The organic layer was concentrated under reduced pressure, and the residue was purified by silica gel column chromatography (solvent: chloroform/methanol=98/2 to 92/8) to yield the titled compound (51.4 mg, 41.9% yield) as a pale yellow solid.

MS (APCI) m/z: 494 [M+H]⁺.

### REFERENCE EXAMPLE 459

### Preparation of ethyl 1-(3-fluoro-7-methoxy-1H-pyrazolo[4,3-d]pyrimidin-5-yl)-1H-pyrazole-4-carboxylate

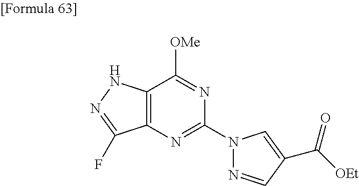 [SMILES: CCOC(=O)c1cnn(-c2nc(OC)c3[nH]nc(F)c3n2)c1]

To a suspension of ethyl 1-(7-methoxy-H-pyrazolo[4,3-d]pyrimidin-5-yl)-1H-pyrazole-4-carboxylate prepared in Reference Example 1 or 2 (1 g) in acetonitrile (20 mL) and acetic acid (4 mL) was added 1-chloromethyl-4-fluoro-1,4-diazoniabicyclo[2.2.2]octane bis(tetrafluoroborate) (3.7 g), and the reaction mixture was stirred for 48 hours under reflux. The reaction mixture was concentrated under reduced pressure, and the residue was purified by silica gel column chromatography (solvent: chloroform/methanol=98/2 to 97/3) to yield the titled compound (852 mg, 80% yield) as a pale yellow solid.

MS (APCI) m/z: 307 [M+H]⁺.

### REFERENCE EXAMPLE 460

### Preparation of ethyl 1-(3-chloro-7-methoxy-1H-pyrazolo[4,3-d]pyrimidin-5-yl)-1H-pyrazole-4-carboxylate

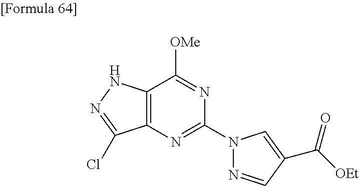 [SMILES: CCOC(=O)c1cnn(-c2nc(OC)c3[nH]nc(Cl)c3n2)c1]

Ethyl 1-(7-methoxy-1H-pyrazolo[4,3-d]pyrimidin-5-yl)-1H-pyrazole-4-carboxylate prepared in Reference Example 1 or 2 was reacted with N-chlorosuccinimide in the same manner as described in Reference Example 461 to yield the titled compound.

MS (APCI) m/z: 323/325 [M+H]⁺

### REFERENCE EXAMPLE 461

### Preparation of ethyl 1-(3-bromo-7-methoxy-1H-pyrazolo[4,3-d]pyrimidin-5-yl)-1H-pyrazole-4-carboxylate

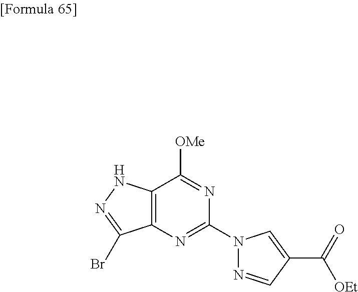 [SMILES: CCOC(=O)c1cnn(-c2nc(OC)c3[nH]nc(Br)c3n2)c1]

To a suspension of ethyl 1-(7-methoxy-1H-pyrazolo[4,3-d]pyrimidin-5-yl)-1H-pyrazole-4-carboxylate prepared in Reference Example 1 or 2 (2 g) in acetonitrile (40 mL) was added N-bromosuccinimide (3.7 g), and the reaction mixture was stirred for 3 hours under reflux. The resulting crystals were collected by filtration and then washed with acetonitrile to yield the titled compound (1.7 g, 67% yield) as a colorless solid.

MS (APCI) m/z: 367/369 [M+H]⁺.

### REFERENCE EXAMPLE 462

### Preparation of ethyl 1-[3-bromo-1-(cyclohexylmethyl)-7-methoxy-1H-pyrazolo[4,3-d]pyrimidin-5-yl]-1H-pyrazole-4-carboxylate

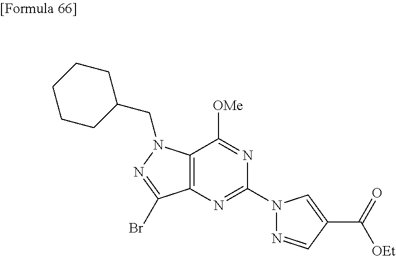 [SMILES: CCOC(=O)c1cnn(-c2nc(OC)c3c(n2)c(Br)nn3CC2CCCCC2)c1]

Ethyl 1-(3-bromo-7-methoxy-1H-pyrazolo[4,3-d]pyrimidin-5-yl)-1H-pyrazole-4-carboxylate prepared in Reference Example 461 was reacted in the same manner as Reference Examples 40, 41 to yield the titled compound was obtained.

MS (APCI) m/z: 463/465 [M+H]⁺.

### REFERENCE EXAMPLES 463 TO 519

The compounds listed in the following Table 10 were obtained from the corresponding starting material in the same manner as described in Reference Example 462.

TABLE 10

| Reference | | material |
|---|---|---|
| Example | Structure | properties |
| 463 | 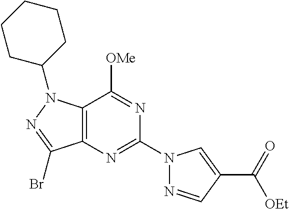 [SMILES: CCOC(=O)c1cnn(-c2nc(OC)c3c(n2)c(Br)nn3C2CCCCC2)c1] | powder MS (APCI) m/z: 449/451 [M + H]⁺ |
| 464 | 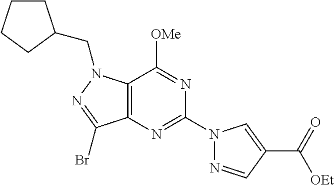 [SMILES: CCOC(=O)c1cnn(-c2nc(OC)c3c(n2)c(Br)nn3CC2CCCC2)c1] | powder MS (APCI) m/z: 449/451 [M + H]⁺ |
| 465 | 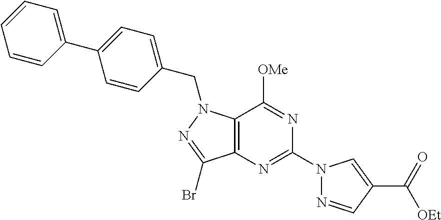 [SMILES: CCOC(=O)c1cnn(-c2nc(OC)c3c(n2)c(Br)nn3Cc2ccc(-c3ccccc3)cc2)c1] | powder MS (APCI) m/z: 533/535 [M + H]⁺ |
| 466 | 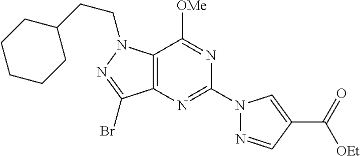 [SMILES: CCOC(=O)c1cnn(-c2nc(OC)c3c(n2)c(Br)nn3CCC2CCCCC2)c1] | powder MS (APCI) m/z: 477/479 [M + H]⁺ |
| 467 | 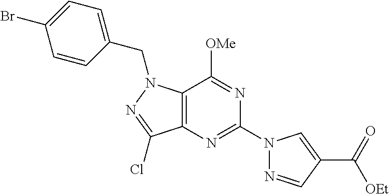 [SMILES: CCOC(=O)c1cnn(-c2nc(OC)c3c(n2)c(Cl)nn3Cc2ccc(Br)cc2)c1] | powder MS (APCI) m/z: 491/493 [M + H]⁺ |
| 468 | 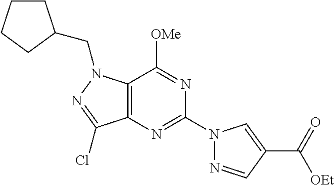 [SMILES: CCOC(=O)c1cnn(-c2nc(OC)c3c(n2)c(Cl)nn3CC2CCCC2)c1] | powder MS (APCI) m/z: 405/407 [M + H]⁺ |
| 469 | 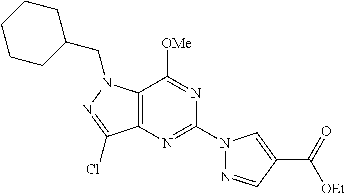 [SMILES: CCOC(=O)c1cnn(-c2nc(OC)c3c(n2)c(Cl)nn3CC2CCCCC2)c1] | powder MS (APCI) m/z: 419/421 [M + H]⁺ |
| 470 | 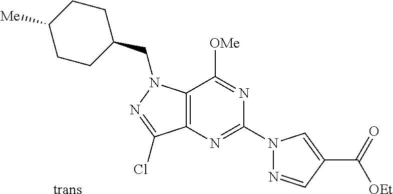 [SMILES: CCOC(=O)c1cnn(-c2nc(OC)c3c(n2)c(Cl)nn3C[C@H]2CC[C@H](C)CC2)c1 ; "trans"] | powder MS (APCI) m/z: 433/435 [M + H]⁺ |
| 471 | 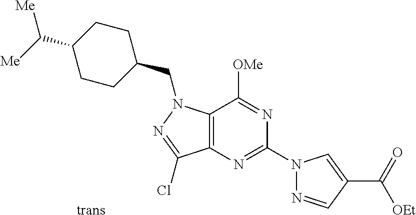 [SMILES: CCOC(=O)c1cnn(-c2nc(OC)c3c(n2)c(Cl)nn3C[C@H]2CC[C@H](C(C)C)CC2)c1 ; "trans"] | powder MS (APCI) m/z: 461/463 [M + H]⁺ |
| 472 | 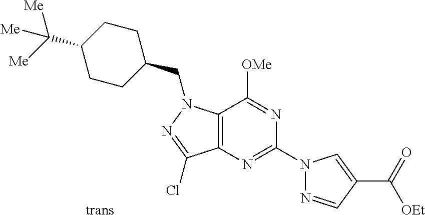 [SMILES: CCOC(=O)c1cnn(-c2nc(OC)c3c(n2)c(Cl)nn3C[C@H]2CC[C@H](C(C)(C)C)CC2)c1 ; "trans"] | powder MS (APCI) m/z: 475/477 [M + H]⁺ |
| 473 | 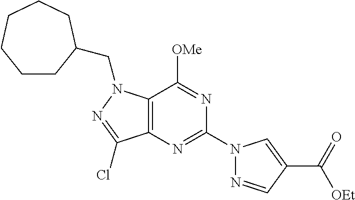 [SMILES: CCOC(=O)c1cnn(-c2nc(OC)c3c(n2)c(Cl)nn3CC2CCCCCC2)c1] | powder MS (APCI) m/z: 433/435 [M + H]⁺ |
| 474 | 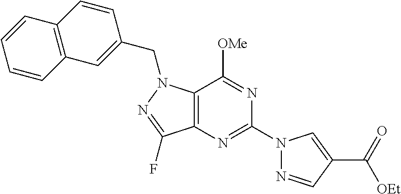 [SMILES: CCOC(=O)c1cnn(-c2nc(OC)c3c(n2)c(F)nn3Cc2ccc3ccccc3c2)c1] | powder MS (APCI) m/z: 447 [M + H]⁺ |
| 475 | 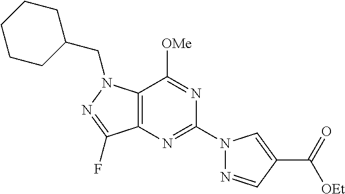 [SMILES: CCOC(=O)c1cnn(-c2nc(OC)c3c(n2)c(F)nn3CC2CCCCC2)c1] | powder MS (APCI) m/z: 403 [M + H]⁺ |
| 476 | 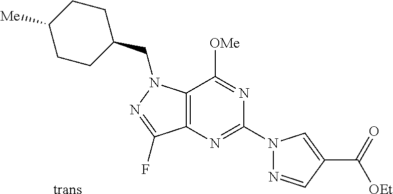 [SMILES: CCOC(=O)c1cnn(-c2nc(OC)c3c(n2)c(F)nn3C[C@H]2CC[C@H](C)CC2)c1 ; "trans"] | powder MS (APCI) m/z: 417 [M + H]⁺ |
| 477 | 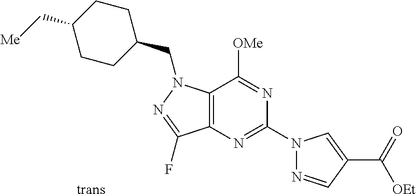 [SMILES: CCOC(=O)c1cnn(-c2nc(OC)c3c(n2)c(F)nn3C[C@H]2CC[C@H](CC)CC2)c1 ; "trans"] | powder MS (APCI) m/z: 431 [M + H]⁺ |
| 478 | 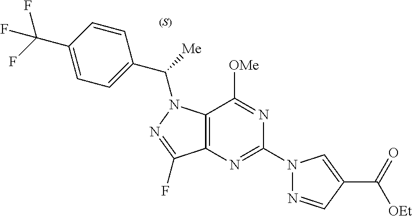 | powder MS (APCI) m/z: 479 [M + H]⁺ |
| 479 | 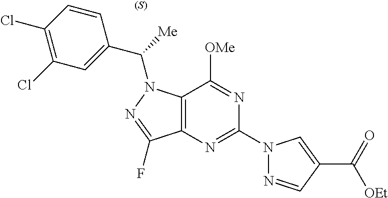 | powder MS (APCI) m/z: 479/481 [M + H]⁺ |
| 480 | 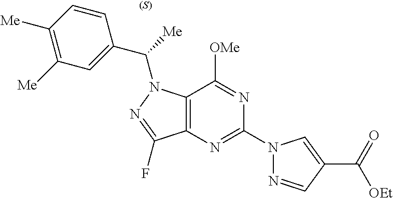 | viscous material MS (APCI) m/z: 439 [M + H]⁺ |
| 481 | 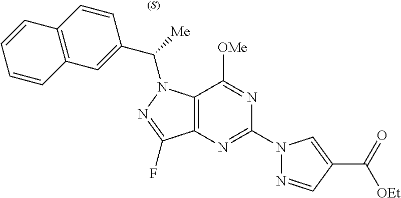 | powder MS (APCI) m/z: 461 [M + H]⁺ |
| 482 | 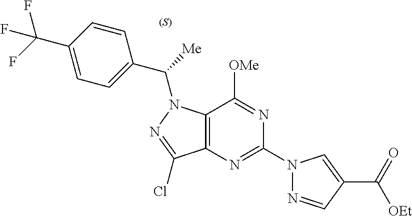 | powder MS (APCI) m/z: 495/497 [M + H]⁺ |
| 483 | 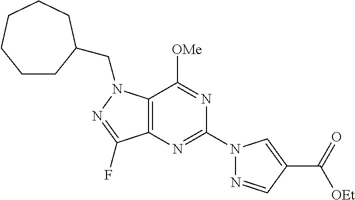 [SMILES: CCOC(=O)c1cnn(-c2nc(OC)c3c(n2)c(F)nn3CC2CCCCCC2)c1] | powder MS (APCI) m/z: 417 [M + H]⁺ |
| 484 | 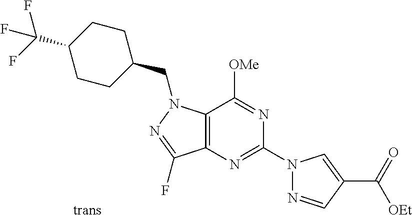 [SMILES: CCOC(=O)c1cnn(-c2nc(OC)c3c(n2)c(F)nn3C[C@H]2CC[C@H](C(F)(F)F)CC2)c1 ; "trans"] | powder MS (APCI) m/z: 471 [M + H]⁺ |
| 485 | 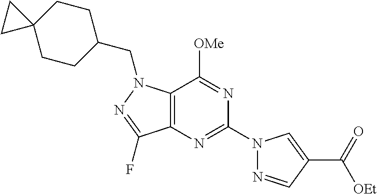 [SMILES: CCOC(=O)c1cnn(-c2nc(OC)c3c(n2)c(F)nn3CC2CCC3(CC2)CC3)c1] | powder MS (APCI) m/z: 429 [M + H]⁺ |
| 486 | 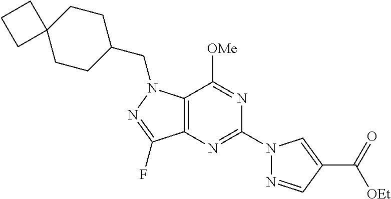 [SMILES: CCOC(=O)c1cnn(-c2nc(OC)c3c(n2)c(F)nn3CC2CCC3(CCC3)CC2)c1] | powder MS (APCI) m/z: 443 [M + H]⁺ |
| 487 | 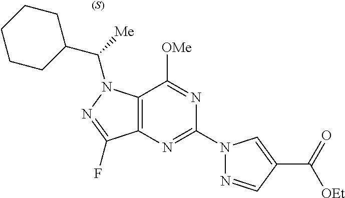 | powder MS (APCI) m/z: 417 [M + H]⁺ |
| 488 | 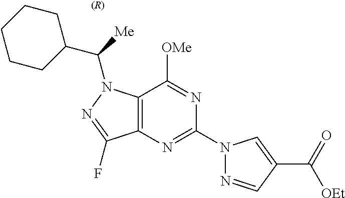 | powder MS (APCI) m/z: 417 [M + H]⁺ |
| 489 | 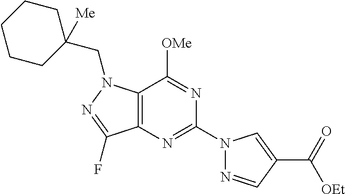 [SMILES: CCOC(=O)c1cnn(-c2nc(OC)c3c(n2)c(F)nn3CC2(C)CCCCC2)c1] | powder MS (APCI) m/z: 417 [M + H]⁺ |
| 490 | 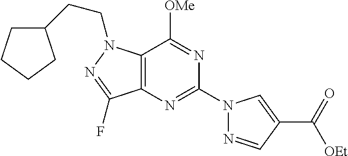 [SMILES: CCOC(=O)c1cnn(-c2nc(OC)c3c(n2)c(F)nn3CCC2CCCC2)c1] | powder MS (APCI) m/z: 403 [M + H]⁺ |
| 491 | 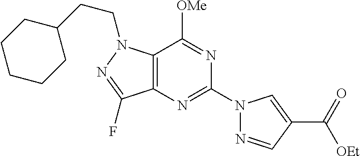 [SMILES: CCOC(=O)c1cnn(-c2nc(OC)c3c(n2)c(F)nn3CCC2CCCCC2)c1] | powder MS (APCI) m/z: 417 [M + H]⁺ |
| 492 | 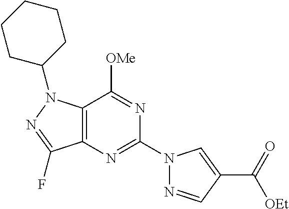 [SMILES: CCOC(=O)c1cnn(-c2nc(OC)c3c(n2)c(F)nn3C2CCCCC2)c1] | powder MS (APCI) m/z: 389 [M + H]⁺ |
| 493 | 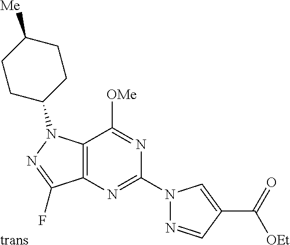 | powder MS (APCI) m/z: 403 [M + H]⁺ |
| 494 | 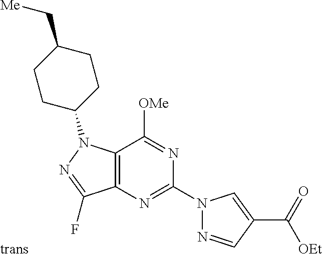 [SMILES: CCOC(=O)c1cnn(-c2nc(OC)c3c(n2)c(F)nn3[C@H]2CC[C@H](CC)CC2)c1 ; "trans"] | powder MS (APCI) m/z: 417 [M + H]⁺ |
| 495 | 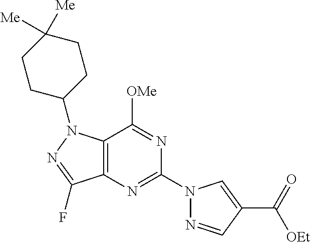 [SMILES: CCOC(=O)c1cnn(-c2nc(OC)c3c(n2)c(F)nn3C2CCC(C)(C)CC2)c1] | powder MS (APCI) m/z: 417 [M + H]⁺ |
| 496 | 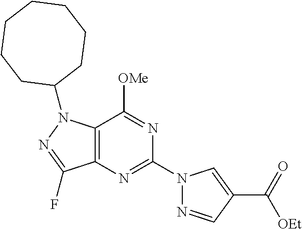 [SMILES: CCOC(=O)c1cnn(-c2nc(OC)c3c(n2)c(F)nn3C2CCCCCCC2)c1] | powder MS (APCI) m/z: 417 [M + H]⁺ |
| 497 | 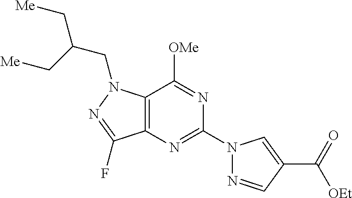 [SMILES: CCOC(=O)c1cnn(-c2nc(OC)c3c(n2)c(F)nn3CC(CC)CC)c1] | powder MS (APCI) m/z: 391 [M + H]⁺ |
| 498 | 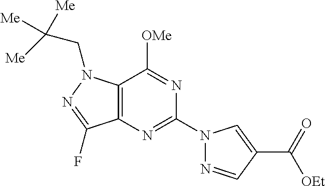 [SMILES: CCOC(=O)c1cnn(-c2nc(OC)c3c(n2)c(F)nn3CC(C)(C)C)c1] | powder MS (APCI) m/z: 377 [M + H]⁺ |
| 499 | 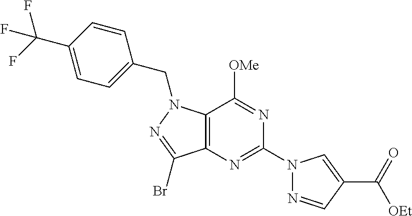 [SMILES: CCOC(=O)c1cnn(-c2nc(OC)c3c(n2)c(Br)nn3Cc2ccc(C(F)(F)F)cc2)c1] | powder MS (APCI) m/z: 525/527 [M + H]⁺ |
| 500 | 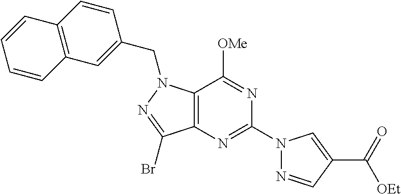 [SMILES: CCOC(=O)c1cnn(-c2nc(OC)c3c(n2)c(Br)nn3Cc2ccc3ccccc3c2)c1] | powder MS (APCI) m/z: 507/509 [M + H]⁺ |
| 501 | 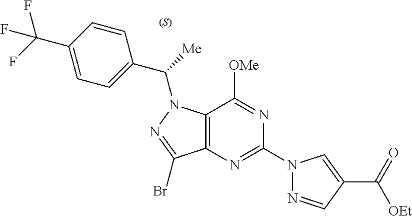 | viscous material MS (APCI) m/z: 539/541 [M + H]⁺ |
| 502 | 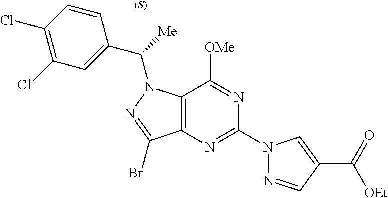 | viscous material MS (APCI) m/z: 539/541 [M + H]⁺ |
| 503 | 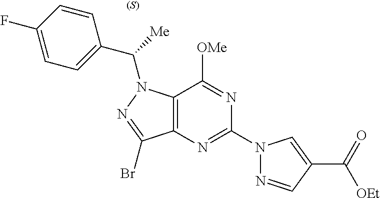 | powder MS (APCI) m/z: 489/491 [M + H]⁺ |
| 504 | 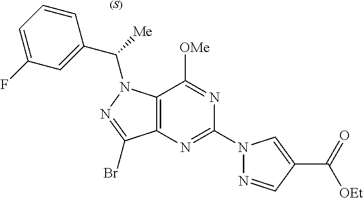 | powder MS (APCI) m/z: 489/491 [M + H]⁺ |
| 505 | 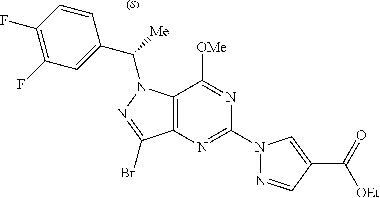 | powder MS (APCI) m/z: 507/509 [M + H]⁺ |
| 506 | 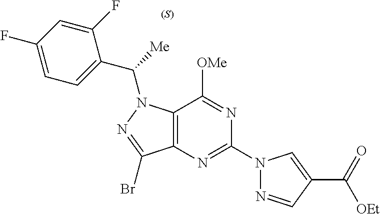 | powder MS (APCI) m/z: 507/509 [M + H]⁺ |
| 507 | 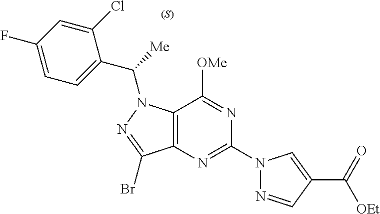 | powder MS (APCI) m/z: 523/525 [M + H]⁺ |
| 508 | 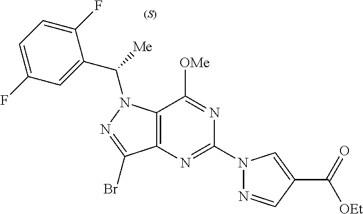 | powder MS (APCI) m/z: 507/509 [M + H]⁺ |
| 509 | 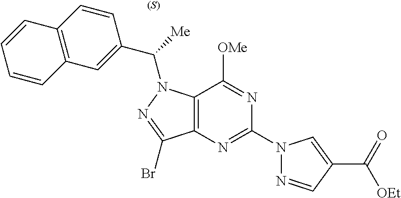 | powder MS (APCI) m/z: 521/523 [M + H]⁺ |
| 510 | 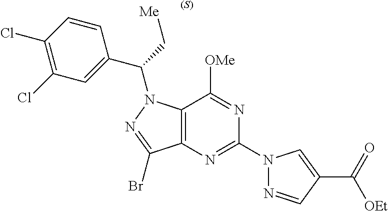 | powder MS (APCI) m/z: 553/555 [M + H]⁺ |
| 511 | 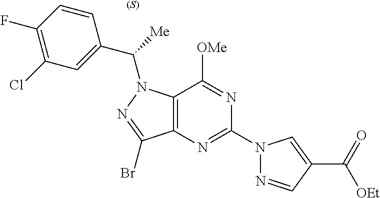 | powder MS (APCI) m/z: 523/525 [M + H]⁺ |
| 512 | 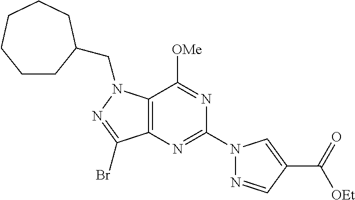 [SMILES: CCOC(=O)c1cnn(-c2nc(OC)c3c(n2)c(Br)nn3CC2CCCCCC2)c1] | powder MS (APCI) m/z: 477/479 [M + H]⁺ |
| 513 | 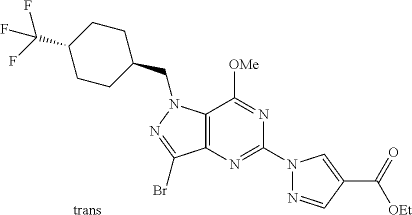 [SMILES: CCOC(=O)c1cnn(-c2nc(OC)c3c(n2)c(Br)nn3C[C@H]2CC[C@H](C(F)(F)F)CC2)c1 ; "trans"] | powder MS (APCI) m/z: 531/533 [M + H]⁺ |
| 514 | 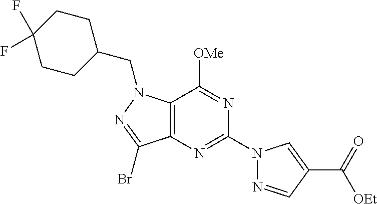 [SMILES: CCOC(=O)c1cnn(-c2nc(OC)c3c(n2)c(Br)nn3CC2CCC(F)(F)CC2)c1] | powder MS (APCI) m/z: 499/501 [M + H]⁺ |
| 515 | 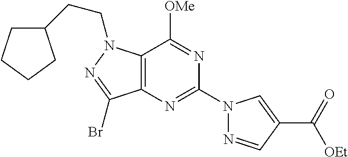 [SMILES: CCOC(=O)c1cnn(-c2nc(OC)c3c(n2)c(Br)nn3CCC2CCCC2)c1] | powder MS (APCI) m/z: 463/465 [M + H]⁺ |
| 516 | 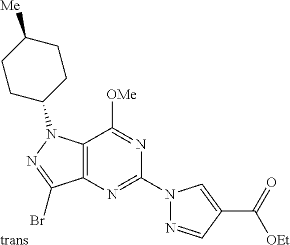 | powder MS (APCI) m/z: 463/465 [M + H]⁺ |
| 517 | 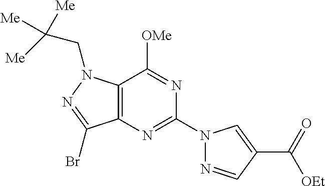 [SMILES: CCOC(=O)c1cnn(-c2nc(OC)c3c(n2)c(Br)nn3CC(C)(C)C)c1] | powder MS (APCI) m/z: 437/439 [M + H]⁺ |
| 518 | 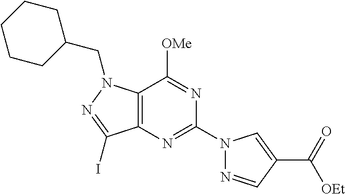 [SMILES: CCOC(=O)c1cnn(-c2nc(OC)c3c(n2)c(I)nn3CC2CCCCC2)c1] | powder MS (APCI) m/z: 511 [M + H]⁺ |
| 519 | 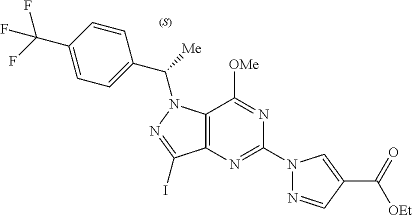 | powder MS (APCI) m/z: 587 [M + H]⁺ |

### REFERENCE EXAMPLE 520

### Preparation of ethyl 1-[1-(cyclohexylmethyl)-7-methoxy-3-methyl-1H-pyrazolo[4,3-d]pyrimidin-5-yl]-1H-pyrazole-4-carboxylate

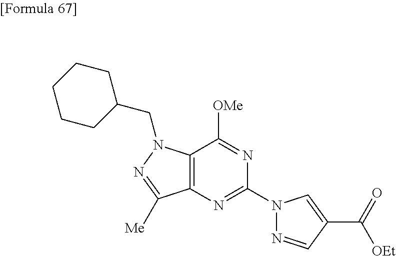 [SMILES: CCOC(=O)c1cnn(-c2nc(OC)c3c(n2)c(C)nn3CC2CCCCC2)c1]

To a suspension of ethyl 1-[3-bromo-1-(cyclohexylmethyl)-7-methoxy-1H-pyrazolo[4,3-d]pyrimidin-5-yl]-1H-pyrazole-4-carboxylate prepared in Reference Example 462 (76 mg), trimethylboroxine (46 μL), tripotassium phosphate (104 mg) in 1,4-dioxane (2 mL) was added bis(di-t-butyl(4-dimethylaminophenyl)phosphine)dichloropalladium(II) (12 mg), and the reaction mixture was stirred for 3 hours at 100° C. NH-silica gel (5 mL) and sodium sulfate (5 g) were added to the reaction mixture, and the insoluble materials were removed by filtration. The filtrate was concentrated under reduced pressure, and the residue was purified by silica gel column chromatography (solvent: hexane/ethyl acetate=85/15 to 80/20) to yield the titled compound (50 mg, 77% yield) as a colorless solid.

MS (APCI) m/z: 399 [M+H]⁺.

### REFERENCE EXAMPLES 521 TO 552

The compounds listed in the following Table 11 were obtained from the corresponding starting material in the same manner as described in Reference Example 520.

TABLE 11

| Reference | | material |
|---|---|---|
| Example | Structure | properties |
| 521 | 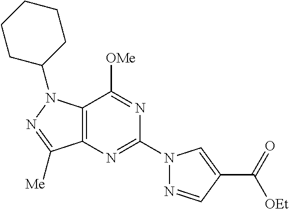 [SMILES: CCOC(=O)c1cnn(-c2nc(OC)c3c(n2)c(C)nn3C2CCCCC2)c1] | powder MS (APCI) m/z: 385 [M + H]⁺ |
| 522 | 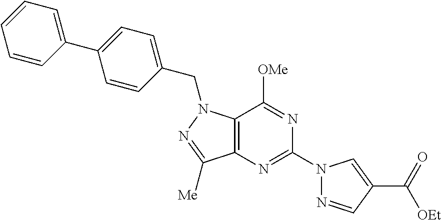 [SMILES: CCOC(=O)c1cnn(-c2nc(OC)c3c(n2)c(C)nn3Cc2ccc(-c3ccccc3)cc2)c1] | powder MS (APCI) m/z: 469 [M + H]⁺ |
| 523 | 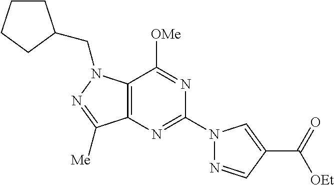 [SMILES: CCOC(=O)c1cnn(-c2nc(OC)c3c(n2)c(C)nn3CC2CCCC2)c1] | powder MS (APCI) m/z: 385 [M + H]⁺ |
| 524 | 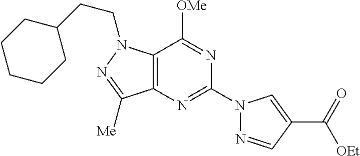 [SMILES: CCOC(=O)c1cnn(-c2nc(OC)c3c(n2)c(C)nn3CCC2CCCCC2)c1] | powder MS (APCI) m/z: 413 [M + H]⁺ |
| 525 | 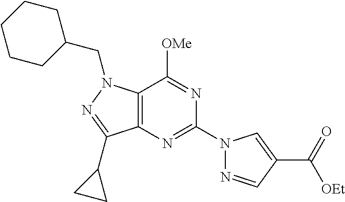 [SMILES: CCOC(=O)c1cnn(-c2nc(OC)c3c(n2)c(C2CC2)nn3CC2CCCCC2)c1] | powder MS (APCI) m/z: 425 [M + H]⁺ |
| 526 | 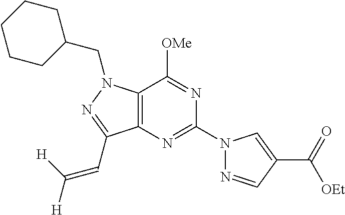 [SMILES: C=Cc1nn(CC2CCCCC2)c2c(OC)nc(-n3cc(C(=O)OCC)cn3)nc12] | powder MS (APCI) m/z: 411 [M + H]⁺ |
| 527 | 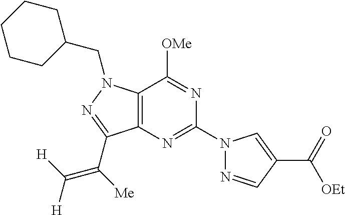 [SMILES: C=C(C)c1nn(CC2CCCCC2)c2c(OC)nc(-n3cc(C(=O)OCC)cn3)nc12] | powder MS (APCI) m/z: 425 [M + H]⁺ |
| 528 | 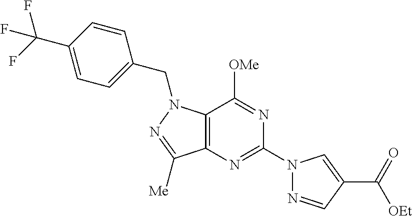 [SMILES: CCOC(=O)c1cnn(-c2nc(OC)c3c(n2)c(C)nn3Cc2ccc(C(F)(F)F)cc2)c1] | powder MS (APCI) m/z: 461 [M + H]⁺ |
| 529 | 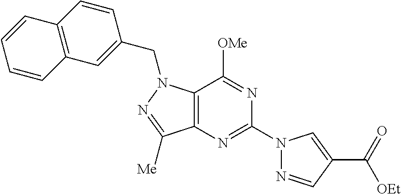 [SMILES: CCOC(=O)c1cnn(-c2nc(OC)c3c(n2)c(C)nn3Cc2ccc3ccccc3c2)c1] | powder MS (APCI) m/z: 443 [M + H]⁺ |
| 530 | 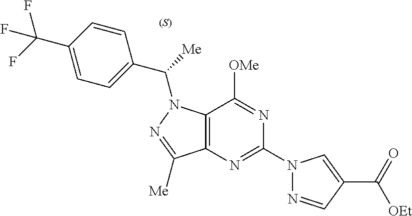 | viscous material MS (APCI) m/z: 475 [M + H]⁺ |
| 531 | 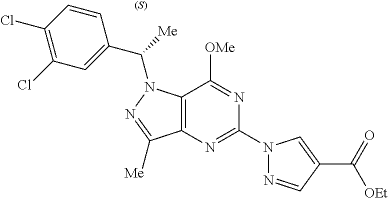 | viscous material MS (APCI) m/z: 475/477 [M + H]⁺ |
| 532 | 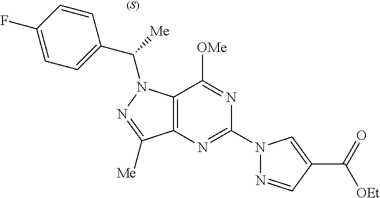 | viscous material MS (APCI) m/z: 425 [M + H]⁺ |
| 533 | 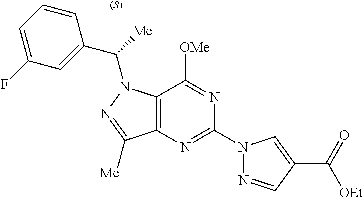 | powder MS (APCI) m/z: 425 [M + H]⁺ |
| 534 | 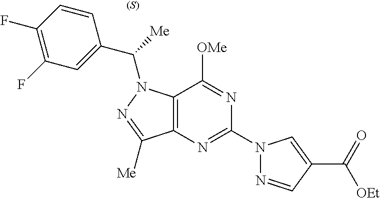 | powder MS (APCI) m/z: 443 [M + H]⁺ |
| 535 | 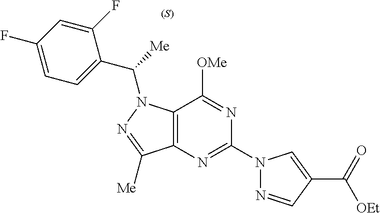 | viscous material MS (APCI) m/z: 443 [M + H]⁺ |
| 536 | 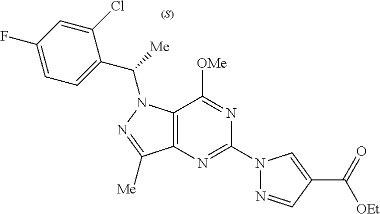 | powder MS (APCI) m/z: 459/461 [M + H]⁺ |
| 537 | 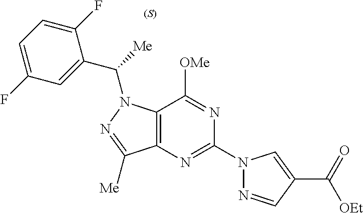 | powder MS (APCI) m/z: 443 [M + H]⁺ |
| 538 | 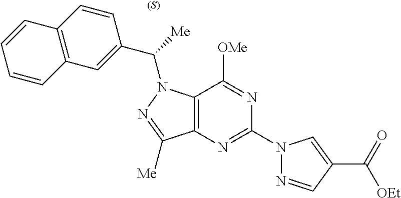 | powder MS (APCI) m/z: 457 [M + H]⁺ |
| 539 | 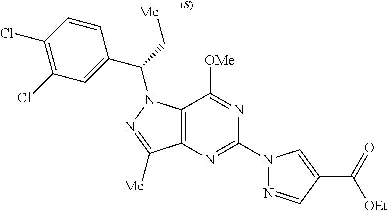 | viscous material MS (APCI) m/z: 489/491 [M + H]⁺ |
| 540 | 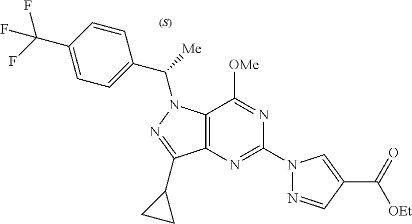 | powder MS (APCI) m/z: 501 [M + H]⁺ |
| 541 | 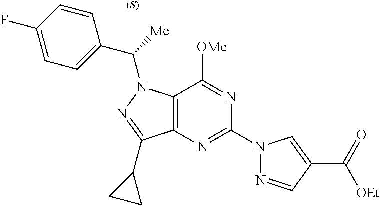 | powder MS (APCI) m/z: 451 [M + H]⁺ |
| 542 | 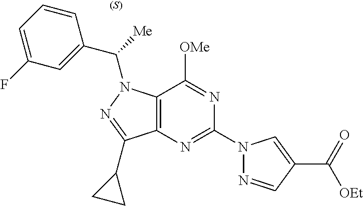 | powder MS (APCI) m/z: 451 [M + H]⁺ |
| 543 | 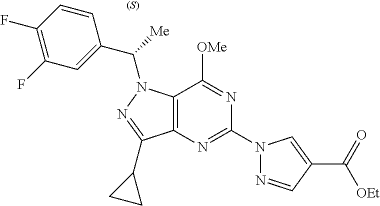 | powder MS (APCI) m/z: 469 [M + H]⁺ |
| 544 | 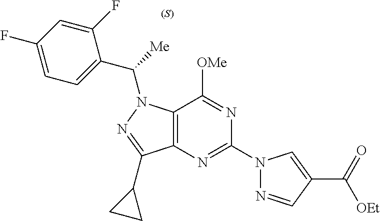 | viscous material MS (APCI) m/z: 469 [M + H]⁺ |
| 545 | 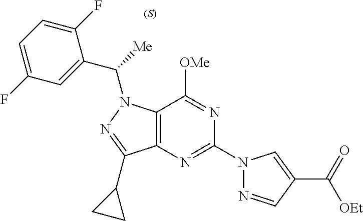 | powder MS (APCI) m/z: 469 [M + H]⁺ |
| 546 | 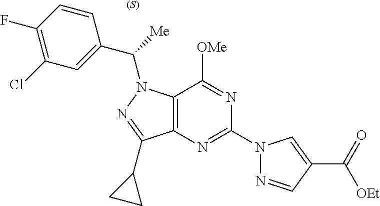 | powder MS (APCI) m/z: 485/487 [M + H]⁺ |
| 547 | 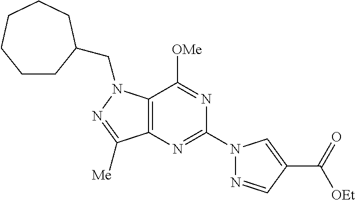 [SMILES: CCOC(=O)c1cnn(-c2nc(OC)c3c(n2)c(C)nn3CC2CCCCCC2)c1] | powder MS (APCI) m/z: 413 [M + H]⁺ |
| 548 | 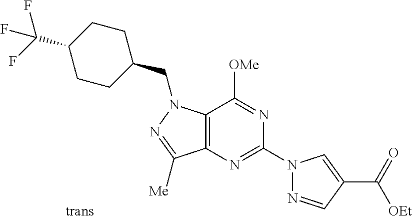 [SMILES: CCOC(=O)c1cnn(-c2nc(OC)c3c(n2)c(C)nn3C[C@H]2CC[C@H](C(F)(F)F)CC2)c1 ; "trans"] | powder MS (APCI) m/z: 467 [M + H]⁺ |
| 549 | 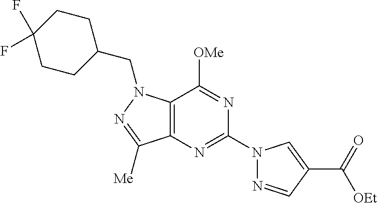 [SMILES: CCOC(=O)c1cnn(-c2nc(OC)c3c(n2)c(C)nn3CC2CCC(F)(F)CC2)c1] | powder MS (APCI) m/z: 435 [M + H]⁺ |
| 550 | 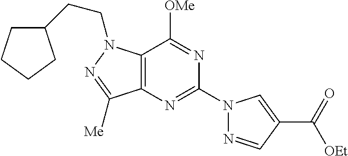 [SMILES: CCOC(=O)c1cnn(-c2nc(OC)c3c(n2)c(C)nn3CCC2CCCC2)c1] | powder MS (APCI) m/z: 399 [M + H]⁺ |
| 551 | 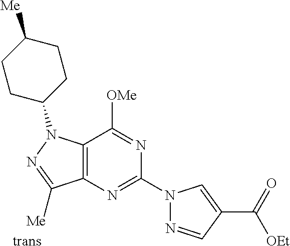 | powder MS (APCI) m/z: 399 [M + H]⁺ |
| 552 | 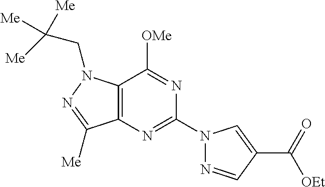 [SMILES: CCOC(=O)c1cnn(-c2nc(OC)c3c(n2)c(C)nn3CC(C)(C)C)c1] | powder MS (APCI) m/z: 373 [M + H]⁺ |

### REFERENCE EXAMPLE 553

### Preparation of ethyl 1-[1-(cyclohexylmethyl)-3-ethyl-7-methoxy-1H-pyrazolo[4,3-d]pyrimidin-5-yl]-1H-pyrazole-4-carboxylate

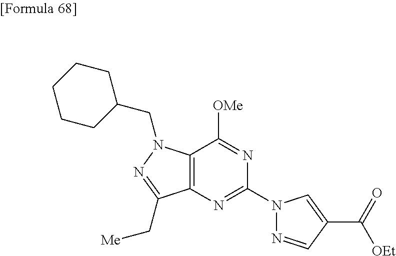 [SMILES: CCOC(=O)c1cnn(-c2nc(OC)c3c(n2)c(CC)nn3CC2CCCCC2)c1]

To a solution of ethyl 1-[1-(cyclohexylmethyl)-7-methoxy-3-vinyl-1H-pyrazolo[4,3-d]pyrimidin-5-yl]-1H-pyrazole-4-carboxylate prepared in Reference Example 526 (52 mg) in methanol (1 mL) was added 10% palladium on carbon (10 mg, 50% wet with water) under hydrogen atmosphere, and the reaction mixture was stirred at room temperature for 7 hours. The insoluble materials in the reaction mixture were removed by filtration through diatomaceous earth, and the filtrate was concentrated under reduced pressure. The residue was purified by silica gel column chromatography (solvent: hexane/ethyl acetate=70/30 to 60/40) to yield the titled compound (42 mg, 80% yield) as a colorless solid.

MS (APCI) m/z: 413 [M+H]⁺.

### REFERENCE EXAMPLE 554

### Preparation of ethyl 1-[1-(cyclohexylmethyl)-3-iso-propyl-7-methoxy-1H-pyrazolo[4,3-d]pyrimidin-5-yl]-1H-pyrazole-4-carboxylate

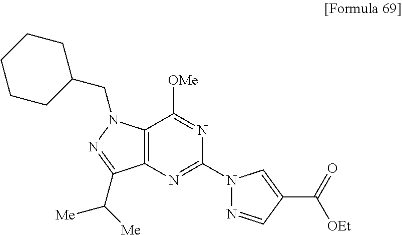 [SMILES: CCOC(=O)c1cnn(-c2nc(OC)c3c(n2)c(C(C)C)nn3CC2CCCCC2)c1]

Ethyl 1-[1-(cyclohexylmethyl)-3-isopropenyl-7-methoxy-1H-pyrazolo[4,3-d]pyrimidin-5-yl]-1H-pyrazole-4-carboxylate obtained in Reference Example 527 was reacted in the same manner as Reference Example 553 to yield the titled compound.

MS (APCI) m/z: 427 [M+H]⁺.

### REFERENCE EXAMPLE 555

### Preparation of ethyl 1-(7-methoxy-1-{3-[(3-methyl-5,6,7,8-tetrahydronaphthalene-2-yl)oxy]propyl}-1H-pyrazolo[4,3-d]pyrimidin-5-yl)-1H-pyrazole-4-carboxylate

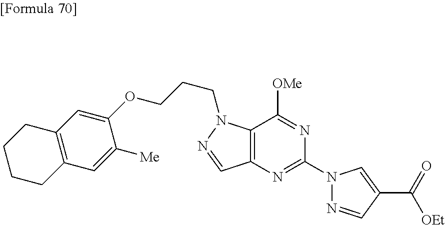 [SMILES: CCOC(=O)c1cnn(-c2nc(OC)c3c(cnn3CCCOc3cc4c(cc3C)CCCC4)n2)c1]

To a suspension of ethyl 1-(1-{3-[(3-bromo-5,6,7,8-tetrahydronaphthalene-2-yl)oxy]propyl}-7-methoxy-1H-pyrazolo[4,3-d]pyrimidin-5-yl)-1H-pyrazole-4-carboxylate prepared in Reference Example 441 (201 mg), 2,4,6-trimethylboroxine (91 mg) and cesium fluoride (550 mg) in 1,4-dioxane (4 mL) was added 1,1′-bis(diphenylphosphino)ferrocene-palladium (II) dichloride dichloromethane complex (44 mg), and the reaction mixture was stirred for 3 hours at 100° C. The reaction mixture was concentrated under reduced pressure, and the residue was purified by silica gel column chromatography (solvent: hexane/ethyl acetate=80/20 to 60/40) to yield the titled compound (170 mg, 96% yield) as a colorless solid.

MS (APCI) m/z: 491 [M+H]⁺.

### REFERENCE EXAMPLE 556

### Preparation of 2-(5,6-dimethyl-2-naphthyl)ethanol

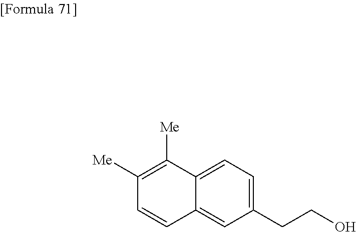 [SMILES: Cc1ccc2cc(CCO)ccc2c1C]

(1) To a solution of methyl(6-methyl-2-naphthyl)acetate (1 g) in dichloromethane (10 mL) was added tin chloride(IV) (1.5 g) under ice-cooling, and the reaction mixture was stirred for 10 min at 5° C. The reaction mixture was added dropwise with a solution of dichloromethyl methyl ether (654 mg) in dichloromethane (1 mL), and the reaction mixture was stirred for 30 minutes at the same temperature and for 1 hour at room temperature. The reaction mixture was poured into 10% hydrochloric acid, and the mixture was extracted with chloroform. The organic layer was washed with water and brine, dried over sodium sulfate, and concentrated under reduced pressure. The resulting residue was filtered through silica gel, and the silica gel was washed with hexane/ethyl acetate (80/20). The filtrate was concentrated under reduced pressure, then added with hexane and ethyl acetate, and filtered to correct the resulting precipitate to yield a crude methyl(5-formyl-6-methyl-2-naphthyl)acetate (791 mg) as a brown powder.

(2) To a solution of the crude product obtained in (1) (791 mg) in ethanol (7.6 mL) was added 10% aqueous sodium hydroxide (1.9 mL), and the reaction mixture was stirred for 1 hour at room temperature. The reaction mixture was concentrated under reduced pressure. The residue was added with water, and the mixture was washed with diethyl ether. The aqueous layer was acidified with concentrated hydrochloric acid, and then added with tetrahydrofuran and ethyl acetate. The resulting precipitates were collected by filtration, washed sequentially with water and diethyl ether, and dried under reduced pressure to yield a crude (5-formyl-6-methyl-2-naphthyl)acetic acid (762 mg).

(3) To a solution of the crude product obtained in (2) (762 mg) in methanol (6 mL) and tetrahydrofuran (6 mL) was added 10% palladium on carbon (50% wet with water) (150 mg), and the reaction mixture was stirred at room temperature for 1. 5 hours under hydrogen atmosphere. The insoluble materials were removed by filtration, and the filtrate was concentrated under reduced pressure. To the resulting residue was added hexane, and the precipitates were collected by filtration to yield a crude (5,6-dimethyl-2-naphthyl)acetic acid (513 mg) as a colorless powder.

(4) To a suspension of lithium aluminum hydride (182 mg) in tetrahydrofuran (3 mL) was added dropwise a solution of the crude product obtained in (3) (513 mg) in tetrahydrofuran (4 mL) over five minutes at room temperature, and the reaction mixture was stirred at room temperature for 1 hour. Under ice-cooling, sodium sulfate (0.6 g) and water (0.6 g) were added to the reaction mixture, and the mixture was stirred for 10 minutes. The insoluble materials were removed by filtration, and the filtrate was concentrated under reduced pressure. The resulting residue was purified by silica gel column chromatography (solvent: hexane/ethyl acetate=90/10 to 70/30) to yield the titled compound (429 mg, 44% yield in 4 steps) as a colorless powder.

¹H NMR (400 MHz, CHLOROFORM-d) δ ppm 7.99 (d, J=8.73 Hz, 1H), 7.63 (br s, 1H), 7.57 (d, J=8.22 Hz, 1H), 7.37 (dd, J=8.73, 2.06 Hz, 1H), 7.29 (d, J=8.22 Hz, 1H), 3.94 (td, J=6.68, 6.17 Hz, 2H), 3.02 (t, J=6.68 Hz, 2H), 2.59 (s, 3H), 2.48 (s, 3H), 1.40 (t, J=6.17 Hz, 1H).

### REFERENCE EXAMPLE 557

### Preparation of 2-(6-methyl-2-naphthyl)ethanol

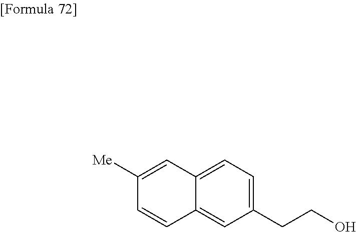 [SMILES: Cc1ccc2cc(CCO)ccc2c1]

(6-methyl-2-naphthyl)acetic acid was reacted in the same manner as Reference Example 556-(4) to yield the titled compound.

¹H NMR (400 MHz, CHLOROFORM-d) δ ppm 7.71 (d, J=8.70 Hz, 1H), 7.69 (d, J=8.70 Hz, 1H), 7.63 (s, 1H), 7.58 (s, 1H), 7.28-7.34 (m, 2H), 3.94 (td, J=6.66, 6.14 Hz, 2H), 3.01 (t, J=6.66 Hz, 2H), 2.50 (s, 3H), 1.41 (t, J=6.14 Hz, 1H).

### REFERENCE EXAMPLE 558

### Preparation of 2-(6-ethyl-2-naphthyl)ethanol

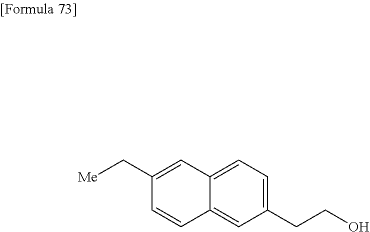 [SMILES: CCc1ccc2cc(CCO)ccc2c1]

(6-ethyl-2-naphthyl)acetic acid was reacted in the same manner as Reference Example 556-(4) to yield the titled compound.

¹H NMR (400 MHz, CHLOROFORM-d) δ ppm 7.74 (d, J=8.73, 1H), 7.72 (d, J=8.73, 1H), 7.64 (s, 1H), 7.60 (s, 1H), 7.30-7.36 (m, 2H), 3.94 (td, J=6.68, 6.17 Hz, 2H), 3.02 (t, J=6.68 Hz, 2H), 2.80 (q, J=7.71 Hz, 2H), 1.40 (t, J=6.17 Hz, 1H), 1.32 (t, J=7.71 Hz, 3H).

### REFERENCE EXAMPLE 559

### Preparation of 2-(trans-4-phenylcyclohexyl)ethanol

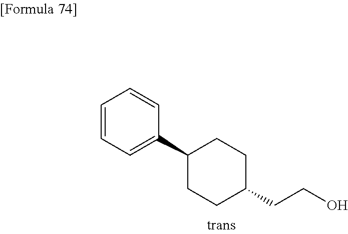 [SMILES: OCC[C@H]1CC[C@H](c2ccccc2)CC1]

(1) To a solution of methyl[trans-4-(4-{[(trifluoromethyl)sulfonyl]oxy}phenyl)cyclohexyl]acetate (700 mg) in methanol (10 mL) was added 10% palladium on carbon (50% wet with water) (424 mg), and the reaction mixture was stirred for 8 hours at room temperature under hydrogen atmosphere. The insoluble materials in the reaction mixture were filtered out using membrane filter, and the filtrate was concentrated under reduced pressure. The resultant residue was added with saturated aqueous sodium hydrogen carbonate, and the mixture was extracted 3 times with hexane. The combined organic layers were washed with brine, and the aqueous layer was extracted with ethyl acetate. The organic layers were combined, dried and concentrated under reduced pressure to yield methyl(trans-4-phenylcyclohexyl)acetate (395 mg, 92% yield) as a colorless viscous material.

¹H NMR (400 MHz, CHLOROFORM-d) δ ppm 7.12-7.39 (m, 5H), 3.68 (s, 3H), 2.41-2.52 (m, 1H), 2.26 (d, J=6.68 Hz, 2H), 1.79-1.99 (m, 5H), 1.42-1.59 (m, 2H), 1.07-1.24 (m, 2H).

(2) The compound obtained in (1) was reacted in the same manner as Reference Example 556-(4) to yield the titled compound.

¹H NMR (400 MHz, CHLOROFORM-d) δ ppm 7.13-7.35 (m, 5H), 3.67-3.80 (m, 2H), 2.42-2.53 (m, 1H), 1.82-2.01 (m, 4H), 1.42-1.59 (m, 5H), 1.05-1.31 (m, 3H).

### REFERENCE EXAMPLE 560

### Preparation of 4-bromo-2-fluoro-5-methylphenyl)methanol

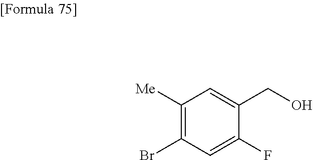 [SMILES: Cc1cc(CO)c(F)cc1Br]

To a solution of 4-bromo-2-fluoro-5-methylbenzaldehyde (3.26 g) in ethanol (35 mL) was added sodium borohydride (1.14 g) under ice-cooling, and the reaction mixture was stirred for 1 hour at room temperature. The reaction mixture was added with saturated aqueous sodium bicarbonate and stirred. The mixture was extracted with ethyl acetate, and the organic layer was dried over sodium sulfate and concentrated under reduced pressure. The residue was purified by silica gel column chromatography (solvent: hexane/ethyl acetate=90/10 to 80/20) to yield the titled compound (3.33 g, 100% yield) as a colorless oil.

MS (APCI) m/z: 216/218 [M+H]⁺.

### REFERENCE EXAMPLE 561

### Preparation of 2-methyl-2-[(7-methyl-2,3-dihydro-1H-indene-4-yl)oxy]propan-1-ol

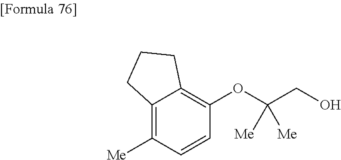 [SMILES: Cc1ccc(OC(C)(C)CO)c2c1CCC2]

To a solution of 2-methyl-2-[(7-methyl-2,3-dihydro-1H-inden-4-yl)oxy]propionic acid (500 mg) in tetrahydrofuran (20 mL) was added borane-tetrahydrofuran complex (5 mL, 1.1 mol/L in tetrahydrofuran), and the reaction mixture was stirred at room temperature for 18 hours. The reaction mixture was added with saturated aqueous sodium hydrogen carbonate. The organic layer was separated, and the aqueous layer was extracted with ethyl acetate. The organic layer was concentrated under reduced pressure to yield the titled compound (357 mg, 83% yield) as a pale yellow solid.

MS (ESI) m/z: 221 [M+H]⁺.

### REFERENCE EXAMPLE 562

### Preparation of 2,2-dimethyl-3-(2-naphthyl)propan-1-ol

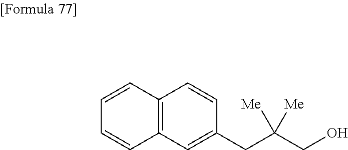 [SMILES: CC(C)(CO)Cc1ccc2ccccc2c1]

2,2-dimethyl-3-(2-naphthyl)propionic acid was reacted in the same manner as Reference Example 561 to yield the titled compound.

¹H NMR (400 MHz, CHLOROFORM-d) δ ppm 7.71-7.96 (m, 3H), 7.61 (s, 1H), 7.37-7.55 (m, 2H), 7.33 (dd, J=1.54, 8.22 Hz, 1H), 3.36 (d, J=5.65 Hz, 2H), 2.75 (s, 2H), 1.40 (t, J=5.65 Hz, 1H), 0.94 (s, 6H).

### REFERENCE EXAMPLE 563

### Preparation of 1-cyclopropyl-5,6,7,8-tetrahydronaphthalene-2-ol

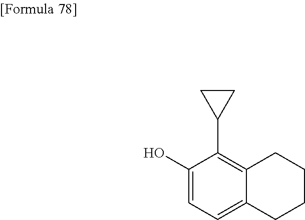 [SMILES: Oc1ccc2c(c1C1CC1)CCCC2]

(1) To a suspension of benzoic acid 1-bromo-5,6,7,8-tetrahydro-2-naphthyl ester (160 mg), cyclopropyl boronic acid (124 mg) and cesium fluoride (367 mg) in 1,4-dioxane (3 mL) was added 1,1′-bis(diphenylphosphino)ferrocene-palladium(II) dichloride dichloromethane complex (39 mg), and the reaction mixture was stirred for 3 hours at 100° C. The reaction mixture was concentrated under reduced pressure, and the residue was purified by silica gel column chromatography (solvent: hexane/ethyl acetate=98/2 to 90/10) to yield benzoic acid 1-cyclopropyl-5,6,7,8-tetrahydro-2-naphthyl ester (124 mg, 88% yield) as a pale yellow viscous material.

MS (ESI) m/z: 293 [M+H]⁺.

(2) To a solution of the compound obtained (1) (124 mg) in ethanol (2 mL) was added 1 mol/L aqueous sodium hydroxide solution (4 mL), and the reaction mixture was stirred for 30 minutes at 60° C. After neutralizing the reaction mixture with 1 mol/L hydrochloric acid, the mixture was extracted with ethyl acetate. The residue was purified by silica gel column chromatography (solvent: hexane/ethyl acetate=98/2 to 95/5) to yield the titled compound (103 mg, 99% yield) as a pale yellow viscous material.

MS (ESI) m/z: 189 [M+H]⁺.

### REFERENCE EXAMPLE 564

### Preparation of ethyl 1-(3-bromo-7-methoxy-1-methyl-1H-pyrazolo[4,3-d]pyrimidin-5-yl)-1H-pyrazole-4-carboxylate

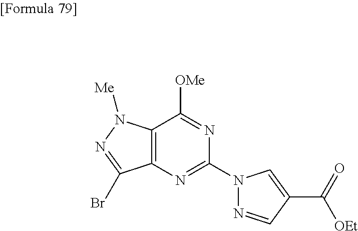 [SMILES: CCOC(=O)c1cnn(-c2nc(OC)c3c(n2)c(Br)nn3C)c1]

Ethyl 1-(3-bromo-7-methoxy-1H-pyrazolo[4,3-d]pyrimidin-5-yl)-1H-pyrazole-4-carboxylate prepared in Reference Example 461 was reacted in the same manner as Reference Example 40, 41 to yield the titled compound.

MS (APCI) m/z: 381/383 [M+H]⁺.

### REFERENCE EXAMPLE 565

### Preparation of ethyl 1-(7-methoxy-1-methyl-3-phenyl-1H-pyrazolo[4,3-d]pyrimidin-5-yl)-1H-pyrazole-4-carboxylate

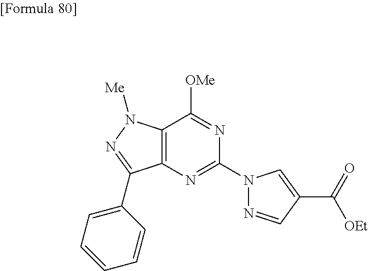 [SMILES: CCOC(=O)c1cnn(-c2nc(OC)c3c(n2)c(-c2ccccc2)nn3C)c1]

To a suspension of ethyl 1-(3-bromo-7-methoxy-1-methyl-1H-pyrazolo[4,3-d]pyrimidin-5-yl)-1H-pyrazole-4-carboxylate prepared in Reference Example 564 (50 mg), phenylboronic acid (32 mg) and tripotassium phosphate (84 mg) in 1,4-dioxane (1 mL) was added bis(di-t-butyl(4-dimethylaminophenyl)phosphine)dichloropalladium(II) (9 mg), and the reaction mixture was stirred for 3 hours at 100° C. The NH-silica gel (5 mL) and sodium sulfate (5 g) was added to the reaction mixture, and the insoluble materials were removed by filtration. The filtrate was concentrated under reduced pressure, and the residue was purified by silica gel column chromatography (solvent: hexane/ethyl acetate=80/20 to 70/30) to yield the titled compound (41 mg, 83% yield) as a colorless solid.

MS (APCI) m/z: 379 [M+H]⁺

### REFERENCE EXAMPLE 566

### Preparation of ethyl 1-(3-cyclohex-1-en-1-yl-7-methoxy-1-methyl-1H-pyrazolo[4,3-d]pyrimidin-5-yl)-1H-pyrazole-4-carboxylate

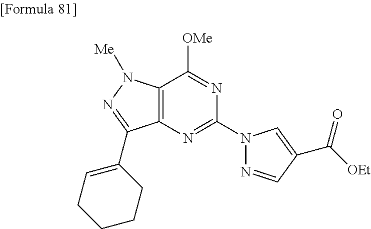 [SMILES: CCOC(=O)c1cnn(-c2nc(OC)c3c(n2)c(C2=CCCCC2)nn3C)c1]

Ethyl 1-(3-bromo-7-methoxy-1-methyl-1H-pyrazolo[4,3-d]pyrimidin-5-yl)-1H-pyrazole-4-carboxylate prepared in Reference Example 564 was reacted in the same manner as Reference Example 565 to yield the titled compound.

MS (APCI) m/z: 383 [M+H]⁺.

### REFERENCE EXAMPLE 567

### Preparation of ethyl 1-(3-cyclohexyl-7-methoxy-1-methyl-1H-pyrazolo[4,3-d]pyrimidin-5-yl)-1H-pyrazole-4-carboxylate

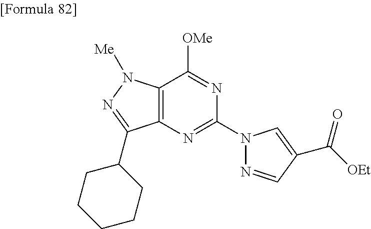 [SMILES: CCOC(=O)c1cnn(-c2nc(OC)c3c(n2)c(C2CCCCC2)nn3C)c1]

To a solution of ethyl 1-(3-cyclohex-1-en-1-yl-7-methoxy-1-methyl-1H-pyrazolo[4,3-d]pyrimidin-5-yl)-1H-pyrazole-4-carboxylate prepared in Reference Example 566 (15 mg) in methanol (1 mL) was added 10% palladium on carbon (50% wet with water) (5 mg), and the reaction mixture was stirred at room temperature for 7 hours under hydrogen atmosphere. The insoluble materials in the reaction mixture were removed by filtration through diatomaceous earth, and the filtrate was concentrated under reduced pressure. The residue was purified by silica gel column chromatography (solvent: hexane/ethyl acetate=80/20 to 70/30) to yield the titled compound (15 mg, 100% yield) as a colorless solid.

MS (APCI) m/z: 385 [M+H]⁺.

### REFERENCE EXAMPLE 568

### Preparation of ethyl 1-(7-methoxy-1,3-dimethyl-1H-pyrazolo[4,3-d]pyrimidin-5-yl)-1H-pyrazole-4-carboxylate

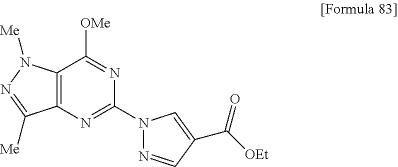 [SMILES: CCOC(=O)c1cnn(-c2nc(OC)c3c(n2)c(C)nn3C)c1]

Ethyl 1-(3-bromo-7-methoxy-1-methyl-1H-pyrazolo[4,3-d]pyrimidin-5-yl)-1H-pyrazole-4-carboxylate prepared in Reference Example 564 was reacted in the same manner as Reference Example 565 to yield the titled compound.

MS (APCI) m/z: 317 [M+H]⁺.

### REFERENCE EXAMPLE 569

### Preparation of ethyl 1-[3-(bromomethyl)-7-methoxy-1-methyl-1H-pyrazolo[4,3-d]pyrimidin-5-yl]-1H-pyrazole-4-carboxylate

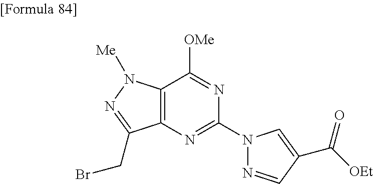 [SMILES: CCOC(=O)c1cnn(-c2nc(OC)c3c(n2)c(CBr)nn3C)c1]

To a suspension of ethyl 1-(7-methoxy-1,3-dimethyl-1H-pyrazolo[4,3-d]pyrimidin-5-yl)-1H-pyrazole-4-carboxylate prepared in Reference Example 568 (165 mg) in carbon tetrachloride (3 mL) were added N-bromosuccinimide (89 mg) and azobisisobutyronitrile (25 mg), and the reaction mixture was stirred for 8 hours under reflux. The reaction mixture was concentrated under reduced pressure, and the residue was purified by silica gel column chromatography (solvent: hexane/ethyl acetate=90/10 to 60/40) to yield the titled compound (60.8 mg, 30% yield) as a colorless solid.

MS (APCI) m/z: 395/397 [M+H]⁺.

### REFERENCE EXAMPLE 570

### Preparation of ethyl 1-(3-benzyl-7-methoxy-1-methyl-1H-pyrazolo[4,3-d]pyrimidin-5-yl)-1H-pyrazole-4-carboxylate

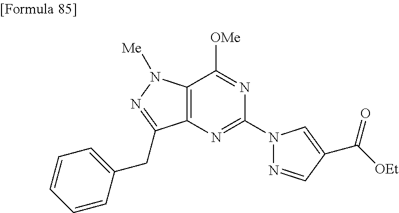 [SMILES: CCOC(=O)c1cnn(-c2nc(OC)c3c(n2)c(Cc2ccccc2)nn3C)c1]

To a suspension of ethyl 1-[3-(bromomethyl)-7-methoxy-1-methyl-1H-pyrazolo[4,3-d]pyrimidin-5-yl]-1H-pyrazole-4-carboxylate prepared in Reference Example 569 (60.8 mg), phenylboronic acid (38 mg) and tripotassium phosphate (98 mg) in 1,4-dioxane (1 mL) was added bis(di-t-butyl(4-dimethylaminophenyl)phosphine)dichloropalladium(II) (11 mg), and the reaction mixture was stirred for 6 hours at 100° C. The NH-silica gel (5 mL) and sodium sulfate (5 g) were added to the reaction mixture, and the insoluble materials were removed by filtration. The filtrate was concentrated under reduced pressure, and the residue was purified by silica gel column chromatography (solvent: hexane/ethyl acetate=70/30). The resulting crude product was re-purified by NH-silica gel column chromatography (solvent: hexane/ethyl acetate=80/20) to yield the titled compound (10.3 mg, 17% yield) as a colorless solid.

MS (APCI) m/z: 393 [M+H]⁺.

### REFERENCE EXAMPLE 571

### Preparation of ethyl 1-[3-(bromomethyl)-1-(cyclohexylmethyl)-7-methoxy-1H-pyrazolo[4,3-d]pyrimidin-5-yl]-1H-pyrazole-4-carboxylate

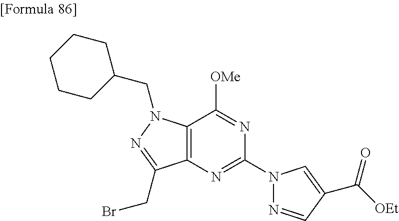 [SMILES: CCOC(=O)c1cnn(-c2nc(OC)c3c(n2)c(CBr)nn3CC2CCCCC2)c1]

Ethyl 1-[1-(cyclohexylmethyl)-7-methoxy-3-methyl-1H-pyrazolo[4,3-d]pyrimidin-5-yl]-1H-pyrazole-4-carboxylate prepared in Reference Example 520 was reacted in the same manner as Reference Example 569 to yield the titled compound.

MS (APCI) m/z: 477/479 [M+H]⁺.

### REFERENCE EXAMPLE 572

### Preparation of ethyl 1-[3-benzyl-1-(cyclohexylmethyl)-7-methoxy-1H-pyrazolo[4,3-d]pyrimidin-5-yl]-1H-pyrazole-4-carboxylate

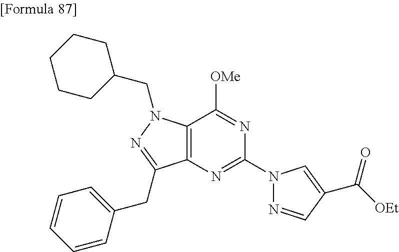 [SMILES: CCOC(=O)c1cnn(-c2nc(OC)c3c(n2)c(Cc2ccccc2)nn3CC2CCCCC2)c1]

Ethyl 1-[3-(bromomethyl)-1-(cyclohexylmethyl)-7-methoxy-1H-pyrazolo[4,3-d]pyrimidin-5-yl]-1H-pyrazole-4-carboxylate prepared in Reference Example 571 was reacted in the same manner as Reference Example 570 to yield the titled compound.

MS (APCI) m/z: 475 [M+H]⁺.

### REFERENCE EXAMPLE 573

### Preparation of ethyl 1-{7-methoxy-1-[4-(2-oxopyrrolidin-1-yl)benzyl]-1H-pyrazolo[4,3-d]pyrimidin-5-yl}-1H-pyrazole-4-carboxylate

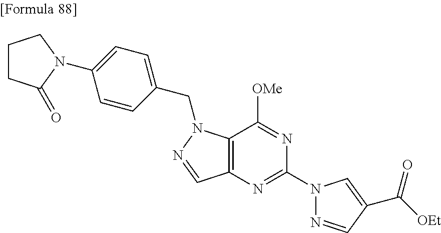 [SMILES: CCOC(=O)c1cnn(-c2nc(OC)c3c(cnn3Cc3ccc(N4CCCC4=O)cc3)n2)c1]

A suspension of ethyl 1-[1-(4-bromobenzyl)-7-methoxy-1H-pyrazolo[4,3-d]pyrimidin-5-yl]-1H-pyrazole-4-carboxylate prepared in Reference Example 118 (100 mg), pyrrolidin-2-one (37.4 mg), tris(dibenzylideneacetone)dipalladium(0) (20 mg), 4,5′-bis(diphenylphosphino)-9,9′-dimethylxanthene (25.5 mg) and cesium carbonate (215 mg) in 1,4-dioxane (6 mL) was stirred for 3 hours at 80° C. under nitrogen atmosphere. NH-silica gel and ethyl acetate were added to the reaction mixture, and the insoluble materials were removed by filtration. The filtrate was concentrated under reduced pressure, and the residue was purified by silica gel column chromatography (solvent: hexane/ethyl acetate=70/30 to 0/100), followed by NH-silica gel column chromatography (solvent: hexane/ethyl acetate=100/0 to 50/50) to yield the titled compound (53 mg, 52% yield) as a colorless solid.

MS (ESI) m/z: 462 [M+H]⁺.

### REFERENCE EXAMPLE 574

### Preparation of ethyl 1-{1-[4-(4,4-dimethyl-2-oxopyrrolidin-1-yl)benzyl]-7-methoxy-1H-pyrazolo[4,3-d]pyrimidin-5-yl}-1H-pyrazole-4-carboxylate

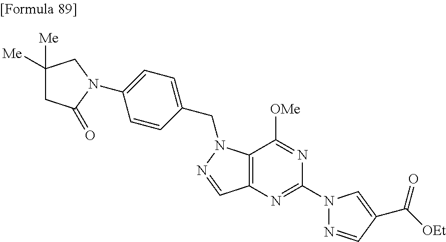 [SMILES: CCOC(=O)c1cnn(-c2nc(OC)c3c(cnn3Cc3ccc(N4CC(C)(C)CC4=O)cc3)n2)c1]

Ethyl 1-[1-(4-bromobenzyl)-7-methoxy-1H-pyrazolo[4,3-d]pyrimidin-5-yl]-1H-pyrazole-4-carboxylate prepared in Reference Example 118 and 4,4-dimethylpyrrolidine-2-one were reacted in the same manner as Reference Example 573 to yield the titled compound.

MS (ESI) m/z: 490 [M+H]⁺.

### REFERENCE EXAMPLE 575

### Preparation of ethyl 1-(3-iodo-7-methoxy-1H-pyrazolo[4,3-d]pyrimidin-5-yl)-1H-pyrazole-4-carboxylate

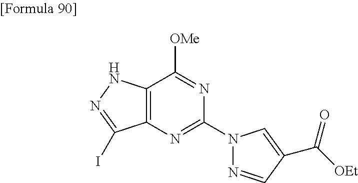 [SMILES: CCOC(=O)c1cnn(-c2nc(OC)c3[nH]nc(I)c3n2)c1]

Ethyl 1-(7-methoxy-1H-pyrazolo[4,3-d]pyrimidin-5-yl)-1H-pyrazole-4-carboxylate prepared in Reference Example 1 or 2 and N-iodosuccinimide were reacted in the same manner as Reference Example 259 to yield the titled compound.

MS (APCI) m/z: 415 [M+H]⁺.

### REFERENCE EXAMPLE 576

### Preparation of ethyl 1-[1-(cyclohexylmethyl)-7-methoxy-3-(trifluoromethyl)-1H-pyrazolo[4,3-d]pyrimidin-5-yl]-1H-pyrazole-4-carboxylate

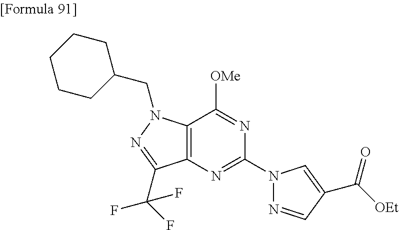 [SMILES: CCOC(=O)c1cnn(-c2nc(OC)c3c(n2)c(C(F)(F)F)nn3CC2CCCCC2)c1]

To a solution of ethyl 1-[1-(cyclohexylmethyl)-3-iodo-7-methoxy-1H-pyrazolo[4,3-d]pyrimidin-5-yl]-1H-pyrazole-4-carboxylate prepared in Reference Example 518 (77 mg) in N,N-dimethylformamide (2 mL) were added copper(I) iodide (72 mg) and methyl(fluorosulfonyl)difluoroacetate (95 μL), and the reaction mixture was stirred at 100° C. for 2.5 hours under nitrogen atmosphere. The insoluble materials in the reaction mixture were removed by filtration through diatomaceous earth, and the filtrate was concentrated under reduced pressure. The residue was purified by silica gel column chromatography (solvent: hexane/ethyl acetate=80/20) to yield the titled compound (31.7 mg, 46% yield) as a colorless solid.

MS (APCI) m/z: 453 [M+H]⁺.

### REFERENCE EXAMPLE 577

### Preparation of ethyl 1-[7-methoxy-3-(trifluoromethyl)-1-{(1S)-1-[4-(trifluoromethyl)phenyl]ethyl}-1H-pyrazolo[4,3-d]pyrimidin-5-yl]-1H-pyrazole-4-carboxylate

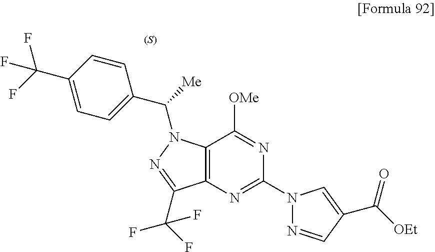

Ethyl 1-(3-iodo-7-methoxy-1-{(1S)-1-[4-(trifluoromethyl)phenyl]ethyl}-1H-pyrazolo[4,3-d]pyrimidin-5-yl)-1H-pyrazole-4-carboxylate prepared in Reference Example 519 was reacted in the same manner as Reference Example 576 to yield the titled compound.

MS (APCI) m/z: 529 [M+H]⁺.

### REFERENCE EXAMPLE 578

### Preparation of (1S)-1-(3-chloro-5-methylphenyl)ethanol

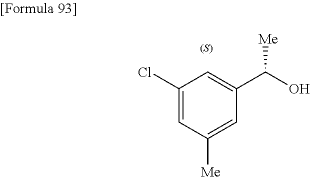

(R)-5,5-diphenyl-2-methyl-3,4-propano-1,3,2-oxyazaborolidine (1 mol/L in tetrahydrofuran, 0.89 mL) was diluted with tetrahydrofuran (3 mL), and borane-dimethyl sulfide complex (2 mol/L in tetrahydrofuran, 2.22 mL) was added dropwise over 5 minutes at 2° C. under nitrogen atmosphere. After stirring for 5 minutes at 2° C., a solution of 1-(3-chloro-5-methylphenyl)ethanone (500 mg) in tetrahydrofuran (5 mL) was added dropwise over 10 minutes at 2° C., and the mixture was stirred for 1.5 hours at the same temperature. Under ice-cooling, saturated aqueous ammonium chloride solution was added to the reaction mixture. The aqueous layer was extracted with ethyl acetate, and the organic layer was washed sequentially with 1 mol/L hydrochloric acid, saturated aqueous sodium hydrogen carbonate and brine, dried over sodium sulfate, and concentrated under reduced pressure. The residue was purified by silica gel column chromatography (solvent: hexane/ethyl acetate=95/5 to 80/20) to yield the titled compound (474 mg, 94% yield) as a colorless viscous material.

¹H NMR (400 MHz, CHLOROFORM-d) δ ppm 7.17 (s, 1H), 7.07-7.05 (m, 2H), 4.84 (qd, J=6.4, 3.9 Hz, 1H), 2.33 (s, 3H), 1.78 (d, J=4.1 Hz, 1H), 1.47 ppm (d, J=6.7 Hz, 3H).

### REFERENCE EXAMPLE 579

### Preparation of (1R)-1-(3-chloro-5-methylphenyl)ethanol

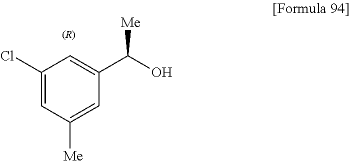

(S)-5,5-diphenyl-2-methyl-3,4-propano-1,3,2-oxyazaborolidine (1 mol/L in toluene, 0.36 mL) was diluted with tetrahydrofuran (2 mL), and borane-dimethyl sulfide complex (2 mol/L in tetrahydrofuran, 0.89 mL) was added dropwise over 5 minutes at 4° C. under nitrogen atmosphere. After stirring for 5 minutes at 4° C., a solution of 1-(3-chloro-5-methylphenyl)ethanone (200 mg) in tetrahydrofuran (3 mL) was added dropwise over 10 minutes at 5° C., and the mixture was stirred for 1.5 hours at the same temperature. Under ice-cooling, saturated aqueous ammonium chloride solution was added to the reaction mixture. The aqueous layer was extracted with ethyl acetate, and the organic layer was washed sequentially with 1 mol/L hydrochloric acid, saturated aqueous sodium hydrogen carbonate and brine, dried over sodium sulfate, and concentrated under reduced pressure. The residue was purified by silica gel column chromatography (solvent: hexane/ethyl acetate=95/5 to 80/20) to yield the titled compound (229 mg, 100% yield) as a colorless viscous material.

¹H NMR (400 MHz, CHLOROFORM-d) δ ppm 7.17 (s, 1H), 7.07 (s, 1H), 7.05 (s, 1H), 4.83 (qd, J=6.68, 3.60 Hz, 1H), 2.33 (s, 3H), 1.78 (d, J=3.60 Hz, 1H), 1.47 (d, J=6.68 Hz, 3H).

### REFERENCE EXAMPLE 580

### Preparation of (1S)-1-[4-methyl-3-(trifluoromethyl)phenyl]ethanol

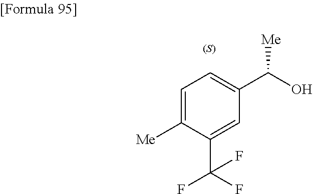

1-[4-methyl-3-(trifluoromethyl)phenyl]ethanone was reacted in the same manner as Reference Example 578 to yield the titled compound.

¹H NMR (400 MHz, CHLOROFORM-d) δ ppm 7.62 (s, 1H), 7.43 (d, J=7.68 Hz, 1H), 7.27 (d, J=7.68 Hz, 1H), 4.93 (qd, J=6.66, 3.58 Hz, 1H), 2.47 (s, 3H), 1.81 (d, J=3.58 Hz, 1H), 1.50 (d, J=6.66 Hz, 3H).

### REFERENCE EXAMPLE 581

### Preparation of (1R)-1-(3-chloro-5-methylphenyl)ethanol

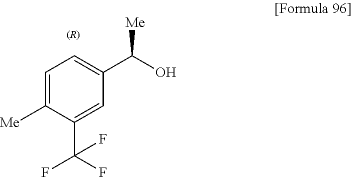

1-[4-methyl-3-(trifluoromethyl)phenyl]ethanone was reacted in the same manner as Reference Example 579 to yield the titled compound.

¹H NMR (400 MHz, CHLOROFORM-d) δ ppm 7.62 (s, 1H), 7.43 (d, J=7.68 Hz, 1H), 7.27 (d, J=7.68 Hz, 1H), 4.93 (qd, J=6.66, 3.07 Hz, 1H), 2.47 (s, 3H), 1.82 (d, J=3.07 Hz, 1H), 1.50 (d, J=6.66 Hz, 3H).

### REFERENCE EXAMPLE 582

### Preparation of (1S)-1-[2-methyl-5-(trifluoromethyl)phenyl]ethanol

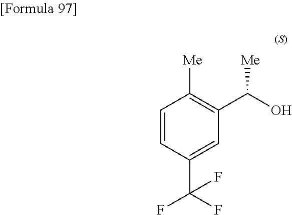

1-[2-methyl-5-(trifluoromethyl)phenyl]ethanone was reacted in the same manner as Reference Example 578 to yield the titled compound.

¹H NMR (400 MHz, CHLOROFORM-d) δ ppm 7.80 (s, 1H), 7.42 (d, J=7.71 Hz, 1H), 7.23 (d, J=7.71 Hz, 1H), 5.16 (qd, J=6.17, 3.60 Hz, 1H), 2.39 (s, 3H), 1.78 (d, J=3.60 Hz, 1H), 1.48 (d, J=6.17 Hz, 3H).

### REFERENCE EXAMPLE 583

### Preparation of (1R)-1-[2-methyl-5-(trifluoromethyl)phenyl]ethanol

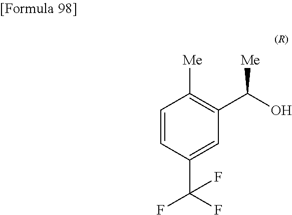

1-[2-methyl-5-(trifluoromethyl)phenyl]ethanone was reacted in the same manner as Reference Example 579 to yield the titled compound.

¹H NMR (400 MHz, CHLOROFORM-d) δ ppm 7.80 (s, 1H), 7.42 (d, J=7.71 Hz, 1H), 7.23 (d, J=7.71 Hz, 1H), 5.16 (qd, J=6.17, 3.60 Hz, 1H), 2.39 (s, 3H), 1.77 (d, J=3.60 Hz, 1H), 1.48 (d, J=6.17 Hz, 3H).

### REFERENCE EXAMPLE 584

### Preparation of (2S)-1-[4-({[t-butyl(dimethyl)silyl]oxy}methyl)phenyl]-2-(trifluoromethyl)pyrrolidine

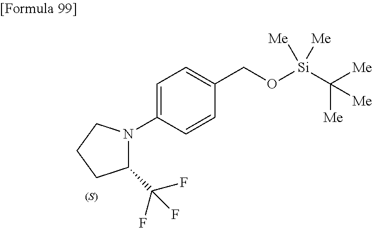

A suspension of [(4-bromobenzyl)oxy](t-butyl)dimethylsilane (1.0 g), (2S)-2-(trifluoromethyl)pyrrolidine (695 mg), tris(dibenzylideneacetone)palladium(0) (302 mg), 2-dicyclohexyl-phosphino-2′,6′-diisopropoxybiphenyl (308 mg) and sodium t-butoxide (638 mg) in 1,2-dimethoxyethane (50 mL) was stirred for 2 hours at 90° C. under nitrogen atmosphere. NH-silica gel and ethyl acetate were added to the reaction mixture, and the insoluble materials were removed by filtration. The filtrate was concentrated under reduced pressure, and the resulting residue was purified by NH-silica gel column chromatography (solvent: hexane/ethyl acetate=100/0 to 50/50), followed by silica gel column chromatography (solvent: hexane/ethyl acetate=90/10 to 20/80) to yield the titled compound (1.12 g, 94% yield). ¹H NMR (400 MHz, CHLOROFORM-d) δ ppm 7.21 (d, J=8.70 Hz, 2H), 6.74 (d, J=8.70 Hz, 2H), 4.65 (s, 2H), 4.21 (m, 1H), 3.60-3.69 (m, 1H), 3.16-3.26 (m, 1H), 2.12-2.29 (m, 2H), 1.95-2.11 (m, 2H), 0.93 (s, 9H), 0.08 (s, 6H).

### REFERENCE EXAMPLE 585

### Preparation of {4-[(2S)-2-(trifluoromethyl)pyrrolidin-1-yl]phenyl}methanol

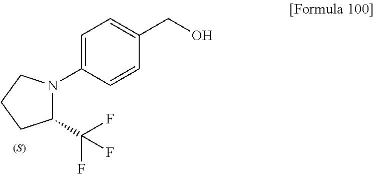

To a solution of (2S)-1-[4({[t-butyl(dimethyl)silyl]oxy}methyl)phenyl]-2-(trifluoromethyl)pyrrolidine prepared in Reference Example 584 (1.1 g) in tetrahydrofuran (20 mL) was added tetrabutylammonium fluoride (1 mol/L in tetrahydrofuran, 6.1 mL) dropwise at room temperature, and the mixture was stirred at the same temperature for 1 hour. The reaction mixture was added with water and ethyl acetate. The organic layer was separated, and the aqueous layer was extracted with ethyl acetate. The organic layer was washed with saturated aqueous ammonium chloride and brine, dried over sodium sulfate, and concentrated under reduced pressure to yield the titled compound (724 mg, 97% yield) as a pale yellow oil.

MS (ESI) m/z: 246 [M+H]⁺.

### REFERENCE EXAMPLE 586

### Preparation of 1-[4-({[t-butyl(dimethyl)silyl]oxy}methyl)phenyl]-4-(trifluoromethyl)piperidine

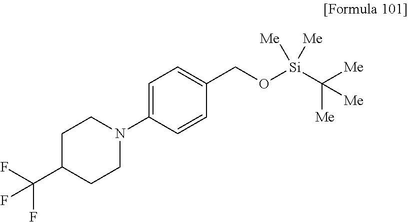 [SMILES: CC(C)(C)[Si](C)(C)OCc1ccc(N2CCC(C(F)(F)F)CC2)cc1]

A suspension of [(4-bromobenzyl)oxy](t-butyl)dimethylsilane (558 mg), 4-(trifluoromethyl)piperidine hydrochloride (527 mg), tris(dibenzylideneacetone)palladium(0) (170 mg), 2-dicyclohexyl-phosphino-2′,6′-diisopropoxybiphenyl (173 mg) and sodium t-butoxide (623 mg) in 1,2-dimethoxyethane (11 mL) was stirred for 2 hours at 90° C. under nitrogen atmosphere. NH-silica gel and silica gel were added to the reaction mixture, and the insoluble materials were removed by filtration. The filtrate was concentrated under reduced pressure, and the resulting residue was purified by silica gel column chromatography (solvent: hexane/ethyl acetate=100/0 to 93/7) to yield the titled compound (635 mg, 92% yield) as a pale yellow solid.

MS (ESI) m/z: 374 [M+H]⁺.

### REFERENCE EXAMPLE 587

### Preparation of {4-[4-(trifluoromethyl)piperidin-1-yl]phenyl}methanol

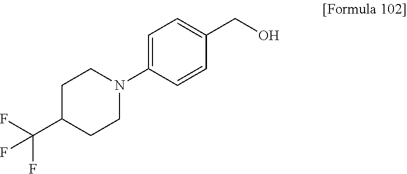 [SMILES: OCc1ccc(N2CCC(C(F)(F)F)CC2)cc1]

To a solution of 1-[4({[t-butyl(dimethyl)silyl]oxy}methyl)phenyl]-4-(trifluoromethyl)piperidine prepared in Reference Example 586 (630 mg) in tetrahydrofuran (13 mL) was added tetrabutylammonium fluoride (1 mol/L in tetrahydrofuran, 3.4 mL) dropwise at room temperature, and the mixture was stirred at the same temperature for 3 hours. The reaction mixture was added with water and ethyl acetate. The organic layer was separated, and the aqueous layer was extracted with ethyl acetate. The organic layer was washed with brine, dried over sodium sulfate, and concentrated under reduced pressure. The residue was purified by silica gel column chromatography (solvent: hexane/ethyl acetate=85/15 to 50/50) to yield the titled compound (424 mg, 97% yield) as a colorless powder.

MS (ESI) m/z: 260 [M+H]⁺.

### REFERENCE EXAMPLE 588

### Preparation of 1-{4-[(1R)-1-{[t-butyl(dimethyl)silyl]oxy}ethyl]phenyl}-4-(trifluoromethyl)piperidine

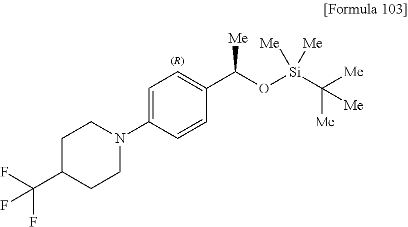

[(1R)-1-(4-bromophenyl)ethoxy](t-butyl)dimethylsilane was reacted with 4-(trifluoromethyl)piperidine hydrochloride in the same manner as Reference Example 586 to yield the titled compound.

MS (ESI) m/z: 388 [M+H]⁺.

### REFERENCE EXAMPLE 589

### Preparation of (1R)-1-{4-[4-(trifluoromethyl)piperidin-1-yl]phenyl}ethanol

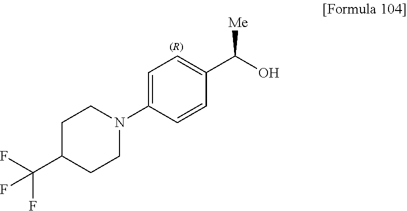

1-{4-[(1R)-1-{[t-butyl(dimethyl)silyl]oxy}ethyl]phenyl}-4-(trifluoromethyl)piperidine prepared in Reference Example 588 was reacted in the same manner as Reference Example 587 to yield the titled compound.

MS (ESI) m/z: 274 [M+H]⁺.

### REFERENCE EXAMPLE 590

### Preparation of (1R)-1-{1-[4-(trifluoromethyl)phenyl]piperidin-4-yl}ethanol

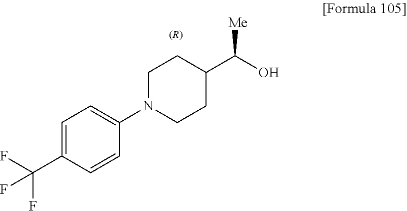

A suspension of (1R)-1-(piperidin-4-yl)ethanol hydrochloride (500 mg), 1-fluoro-4-(trifluoromethyl)benzene (1.92 mL) and potassium carbonate (1.25 g) in dimethylformamide (5 mL) was stirred for 20 hours at 130° C. The reaction mixture was added with water and ethyl acetate. The organic layer was separated, and the aqueous layer was extracted with ethyl acetate. The organic layer was washed with brine, dried over sodium sulfate, and concentrated under reduced pressure. The residue was purified by silica gel column chromatography (solvent: hexane/ethyl acetate=85/15 to 50/50) to yield the titled compound (580 mg, 70% yield) as a colorless solid.

MS (ESI) m/z: 274 [M+H]⁺.

### REFERENCE EXAMPLE 591

### Preparation of (1R)-1-[1-(4-chlorophenyl)piperidin-4-yl]ethanol

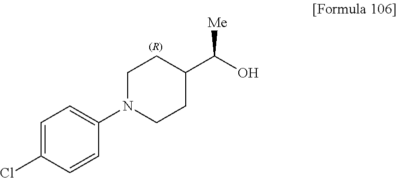

A suspension of (1R)-1-(piperidin-4-yl)ethanol hydrochloride (250 mg), 1-chloro-4-iodobenzene (240 mg), copper(I) bromide (29 mg), 2,2′-bis(diphenylphosphino)-1,1′-binaphthyl (58 mg) and potassium phosphate (427 mg) in N,N-dimethylformamide (2.5 mL) was stirred at 90° C. for 4.5 hours under nitrogen atmosphere. After the reaction mixture was allowed to room temperature, copper(I) bromide (29 mg), 2,2′-bis(diphenylphosphino)-1,1′-binaphthyl (58 mg) and potassium phosphate (427 mg) were added, and the mixture was stirred at 90° C. for further 17 hours under nitrogen atmosphere. The reaction mixture was added with ethyl acetate, and the insoluble materials were removed by filtration through diatomaceous earth. The filtrate was added with water and filtered off again insoluble materials through diatomaceous earth. The organic layer was separated, and the aqueous layer was extracted with ethyl acetate. The organic layer was washed with brine, dried over sodium sulfate, and concentrated under reduced pressure. The residue was purified by silica gel column chromatography (solvent: hexane/ethyl acetate=85/15 to 60/40) to yield the titled compound (152 mg, 63% yield) as a yellow solid.

MS (ESI) m/z: 240/242 [M+H]⁺.

### REFERENCE EXAMPLE 592

### Preparation of (1R)-1-{1-[4-(trifluoromethoxy)phenyl]piperidin-4-yl}ethanol

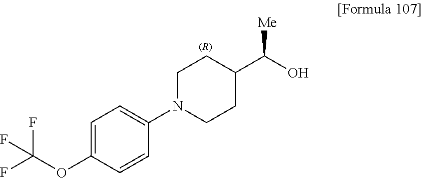

(1R)-1-(piperidin-4-yl)ethanol hydrochloride was reacted with 1-iodo-4-(trifluoromethoxy)benzene in the same manner as Reference Example 591 to yield the titled compound.

MS (ESI) m/z: 290 [M+H]⁺.

### REFERENCE EXAMPLE 593

### Preparation of (1R)-1-{1-[3-(trifluoromethyl)phenyl]piperidin-4-yl}ethanol

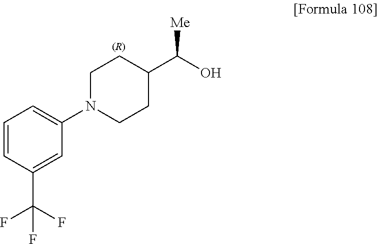

(1R)-1-(piperidin-4-yl)ethanol hydrochloride was reacted with 1-iodo-3-(trifluoromethyl)benzene in the same manner as Reference Example 591 to yield the titled compound.

MS (ESI) m/z: 274 [M+H]⁺.

### REFERENCE EXAMPLE 594

### Preparation of (1R)-1-[1-(3-chlorophenyl)piperidin-4-yl]ethanol

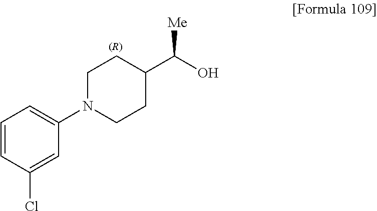

(1R)-1-(piperidin-4-yl)ethanol hydrochloride was reacted with 1-chloro-3-iodobenzene in the same manner as Reference Example 591 to yield the titled compound.

MS (ESI) m/z: 240/242 [M+H]⁺.

### REFERENCE EXAMPLE 595

### Preparation of ethyl 1-{7-methoxy-1-[(1R,2S,5S)-2-methyl-5-(propan-2-yl)cyclohexyl]-1H-pyrazolo[4,3-d]pyrimidin-5-yl}-1H-pyrazole-4-carboxylate

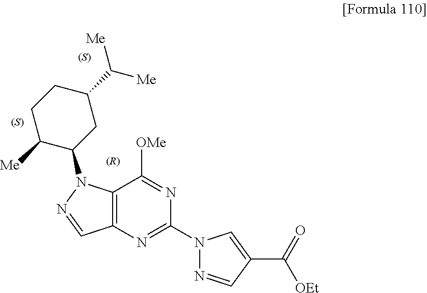

Ethyl 1-{7-methoxy-1-[(1R,2S,5S)-2-methyl-5-(prop-1-en-2-yl)cyclohexyl]-1H-pyrazolo[4,3-d]pyrimidin-5-yl}-1H-pyrazole-4-carboxylate prepared in Reference Example 309 was reacted in the same manner as Reference Example 567 to yield the titled compound.

MS (APCI) m/z: 427 [M+H]⁺.

### REFERENCE EXAMPLE 596

### Preparation of ethyl 1-{7-methoxy-1-[(1S,2R,5R)-2-methyl-5-(propan-2-yl)cyclohexyl]-1H-pyrazolo[4,3-d]pyrimidin-5-yl}-1H-pyrazole-4-carboxylate

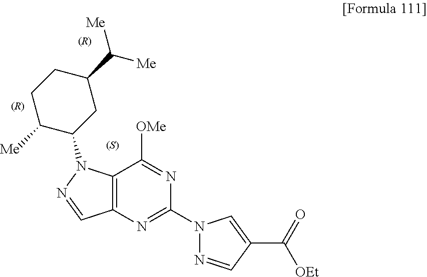

Ethyl 1-{7-methoxy-1-[(1S,2R,5R)-2-methyl-5-(prop-1-en-2-yl)cyclohexyl]-1H-pyrazolo[4,3-d]pyrimidin-5-yl}-1H-pyrazole-4-carboxylate prepared in Reference Example 310 was reacted in the same manner as Reference Example 567 to yield the titled compound.

MS (APCI) m/z: 427 [M+H]⁺.

### REFERENCE EXAMPLE 597

### Preparation of (1R)-1-[3-fluoro-4-(trifluoromethoxy)phenyl]propan-1-ol

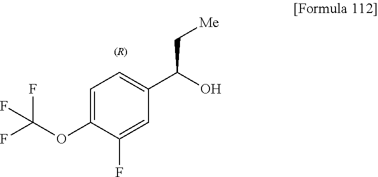

To a suspension of [(4S,5S)-2,2-dimethyl-1,3-dioxolane-4,5-diyl]bis(diphenylmethanol) (90 mg) in hexane (6 mL) was added dropwise titanium tetraisopropoxide (0.34 mL) at room temperature, and the reaction mixture was stirred for 5 minutes at the same temperature. Diethyl zinc (1.0 mol/L in hexane, 2.4 mL) was added dropwise at room temperature, and the reaction mixture was stirred for 20 minutes at the same temperature. The reaction mixture was added dropwise a solution of 3-fluoro-4-(trifluoromethoxy)benzaldehyde (200 mg) in hexane (2 mL) at −35° C., and the reaction mixture was stirred at the same temperature for 1 hour, at −20° C. for 15 hours, and at 0° C. for 1 hour. The reaction mixture was added with saturated aqueous ammonium chloride and water under ice-cooling, and then stirred for 10 minutes. Ethyl acetate was added, and the insoluble materials were removed by filtration through diatomaceous earth. The filtrate was concentrated under reduced pressure, and the residue was added with chloroform. The organic layer was separated, and the aqueous layer was extracted with chloroform. The organic layer was concentrated under reduced pressure, and the residue was purified by silica gel column chromatography (solvent: hexane/ethyl acetate=95/5 to 75/25), and then the resulting crude product was purified by NH-silica gel column chromatography (solvent: hexane/ethyl acetate=95/5 to 75/25) to yield the titled compound (113 mg, 49% yield) as a colorless liquid.

¹H NMR (400 MHz, CHLOROFORM-d) δ ppm 7.20-7.32 (m, 2H), 7.12 (d, J=8.70 Hz, 1H), 4.63 (td, J=6.66, 3.58 Hz, 1H), 1.89 (d, J=3.58 Hz, 1H), 1.69-1.83 (m, 2H), 0.94 (t, J=7.17 Hz, 3H).

### REFERENCE EXAMPLE 598

### Preparation of (1R)-1-[3-chloro-4-(trifluoromethoxy)phenyl]propan-1-ol

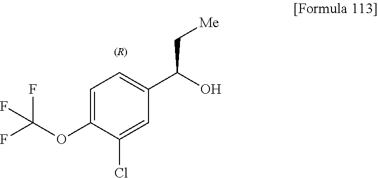

To a solution of [(4S,5S)-2,2-dimethyl-1,3-dioxolane-4,5-diyl]bis[di(naphthalene-2-yl)methanol] (119 mg) in hexane (5 mL) was added dropwise titanium tetraisopropoxide (0.32 mL) at room temperature, and the reaction mixture was stirred for 1.5 hours at the same temperature. The reaction mixture was added dropwise with diethyl zinc (1.0 mol/L in hexane, 2.2 mL) under ice-cooling, and the reaction mixture was stirred for 30 minutes at room temperature. The reaction mixture was added dropwise a solution of 3-chloro-4-(trifluoromethoxy)benzaldehyde (200 mg) in hexane (2.4 mL) at −20° C., and the reaction mixture was stirred at the same temperature for 23 hours. The reaction mixture was added with water and chloroform under ice-cooling. The mixture was stirred and then filtered through diatomaceous earth to remove insoluble materials. The organic layer was separated, and the aqueous layer was extracted with chloroform. The organic layer was concentrated under reduced pressure, and the residue was purified by silica gel column chromatography (solvent: hexane/ethyl acetate=95/5 to 75/25), and then the resulting crude product was purified by NH-silica gel column chromatography (solvent: hexane/ethyl acetate=95/5 to 75/25) to yield the titled compound (98 mg, 43% yield) as a colorless liquid.

¹H NMR (400 MHz, CHLOROFORM-d) δ ppm 7.48 (d, J=1.54 Hz, 1H), 7.20-7.32 (m, 2H), 4.62 (td, J=6.66, 3.58 Hz, 1H), 1.89 (d, J=3.58 Hz, 1H), 1.69-1.84 (m, 2H), 0.94 (t, J=7.17 Hz, 3H).

### REFERENCE EXAMPLE 599

### Preparation of (1R)-1-[3-chloro-4-(trifluoromethoxy)phenyl]ethanol

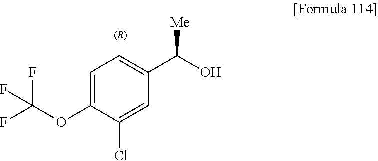

3-chloro-4-(trifluoromethoxy)benzaldehyde was reacted with dimethyl zinc in the same manner as Reference Example 598 to yield the titled compound.

¹H NMR (400 MHz, CHLOROFORM-d) δ ppm 7.51 (s, 1H), 7.29 (s, 2H), 4.91 (qd, J=6.66, 3.58 Hz, 1H), 1.84 (d, J=3.58 Hz, 1H), 1.50 (d, J=6.66 Hz, 3H).

### REFERENCE EXAMPLE 600

### Preparation of (3R)-1-(3,4-dichlorophenyl)pyrrolidine-3-ol

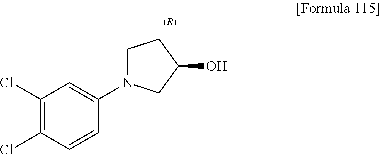

(3R)-pyrrolidine-3-ol was reacted with 1,2-dichloro-4-fluorobenzene in the same manner as Reference Example 590 to yield the titled compound.

MS (ESI) m/z: 232/234 [M+H]⁺.

### PHARMACOLOGICAL EXPERIMENTS

### Test Example 1

### HIF-PHD Inhibition Assay

### Test Compound:

The compounds described in the above Examples were used in the HIF-PHD inhibition assay.

### Test Method:

The activity of the test compound to inhibit human HIF-PHD2 and human HIF-PHD3 was determined.

An enzyme reaction solution containing: 20 mmol/L tris(hydroxymethyl)aminomethane-hydrochloric acid buffer solution (pH8.0), 120 mmol/L sodium chloride, 3.33 mmol/L ascorbic acid, 2-oxoglutaric acid (3.33 μmol/L for human HIF-PHD2, 166 μmol/L for human HIF-PHD3), 166 μmol/L iron(II) chloride, 2.67 μg/mL human VHL-Enlondin B-Enlondin C complex (human VBC complex, CrystalGenomics, Inc.), and 6.67 nmol/L synthetic FAM-HIF-2α peptide (FAM-ACA-ELDLETLAPYIPMDGEDFQL) was prepared, and 15 μL of the solution was dispensed to a 96-well half area plate. A solution of the test compound in dimethyl sulfoxide (5-fold of the final concentration) was added to the plate (5 μL/well), mixed using plate mixer, followed by measuring the fluorescence polarization (ex. 480 nm, em. 535 nm) using enVision (Perkin Elmer Co.). Then, 5 μL of enzyme solution containing either HIF-PHD2 or HIF-PHD3 (CrystalGenomics Ltd.) was added to each well, mixed using plate mixer, and after 20 to 40 minutes later, the fluorescence polarization (ex. 480 nm, em. 535 nm) was measured by using enVision. The value of enzyme activity was calculated by subtracting the value of fluorescence polarization before addition of the enzyme from the value of fluorescence polarization after addition of enzyme (mP value). Taking the activity value of the well containing enzyme alone as 100% and that of containing no enzyme as 0%, the rate of inhibition by sample for each well was calculated in terms of the percent of activity, and the result was fitted to S-curve using Pad Pat Prism (Graph Pad Software, Inc.) to determine the IC₅₀ value.

FAM: 5′-fluoresceinamide
ACA: aminocaproic acid

### Results:

The IC₅₀ values of the test compounds are shown in Table 12.

TABLE 12

| | HIF-PHD Inhibition Assay | |
|---|---|---|
| Test Compound | IC₅₀ (μmol/L) | |
| (Example No.) | HIF-PHD2 | HIF-PHD3 |
| 1 | 0.021 | 0.16 |
| 2 | 0.084 | 1.1 |
| 3 | 0.031 | 0.16 |
| 4 | 0.027 | 0.78 |
| 5 | 0.13 | 0.74 |
| 6 | 0.041 | 0.13 |
| 7 | 0.023 | 0.32 |
| 8 | 0.048 | 0.56 |
| 9 | 0.033 | 0.41 |
| 10 | 0.022 | 0.24 |
| 11 | 0.062 | 0.47 |
| 12 | 0.044 | >3.0 |
| 13 | 0.020 | 0.23 |
| 14 | 0.031 | 0.28 |
| 15 | 0.052 | 0.29 |
| 16 | 0.066 | 0.82 |
| 17 | 0.065 | 0.48 |
| 18 | 0.035 | 0.45 |
| 19 | 0.026 | 0.72 |
| 20 | 0.065 | 1.2 |
| 21 | 0.017 | 0.52 |
| 22 | 0.060 | 0.54 |
| 23 | 0.0089 | 0.22 |
| 24 | 0.044 | 0.99 |
| 25 | 0.070 | 2.9 |
| 26 | 0.066 | 1.5 |
| 27 | 0.020 | 2.6 |
| 28 | 0.035 | 0.36 |
| 29 | 0.037 | 0.20 |
| 30 | 0.072 | 0.46 |
| 31 | 0.013 | 0.27 |
| 32 | 0.014 | 0.32 |
| 33 | 0.010 | 0.20 |
| 34 | 0.017 | 0.24 |
| 35 | 0.018 | 0.25 |
| 36 | 0.015 | 0.17 |
| 37 | 0.026 | 0.12 |
| 38 | 0.15 | 1.8 |
| 39 | 0.025 | 0.35 |
| 40 | 0.011 | 0.14 |
| 41 | 0.022 | 0.34 |
| 42 | 0.014 | 1.4 |
| 43 | 0.015 | 0.20 |
| 44 | 0.049 | 0.39 |
| 45 | 0.050 | 0.17 |
| 46 | 0.068 | 0.085 |
| 47 | 0.077 | 0.65 |
| 48 | 0.070 | 0.34 |
| 49 | 0.030 | 0.20 |
| 50 | 0.051 | 0.32 |
| 51 | 0.034 | 0.19 |
| 52 | 0.022 | 0.25 |
| 53 | 0.025 | 0.41 |
| 54 | 0.018 | 0.25 |
| 55 | 0.0092 | 0.024 |
| 56 | 0.016 | 0.26 |
| 57 | 0.015 | 0.13 |
| 58 | 0.021 | 0.065 |
| 59 | 0.029 | 0.095 |
| 60 | 0.028 | 0.077 |
| 61 | 0.012 | 0.18 |
| 62 | 0.017 | 0.14 |
| 63 | 0.014 | 0.11 |
| 64 | 0.018 | 0.066 |
| 65 | 0.011 | 0.18 |
| 66 | 0.017 | 0.11 |
| 67 | 0.010 | 0.19 |
| 68 | 0.023 | 0.16 |
| 69 | 0.013 | 0.11 |
| 70 | 0.011 | 0.11 |
| 71 | 0.016 | 0.11 |
| 72 | 0.028 | 0.28 |
| 73 | 0.031 | 0.27 |
| 74 | 0.020 | 0.072 |
| 75 | 0.020 | 0.12 |
| 76 | 0.0091 | 0.12 |
| 77 | 0.0087 | 0.13 |
| 78 | 0.0061 | 0.068 |
| 79 | 0.016 | 0.20 |
| 80 | 0.0089 | 0.042 |
| 81 | 0.035 | 0.17 |
| 82 | 0.032 | 0.13 |
| 83 | 0.033 | 0.24 |
| 84 | 0.013 | 0.20 |
| 85 | 0.011 | 0.066 |
| 86 | 0.021 | 0.33 |
| 87 | 0.050 | 0.26 |
| 88 | 0.0086 | 0.044 |
| 89 | 0.048 | 1.3 |
| 90 | 0.020 | 0.036 |
| 91 | 0.024 | 0.028 |
| 92 | 0.011 | 0.050 |
| 93 | 0.025 | 0.13 |
| 94 | 0.065 | 0.30 |
| 95 | 0.024 | 0.086 |
| 96 | 0.013 | 0.11 |
| 97 | 0.034 | 0.30 |
| 98 | 0.020 | 0.19 |
| 99 | 0.037 | 0.20 |
| 100 | 0.016 | 0.089 |
| 101 | 0.015 | 0.12 |
| 102 | 0.019 | 0.15 |
| 103 | 0.018 | 0.22 |
| 104 | 0.010 | 0.11 |
| 105 | 0.012 | 0.11 |
| 106 | 0.0074 | 0.028 |
| 107 | 0.015 | 0.63 |
| 108 | 0.022 | 0.71 |
| 109 | 0.024 | 0.26 |
| 110 | 0.027 | 0.063 |
| 111 | 0.025 | >1.0 |
| 112 | 0.013 | 0.087 |
| 113 | 0.0053 | 0.085 |
| 114 | 0.020 | 0.30 |
| 115 | 0.024 | 0.22 |
| 116 | 0.021 | 0.19 |
| 117 | 0.037 | 0.52 |
| 118 | 0.045 | 0.32 |
| 119 | 0.24 | 1.0 |
| 120 | 0.054 | 0.89 |
| 121 | 0.21 | 0.61 |
| 122 | 0.074 | 3.0 |
| 123 | 0.13 | 0.58 |
| 124 | 0.055 | 0.93 |
| 125 | 0.11 | 0.91 |
| 126 | 0.051 | 0.42 |
| 127 | 0.0062 | 0.062 |
| 128 | 0.012 | 0.18 |
| 129 | 0.021 | 0.16 |
| 130 | 0.0060 | 0.13 |
| 131 | 0.072 | 0.70 |
| 132 | 0.067 | 0.40 |
| 133 | 0.11 | 0.37 |
| 134 | 0.023 | 0.36 |
| 135 | 0.074 | 0.38 |
| 136 | 0.031 | 0.32 |
| 137 | 0.020 | 0.26 |
| 138 | 0.16 | 0.60 |
| 139 | 0.030 | 0.52 |
| 140 | 0.014 | 0.83 |
| 141 | 0.014 | 0.27 |
| 142 | 0.025 | 0.36 |
| 143 | 0.015 | 1.2 |
| 144 | 0.015 | 1.3 |
| 145 | 0.030 | 1.7 |
| 146 | 0.015 | 0.30 |
| 147 | 0.0053 | 0.33 |
| 148 | 0.010 | 0.25 |
| 149 | 0.017 | 0.054 |
| 150 | 0.039 | 0.41 |
| 151 | 0.013 | 0.22 |
| 152 | 0.020 | 0.14 |
| 153 | 0.059 | 0.90 |
| 154 | 0.029 | 0.29 |
| 155 | 0.010 | 0.070 |
| 156 | 0.020 | 0.16 |
| 157 | 0.040 | 0.72 |
| 158 | 0.024 | 0.31 |
| 159 | 0.11 | 0.38 |
| 160 | 0.071 | 0.38 |
| 161 | 0.031 | 0.15 |
| 162 | 0.052 | 0.40 |
| 163 | 0.019 | 0.12 |
| 164 | 0.021 | 0.21 |
| 165 | 0.012 | 0.21 |
| 166 | 0.031 | 0.13 |
| 167 | 0.017 | 0.14 |
| 168 | 0.023 | 0.20 |
| 169 | 0.30 | 2.1 |
| 170 | 0.019 | 0.28 |
| 171 | 0.014 | 0.16 |
| 172 | 0.034 | 0.25 |
| 173 | 0.013 | 0.19 |
| 174 | 0.032 | 0.57 |
| 175 | 0.021 | 0.39 |
| 176 | 0.019 | 0.17 |
| 177 | 0.028 | 0.17 |
| 178 | 0.024 | 0.57 |
| 179 | 0.045 | 0.16 |
| 180 | 0.019 | 0.17 |
| 181 | 0.035 | 0.22 |
| 182 | 0.022 | 0.056 |
| 183 | 0.063 | 0.34 |
| 184 | 0.010 | 0.36 |
| 185 | 0.015 | 0.33 |
| 186 | 0.020 | 0.43 |
| 187 | 0.012 | 0.22 |
| 188 | 0.014 | 0.54 |
| 189 | 0.020 | 0.15 |
| 190 | 0.014 | 0.26 |
| 191 | 0.13 | 0.54 |
| 192 | 0.028 | 0.35 |
| 193 | 0.085 | 0.19 |
| 194 | 0.014 | 0.053 |
| 195 | 0.022 | 0.46 |
| 196 | 0.020 | 0.30 |
| 197 | 0.19 | 0.76 |
| 198 | 0.089 | 1.2 |
| 199 | 0.13 | 1.1 |
| 200 | 0.012 | 1.8 |
| 201 | 0.13 | 0.86 |
| 202 | 0.059 | 0.30 |
| 203 | 0.065 | 0.17 |
| 204 | 0.019 | 0.65 |
| 205 | 0.013 | 0.28 |
| 206 | 0.032 | 0.51 |
| 207 | 0.013 | 0.16 |
| 208 | 0.014 | 0.24 |
| 209 | 0.033 | 0.23 |
| 210 | 0.021 | 0.22 |
| 211 | 0.026 | 0.18 |
| 212 | 0.026 | 0.31 |
| 213 | 0.017 | 0.11 |
| 214 | 0.017 | 0.22 |
| 215 | 0.014 | 0.29 |
| 216 | 0.015 | 0.30 |
| 217 | 0.032 | 0.92 |
| 218 | 0.015 | 0.75 |
| 219 | 0.011 | 1.4 |
| 220 | 0.014 | 0.34 |
| 221 | 0.044 | 0.67 |
| 222 | 0.048 | 0.45 |
| 223 | 0.028 | 0.95 |
| 224 | 0.011 | 0.091 |
| 225 | 0.022 | 0.15 |
| 226 | 0.047 | 1.4 |
| 227 | 0.13 | 3.8 |
| 228 | 0.010 | 0.068 |
| 229 | 0.050 | 2.8 |
| 230 | 0.083 | 1.0 |
| 231 | 0.021 | 0.47 |
| 232 | 0.15 | 1.4 |
| 233 | 0.12 | 2.8 |
| 234 | 0.091 | 0.66 |
| 235 | 0.0030 | 0.013 |
| 236 | 0.011 | 0.077 |
| 237 | 0.012 | 0.038 |
| 238 | 0.010 | 0.77 |
| 239 | 0.0055 | 0.099 |
| 240 | 0.017 | 0.14 |
| 241 | 0.0087 | 0.37 |
| 242 | 0.017 | 0.15 |
| 243 | 0.057 | 0.26 |
| 244 | 0.063 | 0.18 |
| 245 | 0.0023 | 0.068 |
| 246 | 0.021 | 0.16 |
| 247 | 0.017 | 0.27 |
| 248 | 0.015 | 0.099 |
| 249 | 0.026 | 0.28 |
| 250 | 0.049 | 0.18 |
| 251 | 0.055 | 0.47 |
| 252 | 0.099 | 0.63 |
| 253 | 0.071 | 0.60 |
| 254 | 0.097 | 0.51 |
| 255 | 0.064 | 0.54 |
| 256 | 0.16 | 1.0 |
| 257 | 0.20 | 0.87 |
| 258 | 0.038 | 0.42 |
| 259 | 0.044 | 0.69 |
| 260 | 0.048 | 0.95 |
| 261 | 0.072 | 1.0 |
| 262 | 0.029 | 0.36 |
| 263 | 0.054 | 0.61 |
| 264 | 0.084 | 0.49 |
| 265 | 0.16 | 0.78 |
| 266 | 0.073 | 1.0 |
| 267 | 0.086 | 0.53 |
| 268 | 0.10 | 0.89 |
| 269 | 0.042 | 0.37 |
| 270 | 0.048 | 0.44 |
| 271 | 0.012 | 0.26 |
| 272 | 0.079 | 0.98 |
| 273 | 0.092 | 0.52 |
| 274 | 0.038 | 0.38 |
| 275 | 0.12 | 0.92 |
| 276 | 0.015 | 0.38 |
| 277 | 0.061 | 0.61 |
| 278 | 0.12 | 0.42 |
| 279 | 0.12 | 0.56 |
| 280 | 0.062 | 0.85 |
| 281 | 0.020 | 0.45 |
| 282 | 0.077 | 1.4 |
| 283 | 0.068 | >3.0 |
| 284 | 0.15 | 0.83 |
| 285 | 0.076 | 0.87 |
| 286 | 0.096 | 0.82 |
| 287 | 0.048 | 0.84 |
| 288 | 0.18 | 0.48 |
| 289 | 0.030 | 0.21 |
| 290 | 0.038 | 0.40 |
| 291 | 0.072 | 0.73 |
| 292 | 0.039 | 0.59 |
| 293 | 0.061 | 0.51 |
| 294 | 0.022 | 0.47 |
| 295 | 0.064 | 0.70 |
| 296 | 0.055 | 0.48 |
| 297 | 0.066 | 0.66 |
| 298 | 0.029 | 0.30 |
| 299 | 0.079 | 0.70 |
| 300 | 0.059 | 0.49 |
| 301 | 0.054 | 0.71 |
| 302 | 0.050 | 0.57 |
| 303 | 0.078 | 0.61 |
| 304 | 0.015 | 0.23 |
| 305 | 0.059 | 0.59 |
| 306 | 0.062 | 0.45 |
| 307 | 0.045 | 0.50 |
| 308 | 0.054 | 0.51 |
| 309 | 0.094 | 0.68 |
| 310 | 0.057 | 0.47 |
| 311 | 0.067 | 0.77 |
| 312 | 0.11 | 0.51 |
| 313 | 0.040 | 0.90 |
| 314 | 0.061 | 0.80 |
| 315 | 0.045 | 0.88 |
| 316 | 0.029 | 0.47 |
| 317 | 0.019 | 0.52 |
| 318 | 0.016 | 0.29 |
| 319 | 0.085 | 0.52 |
| 320 | 0.018 | 0.48 |
| 321 | 0.011 | 0.19 |
| 322 | 0.048 | 0.18 |
| 323 | 0.022 | 0.30 |
| 324 | 0.095 | 0.65 |
| 325 | 0.036 | 0.63 |
| 326 | 0.053 | 0.36 |
| 327 | 0.29 | 1.2 |
| 328 | 0.060 | 0.53 |
| 329 | 0.12 | 0.59 |
| 330 | 0.036 | 0.58 |
| 331 | 0.019 | 0.42 |
| 332 | 0.045 | 0.55 |
| 333 | 0.046 | 0.32 |
| 334 | 0.26 | 2.5 |
| 335 | 0.076 | 1.0 |
| 336 | 0.023 | 0.17 |
| 337 | 0.032 | 0.42 |
| 338 | 0.028 | 0.42 |
| 339 | 0.036 | 0.37 |
| 340 | 0.016 | 0.30 |
| 341 | 0.052 | 0.79 |
| 342 | 0.036 | 0.48 |
| 343 | 0.092 | 0.61 |
| 344 | 0.025 | 0.53 |
| 345 | 0.053 | 0.83 |
| 346 | 0.065 | 0.53 |
| 347 | 0.038 | 0.40 |
| 348 | 0.11 | 1.0 |
| 349 | 0.067 | 0.39 |
| 350 | 0.049 | 0.53 |
| 351 | 0.024 | 0.21 |
| 352 | 0.032 | 0.42 |
| 353 | 0.014 | 0.49 |
| 354 | 0.14 | 0.68 |
| 355 | 0.12 | 0.54 |
| 356 | 0.053 | 0.71 |
| 357 | 0.077 | 0.39 |
| 358 | 0.045 | 0.70 |
| 359 | 0.023 | 0.34 |
| 360 | 0.064 | 0.77 |
| 361 | 0.061 | 0.76 |
| 362 | 0.10 | 1.5 |
| 363 | 0.057 | 1.3 |
| 364 | 0.039 | 0.78 |
| 365 | 0.012 | 0.46 |
| 366 | 0.12 | 0.54 |
| 367 | 0.034 | 0.35 |
| 368 | 0.072 | 0.41 |
| 369 | 0.035 | 0.40 |
| 370 | 0.056 | 0.38 |
| 371 | 0.041 | 0.40 |
| 372 | 0.045 | 0.72 |
| 373 | 0.036 | 0.65 |
| 374 | 0.051 | 0.63 |
| 375 | 0.052 | 0.50 |
| 376 | 0.072 | 0.77 |
| 377 | 0.066 | 0.34 |
| 378 | 0.023 | 0.29 |
| 379 | 0.016 | 0.20 |
| 380 | 0.020 | 0.40 |
| 381 | 0.046 | 0.50 |
| 382 | 0.015 | 0.41 |
| 383 | 0.11 | 0.57 |
| 384 | 0.060 | 0.50 |
| 385 | 0.13 | 0.38 |
| 386 | 0.038 | 0.84 |
| 387 | 0.030 | 0.78 |
| 388 | 0.047 | 0.51 |
| 389 | 0.076 | 0.79 |
| 390 | 0.028 | 0.66 |
| 391 | 0.072 | 1.1 |
| 392 | 0.042 | 1.3 |
| 393 | 0.041 | 0.31 |
| 394 | 0.033 | 0.42 |
| 395 | 0.064 | 0.43 |
| 396 | 0.047 | 0.34 |
| 397 | 0.046 | 0.72 |
| 398 | 0.052 | 1.4 |
| 399 | 0.054 | 0.59 |
| 400 | 0.023 | 0.60 |
| 401 | 0.048 | 1.2 |
| 402 | 0.024 | 0.71 |
| 403 | 0.14 | 0.94 |
| 404 | 0.081 | 0.84 |
| 405 | 0.11 | 0.46 |
| 406 | 0.17 | 0.68 |
| 407 | 0.037 | 0.46 |
| 408 | 0.038 | 0.40 |
| 409 | 0.049 | 0.42 |
| 410 | 0.042 | 0.43 |
| 411 | 0.040 | 0.55 |
| 412 | 0.043 | 0.59 |
| 413 | 0.042 | 0.68 |
| 414 | 0.033 | 0.82 |
| 415 | 0.040 | 0.69 |
| 416 | 0.018 | 0.53 |
| 417 | 0.070 | 1.3 |
| 418 | 0.083 | 0.91 |
| 419 | 0.044 | 1.0 |
| 420 | 0.062 | 0.49 |
| 421 | 0.035 | 0.35 |
| 422 | 0.038 | 0.42 |
| 423 | 0.031 | 0.74 |
| 424 | 0.044 | 0.48 |
| 425 | 0.048 | 0.39 |
| 426 | 0.015 | 0.14 |
| 427 | 0.025 | 0.31 |
| 428 | 0.021 | 0.23 |
| 429 | 0.022 | 0.54 |
| 430 | 0.019 | 0.24 |
| 431 | 0.017 | 0.28 |
| 432 | 0.017 | 0.27 |
| 433 | 0.031 | 0.54 |
| 434 | 0.028 | 0.42 |
| 435 | 0.015 | 0.17 |
| 436 | 0.010 | 0.33 |
| 437 | 0.0025 | 0.18 |
| 438 | 0.013 | 0.14 |
| 439 | 0.022 | 0.55 |
| 440 | 0.017 | 0.16 |
| 441 | 0.060 | 0.43 |
| 442 | 0.011 | 0.36 |
| 443 | 0.016 | 0.25 |
| 444 | 0.037 | 0.36 |
| 445 | 0.020 | 0.40 |
| 446 | 0.018 | 0.48 |
| 447 | 0.020 | 0.56 |
| 448 | 0.062 | 1.9 |
| 449 | >0.30 | 1.2 |
| 450 | 0.064 | 0.58 |
| 451 | 0.091 | 0.84 |
| 452 | 0.11 | 1.6 |
| 453 | 0.25 | 1.5 |
| 454 | 0.030 | 0.73 |
| 455 | 0.065 | 0.79 |
| 456 | 0.053 | 0.43 |
| 457 | 0.11 | 0.71 |
| 458 | >0.30 | 1.1 |
| 459 | 0.11 | 0.43 |
| 460 | 0.18 | 1.0 |
| 461 | 0.23 | 1.6 |
| 462 | 0.081 | 0.99 |
| 463 | 0.065 | 0.93 |
| 464 | 0.017 | 0.44 |
| 465 | 0.011 | 0.21 |
| 466 | 0.012 | 0.24 |
| 467 | 0.015 | 0.27 |
| 468 | 0.025 | 0.30 |
| 469 | 0.076 | 1.2 |
| 470 | 0.048 | 0.26 |
| 471 | 0.015 | 0.17 |
| 472 | 0.017 | 0.35 |
| 473 | 0.048 | 1.2 |
| 474 | 0.030 | 0.19 |
| 475 | 0.034 | 0.27 |
| 476 | 0.036 | 0.57 |
| 477 | 0.18 | 0.83 |
| 478 | 0.045 | 0.37 |
| 479 | 0.042 | 0.26 |
| 480 | 0.047 | 0.67 |
| 481 | 0.071 | 0.48 |
| 482 | 0.043 | 1.3 |
| 483 | 0.11 | 1.2 |
| 484 | 0.035 | >1.0 |
| 485 | 0.14 | >3.0 |
| 486 | 0.12 | 2.2 |
| 487 | 0.081 | 0.53 |
| 488 | 0.059 | 0.84 |
| 489 | 0.15 | 0.90 |
| 490 | 0.094 | 1.1 |
| 491 | 0.099 | 0.63 |
| 492 | 0.11 | 0.44 |
| 493 | 0.086 | 0.55 |
| 494 | 0.089 | 0.87 |
| 495 | 0.068 | 0.50 |
| 496 | 0.041 | 0.53 |
| 497 | 0.052 | 0.89 |
| 498 | 0.049 | 0.31 |
| 499 | 0.13 | 0.76 |
| 500 | >0.30 | 0.75 |
| 501 | >0.30 | >3.0 |
| 502 | >0.30 | 1.2 |
| 503 | >0.30 | >3.0 |
| 504 | 0.16 | 1.0 |
| 505 | 0.051 | 0.44 |
| 506 | 0.10 | 0.92 |

### Test Example 2

### EPO Production Assay

### Test Compound:

The compounds described in the above Examples were used in the EPO production assay.

### Test Method:

The stimulatory effect of the compound onEPO production was determined as follows using Hep3B cells.

Cells were seeded on 96 well plates at 40000 cells/well, and the plate was incubated overnight in MEM medium containing 10% fetal bovine serum (FBS) at 37° C. under 5% CO₂. The next day, the medium was replaced with MEM containing 0.5% FBS, followed by addition of test compound. The test compound was dissolved in dimethyl sulfoxide and added to the cell so that the final concentration of dimethyl sulfoxide was 0.1%. After incubation for additional 48 hours, the culture supernatant was collected, and EPO contained in the supernatant was determined using ELISA kit (EPO ELISA Kit 11-693-417-001, Roche Co.). The results were expressed as stimulation rate of EPO production (fold), which was calculated as the relative value of the EPO production with 3 μmol/L of the test compound compared to the EPO production without stimulation (control).

Stimulation rate of EPO production (fold)=(EPO production with addition of test compound)/(EPO production of control)

### Results:

The stimulation rates of EPO production of the test compounds are shown in Table 13.

TABLE 13

| | EPO production |
|---|---|
| | stimulation rate with |
| Test Compound | 3 μmol/L test compound |
| (Example No.) | (fold) |
| Control (0.1% DMSO) | 1.0 |
| 1 | 4.6 |
| 3 | 2.1 |
| 4 | 1.7 |
| 5 | 2.9 |
| 6 | 6.1 |
| 7 | 3.4 |
| 8 | 5.4 |
| 9 | 2.8 |
| 10 | 3.4 |
| 11 | 2.5 |
| 12 | 2.3 |
| 13 | 4.3 |
| 14 | 1.4 |
| 15 | 1.7 |
| 16 | 3.1 |
| 17 | 3.2 |
| 18 | 5.6 |
| 19 | 10 |
| 20 | 5.5 |
| 21 | 7.5 |
| 22 | 6.9 |
| 23 | 3.2 |
| 24 | 2.8 |
| 25 | 2.9 |
| 26 | 3.4 |
| 27 | 26 |
| 29 | 4.2 |
| 30 | 5.5 |
| 31 | 4.7 |
| 32 | 5.7 |
| 34 | 1.2 |
| 35 | 5.7 |
| 36 | 1.4 |
| 37 | 8.3 |
| 38 | 2.9 |
| 39 | 5.0 |
| 40 | 5.0 |
| 41 | 9.5 |
| 42 | 9.1 |
| 43 | 6.5 |
| 44 | 4.9 |
| 45 | 8.9 |
| 46 | 3.7 |
| 47 | 4.4 |
| 48 | 6.6 |
| 49 | 7.6 |
| 50 | 6.3 |
| 51 | 3.9 |
| 52 | 3.3 |
| 53 | 3.3 |
| 54 | 5.6 |
| 55 | 34 |
| 56 | 19 |
| 57 | 9.1 |
| 58 | 9.3 |
| 61 | 6.0 |
| 62 | 12 |
| 63 | 18 |
| 64 | 23 |
| 65 | 20 |
| 66 | 33 |
| 67 | 19 |
| 68 | 9.5 |
| 69 | 7.8 |
| 70 | 24 |
| 76 | 6.9 |
| 77 | 9.8 |
| 78 | 30 |
| 79 | 14 |
| 80 | 9.5 |
| 81 | 5.4 |
| 83 | 6.2 |
| 84 | 4.3 |
| 85 | 4.0 |
| 86 | 2.5 |
| 87 | 5.3 |
| 89 | 16 |
| 91 | 8.6 |
| 93 | 23 |
| 94 | 17 |
| 95 | 4.2 |
| 96 | 6.8 |
| 97 | 5.2 |
| 98 | 8.6 |
| 99 | 3.1 |
| 101 | 12 |
| 102 | 9.8 |
| 104 | 5.4 |
| 105 | 9.5 |
| 106 | 5.2 |
| 109 | 20 |
| 110 | 56 |
| 111 | 36 |
| 112 | 28 |
| 113 | 7.5 |
| 114 | 5.2 |
| 115 | 5.1 |
| 116 | 4.0 |
| 117 | 2.0 |
| 118 | 17 |
| 119 | 3.7 |
| 120 | 9.7 |
| 124 | 3.4 |
| 126 | 5.2 |
| 127 | 17 |
| 128 | 168 |
| 129 | 162 |
| 130 | 19 |
| 131 | 8.6 |
| 132 | 2.6 |
| 133 | 3.9 |
| 134 | 25 |
| 135 | 6.1 |
| 136 | 8.5 |
| 137 | 9.8 |
| 138 | 2.9 |
| 139 | 1.6 |
| 140 | 6.2 |
| 141 | 1.2 |
| 142 | 15 |
| 144 | 4.3 |
| 146 | 2.6 |
| 147 | 4.0 |
| 148 | 11 |
| 149 | 22 |
| 150 | 9.0 |
| 151 | 38 |
| 152 | 20 |
| 153 | 1.5 |
| 154 | 5.4 |
| 155 | 11 |
| 156 | 24 |
| 157 | 4.0 |
| 158 | 1.2 |
| 159 | 4.5 |
| 160 | 3.6 |
| 161 | 3.1 |
| 162 | 18 |
| 163 | 13 |
| 164 | 8.8 |
| 165 | 15 |
| 166 | 8.1 |
| 167 | 2.1 |
| 168 | 66 |
| 170 | 19 |
| 171 | 11 |
| 172 | 19 |
| 173 | 7.7 |
| 174 | 9.9 |
| 175 | 15 |
| 176 | 22 |
| 177 | 9.1 |
| 178 | 37 |
| 179 | 14 |
| 181 | 34 |
| 182 | 9.1 |
| 183 | 7.5 |
| 187 | 10 |
| 188 | 23 |
| 189 | 8.7 |
| 191 | 1.6 |
| 192 | 3.5 |
| 193 | 9.8 |
| 194 | 18 |
| 196 | 25 |
| 197 | 5.8 |
| 198 | 3.2 |
| 199 | 5.0 |
| 200 | 1.4 |
| 201 | 3.2 |
| 202 | 3.6 |
| 203 | 4.5 |
| 204 | 12 |
| 205 | 20 |
| 206 | 2.2 |
| 207 | 2.5 |
| 208 | 7.1 |
| 209 | 2.2 |
| 210 | 19 |
| 211 | 8.9 |
| 212 | 2.7 |
| 213 | 6.4 |
| 214 | 6.8 |
| 215 | 6.7 |
| 216 | 1.5 |
| 217 | 2.4 |
| 218 | 3.0 |
| 220 | 4.0 |
| 222 | 1.5 |
| 223 | 3.2 |
| 224 | 8.4 |
| 226 | 2.2 |
| 228 | 3.9 |
| 230 | 2.0 |
| 234 | 5.7 |
| 235 | 1.9 |
| 236 | 0.91 |
| 237 | 4.3 |
| 238 | 0.70 |
| 239 | 1.4 |
| 240 | 1.0 |
| 241 | 1.2 |
| 242 | 1.7 |
| 243 | 1.6 |
| 244 | 1.4 |
| 245 | 6.2 |
| 246 | 1.8 |
| 247 | 1.3 |
| 248 | 3.2 |
| 323 | 3.8 |
| 326 | 7.7 |
| 327 | 9.4 |
| 378 | 9.6 |
| 379 | 10 |
| 380 | 3.9 |
| 381 | 6.3 |
| 439 | 5.3 |
| 440 | 2.6 |
| 442 | 3.3 |
| 443 | 2.9 |
| 444 | 2.2 |
| 446 | 6.3 |
| 447 | 5.4 |
| 454 | 2.7 |
| 465 | 2.5 |
| 466 | 2.6 |
| 467 | 3.2 |
| 468 | 3.1 |
| 470 | 1.7 |
| 471 | 1.4 |
| 472 | 2.4 |
| 473 | 2.0 |
| 474 | 2.8 |
| 476 | 2.7 |
| 477 | 2.5 |
| 478 | 1.8 |
| 484 | 1.6 |
| 493 | 2.8 |
| 495 | 18 |
| 498 | 2.7 |

### INDUSTRIAL APPLICABILITY

The compound (I) or a pharmaceutically acceptable salt thereof of the present invention exhibits inhibitory effect on HIF-PHD. Thus, the compound (I) or a pharmaceutically acceptable salt thereof of the present invention is useful for the prevention and treatment of various diseases associated with HIF-PHD, such as renal anemia.

1. A compound represented by the formula (I): 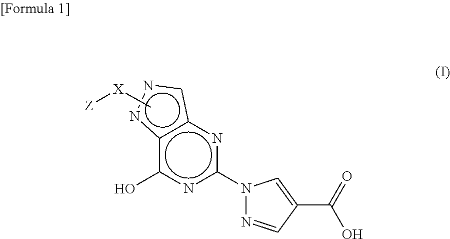
wherein, 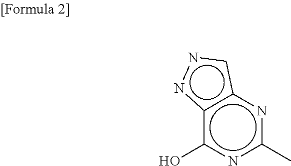
represents an optionally substituted 7-hydroxypyrazolo[4,3-d]pyrimidin-5-yl;
X represents a single bond or an optionally substituted straight chain alkylene;
Z represents hydrogen atom or the formula (i), (ii) or (iii): 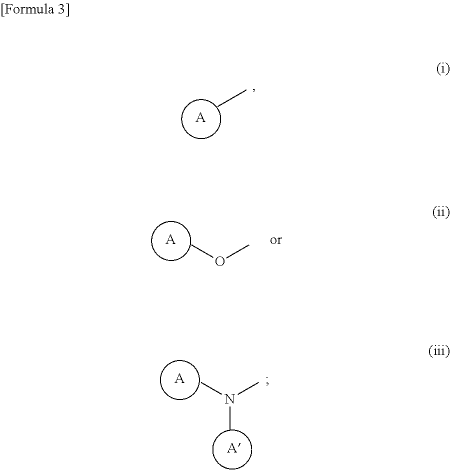
ring A and ring A′ are each independently an optionally substituted aryl, an optionally substituted heteroaryl, an optionally substituted alicyclic hydrocarbon or an optionally substituted non-aromatic heterocycle,
or a pharmaceutically acceptable salt thereof.

2. The compound according to claim 1 represented by the formula (I-A): 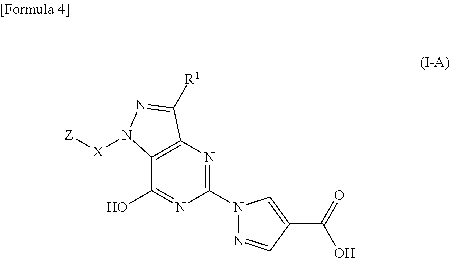
wherein R¹ is hydrogen atom, alkyl, fluoroalkyl, cycloalkyl, halogen or cyano, and the other symbols are as defined in claim 1,
or a pharmaceutically acceptable salt thereof.

3. The compound according to claim 1 represented by the formula (I-B): 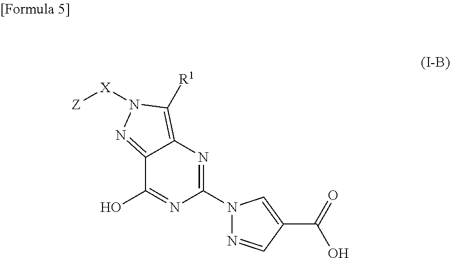
wherein R¹ is hydrogen atom, alkyl, fluoroalkyl, cycloalkyl, halogen or cyano,
or a pharmaceutically acceptable salt thereof.

4. The compound according to claim 1 represented by the formula (I-C): 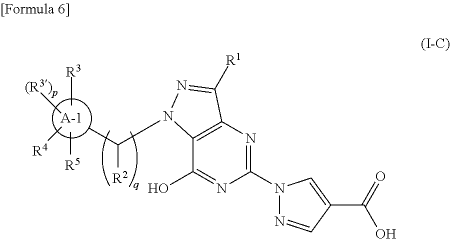
wherein
ring A-1 is aryl, heteroaryl, alicyclic hydrocarbon or non-aromatic heterocycle;
R² is hydrogen atom, alkyl, halogenoalkyl, cycloalkyl, phenyl or halogenophenyl;
R³, R^{3′} and R⁴ are each independently hydrogen atom, an optionally substituted alkyl, an optionally substituted alkenyl, an optionally substituted cycloalkyl, an optionally substituted alkoxy, halogen or cyano;
R⁵ is hydrogen atom, an optionally substituted alkyl, an optionally substituted alkenyl, an optionally substituted cycloalkyl, an optionally substituted alkoxy, halogen, cyano, an optionally substituted phenyl, an optionally substituted phenoxy, an optionally substituted monocyclic heteroaryl or non-aromatic heterocycle;
p represents 0 or 1;
q represents 0 or 1; and
R¹ is hydrogen atom, alkyl, fluoroalkyl, cycloalkyl, halogen or cyano,
or a pharmaceutically acceptable salt thereof.

5. The compound according to claim 4
wherein
ring A-1 is phenyl, naphthyl, tetrahydronaphthyl, indanyl, thienyl, pyridyl, cyclopropyl, cyclopentyl, cyclohexyl, cycloheptyl, cyclooctyl, spiro[5.2]octyl, spiro[5.3]nonyl, adamantyl, pyrrolidinyl, or piperidinyl;
R¹ is hydrogen atom, C₁-C₆ alkyl, C₁-C₆ fluoroalkyl, C₃-C₈ cycloalkyl, or halogen;
R² is hydrogen atom, C₁-C₆ alkyl, C₃-C₈ cycloalkyl, or halogenophenyl;
R³, R^{3′} and R⁴ are independently hydrogen atom; C₁-C₆ alkyl optionally substituted with 1 to 7 substituent groups selected from halogen, halogenophenyl, tetrahydronaphthyloxy, and halogenophenoxy; C₃-C₈ cycloalkyl; C₁-C₆ alkoxy optionally substituted with 1 to 7 halogens; or halogen;
R⁵ is hydrogen atom; C₁-C₆ alkyl optionally substituted with 1 to 7 substituent groups selected from halogen, halogenophenyl, tetrahydronaphthyloxy and halogenophenoxy; C₃-C₈ cycloalkyl; C₁-C₆ alkoxy optionally substituted with 1 to 7 halogens; halogen; phenyl optionally substituted with 1, 2 or 3 substituent groups selected from the group consisting of C₁-C₆ alkyl, C₁-C₆ halogenoalkyl, C₃-C₈cycloalkyl, C₁-C₆ alkoxy, C₁-C₆ halogenoalkoxy, cyano and halogen; phenoxy optionally substituted with 1, 2 or 3 substituent groups selected from the group consisting of C₁-C₆ alkyl, C₁-C₆ halogenoalkyl and halogen; pyridyl optionally substituted with 1, 2 or 3 substituent groups selected from the group consisting of C₁-C₆ alkyl, C₁-C₆ halogenoalkyl and halogen; or non-aromatic heterocycle optionally substituted with 1 to 5 substituent groups selected from the group consisting of C₁-C₆ alkyl, C₁-C₆ halogenoalkyl and oxo,
or a pharmaceutically acceptable salt thereof.

6. The compound according to claim 4
wherein
ring A-1 is C₃-C₁₄ monocyclic, bicyclic or tricyclic alicyclic hydrocarbon;
R¹ is hydrogen atom;
R² is hydrogen atom or C₁-C₆ alkyl;
R³, R^{3′} and R⁴ are independently hydrogen atom, C₁-C₆ alkyl, C₁-C₆ halogenoalkyl or halogen;
R⁵ is hydrogen atom; C₁-C₆ alkyl; C₁-C₆ halogenoalkyl; halogen; phenyl optionally substituted with 1, 2 or 3 halogens; or phenoxy optionally substituted with 1, 2 or 3 substituent groups selected from the group consisting of C₁-C₆ alkyl, C₁-C₆ halogenoalkyl and halogen;
p is 1; and
q is 0 or 1,
or a pharmaceutically acceptable salt thereof.

7. The compound according to claim 1 represented by the formula (I-E): 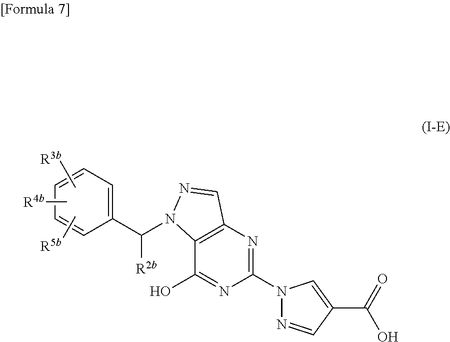
wherein
R^{2b} is hydrogen atom, C₁-C₆ alkyl or C₃-C₈ cycloalkyl;
R^{3b}, R^{4b} and R^{5b} are independently hydrogen atom, C₁-C₆ alkyl, C₁-C₆ halogenoalkyl, C₃-C₈ cycloalkyl, C₁-C₆ alkoxy, C₁-C₆ halogenoalkoxy or halogen,
or a pharmaceutically acceptable salt thereof.

8. A compound selected from the group consisting of:
1-(7-hydroxy-1-{1-[4-(trifluoromethyl)phenyl]ethyl}-1H-pyrazolo[4,3-d]pyrimidin-5-yl)-1H-pyrazole-4-carboxylic acid;
1-{1-[1-(4-chlorophenyl)ethyl]-7-hydroxy-1H-pyrazolo[4,3-d]pyrimidin-5-yl}-1H-pyrazole-4-carboxylic acid;
1-{1-[1-(3,4-dichlorophenyl)ethyl]-7-hydroxy-1H-pyrazolo[4,3-d]pyrimidin-5-yl}-1H-pyrazole-4-carboxylic acid;
1-[7-hydroxy-1-(2-naphthylmethyl)-1H-pyrazolo[4,3-d]pyrimidin-5-yl]-1H-pyrazole-4-carboxylic acid;
1-{7-hydroxy-1-[1-(2-naphthyl)ethyl]-1H-pyrazolo[4,3-d]pyrimidin-5-yl}-1H-pyrazole-4-carboxylic acid;
1-[1-(biphenyl-4-ylmethyl)-7-hydroxy-1H-pyrazolo[4,3-d]pyrimidin-5-yl]-1H-pyrazole-4-carboxylic acid;
1-{1-[(2′-fluorobiphenyl-4-yl)methyl]-7-hydroxy-1H-pyrazolo[4,3-d]pyrimidin-5-yl}-1H-pyrazole-4-carboxylic acid;
1-{1-[(3-fluorobiphenyl-4-yl)methyl]-7-hydroxy-1H-pyrazolo[4,3-d]pyrimidin-5-yl}-1H-pyrazole-4-carboxylic acid;
1-{1-[(2-fluorobiphenyl-4-yl)methyl]-7-hydroxy-1H-pyrazolo[4,3-d]pyrimidin-5-yl}-1H-pyrazole-4-carboxylic acid;
1-{1-[(2,2′-difluorobiphenyl-4-yl)methyl]-7-hydroxy-1H-pyrazolo[4,3-d]pyrimidin-5-yl}-1H-pyrazole-4-carboxylic acid;
1-{1-[(2′-fluoro-2-methylbiphenyl-4-yl)methyl]-7-hydroxy-1H-pyrazolo[4,3-d]pyrimidin-5-yl}-1H-pyrazole-4-carboxylic acid;
1-{1-[(2′-fluoro-2-methoxybiphenyl-4-yl)methyl]-7-hydroxy-1H-pyrazolo[4,3-d]pyrimidin-5-yl}-1H-pyrazole-4-carboxylic acid;
1-{1-[1-biphenyl-4-ylethyl]-7-hydroxy-1H-pyrazolo[4,3-d]pyrimidin-5-yl}-1H-pyrazole-4-carboxylic acid;
1-[1-(cyclohexylmethyl)-7-hydroxy-1H-pyrazolo[4,3-d]pyrimidin-5-yl]-1H-pyrazole-4-carboxylic acid;
1-{7-hydroxy-1-[(trans-4-methylcyclohexyl)methyl]-1H-pyrazolo[4,3-d]pyrimidin-5-yl}-1H-pyrazole-4-carboxylic acid;
1-{7-hydroxy-1-[(trans-4-phenylcyclohexyl)methyl]-1H-pyrazolo[4,3-d]pyrimidin-5-yl}-1H-pyrazole-4-carboxylic acid;
1-(1-{[trans-4-(4-chlorophenyl)cyclohexyl]methyl}-7-hydroxy-1H-pyrazolo[4,3-d]pyrimidin-5-yl)-1H-pyrazole-4-carboxylic acid;
1-[1-(cycloheptylmethyl)-7-hydroxy-1H-pyrazolo[4,3-d]pyrimidin-5-yl]-1H-pyrazole-4-carboxylic acid;
1-[1-(cyclooctylmethyl)-7-hydroxy-1H-pyrazolo[4,3-d]pyrimidin-5-yl]-1H-pyrazole-4-carboxylic acid;
1-{1-[1-(2,5-dichlorophenyl)ethyl]-7-hydroxy-1H-pyrazolo[4,3-d]pyrimidin-5-yl}-1H-pyrazole-4-carboxylic acid;
1-(1-{1-[3-fluoro-4-(trifluoromethyl)phenyl]ethyl}-7-hydroxy-1H-pyrazolo[4,3-d]pyrimidin-5-yl)-1H-pyrazole-4-carboxylic acid;
1-{1-[1-(2,5-dimethylphenyl)ethyl]-7-hydroxy-1H-pyrazolo[4,3-d]pyrimidin-5-yl}-1H-pyrazole-4-carboxylic acid;
1-{1-[1-(5-fluoro-2-methylphenyl)ethyl]-7-hydroxy-1H-pyrazolo[4,3-d]pyrimidin-5-yl}-1H-pyrazole-4-carboxylic acid;
1-{1-[1-(3-fluoro-5-methylphenyl)ethyl]-7-hydroxy-1H-pyrazolo[4,3-d]pyrimidin-5-yl)}-1H-pyrazole-4-carboxylic acid;
1-{1-[1-(4-fluoro-3-methylphenyl)ethyl]-7-hydroxy-1H-pyrazolo[4,3-d]pyrimidin-5-yl}-1H-pyrazole-4-carboxylic acid;
1-{1-[1-(3,5-dimethylphenyl)ethyl]-7-hydroxy-1H-pyrazolo[4,3-d]pyrimidin-5-yl}-1H-pyrazole-4-carboxylic acid;
1-(1-{1-[4-chloro-3-(trifluoromethyl)phenyl]ethyl}-7-hydroxy-1H-pyrazolo[4,3-d]pyrimidin-5-yl)-1H-pyrazole-4-carboxylic acid;
1-(1-{1-[3-fluoro-4-(trifluoromethoxy)phenyl]ethyl}-7-hydroxy-1H-pyrazolo[4,3-d]pyrimidin-5-yl)-1H-pyrazole-4-carboxylic acid;
1-(1-{1-[3-chloro-4-(trifluoromethoxy)phenyl]ethyl}-7-hydroxy-1H-pyrazolo[4,3-d]pyrimidin-5-yl)-1H-pyrazole-4-carboxylic acid;
1-[7-hydroxy-1-(3,3,5,5-tetramethylcyclohexyl)-1H-pyrazolo[4,3-d]pyrimidin-5-yl]-1H-pyrazole-4-carboxylic acid;
1-(1-cycloheptyl-7-hydroxy-1H-pyrazolo[4,3-d]pyrimidin-5-yl)-1H-pyrazole-4-carboxylic acid;
1-(1-cyclooctyl-7-hydroxy-1H-pyrazolo[4,3-d]pyrimidin-5-yl)-1H-pyrazole-4-carboxylic acid;
1-{7-hydroxy-1-[trans-3-methylcyclohexyl]-1H-pyrazolo[4,3-d]pyrimidin-5-yl}-1H-pyrazole-4-carboxylic acid;
1-{7-hydroxy-1-[cis-3-methylcyclohexyl]-1H-pyrazolo[4,3-d]pyrimidin-5-yl}-1H-pyrazole-4-carboxylic acid;
1-{r-1-[t-3,t-5-dimethylcyclohexyl]-7-hydroxy-1H-pyrazolo[4,3-d]pyrimidin-5-yl}-1H-pyrazole-4-carboxylic acid; and
1-{7-hydroxy-1-[cis-3,3,5-trimethylcyclohexyl]-1H-pyrazolo[4,3-d]pyrimidin-5-yl}-1H-pyrazole-4-carboxylic acid,
or a pharmaceutically acceptable salt thereof.

9. A pharmaceutical composition comprising a compound or a pharmaceutically acceptable salt thereof according to claim 1.

10. Use of a compound or a pharmaceutically acceptable salt thereof according to claim 1 in the manufacture of a medicament.
